SEQUENCE LISTING (366 sequences)

SEQ ID NO: 1 - protein, 178 aa, Homo sapiens
KKVVLGKKGDTVELTCTASQKKSIQFHWKNSNQIKILGNQGSFLTKGPSKLNDRADSRRSLWDQGNFPLIIKNLKIEDSDTYICEVEDQKEEVQLLVFGLTANSDTHLLQGQSLTLTLESPPGSSPSVQCRSPRGKNIQGGKTLSVSQLELQDSGTWTCTVLQNQKKVEFKIDIVVLA

SEQ ID NO: 2 - protein, 179 aa, synthetic construct
KKVVLGKKGDTVELTCTASQKKNIQFHWKNSNQIKILGNQGSFLTKGPSKLNDRVDSRRSLWDQGNFPLIIKNLKIEDSDTYICEVEDQKEEVQLVVFGLTANSDTHLLQGQSLTLTLESPPGSSPSVQCRSPRGKNIQGGKTLSVSQLELQDSGTWTCTVLQNQKKVEFKIDIVVLAF

SEQ ID NO: 3 - protein, 99 aa, Homo sapiens
KKVVLGKKGDTVELTCTASQKKSIQFHWKNSNQIKILGNQGSFLTKGPSKLNDRADSRRSLWDQGNFPLIIKNLKIEDSDTYICEVEDQKEEVQLLVFG

SEQ ID NO: 4 - protein, 99 aa, synthetic construct
KKVVYGKKGDTVELTCTASQKKNIQFHWKNSNQIKILGNQGSFLTKGPSKLNDRVDSRRSLWDQGNFPLIIKNLKPEDSDTYICEVEDQKEEVQLVVVG

SEQ ID NO: 5 - protein, 99 aa, synthetic construct
KKVVYGKVGDTVELTCTASQKKNIQFHWKNSNQIKILGNQGSFLTKGPSKLNDRVDSRRSLWDQGNFPLIIKNLKPEDSDTYICEVEDQKEEVQLVVVG

SEQ ID NO: 6 - protein, 99 aa, synthetic construct
KKVVYGKKGDTVELTCTASQKKNIQFHWKNSNQIKILGNQGSFLTKGPSKLNDRVDSRRSLWDQGNFPLIIKNLKPEDSDTYICEVEDQKQEVQLVVVG

SEQ ID NO: 7 - protein, 99 aa, synthetic construct
KKVVYGKKGDTVELTCTASQKKNIQFHWKNSNQIKILGNQGSFLTKGPSKLNDRVDSRRSLWDQGNFPLIIKNLKPEDSDTYICEVEDQKHEVQLVVVG

SEQ ID NO: 8 - protein, 99 aa, synthetic construct
KKVVYGKKGDTVELTCTASQKKNIQFHWKNSNQIKILGNQGSFLTKGPSKLNDRVDSRRSLWDQGNFPLIIKNLKPEDSDTYICEVGDQKEEVQLVVVG

SEQ ID NO: 9 - protein, 99 aa, synthetic construct
KKVVYGKKGDTVELTCTASQKKNIQFHWKNSNQIKILGNQGSFLTKGPSKLWDRVDSRRSLWDQGNFPLIIKNLKPEDSDTYICEVEDQKEEVQLVVVG

SEQ ID NO: 10 - protein, 99 aa, synthetic construct
KKVVYGKIGDTVELTCTASQKKNIQFHWKNSNQIKILGNQGSFLTKGPSKLNDRVDSRRSLWDQGNFPLIIKNLKPEDSDTYICEVEDQKEEVQLVVVG

SEQ ID NO: 11 - protein, 99 aa, synthetic construct
KKVVYGKCGDTVELTCTASQKKNIQFHWKNSNQIKILGNQGSFLTKGPSKLNDRVDSRRSLWDQGNFPLIIKNLKPEDSDTYICEVEDQKEEVQLVVVC

SEQ ID NO: 12 - protein, 99 aa, synthetic construct
KKVVYGKKGDCVELTCTASQKKNIQFHWKNSNQIKILGNQGSFLTKGPSKLNDRVDSRRSLWDQGNFPLIICNLKPEDSDTYICEVEDQKEEVQLVVVG

SEQ ID NO: 13 - protein, 99 aa, synthetic construct
KKVVYGKKGDTVCLTCTASQKKNIQFHWKNSNQIKILGNQGSFLTKGPSKLNDRVDSRRSLWDQGNFPLCIKNLKPEDSDTYICEVEDQKEEVQLVVVG

SEQ ID NO: 14 - protein, 99 aa, synthetic construct
KKVVYGKKGDTVELTCTASQKKNIQFCWKNSNQIKILCNQGSFLTKGPSKLNDRVDSRRSLWDQGNFPLIIKNLKPEDSDTYICEVEDQKEEVQLVVVG

SEQ ID NO: 15 - protein, 99 aa, synthetic construct
KKVVYGKKGDTVELTCTASQCKNIQFHWKNSNQIKILGNQGSFLTKGPSKLNDRVDSRRSLWDQCNFPLIIKNLKPEDSDTYICEVEDQKEEVQLVVVG

SEQ ID NO: 16 - protein, 99 aa, synthetic construct
KKVVYGKKGDTVELTCTASQKKNIEFHWKNSNQIKILGNQGSFLTKGPSKLNDRVDSRRSLWDQGNFPLIIKNLKPEDSDTYICEVEDQKEEVQLVVVG

SEQ ID NO: 17 - protein, 99 aa, synthetic construct
KKVVYGKKGDTVELTCTASQKKNIQFDWKNSNQIKILGNQGSFLTKGPSKLNDRVDSRRSLWDQGNFPLIIKNLKPEDSDTYICEVEDQKEEVQLVVVG

SEQ ID NO: 18 - protein, 99 aa, synthetic construct
KKVVYGKKGDTVELTCTASQKKNIQFHWKNSNQIKILGNQGSFLTKGPSKLNDRVDSVRSLWDQGNFPLIIKNLKPEDSDTYICEVEDQKEEVQLVVVG

SEQ ID NO: 19 - protein, 99 aa, synthetic construct
KKVVYGKKGDTVELTCTASQKKNIQFHWKNSNQIKILGNQGSFLTKGPSKLNDRVDSNRSLWDQGNFPLIIKNLKPEDSDTYICEVEDQKEEVQLVVVG

SEQ ID NO: 20 - protein, 99 aa, synthetic construct
KKVVYGKKGDTVELTCTASQKKNIQFHWKNSNQIKILGNQGSFLTKGPSKLNDRVDSTRSLWDQGNFPLIIKNLKPEDSDTYICEVEDQKEEVQLVVVG

SEQ ID NO: 21 - protein, 99 aa, synthetic construct
KKVVYGKKGDTVELTCTASQKKNIQFHWKNSNQIKILGNQGSFLTKGPSKLNDRVDSRRSMWDQGNFPLIIKNLKPEDSDTYICEVEDQKEEVQLVVVG

SEQ ID NO: 22 - protein, 7 aa, synthetic construct
ACNSFWG

SEQ ID NO: 23 - protein, 22 aa, synthetic construct
SLSHCASYWNRGWTYHNPSLKS

SEQ ID NO: 24 - protein, 19 aa, synthetic construct
FGGEVLRYTDWPKPAWVDL

SEQ ID NO: 25 - protein, 9 aa, synthetic construct
TGTSNNFVS

SEQ ID NO: 26 - protein, 7 aa, synthetic construct
DVNKRPS

SEQ ID NO: 27 - protein, 10 aa, synthetic construct
GSLVGNWDVI

SEQ ID NO: 28 - protein, 5 aa, synthetic construct
DSYWS

SEQ ID NO: 29 - protein, 16 aa, synthetic construct
YVHKSGDTNYSPSLKS

SEQ ID NO: 30 - protein, 24 aa, synthetic construct
TLHGRRIYGIVAFNEWFTYFYMDV

SEQ ID NO: 31 - protein, 11 aa, synthetic construct
GEKSLGSRAVQ

SEQ ID NO: 32 - protein, 7 aa, synthetic construct
NNQDRPS

SEQ ID NO: 33 - protein, 12 aa, synthetic construct
HIWDSRVPTKWV

SEQ ID NO: 34 - protein, 6 aa, synthetic construct
SDHSWT

SEQ ID NO: 35 - protein, 16 aa, synthetic construct
DIHYNGATTYNPSLRS

SEQ ID NO: 36 - protein, 21 aa, synthetic construct
NAIRIYGVVALGEWFHYGMDV

SEQ ID NO: 37 - protein, 11 aa, synthetic construct
SGAPLTSRFTY

SEQ ID NO: 38 - protein, 7 aa, synthetic construct
RSSQRSS

SEQ ID NO: 39 - protein, 11 aa, synthetic construct
QSSDTSDSYKM

SEQ ID NO: 40 - protein, 5 aa, synthetic construct
NYYWT

SEQ ID NO: 41 - protein, 16 aa, synthetic construct
YISDRESATYNPSLNS

SEQ ID NO: 42 - protein, 24 aa, synthetic construct
ARRGQRIYGVVSFGEFFYYYSMDV

SEQ ID NO: 43 - protein, 11 aa, synthetic construct
GRQALGSRAVQ

SEQ ID NO: 44 - protein, 7 aa, synthetic construct
NNQDRPS

SEQ ID NO: 45 - protein, 12 aa, synthetic construct
HMWDSRSGFSWS

SEQ ID NO: 46 - protein, 12 aa, synthetic construct
GGEWGDKDYHWG

SEQ ID NO: 47 - protein, 16 aa, synthetic construct
SIHWRGTTHYKESLRR

SEQ ID NO: 48 - protein, 18 aa, synthetic construct
HRHHDVFMLVPIAGWFDV

SEQ ID NO: 49 - protein, 11 aa, synthetic construct
RASQNINKNLA

SEQ ID NO: 50 - protein, 7 aa, synthetic construct
ETYSKIA

SEQ ID NO: 51 - protein, 9 aa, synthetic construct
QQYEEWPRT

SEQ ID NO: 52 - protein, 5 aa, synthetic construct
DFYIH

SEQ ID NO: 53 - protein, 17 aa, synthetic construct
WMNPQTGRTNTARNFQG

SEQ ID NO: 54 - protein, 18 aa, synthetic construct
GGWISLYYDSSYYPNFDH

SEQ ID NO: 55 - protein, 14 aa, synthetic construct
TGTKYDVGSHDLVS

SEQ ID NO: 56 - protein, 7 aa, synthetic construct
EVNKRPS

SEQ ID NO: 57 - protein, 10 aa, synthetic construct
CSFGGSATVV

SEQ ID NO: 58 - protein, 135 aa, synthetic construct
QPQLQESGPTLVEASETLSLTCAVSGDSTAACNSFWGWVRQPPGKGLEWVGSLSHCASYWNRGWTYHNPSLKSRLTLALDTPKNLVFLKLNSVTAADTATYYCARFGGEVLRYTDWPKPAWVDLWGRGTLVTVSS

SEQ ID NO: 59 - protein, 105 aa, synthetic construct
QSALTQPPSASGSPGQSITISCTGTSNNFVSWYQQHAGKAPKLVIYDVNKRPSGVPDRFSGSKSGNTASLTVSGLQTDDEAVYYCGSLVGNWDVIFGGGTKLTVL

SEQ ID NO: 60 - protein, 105 aa, synthetic construct
QSALTQPPSASGSPGQSITISCTGTSNNYVSWYQQHAGKAPKLVIYDVNKRPSGVPDRFSGSKSGNTASLTVSGLQTDDEAVYYCGSLVGNWDVIFGGGTKLTVL

SEQ ID NO: 61 - protein, 465 aa, synthetic construct
QPQLQESGPTLVEASETLSLTCAVSGDSTAACNSFWGWVRQPPGKGLEWVGSLSHCASYWNRGWTYHNPSLKSRLTLALDTPKNLVFLKLNSVTAADTATYYCARFGGEVLRYTDWPKPAWVDLWGRGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 62 - protein, 465 aa, synthetic construct
QPQLQESGPTLVEASETLSLTCAVSGDSTAACNSFWGWVRQPPGKGLEWVGSLSHCASYWNRGWTYHNPSLKSRLTLALDTPKNLVFLKLNSVTAADTATYYCARFGGEVLRYTDWPKPAWVDLWGRGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 63 - protein, 211 aa, synthetic construct
QSALTQPPSASGSPGQSITISCTGTSNNFVSWYQQHAGKAPKLVIYDVNKRPSGVPDRFSGSKSGNTASLTVSGLQTDDEAVYYCGSLVGNWDVIFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 64 - protein, 211 aa, synthetic construct
QSALTQPPSASGSPGQSITISCTGTSNNYVSWYQQHAGKAPKLVIYDVNKRPSGVPDRFSGSKSGNTASLTVSGLQTDDEAVYYCGSLVGNWDVIFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 65 - protein, 132 aa, synthetic construct
QMQLQESGPGLVKPSETLSLTCSVSGASISDSYWSWIRRSPGKGLEWIGYVHKSGDTNYSPSLKSRVNLSLDTSKNQVSLSLVAATAADSGKYYCARTLHGRRIYGIVAFNEWFTYFYMDVWGNGTQVTVSS

SEQ ID NO: 66 - protein, 105 aa, synthetic construct
SDISVAPGETARISCGEKSLGSRAVQWYQHRAGQAPSLIIYNNQDRPSGIPERFSGSPDSPFGTTATLTITSVEAGDEADYYCHIWDSRVPTKWVFGGGTTLTVL

SEQ ID NO: 67 - protein, 462 aa, synthetic construct
QMQLQESGPGLVKPSETLSLTCSVSGASISDSYWSWIRRSPGKGLEWIGYVHKSGDTNYSPSLKSRVNLSLDTSKNQVSLSLVAATAADSGKYYCARTLHGRRIYGIVAFNEWFTYFYMDVWGNGTQVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 68 - protein, 462 aa, synthetic construct
QMQLQESGPGLVKPSETLSLTCSVSGASISDSYWSWIRRSPGKGLEWIGYVHKSGDTNYSPSLKSRVNLSLDTSKNQVSLSLVAATAADSGKYYCARTLHGRRIYGIVAFNEWFTYFYMDVWGNGTQVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 69 - protein, 211 aa, synthetic construct
SDISVAPGETARISCGEKSLGSRAVQWYQHRAGQAPSLIIYNNQDRPSGIPERFSGSPDSPFGTTATLTITSVEAGDEADYYCHIWDSRVPTKWVFGGGTTLTVLGQPKAAPSVTLFPPSSEELQANKATLVCLISDFYPGAVTVAWKADSSPVKAGVETTTPSKQSNNKYAASSYLSLTPEQWKSHKSYSCQVTHEGSTVEKTVAPTECS

SEQ ID NO: 70 - protein, 130 aa, synthetic construct
QVQLRESGPGLVKPSETLSLSCTVSNDSRPSDHSWTWVRQSPGKALEWIGDIHYNGATTYNPSLRSRVRIELDQSIPRFSLKMTSMTAADTGMYYCARNAIRIYGVVALGEWFHYGMDVWGQGTAVTVSS

SEQ ID NO: 71 - protein, 108 aa, synthetic construct
SSELTQPPSVSVSPGQTARITCSGAPLTSRFTYWYRQKPGQAPVLIISRSSQRSSGWSGRFSASWSGTTVTLTIRGVQADDEADYYCQSSDTSDSYKMFGGGTKLTVL

SEQ ID NO: 72 - protein, 460 aa, synthetic construct
QVQLRESGPGLVKPSETLSLSCTVSNDSRPSDHSWTWVRQSPGKALEWIGDIHYNGATTYNPSLRSRVRIELDQSIPRFSLKMTSMTAADTGMYYCARNAIRIYGVVALGEWFHYGMDVWGQGTAVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 73 - protein, 460 aa, synthetic construct
QVQLRESGPGLVKPSETLSLSCTVSNDSRPSDHSWTWVRQSPGKALEWIGDIHYNGATTYNPSLRSRVRIELDQSIPRFSLKMTSMTAADTGMYYCARNAIRIYGVVALGEWFHYGMDVWGQGTAVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 74 - protein, 213 aa, synthetic construct
SSELTQPPSVSVSPGQTARITCSGAPLTSRFTYWYRQKPGQAPVLIISRSSQRSSGWSGRFSASWSGTTVTLTIRGVQADDEADYYCQSSDTSDSYKMFGGGTKLTVLGQPAAAPSVTLFPPSSEELQANKATLVCLISDFYPGAVTVAWKADSSPVKAGVETTTPSKQSNNKYAASSYLSLTPEQWKSHKSYSCQVTHEGSTVEKTVAPTEC

SEQ ID NO: 75 - protein, 132 aa, synthetic construct
QVQLQESGPGLVKPSETLSVTCSVSGDSMNNYYWTWIRQSPGKGLEWIGYISDRESATYNPSLNSRVVISRDTSKNQLSLKLNSVTPADTAVYYCATARRGQRIYGVVSFGEFFYYYSMDVWGKGTTVTVSS

SEQ ID NO: 76 - protein, 108 aa, synthetic construct
SYVRPLSVALGETARISCGRQALGSRAVQWYQHRPGQAPILLIYNNQDRPSGIPERFSGTPDINFGTRATLTISGVEAGDEADYYCHMWDSRSGFSWSFGGATRLTVL

SEQ ID NO: 77 - protein, 462 aa, synthetic construct
QVQLQESGPGLVKPSETLSVTCSVSGDSMNNYYWTWIRQSPGKGLEWIGYISDRESATYNPSLNSRVVISRDTSKNQLSLKLNSVTPADTAVYYCATARRGQRIYGVVSFGEFFYYYSMDVWGKGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 78 - protein, 462 aa, synthetic construct
QVQLQESGPGLVKPSETLSVTCSVSGDSMNNYYWTWIRQSPGKGLEWIGYISDRESATYNPSLNSRVVISRDTSKNQLSLKLNSVTPADTAVYYCATARRGQRIYGVVSFGEFFYYYSMDVWGKGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 79 - protein, 214 aa, synthetic construct
SYVRPLSVALGETARISCGRQALGSRAVQWYQHRPGQAPILLIYNNQDRPSGIPERFSGTPDINFGTRATLTISGVEAGDEADYYCHMWDSRSGFSWSFGGATRLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 80 - protein, 133 aa, synthetic construct [sequence omitted]

SEQ ID NO: 81 - protein, 107 aa, synthetic construct
EIVMTQSPDTLSVSPGETVTLSCRASQNINKNLAWYQYKPGQSPRLVIFETYSKIAAFPARFVASGSGTEFTLTINNMQSEDVAVYYCQQYEEWPRTFGQGTKVDIK

SEQ ID NO: 82 - protein, 463 aa, synthetic construct
QLQMQESGPGLVKPSETLSLSCTVSGDSIRGGEWGDKDYHWGWVRHSAGKGLEWIGSIHWRGTTHYKESLRRRVSMSIDTSRNWFSLRLASVTAADTAVYFCARHRHHDVFMLVPIAGWFDVWGPGVQVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 83 - protein, 463 aa, synthetic construct
QLQMQESGPGLVKPSETLSLSCTVSGDSIRGGEWGDKDYHWGWVRHSAGKGLEWIGSIHWRGTTHYKESLRRRVSMSIDTSRNWFSLRLASVTAADTAVYFCARHRHHDVFMLVPIAGWFDVWGPGVQVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 84 - protein, 214 aa, synthetic construct
EIVMTQSPDTLSVSPGETVTLSCRASQNINKNLAWYQYKPGQSPRLVIFETYSKIAAFPARFVASGSGTEFTLTINNMQSEDVAVYYCQQYEEWPRTFGQGTKVDIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 85 - protein, 127 aa, synthetic construct
QVQLVQSGAQMKNPGASVKVSCAPSGYTFTDFYIHWLRQAPGQGLQWMGWMNPQTGRTNTARNFQGRVTMTRDTSIGTAYMELRSLTSDDTAIYYCTTGGWISLYYDSSYYPNFDHWGQGTLLTVSS

SEQ ID NO: 86 - protein, 110 aa, synthetic construct
QSALTQPASVSGSPGQSITISCTGTKYDVGSHDLVSWYQQYPGKVPKYMIYEVNKRPSGVSNRFSGSKSGNTASLTISGLRAEDEADYYCCSFGGSATVVCGGGTKVTVL

SEQ ID NO: 87 - protein, 457 aa, synthetic construct
QVQLVQSGAQMKNPGASVKVSCAPSGYTFTDFYIHWLRQAPGQGLQWMGWMNPQTGRTNTARNFQGRVTMTRDTSIGTAYMELRSLTSDDTAIYYCTTGGWISLYYDSSYYPNFDHWGQGTLLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 88 - protein, 457 aa, synthetic construct
QVQLVQSGAQMKNPGASVKVSCAPSGYTFTDFYIHWLRQAPGQGLQWMGWMNPQTGRTNTARNFQGRVTMTRDTSIGTAYMELRSLTSDDTAIYYCTTGGWISLYYDSSYYPNFDHWGQGTLLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 89 - protein, 216 aa, synthetic construct
QSALTQPASVSGSPGQSITISCTGTKYDVGSHDLVSWYQQYPGKVPKYMIYEVNKRPSGVSNRFSGSKSGNTASLTISGLRAEDEADYYCCSFGGSATVVCGGGTKVTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 90 - protein, 5 aa, synthetic construct
GGGGS

SEQ ID NO: 91 - protein, 10 aa, synthetic construct
GGGGSGGGGS

SEQ ID NO: 92 - protein, 15 aa, synthetic construct
GGGGSGGGGSGGGGS

SEQ ID NO: 93 - protein, 20 aa, synthetic construct
GGGGSGGGGSGGGGSGGGGS

SEQ ID NO: 94 - protein, 25 aa, synthetic construct
GGGGSGGGGSGGGGSGGGGSGGGGS

SEQ ID NO: 95 - protein, 30 aa, synthetic construct
GGGGSGGGGSGGGGSGGGGSGGGGSGGGGS

SEQ ID NO: 96 - protein, 644 aa, synthetic construct
KKVVLGKKGDTVELTCTASQKKNIQFHWKNSNQIKILGNQGSFLTKGPSKLNDRVDSRRSLWDQGNFPLIIKNLKIEDSDTYICEVEDQKEEVQLVVFGLTANSDTHLLQGQSLTLTLESPPGSSPSVQCRSPRGKNIQGGKTLSVSQLELQDSGTWTCTVLQNQKKVEFKIDIVVLAFQPQLQESGPTLVEASETLSLTCAVSGDSTAACNSFWGWVRQPPGKGLEWVGSLSHCASYWNRGWTYHNPSLKSRLTLALDTPKNLVFLKLNSVTAADTATYYCARFGGEVLRYTDWPKPAWVDLWGRGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 97 - protein, 649 aa, synthetic construct
KKVVLGKKGDTVELTCTASQKKNIQFHWKNSNQIKILGNQGSFLTKGPSKLNDRVDSRRSLWDQGNFPLIIKNLKIEDSDTYICEVEDQKEEVQLVVFGLTANSDTHLLQGQSLTLTLESPPGSSPSVQCRSPRGKNIQGGKTLSVSQLELQDSGTWTCTVLQNQKKVEFKIDIVVLAFGGGGSQPQLQESGPTLVEASETLSLTCAVSGDSTAACNSFWGWVRQPPGKGLEWVGSLSHCASYWNRGWTYHNPSLKSRLTLALDTPKNLVFLKLNSVTAADTATYYCARFGGEVLRYTDWPKPAWVDLWGRGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 98 - protein, 654 aa, synthetic construct
KKVVLGKKGDTVELTCTASQKKNIQFHWKNSNQIKILGNQGSFLTKGPSKLNDRVDSRRSLWDQGNFPLIIKNLKIEDSDTYICEVEDQKEEVQLVVFGLTANSDTHLLQGQSLTLTLESPPGSSPSVQCRSPRGKNIQGGKTLSVSQLELQDSGTWTCTVLQNQKKVEFKIDIVVLAFGGGGSGGGGSQPQLQESGPTLVEASETLSLTCAVSGDSTAACNSFWGWVRQPPGKGLEWVGSLSHCASYWNRGWTYHNPSLKSRLTLALDTPKNLVFLKLNSVTAADTATYYCARFGGEVLRYTDWPKPAWVDLWGRGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 99 - protein, 659 aa, synthetic construct
KKVVLGKKGDTVELTCTASQKKNIQFHWKNSNQIKILGNQGSFLTKGPSKLNDRVDSRRSLWDQGNFPLIIKNLKIEDSDTYICEVEDQKEEVQLVVFGLTANSDTHLLQGQSLTLTLESPPGSSPSVQCRSPRGKNIQGGKTLSVSQLELQDSGTWTCTVLQNQKKVEFKIDIVVLAFGGGGSGGGGSGGGGSQPQLQESGPTLVEASETLSLTCAVSGDSTAACNSFWGWVRQPPGKGLEWVGSLSHCASYWNRGWTYHNPSLKSRLTLALDTPKNLVFLKLNSVTAADTATYYCARFGGEVLRYTDWPKPAWVDLWGRGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 100 - protein, 664 aa, synthetic construct
KKVVLGKKGDTVELTCTASQKKNIQFHWKNSNQIKILGNQGSFLTKGPSKLNDRVDSRRSLWDQGNFPLIIKNLKIEDSDTYICEVEDQKEEVQLVVFGLTANSDTHLLQGQSLTLTLESPPGSSPSVQCRSPRGKNIQGGKTLSVSQLELQDSGTWTCTVLQNQKKVEFKIDIVVLAFGGGGSGGGGSGGGGSGGGGSQPQLQESGPTLVEASETLSLTCAVSGDSTAACNSFWGWVRQPPGKGLEWVGSLSHCASYWNRGWTYHNPSLKSRLTLALDTPKNLVFLKLNSVTAADTATYYCARFGGEVLRYTDWPKPAWVDLWGRGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 101 - protein, 564 aa, synthetic construct
KKVVYGKKGDTVELTCTASQKKNIQFHWKNSNQIKILGNQGSFLTKGPSKLNDRVDSRRSLWDQGNFPLIIKNLKPEDSDTYICEVEDQKEEVQLVVVGQPQLQESGPTLVEASETLSLTCAVSGDSTAACNSFWGWVRQPPGKGLEWVGSLSHCASYWNRGWTYHNPSLKSRLTLALDTPKNLVFLKLNSVTAADTATYYCARFGGEVLRYTDWPKPAWVDLWGRGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 102 - protein, 569 aa, synthetic construct
KKVVYGKKGDTVELTCTASQKKNIQFHWKNSNQIKILGNQGSFLTKGPSKLNDRVDSRRSLWDQGNFPLIIKNLKPEDSDTYICEVEDQKEEVQLVVVGGGGGSQPQLQESGPTLVEASETLSLTCAVSGDSTAACNSFWGWVRQPPGKGLEWVGSLSHCASYWNRGWTYHNPSLKSRLTLALDTPKNLVFLKLNSVTAADTATYYCARFGGEVLRYTDWPKPAWVDLWGRGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 103 - protein, 574 aa, synthetic construct
KKVVYGKKGDTVELTCTASQKKNIQFHWKNSNQIKILGNQGSFLTKGPSKLNDRVDSRRSLWDQGNFPLIIKNLKPEDSDTYICEVEDQKEEVQLVVVGGGGGSGGGGSQPQLQESGPTLVEASETLSLTCAVSGDSTAACNSFWGWVRQPPGKGLEWVGSLSHCASYWNRGWTYHNPSLKSRLTLALDTPKNLVFLKLNSVTAADTATYYCARFGGEVLRYTDWPKPAWVDLWGRGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 104 - protein, 579 aa, synthetic construct
KKVVYGKKGDTVELTCTASQKKNIQFHWKNSNQIKILGNQGSFLTKGPSKLNDRVDSRRSLWDQGNFPLIIKNLKPEDSDTYICEVEDQKEEVQLVVVGGGGGSGGGGSGGGGSQPQLQESGPTLVEASETLSLTCAVSGDSTAACNSFWGWVRQPPGKGLEWVGSLSHCASYWNRGWTYHNPSLKSRLTLALDTPKNLVFLKLNSVTAADTATYYCARFGGEVLRYTDWPKPAWVDLWGRGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 105 - protein, 584 aa, synthetic construct
KKVVYGKKGDTVELTCTASQKKNIQFHWKNSNQIKILGNQGSFLTKGPSKLNDRVDSRRSLWDQGNFPLIIKNLKPEDSDTYICEVEDQKEEVQLVVVGGGGGSGGGGSGGGGSGGGGSQPQLQESGPTLVEASETLSLTCAVSGDSTAACNSFWGWVRQPPGKGLEWVGSLSHCASYWNRGWTYHNPSLKSRLTLALDTPKNLVFLKLNSVTAADTATYYCARFGGEVLRYTDWPKPAWVDLWGRGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 106 - protein, 589 aa, synthetic construct
KKVVYGKKGDTVELTCTASQKKNIQFHWKNSNQIKILGNQGSFLTKGPSKLNDRVDSRRSLWDQGNFPLIIKNLKPEDSDTYICEVEDQKEEVQLVVVGGGGGSGGGGSGGGGSGGGGSGGGGSQPQLQESGPTLVEASETLSLTCAVSGDSTAACNSFWGWVRQPPGKGLEWVGSLSHCASYWNRGWTYHNPSLKSRLTLALDTPKNLVFLKLNSVTAADTATYYCARFGGEVLRYTDWPKPAWVDLWGRGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 107 - protein, 594 aa, synthetic construct
KKVVYGKKGDTVELTCTASQKKNIQFHWKNSNQIKILGNQGSFLTKGPSKLNDRVDSRRSLWDQGNFPLIIKNLKPEDSDTYICEVEDQKEEVQLVVVGGGGGSGGGGSGGGGSGGGGSGGGGSGGGGSQPQLQESGPTLVEASETLSLTCAVSGDSTAACNSFWGWVRQPPGKGLEWVGSLSHCASYWNRGWTYHNPSLKSRLTLALDTPKNLVFLKLNSVTAADTATYYCARFGGEVLRYTDWPKPAWVDLWGRGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 108 - protein, 310 aa, synthetic construct
KKVVYGKKGDTVELTCTASQKKNIQFHWKNSNQIKILGNQGSFLTKGPSKLNDRVDSRRSLWDQGNFPLIIKNLKPEDSDTYICEVEDQKEEVQLVVVGQSALTQPPSASGSPGQSITISCTGTSNNFVSWYQQHAGKAPKLVIYDVNKRPSGVPDRFSGSKSGNTASLTVSGLQTDDEAVYYCGSLVGNWDVIFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 109 - protein, 315 aa, synthetic construct
KKVVYGKKGDTVELTCTASQKKNIQFHWKNSNQIKILGNQGSFLTKGPSKLNDRVDSRRSLWDQGNFPLIIKNLKPEDSDTYICEVEDQKEEVQLVVVGGGGGSQSALTQPPSASGSPGQSITISCTGTSNNFVSWYQQHAGKAPKLVIYDVNKRPSGVPDRFSGSKSGNTASLTVSGLQTDDEAVYYCGSLVGNWDVIFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 110 - protein, 320 aa, synthetic construct
KKVVYGKKGDTVELTCTASQKKNIQFHWKNSNQIKILGNQGSFLTKGPSKLNDRVDSRRSLWDQGNFPLIIKNLKPEDSDTYICEVEDQKEEVQLVVVGGGGGSGGGGSQSALTQPPSASGSPGQSITISCTGTSNNFVSWYQQHAGKAPKLVIYDVNKRPSGVPDRFSGSKSGNTASLTVSGLQTDDEAVYYCGSLVGNWDVIFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 111 - protein, 325 aa, synthetic construct
KKVVYGKKGDTVELTCTASQKKNIQFHWKNSNQIKILGNQGSFLTKGPSKLNDRVDSRRSLWDQGNFPLIIKNLKPEDSDTYICEVEDQKEEVQLVVVGGGGGSGGGGSGGGGSQSALTQPPSASGSPGQSITISCTGTSNNFVSWYQQHAGKAPKLVIYDVNKRPSGVPDRFSGSKSGNTASLTVSGLQTDDEAVYYCGSLVGNWDVIFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 112 - protein, 330 aa, synthetic construct
KKVVYGKKGDTVELTCTASQKKNIQFHWKNSNQIKILGNQGSFLTKGPSKLNDRVDSRRSLWDQGNFPLIIKNLKPEDSDTYICEVEDQKEEVQLVVVGGGGGSGGGGSGGGGSGGGGSQSALTQPPSASGSPGQSITISCTGTSNNFVSWYQQHAGKAPKLVIYDVNKRPSGVPDRFSGSKSGNTASLTVSGLQTDDEAVYYCGSLVGNWDVIFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 113 - protein, 335 aa, synthetic construct
KKVVYGKKGDTVELTCTASQKKNIQFHWKNSNQIKILGNQGSFLTKGPSKLNDRVDSRRSLWDQGNFPLIIKNLKPEDSDTYICEVEDQKEEVQLVVVGGGGGSGGGGSGGGGSGGGGSGGGGSQSALTQPPSASGSPGQSITISCTGTSNNFVSWYQQHAGKAPKLVIYDVNKRPSGVPDRFSGSKSGNTASLTVSGLQTDDEAVYYCGSLVGNWDVIFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 114 - protein, 340 aa, synthetic construct
KKVVYGKKGDTVELTCTASQKKNIQFHWKNSNQIKILGNQGSFLTKGPSKLNDRVDSRRSLWDQGNFPLIIKNLKPEDSDTYICEVEDQKEEVQLVVVGGGGGSGGGGSGGGGSGGGGSGGGGSGGGGSQSALTQPPSASGSPGQSITISCTGTSNNFVSWYQQHAGKAPKLVIYDVNKRPSGVPDRFSGSKSGNTASLTVSGLQTDDEAVYYCGSLVGNWDVIFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 115 - protein, 315 aa, synthetic construct
KKVVYGKCGDTVELTCTASQKKNIQFHWKNSNQIKILGNQGSFLTKGPSKLNDRVDSRRSLWDQGNFPLIIKNLKPEDSDTYICEVEDQKEEVQLVVVCGGGGSQSALTQPPSASGSPGQSITISCTGTSNNFVSWYQQHAGKAPKLVIYDVNKRPSGVPDRFSGSKSGNTASLTVSGLQTDDEAVYYCGSLVGNWDVIFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 116 - protein, 574 aa, synthetic construct
QPQLQESGPTLVEASETLSLTCAVSGDSTAACNSFWGWVRQPPGKGLEWVGSLSHCASYWNRGWTYHNPSLKSRLTLALDTPKNLVFLKLNSVTAADTATYYCARFGGEVLRYTDWPKPAWVDLWGRGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCGGGGSKKVVYGKCGDTVELTCTASQKKNIQFHWKNSNQIKILGNQGSFLTKGPSKLNDRVDSRRSLWDQGNFPLIIKNLKPEDSDTYICEVEDQKEEVQLVVVCGGGGSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 117 - protein, 568 aa, synthetic construct
QPQLQESGPTLVEASETLSLTCAVSGDSTAACNSFWGWVRQPPGKGLEWVGSLSHCASYWNRGWTYHNPSLKSRLTLALDTPKNLVFLKLNSVTAADTATYYCARFGGEVLRYTDWPKPAWVDLWGRGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGGGGGSKKVVYGKCGDTVELTCTASQKKNIQFHWKNSNQIKILGNQGSFLTKGPSKLNDRVDSRRSLWDQGNFPLIIKNLKPEDSDTYICEVEDQKEEVQLVVVC

SEQ ID NO: 118 - protein, 315 aa, synthetic construct
QSALTQPPSASGSPGQSITISCTGTSNNFVSWYQQHAGKAPKLVIYDVNKRPSGVPDRFSGSKSGNTASLTVSGLQTDDEAVYYCGSLVGNWDVIFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECSGGGGSKKVVYGKCGDTVELTCTASQKKNIQFHWKNSNQIKILGNQGSFLTKGPSKLNDRVDSRRSLWDQGNFPLIIKNLKPEDSDTYICEVEDQKEEVQLVVVC

SEQ ID NO: 119 - protein, 569 aa, synthetic construct
KKVVYGKIGDTVELTCTASQKKNIQFHWKNSNQIKILGNQGSFLTKGPSKLNDRVDSRRSLWDQGNFPLIIKNLKPEDSDTYICEVEDQKEEVQLVVVGGGGGSQPQLQESGPTLVEASETLSLTCAVSGDSTAACNSFWGWVRQPPGKGLEWVGSLSHCASYWNRGWTYHNPSLKSRLTLALDTPKNLVFLKLNSVTAADTATYYCARFGGEVLRYTDWPKPAWVDLWGRGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 120 - protein, 569 aa, synthetic construct
KKVVYGKVGDTVELTCTASQKKNIQFHWKNSNQIKILGNQGSFLTKGPSKLNDRVDSRRSLWDQGNFPLIIKNLKPEDSDTYICEVEDQKEEVQLVVVGGGGGSQPQLQESGPTLVEASETLSLTCAVSGDSTAACNSFWGWVRQPPGKGLEWVGSLSHCASYWNRGWTYHNPSLKSRLTLALDTPKNLVFLKLNSVTAADTATYYCARFGGEVLRYTDWPKPAWVDLWGRGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 121 - protein, 569 aa, synthetic construct
KKVVYGKCGDTVELTCTASQKKNIQFHWKNSNQIKILGNQGSFLTKGPSKLNDRVDSRRSLWDQGNFPLIIKNLKPEDSDTYICEVEDQKEEVQLVVVCGGGGSQPQLQESGPTLVEASETLSLTCAVSGDSTAACNSFWGWVRQPPGKGLEWVGSLSHCASYWNRGWTYHNPSLKSRLTLALDTPKNLVFLKLNSVTAADTATYYCARFGGEVLRYTDWPKPAWVDLWGRGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 122 - protein, 569 aa, synthetic construct
KKVVYGKKGDCVELTCTASQKKNIQFHWKNSNQIKILGNQGSFLTKGPSKLNDRVDSRRSLWDQGNFPLIICNLKPEDSDTYICEVEDQKEEVQLVVVGGGGGSQPQLQESGPTLVEASETLSLTCAVSGDSTAACNSFWGWVRQPPGKGLEWVGSLSHCASYWNRGWTYHNPSLKSRLTLALDTPKNLVFLKLNSVTAADTATYYCARFGGEVLRYTDWPKPAWVDLWGRGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 123 - protein, 569 aa, synthetic construct
KKVVYGKKGDTVELTCTASQKKNIQFHWKNSNQIKILGNQGSFLTKGPSKLNDRVDSRRSLWDQGNFPLIIKNLKPEDSDTYICEVEDQKQEVQLVVVGGGGGSQPQLQESGPTLVEASETLSLTCAVSGDSTAACNSFWGWVRQPPGKGLEWVGSLSHCASYWNRGWTYHNPSLKSRLTLALDTPKNLVFLKLNSVTAADTATYYCARFGGEVLRYTDWPKPAWVDLWGRGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 124 - protein, 569 aa, synthetic construct
KKVVYGKKGDTVELTCTASQKKNIQFHWKNSNQIKILGNQGSFLTKGPSKLNDRVDSRRSLWDQGNFPLIIKNLKPEDSDTYICEVEDQKHEVQLVVVGGGGGSQPQLQESGPTLVEASETLSLTCAVSGDSTAACNSFWGWVRQPPGKGLEWVGSLSHCASYWNRGWTYHNPSLKSRLTLALDTPKNLVFLKLNSVTAADTATYYCARFGGEVLRYTDWPKPAWVDLWGRGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 125 - protein, 569 aa, synthetic construct
KKVVYGKKGDTVELTCTASQKKNIEFHWKNSNQIKILGNQGSFLTKGPSKLNDRVDSRRSLWDQGNFPLIIKNLKPEDSDTYICEVEDQKEEVQLVVVGGGGGSQPQLQESGPTLVEASETLSLTCAVSGDSTAACNSFWGWVRQPPGKGLEWVGSLSHCASYWNRGWTYHNPSLKSRLTLALDTPKNLVFLKLNSVTAADTATYYCARFGGEVLRYTDWPKPAWVDLWGRGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 126 - protein, 569 aa, synthetic construct
KKVVYGKKGDTVELTCTASQKKNIQFDWKNSNQIKILGNQGSFLTKGPSKLNDRVDSRRSLWDQGNFPLIIKNLKPEDSDTYICEVEDQKEEVQLVVVGGGGGSQPQLQESGPTLVEASETLSLTCAVSGDSTAACNSFWGWVRQPPGKGLEWVGSLSHCASYWNRGWTYHNPSLKSRLTLALDTPKNLVFLKLNSVTAADTATYYCARFGGEVLRYTDWPKPAWVDLWGRGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 127 - protein, 569 aa, synthetic construct
KKVVYGKKGDTVELTCTASQKKNIQFHWKNSNQIKILGNQGSFLTKGPSKLNDRVDSVRSLWDQGNFPLIIKNLKPEDSDTYICEVEDQKEEVQLVVVGGGGGSQPQLQESGPTLVEASETLSLTCAVSGDSTAACNSFWGWVRQPPGKGLEWVGSLSHCASYWNRGWTYHNPSLKSRLTLALDTPKNLVFLKLNSVTAADTATYYCARFGGEVLRYTDWPKPAWVDLWGRGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 128 - protein, 569 aa, synthetic construct
KKVVYGKKGDTVELTCTASQKKNIQFHWKNSNQIKILGNQGSFLTKGPSKLNDRVDSNRSLWDQGNFPLIIKNLKPEDSDTYICEVEDQKEEVQLVVVGGGGGSQPQLQESGPTLVEASETLSLTCAVSGDSTAACNSFWGWVRQPPGKGLEWVGSLSHCASYWNRGWTYHNPSLKSRLTLALDTPKNLVFLKLNSVTAADTATYYCARFGGEVLRYTDWPKPAWVDLWGRGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 129 - protein, 569 aa, synthetic construct
KKVVYGKKGDTVELTCTASQKKNIQFHWKNSNQIKILGNQGSFLTKGPSKLNDRVDSTRSLWDQGNFPLIIKNLKPEDSDTYICEVEDQKEEVQLVVVGGGGGSQPQLQESGPTLVEASETLSLTCAVSGDSTAACNSFWGWVRQPPGKGLEWVGSLSHCASYWNRGWTYHNPSLKSRLTLALDTPKNLVFLKLNSVTAADTATYYCARFGGEVLRYTDWPKPAWVDLWGRGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 130 - protein, 569 aa, synthetic construct
KKVVYGKKGDTVELTCTASQKKNIQFHWKNSNQIKILGNQGSFLTKGPSKLNDRVDSRRSMWDQGNFPLIIKNLKPEDSDTYICEVEDQKEEVQLVVVGGGGGSQPQLQESGPTLVEASETLSLTCAVSGDSTAACNSFWGWVRQPPGKGLEWVGSLSHCASYWNRGWTYHNPSLKSRLTLALDTPKNLVFLKLNSVTAADTATYYCARFGGEVLRYTDWPKPAWVDLWGRGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 131 - protein, 569 aa, synthetic construct
KKVVYGKKGDTVCLTCTASQKKNIQFHWKNSNQIKILGNQGSFLTKGPSKLNDRVDSRRSLWDQGNFPLCIKNLKPEDSDTYICEVEDQKEEVQLVVVGGGGGSQPQLQESGPTLVEASETLSLTCAVSGDSTAACNSFWGWVRQPPGKGLEWVGSLSHCASYWNRGWTYHNPSLKSRLTLALDTPKNLVFLKLNSVTAADTATYYCARFGGEVLRYTDWPKPAWVDLWGRGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 132 - protein, 569 aa, synthetic construct
KKVVYGKKGDTVELTCTASQKKNIQFCWKNSNQIKILCNQGSFLTKGPSKLNDRVDSRRSLWDQGNFPLIIKNLKPEDSDTYICEVEDQKEEVQLVVVGGGGGSQPQLQESGPTLVEASETLSLTCAVSGDSTAACNSFWGWVRQPPGKGLEWVGSLSHCASYWNRGWTYHNPSLKSRLTLALDTPKNLVFLKLNSVTAADTATYYCARFGGEVLRYTDWPKPAWVDLWGRGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 133 - protein, 569 aa, synthetic construct
KKVVYGKKGDTVELTCTASQCKNIQFHWKNSNQIKILGNQGSFLTKGPSKLNDRVDSRRSLWDQCNFPLIIKNLKPEDSDTYICEVEDQKEEVQLVVVGGGGGSQPQLQESGPTLVEASETLSLTCAVSGDSTAACNSFWGWVRQPPGKGLEWVGSLSHCASYWNRGWTYHNPSLKSRLTLALDTPKNLVFLKLNSVTAADTATYYCARFGGEVLRYTDWPKPAWVDLWGRGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 134 - protein, 569 aa, synthetic construct
KKVVYGKKGDTVELTCTASQKKNIQFHWKNSNQIKILGNQGSFLTKGPSKLNDRVDSRRSLWDQGNFPLIIKNLKPEDSDTYICEVGDQKEEVQLVVVGGGGGSQPQLQESGPTLVEASETLSLTCAVSGDSTAACNSFWGWVRQPPGKGLEWVGSLSHCASYWNRGWTYHNPSLKSRLTLALDTPKNLVFLKLNSVTAADTATYYCARFGGEVLRYTDWPKPAWVDLWGRGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 135 - protein, 569 aa, synthetic construct
KKVVYGKKGDTVELTCTASQKKNIQFHWKNSNQIKILGNQGSFLTKGPSKLWDRVDSRRSLWDQGNFPLIIKNLKPEDSDTYICEVEDQKEEVQLVVVGGGGGSQPQLQESGPTLVEASETLSLTCAVSGDSTAACNSFWGWVRQPPGKGLEWVGSLSHCASYWNRGWTYHNPSLKSRLTLALDTPKNLVFLKLNSVTAADTATYYCARFGGEVLRYTDWPKPAWVDLWGRGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 136 - protein, 566 aa, synthetic construct
KKVVYGKCGDTVELTCTASQKKNIQFHWKNSNQIKILGNQGSFLTKGPSKLNDRVDSRRSLWDQGNFPLIIKNLKPEDSDTYICEVEDQKEEVQLVVVCGGGGSQMQLQESGPGLVKPSETLSLTCSVSGASISDSYWSWIRRSPGKGLEWIGYVHKSGDTNYSPSLKSRVNLSLDTSKNQVSLSLVAATAADSGKYYCARTLHGRRIYGIVAFNEWFTYFYMDVWGNGTQVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 137 - protein, 571 aa, synthetic construct
KKVVYGKCGDTVELTCTASQKKNIQFHWKNSNQIKILGNQGSFLTKGPSKLNDRVDSRRSLWDQGNFPLIIKNLKPEDSDTYICEVEDQKEEVQLVVVCGGGGSGGGGSQMQLQESGPGLVKPSETLSLTCSVSGASISDSYWSWIRRSPGKGLEWIGYVHKSGDTNYSPSLKSRVNLSLDTSKNQVSLSLVAATAADSGKYYCARTLHGRRIYGIVAFNEWFTYFYMDVWGNGTQVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 138 - protein, 576 aa, synthetic construct
KKVVYGKCGDTVELTCTASQKKNIQFHWKNSNQIKILGNQGSFLTKGPSKLNDRVDSRRSLWDQGNFPLIIKNLKPEDSDTYICEVEDQKEEVQLVVVCGGGGSGGGGSGGGGSQMQLQESGPGLVKPSETLSLTCSVSGASISDSYWSWIRRSPGKGLEWIGYVHKSGDTNYSPSLKSRVNLSLDTSKNQVSLSLVAATAADSGKYYCARTLHGRRIYGIVAFNEWFTYFYMDVWGNGTQVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 139 - protein, 581 aa, synthetic construct
KKVVYGKCGDTVELTCTASQKKNIQFHWKNSNQIKILGNQGSFLTKGPSKLNDRVDSRRSLWDQGNFPLIIKNLKPEDSDTYICEVEDQKEEVQLVVVCGGGGSGGGGSGGGGSGGGGSQMQLQESGPGLVKPSETLSLTCSVSGASISDSYWSWIRRSPGKGLEWIGYVHKSGDTNYSPSLKSRVNLSLDTSKNQVSLSLVAATAADSGKYYCARTLHGRRIYGIVAFNEWFTYFYMDVWGNGTQVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 140 - protein, 571 aa, synthetic construct
QMQLQESGPGLVKPSETLSLTCSVSGASISDSYWSWIRRSPGKGLEWIGYVHKSGDTNYSPSLKSRVNLSLDTSKNQVSLSLVAATAADSGKYYCARTLHGRRIYGIVAFNEWFTYFYMDVWGNGTQVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCGGGGSKKVVYGKCGDTVELTCTASQKKNIQFHWKNSNQIKILGNQGSFLTKGPSKLNDRVDSRRSLWDQGNFPLIIKNLKPEDSDTYICEVEDQKEEVQLVVVCGGGGSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 141 - protein, 565 aa, synthetic construct
QMQLQESGPGLVKPSETLSLTCSVSGASISDSYWSWIRRSPGKGLEWIGYVHKSGDTNYSPSLKSRVNLSLDTSKNQVSLSLVAATAADSGKYYCARTLHGRRIYGIVAFNEWFTYFYMDVWGNGTQVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGGGGGSKKVVYGKCGDTVELTCTASQKKNIQFHWKNSNQIKILGNQGSFLTKGPSKLNDRVDSRRSLWDQGNFPLIIKNLKPEDSDTYICEVEDQKEEVQLVVVC

SEQ ID NO: 142 - protein, 315 aa, synthetic construct
KKVVYGKCGDTVELTCTASQKKNIQFHWKNSNQIKILGNQGSFLTKGPSKLNDRVDSRRSLWDQGNFPLIIKNLKPEDSDTYICEVEDQKEEVQLVVVCGGGGSSDISVAPGETARISCGEKSLGSRAVQWYQHRAGQAPSLIIYNNQDRPSGIPERFSGSPDSPFGTTATLTITSVEAGDEADYYCHIWDSRVPTKWVFGGGTTLTVLGQPKAAPSVTLFPPSSEELQANKATLVCLISDFYPGAVTVAWKADSSPVKAGVETTTPSKQSNNKYAASSYLSLTPEQWKSHKSYSCQVTHEGSTVEKTVAPTECS

SEQ ID NO: 143 - protein, 315 aa, synthetic construct
SDISVAPGETARISCGEKSLGSRAVQWYQHRAGQAPSLIIYNNQDRPSGIPERFSGSPDSPFGTTATLTITSVEAGDEADYYCHIWDSRVPTKWVFGGGTTLTVLGQPKAAPSVTLFPPSSEELQANKATLVCLISDFYPGAVTVAWKADSSPVKAGVETTTPSKQSNNKYAASSYLSLTPEQWKSHKSYSCQVTHEGSTVEKTVAPTECSGGGGSKKVVYGKCGDTVELTCTASQKKNIQFHWKNSNQIKILGNQGSFLTKGPSKLNDRVDSRRSLWDQGNFPLIIKNLKPEDSDTYICEVEDQKEEVQLVVVC

SEQ ID NO: 144 - protein, 564 aa, synthetic construct
KKVVYGKCGDTVELTCTASQKKNIQFHWKNSNQIKILGNQGSFLTKGPSKLNDRVDSRRSLWDQGNFPLIIKNLKPEDSDTYICEVEDQKEEVQLVVVCGGGGSQVQLRESGPGLVKPSETLSLSCTVSNDSRPSDHSWTWVRQSPGKALEWIGDIHYNGATTYNPSLRSRVRIELDQSIPRFSLKMTSMTAADTGMYYCARNAIRIYGVVALGEWFHYGMDVWGQGTAVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 145 - protein, 579 aa, synthetic construct
KKVVYGKCGDTVELTCTASQKKNIQFHWKNSNQIKILGNQGSFLTKGPSKLNDRVDSRRSLWDQGNFPLIIKNLKPEDSDTYICEVEDQKEEVQLVVVCGGGGSGGGGSGGGGSGGGGSQVQLRESGPGLVKPSETLSLSCTVSNDSRPSDHSWTWVRQSPGKALEWIGDIHYNGATTYNPSLRSRVRIELDQSIPRFSLKMTSMTAADTGMYYCARNAIRIYGVVALGEWFHYGMDVWGQGTAVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 146 - protein, 566 aa, synthetic construct
KKVVYGKCGDTVELTCTASQKKNIQFHWKNSNQIKILGNQGSFLTKGPSKLNDRVDSRRSLWDQGNFPLIIKNLKPEDSDTYICEVEDQKEEVQLVVVCGGGGSQVQLQESGPGLVKPSETLSVTCSVSGDSMNNYYWTWIRQSPGKGLEWIGYISDRESATYNPSLNSRVVISRDTSKNQLSLKLNSVTPADTAVYYCATARRGQRIYGVVSFGEFFYYYSMDVWGKGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 147 - protein, 581 aa, synthetic construct
KKVVYGKCGDTVELTCTASQKKNIQFHWKNSNQIKILGNQGSFLTKGPSKLNDRVDSRRSLWDQGNFPLIIKNLKPEDSDTYICEVEDQKEEVQLVVVCGGGGSGGGGSGGGGSGGGGSQVQLQESGPGLVKPSETLSVTCSVSGDSMNNYYWTWIRQSPGKGLEWIGYISDRESATYNPSLNSRVVISRDTSKNQLSLKLNSVTPADTAVYYCATARRGQRIYGVVSFGEFFYYYSMDVWGKGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 148 - protein, 567 aa, synthetic construct
KKVVYGKCGDTVELTCTASQKKNIQFHWKNSNQIKILGNQGSFLTKGPSKLNDRVDSRRSLWDQGNFPLIIKNLKPEDSDTYICEVEDQKEEVQLVVVCGGGGSQLQMQESGPGLVKPSETLSLSCTVSGDSIRGGEWGDKDYHWGWVRHSAGKGLEWIGSIHWRGTTHYKESLRRRVSMSIDTSRNWFSLRLASVTAADTAVYFCARHRHHDVFMLVPIAGWFDVWGPGVQVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 149 - protein, 582 aa, synthetic construct
KKVVYGKCGDTVELTCTASQKKNIQFHWKNSNQIKILGNQGSFLTKGPSKLNDRVDSRRSLWDQGNFPLIIKNLKPEDSDTYICEVEDQKEEVQLVVVCGGGGSGGGGSGGGGSGGGGSQLQMQESGPGLVKPSETLSLSCTVSGDSIRGGEWGDKDYHWGWVRHSAGKGLEWIGSIHWRGTTHYKESLRRRVSMSIDTSRNWFSLRLASVTAADTAVYFCARHRHHDVFMLVPIAGWFDVWGPGVQVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 150 - protein, 656 aa, synthetic construct
KKVVLGKKGDTVELTCTASQKKNIQFHWKNSNQIKILGNQGSFLTKGPSKLNDRVDSRRSLWDQGNFPLIIKNLKIEDSDTYICEVEDQKEEVQLVVFGLTANSDTHLLQGQSLTLTLESPPGSSPSVQCRSPRGKNIQGGKTLSVSQLELQDSGTWTCTVLQNQKKVEFKIDIVVLAFGGGGSGGGGSGGGGSGGGGSQVQLVQSGAQMKNPGASVKVSCAPSGYTFTDFYIHWLRQAPGQGLQWMGWMNPQTGRTNTARNFQGRVTMTRDTSIGTAYMELRSLTSDDTAIYYCTTGGWISLYYDSSYYPNFDHWGQGTLLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 151 - protein, 576 aa, synthetic construct
KKVVYGKKGDTVELTCTASQKKNIQFHWKNSNQIKILGNQGSFLTKGPSKLNDRVDSRRSLWDQGNFPLIIKNLKPEDSDTYICEVEDQKEEVQLVVVGGGGGSGGGGSGGGGSGGGGSQVQLVQSGAQMKNPGASVKVSCAPSGYTFTDFYIHWLRQAPGQGLQWMGWMNPQTGRTNTARNFQGRVTMTRDTSIGTAYMELRSLTSDDTAIYYCTTGGWISLYYDSSYYPNFDHWGQGTLLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 152 - protein, 616 aa, synthetic construct
KKVVYGKKGDTVELTCTASQKKNIQFHWKNSNQIKILGNQGSFLTKGPSKLNDRVDSRRSLWDQGNFPLIIKNLKPEDSDTYICEVEDQKEEVQLVVVGGGGGSGGGGSGGGGSGGGGSQPQLQESGPTLVEASETLSLTCAVSGDSTAACNSFWGWVRQPPGKCLEWVGSLSHCASYWNRGWTYHNPSLKSRLTLALDTPKNLVFLKLNSVTAADTATYYCARFGGEVLRYTDWPKPAWVDLWGRGTLVTVSSGGGGSGGGGSGGGGSGGGGSQSALTQPPSASGSPGQSITISCTGTSNNFVSWYQQHAGKAPKLVIYDVNKRPSGVPDRFSGSKSGNTASLTVSGLQTDDEAVYYCGSLVGNWDVIFGCGTKLTVLGGGGSGGGGSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 153 - protein, 611 aa, synthetic construct
KKVVYGKKGDTVELTCTASQKKNIQFHWKNSNQIKILGNQGSFLTKGPSKLNDRVDSRRSLWDQGNFPLIIKNLKPEDSDTYICEVEDQKEEVQLVVVGGGGGSGGGGSGGGGSQPQLQESGPTLVEASETLSLTCAVSGDSTAACNSFWGWVRQPPGKCLEWVGSLSHCASYWNRGWTYHNPSLKSRLTLALDTPKNLVFLKLNSVTAADTATYYCARFGGEVLRYTDWPKPAWVDLWGRGTLVTVSSGGGGSGGGGSGGGGSGGGGSQSALTQPPSASGSPGQSITISCTGTSNNFVSWYQQHAGKAPKLVIYDVNKRPSGVPDRFSGSKSGNTASLTVSGLQTDDEAVYYCGSLVGNWDVIFGCGTKLTVLGGGGSGGGGSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 154 - protein, 606 aa, synthetic construct
KKVVYGKKGDTVELTCTASQKKNIQFHWKNSNQIKILGNQGSFLTKGPSKLNDRVDSRRSLWDQGNFPLIIKNLKPEDSDTYICEVEDQKEEVQLVVVGGGGGSGGGGSQPQLQESGPTLVEASETLSLTCAVSGDSTAACNSFWGWVRQPPGKCLEWVGSLSHCASYWNRGWTYHNPSLKSRLTLALDTPKNLVFLKLNSVTAADTATYYCARFGGEVLRYTDWPKPAWVDLWGRGTLVTVSSGGGGSGGGGSGGGGSGGGGSQSALTQPPSASGSPGQSITISCTGTSNNFVSWYQQHAGKAPKLVIYDVNKRPSGVPDRFSGSKSGNTASLTVSGLQTDDEAVYYCGSLVGNWDVIFGCGTKLTVLGGGGSGGGGSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 155 - protein, 601 aa, synthetic construct
KKVVYGKKGDTVELTCTASQKKNIQFHWKNSNQIKILGNQGSFLTKGPSKLNDRVDSRRSLWDQGNFPLIIKNLKPEDSDTYICEVEDQKEEVQLVVVGGGGGSQPQLQESGPTLVEASETLSLTCAVSGDSTAACNSFWGWVRQPPGKCLEWVGSLSHCASYWNRGWTYHNPSLKSRLTLALDTPKNLVFLKLNSVTAADTATYYCARFGGEVLRYTDWPKPAWVDLWGRGTLVTVSSGGGGSGGGGSGGGGSGGGGSQSALTQPPSASGSPGQSITISCTGTSNNFVSWYQQHAGKAPKLVIYDVNKRPSGVPDRFSGSKSGNTASLTVSGLQTDDEAVYYCGSLVGNWDVIFGCGTKLTVLGGGGSGGGGSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 156 - protein, 621 aa, synthetic construct
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGKGGGGSGGGGSGGGGSGGGGSKKVVYGKKGDTVELTCTASQKKNIQFHWKNSNQIKILGNQGSFLTKGPSKLNDRVDSRRSLWDQGNFPLIIKNLKPEDSDTYICEVEDQKEEVQLVVVGGGGGSGGGGSGGGGSQPQLQESGPTLVEASETLSLTCAVSGDSTAACNSFWGWVRQPPGKCLEWVGSLSHCASYWNRGWTYHNPSLKSRLTLALDTPKNLVFLKLNSVTAADTATYYCARFGGEVLRYTDWPKPAWVDLWGRGTLVTVSSGGGGSGGGGSGGGGSGGGGSQSALTQPPSASGSPGQSITISCTGTSNNFVSWYQQHAGKAPKLVIYDVNKRPSGVPDRFSGSKSGNTASLTVSGLQTDDEAVYYCGSLVGNWDVIFGCGTKLTVL

SEQ ID NO: 157 - protein, 621 aa, synthetic construct
QSALTQPPSASGSPGQSITISCTGTSNNFVSWYQQHAGKAPKLVIYDVNKRPSGVPDRFSGSKSGNTASLTVSGLQTDDEAVYYCGSLVGNWDVIFGCGTKLTVLGGGGSGGGGSGGGGSGGGGSQPQLQESGPTLVEASETLSLTCAVSGDSTAACNSFWGWVRQPPGKCLEWVGSLSHCASYWNRGWTYHNPSLKSRLTLALDTPKNLVFLKLNSVTAADTATYYCARFGGEVLRYTDWPKPAWVDLWGRGTLVTVSSGGGGSGGGGSGGGGSGGGGSKKVVYGKKGDTVELTCTASQKKNIQFHWKNSNQIKILGNQGSFLTKGPSKLNDRVDSRRSLWDQGNFPLIIKNLKPEDSDTYICEVEDQKEEVQLVVVGGGGGSGGGGSGGGGSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 158 - protein, 346 aa, synthetic construct
KKVVYGKCGDTVELTCTASQKKNIQFHWKNSNQIKILGNQGSFLTKGPSKLNDRVDSRRSLWDQGNFPLIIKNLKPEDSDTYICEVEDQKEEVQLVVVCGGGGSGGGGSGGGGSGGGGSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 159 - protein, 5 aa, synthetic construct
DNYWS

SEQ ID NO: 160 - protein, 16 aa, synthetic construct
YVHDSGDTNYNPSLKS

SEQ ID NO: 161 - protein, 24 aa, synthetic construct
TKHGRRIYGVVAFKEWFTYFYMDV

SEQ ID NO: 162 - protein, 11 aa, synthetic construct
GEESLGSRSVI

SEQ ID NO: 163 - protein, 7 aa, synthetic construct
NNNDRPS

SEQ ID NO: 164 - protein, 12 aa, synthetic construct
HIWDSRRPTNWV

SEQ ID NO: 165 - protein, 5 aa, synthetic construct
DAYWS

SEQ ID NO: 166 - protein, 16 aa, synthetic construct
YVHHSGDTNYNPSLKR

SEQ ID NO: 167 - protein, 24 aa, synthetic construct
ALHGKRIYGIVALGELFTYFYMDV

SEQ ID NO: 168 - protein, 11 aa, synthetic construct
GKESIGSRAVQ

SEQ ID NO: 169 - protein, 7 aa, synthetic construct
NNQDRPA

SEQ ID NO: 170 - protein, 12 aa, synthetic construct
HIYDARGGTNWV

SEQ ID NO: 171 - protein, 7 aa, synthetic construct
ACTYFWG

SEQ ID NO: 172 - protein, 22 aa, synthetic construct
SLSHCQSFWGSGWTFHNPSLKS

SEQ ID NO: 173 - protein, 19 aa, synthetic construct
FDGEVLVYNHWPKPAWVDL

SEQ ID NO: 174 - protein, 9 aa, synthetic construct
NGTATNFVS

SEQ ID NO: 175 - protein, 7 aa, synthetic construct
GVDKRPP

SEQ ID NO: 176 - protein, 10 aa, synthetic construct
GSLVGNWDVI

SEQ ID NO: 177 - protein, 7 aa, synthetic construct
ACDYFWG

SEQ ID NO: 178 - protein, 22 aa, synthetic construct
GLSHCAGYYNTGWTYHNPSLKS

SEQ ID NO: 179 - protein, 19 aa, synthetic construct
FDGEVLVYHDWPKPAWVDL

SEQ ID NO: 180 - protein, 9 aa, synthetic construct
TGTSNRFVS

SEQ ID NO: 181 - protein, 7 aa, synthetic construct
GVNKRPS

SEQ ID NO: 182 - protein, 10 aa, synthetic construct
SSLVGNWDVI

SEQ ID NO: 183 - protein, 7 aa, synthetic construct
RCNYFWG

SEQ ID NO: 184 - protein, 22 aa, synthetic construct
SLSHCRSYYNTDWTYHNPSLKS

SEQ ID NO: 185 - protein, 19 aa, synthetic construct
FGGEVLVYRDWPKPAWVDL

SEQ ID NO: 186 - protein, 9 aa, synthetic construct
TGTSNNFVS

SEQ ID NO: 187 - protein, 7 aa, synthetic construct
EVNKRPS

SEQ ID NO: 188 - protein, 10 aa, synthetic construct
SSLVGNWDVI

SEQ ID NO: 189 - protein, 7 aa, synthetic construct
TGHYYWG

SEQ ID NO: 190 - protein, 16 aa, synthetic construct
HIHYTTAVLHNPSLKS

SEQ ID NO: 191 - protein, 19 aa, synthetic construct
SGGDILYYYEWQKPHWFSP

SEQ ID NO: 192 - protein, 14 aa, synthetic construct
NGTSSDIGGWNFVS

SEQ ID NO: 193 - protein, 7 aa, synthetic construct
EVNKRPS

SEQ ID NO: 194 - protein, 10 aa, synthetic construct
SSLFGRWDVV

SEQ ID NO: 195 - protein, 7 aa, synthetic construct
TGHHYWG

SEQ ID NO: 196 - protein, 16 aa, synthetic construct
HIHYNTAVLHNPALKS

SEQ ID NO: 197 - protein, 19 aa, synthetic construct
SGGDILYYIEWQKPHWFYP

SEQ ID NO: 198 - protein, 14 aa, synthetic construct
SGTGSDIGSWNFVS

SEQ ID NO: 199 - protein, 7 aa, synthetic construct
EVNRRRS

SEQ ID NO: 200 - protein, 10 aa, synthetic construct
SSLSGRWDIV

SEQ ID NO: 201 - protein, 12 aa, synthetic construct
GTDWGENDFHYG

SEQ ID NO: 202 - protein, 17 aa, synthetic construct
SIHWRGRTTHYKTSFRS

SEQ ID NO: 203 - protein, 18 aa, synthetic construct
HKYHDIFRVVPVAGWFDP

SEQ ID NO: 204 - protein, 11 aa, synthetic construct
RASQNVKNNLA

SEQ ID NO: 205 - protein, 7 aa, synthetic construct
DASSRAG

SEQ ID NO: 206 - protein, 9 aa, synthetic construct
QQYEEWPRT

SEQ ID NO: 207 - protein, 12 aa, synthetic construct
GGEWGDSDYHWG

SEQ ID NO: 208 - protein, 16 aa, synthetic construct
SIHWRGTTHYNAPFRG

SEQ ID NO: 209 - protein, 18 aa, synthetic construct
HKYHDIVMVVPIAGWFDP

SEQ ID NO: 210 - protein, 11 aa, synthetic construct
RASQSVKNNLA

SEQ ID NO: 211 - protein, 7 aa, synthetic construct
DTSSRAS

SEQ ID NO: 212 - protein, 9 aa, synthetic construct
QQYEEWPRT

SEQ ID NO: 213 - protein, 5 aa, synthetic construct
DVWLN

SEQ ID NO: 214 - protein, 19 aa, synthetic construct
RIKSRTDGGTTDYAASVKG

SEQ ID NO: 215 - protein, 20 aa, synthetic construct
DGFIMIRGVSEDYYYYYMDV

SEQ ID NO: 216 - protein, 13 aa, synthetic construct
SGSSSNIGNNYVL

SEQ ID NO: 217 - protein, 7 aa, synthetic construct
GNNKRPS

SEQ ID NO: 218 - protein, 12 aa, synthetic construct
ATWDSGLSADWV

SEQ ID NO: 219 - protein, 5 aa, synthetic construct
SYVMH

SEQ ID NO: 220 - protein, 17 aa, synthetic construct
AISSDGETTYHANSVKG

SEQ ID NO: 221 - protein, 14 aa, synthetic construct
DRYYETSGSNAFDV

SEQ ID NO: 222 - protein, 11 aa, synthetic construct
QASQDISNYLN

SEQ ID NO: 223 - protein, 7 aa, synthetic construct
TASNLET

SEQ ID NO: 224 - protein, 10 aa, synthetic construct
QQYDNLGDLS

SEQ ID NO: 225 - protein, 5 aa, synthetic construct
NFAIH

SEQ ID NO: 226 - protein, 17 aa, synthetic construct
GRVPVVGIYKYGKKFHD

SEQ ID NO: 227 - protein, 21 aa, synthetic construct
WRGCGMCPYDTSSYYNDASDV

SEQ ID NO: 228 - protein, 12 aa, synthetic construct
RASQNISSSWIA

SEQ ID NO: 229 - protein, 7 aa, synthetic construct
AASARAA

SEQ ID NO: 230 - protein, 9 aa, synthetic construct
QYYGGSFFT

SEQ ID NO: 231 - protein, 5 aa, synthetic construct
AHTMN

SEQ ID NO: 232 - protein, 17 aa, synthetic construct
SISTSSTYRDYADAVKG

SEQ ID NO: 233 - protein, 14 aa, synthetic construct
KGSDRLSDNDPFDA

SEQ ID NO: 234 - protein, 11 aa, synthetic construct
RASQSIETWLA

SEQ ID NO: 235 - protein, 7 aa, synthetic construct
KASTLKT

SEQ ID NO: 236 - protein, 9 aa, synthetic construct
QHYAGYSAT

SEQ ID NO: 237 - protein, 5 aa, synthetic construct
SSYWS

SEQ ID NO: 238 - protein, 16 aa, synthetic construct
YTHHSGDTNYAPSLKS

SEQ ID NO: 239 - protein, 24 aa, synthetic construct
TLHGRRIYGVVAFNEFFTYFYWEV

SEQ ID NO: 240 - protein, 11 aa, synthetic construct
GGESIGSRAVQ

SEQ ID NO: 241 - protein, 7 aa, synthetic construct
NNQDRPP

SEQ ID NO: 242 - protein, 12 aa, synthetic construct
HIWDSRRPTNWV

SEQ ID NO: 243 - protein, 5 aa, synthetic construct
SSYWS

SEQ ID NO: 244 - protein, 16 aa, synthetic construct
YTHHSGDTNYAPSLKS

SEQ ID NO: 245 - protein, 24 aa, synthetic construct
TLHGRRIYGVVAFNEYYTYFYWPT

SEQ ID NO: 246 - protein, 11 aa, synthetic construct
GGESIGSRAVQ

SEQ ID NO: 247 - protein, 7 aa, synthetic construct
NNQDRPP

SEQ ID NO: 248 - protein, 12 aa, synthetic construct
HIWDSRRPTNWE

SEQ ID NO: 249 - protein, 5 aa, synthetic construct
SSYWS

SEQ ID NO: 250 - protein, 16 aa, synthetic construct
YTHHSGDTNYAPSLKS

SEQ ID NO: 251 - protein, 24 aa, synthetic construct
TLHGRRIYGVVAFNEYYTYFYWPT

SEQ ID NO: 252 - protein, 14 aa, synthetic construct
TGTSSDIGASDYVS

SEQ ID NO: 253 - protein, 7 aa, synthetic construct
DVTKRPS

SEQ ID NO: 254 - protein, 10 aa, synthetic construct
SSDAGRHTLL

SEQ ID NO: 255 - protein, 132 aa, synthetic construct
QVHLQESGPGLVKPSETLSLTCNVSGTLVRDNYWSWIRQPLGKQPEWIGYVHDSGDTNYNPSLKSRVHLSLDKSKNLVSLRLTGVTAADSAIYYCATTKHGRRIYGVVAFKEWFTYFYMDVWGKGTSVTVSS

SEQ ID NO: 256 - protein, 105 aa, synthetic construct
TFVSVAPGQTARITCGEESLGSRSVIWYQQRPGQAPSLIIYNNNDRPSGIPDRFSGSPGSTFGTTATLTITSVEAGDEADYYCHIWDSRRPTNWVFGEGTTLIVL

SEQ ID NO: 257 - protein, 462 aa, synthetic construct
QVHLQESGPGLVKPSETLSLTCNVSGTLVRDNYWSWIRQPLGKQPEWIGYVHDSGDTNYNPSLKSRVHLSLDKSKNLVSLRLTGVTAADSAIYYCATTKHGRRIYGVVAFKEWFTYFYMDVWGKGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 258 - protein, 462 aa, synthetic construct
QVHLQESGPGLVKPSETLSLTCNVSGTLVRDNYWSWIRQPLGKQPEWIGYVHDSGDTNYNPSLKSRVHLSLDKSKNLVSLRLTGVTAADSAIYYCATTKHGRRIYGVVAFKEWFTYFYMDVWGKGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 259 - protein, 211 aa, synthetic construct
TFVSVAPGQTARITCGEESLGSRSVIWYQQRPGQAPSLIIYNNNDRPSGIPDRFSGSPGSTFGTTATLTITSVEAGDEADYYCHIWDSRRPTNWVFGEGTTLIVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 260 - protein, 132 aa, synthetic construct
QLHLQESGPGLVKPPETLSLTCSVSGASINDAYWSWIRQSPGKRPEWVGYVHHSGDTNYNPSLKRRVTFSLDTAKNEVSLKLVDLTAADSATYFCARALHGKRIYGIVALGELFTYFYMDVWGKGTAVTVSS

SEQ ID NO: 261 - protein, 105 aa, synthetic construct
SSMSVSPGETAKISCGKESIGSRAVQWYQQKPGQPPSLIIYNNQDRPAGVPERFSASPDFRPGTTATLTITNVDAEDEADYYCHIYDARGGTNWVFDRGTTLTVL

SEQ ID NO: 262 - protein, 462 aa, synthetic construct
QLHLQESGPGLVKPPETLSLTCSVSGASINDAYWSWIRQSPGKRPEWVGYVHHSGDTNYNPSLKRRVTFSLDTAKNEVSLKLVDLTAADSATYFCARALHGKRIYGIVALGELFTYFYMDVWGKGTAVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 263 - protein, 462 aa, synthetic construct
QLHLQESGPGLVKPPETLSLTCSVSGASINDAYWSWIRQSPGKRPEWVGYVHHSGDTNYNPSLKRRVTFSLDTAKNEVSLKLVDLTAADSATYFCARALHGKRIYGIVALGELFTYFYMDVWGKGTAVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 264 - protein, 211 aa, synthetic construct
SSMSVSPGETAKISCGKESIGSRAVQWYQQKPGQPPSLIIYNNQDRPAGVPERFSASPDFRPGTTATLTITNVDAEDEADYYCHIYDARGGTNWVFDRGTTLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 265 - protein, 137 aa, synthetic construct
QSQLQESGPRLVEASETLSLTCNVSGESTGACTYFWGWVRQAPGKGLEWIGSLSHCQSFWGSGWTFHNPSLKSRLTISLDTPKNQVFLKLTSLTAADTATYYCARFDGEVLVYNHWPKPAWVDLWGRGIPVTVTVSS

SEQ ID NO: 266 - protein, 105 aa, synthetic construct
QSALTQPPSASGSPGQSITISCNGTATNFVSWYQQFPDKAPKLIIFGVDKRPPGVPDRFSGSRSGTTASLTVSRLQTDDEAVYYCGSLVGNWDVIFGGGTTLTVL

SEQ ID NO: 267 - protein, 467 aa, synthetic construct
QSQLQESGPRLVEASETLSLTCNVSGESTGACTYFWGWVRQAPGKGLEWIGSLSHCQSFWGSGWTFHNPSLKSRLTISLDTPKNQVFLKLTSLTAADTATYYCARFDGEVLVYNHWPKPAWVDLWGRGIPVTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 268 - protein, 467 aa, synthetic construct
QSQLQESGPRLVEASETLSLTCNVSGESTGACTYFWGWVRQAPGKGLEWIGSLSHCQSFWGSGWTFHNPSLKSRLTISLDTPKNQVFLKLTSLTAADTATYYCARFDGEVLVYNHWPKPAWVDLWGRGIPVTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 269 - protein, 211 aa, synthetic construct
QSALTQPPSASGSPGQSITISCNGTATNFVSWYQQFPDKAPKLIIFGVDKRPPGVPDRFSGSRSGTTASLTVSRLQTDDEAVYYCGSLVGNWDVIFGGGTTLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 270 - protein, 137 aa, synthetic construct
QPQLQESGPGLVEASETLSLTCTVSGDSTAACDYFWGWVRQPPGKGLEWIGGLSHCAGYYNTGWTYHNPSLKSRLTISLDTPKNQVFLKLNSVTAADTAIYYCARFDGEVLVYHDWPKPAWVDLWGRGTLVTVTVSS

SEQ ID NO: 271 - protein, 105 aa, synthetic construct
QSALTQPPSASGSPGQSISISCTGTSNRFVSWYQQHPGKAPKLVIYGVNKRPSGVPDRFSGSKSGNTASLTVSGLQTDDEAVYYCSSLVGNWDVIFGGGTKLTVL

SEQ ID NO: 272 - protein, 467 aa, synthetic construct
QPQLQESGPGLVEASETLSLTCTVSGDSTAACDYFWGWVRQPPGKGLEWIGGLSHCAGYYNTGWTYHNPSLKSRLTISLDTPKNQVFLKLNSVTAADTAIYYCARFDGEVLVYHDWPKPAWVDLWGRGTLVTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 273 - protein, 467 aa, synthetic construct
QPQLQESGPGLVEASETLSLTCTVSGDSTAACDYFWGWVRQPPGKGLEWIGGLSHCAGYYNTGWTYHNPSLKSRLTISLDTPKNQVFLKLNSVTAADTAIYYCARFDGEVLVYHDWPKPAWVDLWGRGTLVTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 274 - protein, 211 aa, synthetic construct
QSALTQPPSASGSPGQSISISCTGTSNRFVSWYQQHPGKAPKLVIYGVNKRPSGVPDRFSGSKSGNTASLTVSGLQTDDEAVYYCSSLVGNWDVIFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 275 - protein, 135 aa, synthetic construct
QPQLQESGPGLVEASETLSLTCTVSGDSTGRCNYFWGWVRQPPGKGLEWIGSLSHCRSYYNTDWTYHNPSLKSRLTISLDTPKNQVFLRLTSVTAADTATYYCARFGGEVLVYRDWPKPAWVDLWGRGTLVTVSS

SEQ ID NO: 276 - protein, 105 aa, synthetic construct
QSALTQPPSASGSPGQSITISCTGTSNNFVSWYQQYPGKAPKLVIYEVNKRPSGVPDRFSGSKSGSTASLTVSGLQADDEGVYYCSSLVGNWDVIFGGGTKLTVL

SEQ ID NO: 277 - protein, 465 aa, synthetic construct
QPQLQESGPGLVEASETLSLTCTVSGDSTGRCNYFWGWVRQPPGKGLEWIGSLSHCRSYYNTDWTYHNPSLKSRLTISLDTPKNQVFLRLTSVTAADTATYYCARFGGEVLVYRDWPKPAWVDLWGRGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 278 - protein, 465 aa, synthetic construct
QPQLQESGPGLVEASETLSLTCTVSGDSTGRCNYFWGWVRQPPGKGLEWIGSLSHCRSYYNTDWTYHNPSLKSRLTISLDTPKNQVFLRLTSVTAADTATYYCARFGGEVLVYRDWPKPAWVDLWGRGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 279 - protein, 211 aa, synthetic construct
QSALTQPPSASGSPGQSITISCTGTSNNFVSWYQQYPGKAPKLVIYEVNKRPSGVPDRFSGSKSGSTASLTVSGLQADDEGVYYCSSLVGNWDVIFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 280 - protein, 129 aa, synthetic construct
QVQLQESGPGLVKPAETLSLTCSVSGESINTGHYYWGWVRQVPGKGLEWIGHIHYTTAVLHNPSLKSRLTIKIYTLRNQITLRLSNVTAADTAVYHCVRSGGDILYYYEWQKPHWFSPWGPGIHVTVSS

SEQ ID NO: 281 - protein, 110 aa, synthetic construct
QSALTQPPSASGSLGQSVTISCNGTSSDIGGWNFVSWYQQFPGRAPRLIIFEVNKRPSGVPGRFSGSKSGNSASLTVSGLQSDDEGQYFCSSLFGRWDVVFGGGTKLTVL

SEQ ID NO: 282 - protein, 459 aa, synthetic construct
QVQLQESGPGLVKPAETLSLTCSVSGESINTGHYYWGWVRQVPGKGLEWIGHIHYTTAVLHNPSLKSRLTIKIYTLRNQITLRLSNVTAADTAVYHCVRSGGDILYYYEWQKPHWFSPWGPGIHVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 283 - protein, 459 aa, synthetic construct
QVQLQESGPGLVKPAETLSLTCSVSGESINTGHYYWGWVRQVPGKGLEWIGHIHYTTAVLHNPSLKSRLTIKIYTLRNQITLRLSNVTAADTAVYHCVRSGGDILYYYEWQKPHWFSPWGPGIHVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 284 - protein, 216 aa, synthetic construct
QSALTQPPSASGSLGQSVTISCNGTSSDIGGWNFVSWYQQFPGRAPRLIIFEVNKRPSGVPGRFSGSKSGNSASLTVSGLQSDDEGQYFCSSLFGRWDVVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 285 - protein, 129 aa, synthetic construct
QVQLQESGPGLVKPSETLSLTCTVSGDSINTGHHYWGWVRQVPGKGPEWIAHIHYNTAVLHNPALKSRVTISIFTLKNLITLSLSNVTAADTAVYFCVRSGGDILYYIEWQKPHWFYPWGPGILVTVSS

SEQ ID NO: 286 - protein, 110 aa, synthetic construct
QSALTQPPSASGSLGQSLTISCSGTGSDIGSWNFVSWYQQFPGRAPNLIIFEVNRRRSGVPDRFSGSKSGNTASLTVSGLRSEDEAEYFCSSLSGRWDIVFGGGTKVTVL

SEQ ID NO: 287 - protein, 459 aa, synthetic construct
QVQLQESGPGLVKPSETLSLTCTVSGDSINTGHHYWGWVRQVPGKGPEWIAHIHYNTAVLHNPALKSRVTISIFTLKNLITLSLSNVTAADTAVYFCVRSGGDILYYIEWQKPHWFYPWGPGILVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 288 - protein, 459 aa, synthetic construct
QVQLQESGPGLVKPSETLSLTCTVSGDSINTGHHYWGWVRQVPGKGPEWIAHIHYNTAVLHNPALKSRVTISIFTLKNLITLSLSNVTAADTAVYFCVRSGGDILYYIEWQKPHWFYPWGPGILVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 289 - protein, 216 aa, synthetic construct
QSALTQPPSASGSLGQSLTISCSGTGSDIGSWNFVSWYQQFPGRAPNLIIFEVNRRRSGVPDRFSGSKSGNTASLTVSGLRSEDEAEYFCSSLSGRWDIVFGGGTKVTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 290 - protein, 134 aa, synthetic construct
QLQLQESGPGLVKPSETLSLTCTVSGGSMRGTDWGENDFHYGWIRQSSAKGLEWIGSIHWRGRTTHYKTSFRSRATLSIDTSNNRFSLTFSFVTAADTAVYYCARHKYHDIFRVVPVAGWFDPWGQGLLVTVSS

SEQ ID NO: 291 - protein, 107 aa, synthetic construct
EIVMTQSPPTLSVSPGETATLSCRASQNVKNNLAWYQLKPGQAPRLLIFDASSRAGGIPDRFSGSGYGTDFTLTVNSVQSEDFGDYFCQQYEEWPRTFGQGTKVDIK

SEQ ID NO: 292 - protein, 464 aa, synthetic construct
QLQLQESGPGLVKPSETLSLTCTVSGGSMRGTDWGENDFHYGWIRQSSAKGLEWIGSIHWRGRTTHYKTSFRSRATLSIDTSNNRFSLTFSFVTAADTAVYYCARHKYHDIFRVVPVAGWFDPWGQGLLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 293 - protein, 464 aa, synthetic construct
QLQLQESGPGLVKPSETLSLTCTVSGGSMRGTDWGENDFHYGWIRQSSAKGLEWIGSIHWRGRTTHYKTSFRSRATLSIDTSNNRFSLTFSFVTAADTAVYYCARHKYHDIFRVVPVAGWFDPWGQGLLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 294 - protein, 214 aa, synthetic construct
EIVMTQSPPTLSVSPGETATLSCRASQNVKNNLAWYQLKPGQAPRLLIFDASSRAGGIPDRFSGSGYGTDFTLTVNSVQSEDFGDYFCQQYEEWPRTFGQGTKVDIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 295 - protein, 133 aa, synthetic construct
EVHLEESGPGLVRPSETLSLTCTASGGSIRGGEWGDSDYHWGWVRHSPEKGLEWIGSIHWRGTTHYNAPFRGRGRLSIDLSRNQFSLRLTSVTAEDTAVYYCVKHKYHDIVMVVPIAGWFDPWGQGLQVTVSS

SEQ ID NO: 296 - protein, 107 aa, synthetic construct
EIMMTQSPAILSVSPGDRATLSCRASQSVKNNLAWYQKRPGQAPRLLIFDTSSRASGIPARFSGGGSGTEFTLTVNSMQSEDFATYYCQQYEEWPRTFGQGTKVEIK

SEQ ID NO: 297 - protein, 463 aa, synthetic construct
EVHLEESGPGLVRPSETLSLTCTASGGSIRGGEWGDSDYHWGWVRHSPEKGLEWIGSIHWRGTTHYNAPFRGRGRLSIDLSRNQFSLRLTSVTAEDTAVYYCVKHKYHDIVMVVPIAGWFDPWGQGLQVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 298 - protein, 463 aa, synthetic construct
EVHLEESGPGLVRPSETLSLTCTASGGSIRGGEWGDSDYHWGWVRHSPEKGLEWIGSIHWRGTTHYNAPFRGRGRLSIDLSRNQFSLRLTSVTAEDTAVYYCVKHKYHDIVMVVPIAGWFDPWGQGLQVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 299 - protein, 214 aa, synthetic construct
EIMMTQSPAILSVSPGDRATLSCRASQSVKNNLAWYQKRPGQAPRLLIFDTSSRASGIPARFSGGGSGTEFTLTVNSMQSEDFATYYCQQYEEWPRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 300 - protein, 131 aa, synthetic construct
EVQLVESGGGLVKPGGSLRLTCVASGFTFSDVWLNWVRQAPGKGLEWVGRIKSRTDGGTTDYAASVKGRFTISRDDSKNTLYLQMNSLKTEDTAVYSCTTDGFIMIRGVSEDYYYYYMDVWGKGTTVTVSS

SEQ ID NO: 301 - protein, 111 aa, synthetic construct
QSVLTQPPSVSAAPGQKVTISCSGSSSNIGNNYVLWYQQFPGTAPKLLIYGNNKRPSGIPDRFSGSKSGTSATLGITGLQTGDEADYFCATWDSGLSADWVFGGGTKLTVL

SEQ ID NO: 302 - protein, 461 aa, synthetic construct
EVQLVESGGGLVKPGGSLRLTCVASGFTFSDVWLNWVRQAPGKGLEWVGRIKSRTDGGTTDYAASVKGRFTISRDDSKNTLYLQMNSLKTEDTAVYSCTTDGFIMIRGVSEDYYYYYMDVWGKGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 303 - protein, 461 aa, synthetic construct
EVQLVESGGGLVKPGGSLRLTCVASGFTFSDVWLNWVRQAPGKGLEWVGRIKSRTDGGTTDYAASVKGRFTISRDDSKNTLYLQMNSLKTEDTAVYSCTTDGFIMIRGVSEDYYYYYMDVWGKGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 304 - protein, 217 aa, synthetic construct
QSVLTQPPSVSAAPGQKVTISCSGSSSNIGNNYVLWYQQFPGTAPKLLIYGNNKRPSGIPDRFSGSKSGTSATLGITGLQTGDEADYFCATWDSGLSADWVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 305 - protein, 123 aa, synthetic construct
QVQLVQSGGGLVQPGGSLRLSCAAFGFNFSSYVMHWVRQAPGQGLEYLSAISSDGETTYHANSVKGRFTSSRDNSKNTLFLQMGSLRTEDVAVYYCARDRYYETSGSNAFDVWGQGTMVVVSS

SEQ ID NO: 306 - protein, 108 aa, synthetic construct
NSVLTQSPSSLSASVGDRVTITCQASQDISNYLNWYQHKPGKAPKLLIYTASNLETGVPSRFSGGGSGTHFSFTITSLQPEDAATYFCQQYDNLGDLSFGGGTKVEIK

SEQ ID NO: 307 - protein, 453 aa, synthetic construct
QVQLVQSGGGLVQPGGSLRLSCAAFGFNFSSYVMHWVRQAPGQGLEYLSAISSDGETTYHANSVKGRFTSSRDNSKNTLFLQMGSLRTEDVAVYYCARDRYYETSGSNAFDVWGQGTMVVVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 308 - protein, 453 aa, synthetic construct
QVQLVQSGGGLVQPGGSLRLSCAAFGFNFSSYVMHWVRQAPGQGLEYLSAISSDGETTYHANSVKGRFTSSRDNSKNTLFLQMGSLRTEDVAVYYCARDRYYETSGSNAFDVWGQGTMVVVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 309 - protein, 215 aa, synthetic construct
NSVLTQSPSSLSASVGDRVTITCQASQDISNYLNWYQHKPGKAPKLLIYTASNLETGVPSRFSGGGSGTHFSFTITSLQPEDAATYFCQQYDNLGDLSFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 310 - protein, 130 aa, synthetic construct
QVQLVQSGAEVRKPGSSVTISCKPVGGTFTNFAIHWVRQAPGQGLEWVGGRVPVVGIYKYGKKFHDRLRLYEDDPMKTVFLELRSLTSDDTGVYYCTRWRGCGMCPYDTSSYYNDASDVWGPGTKVIVSA

SEQ ID NO: 311 - protein, 108 aa, synthetic construct
EIVLTQSPVTLSLSSGETGTLSCRASQNISSSWIAWYQQRRGQVPRLLISAASARAAGIPDRFTGRGSGTDFTLTITRLEPEDFGVYSCQYYGGSFFTFGPGTQVDVK

SEQ ID NO: 312 - protein, 460 aa, synthetic construct
QVQLVQSGAEVRKPGSSVTISCKPVGGTFTNFAIHWVRQAPGQGLEWVGGRVPVVGIYKYGKKFHDRLRLYEDDPMKTVFLELRSLTSDDTGVYYCTRWRGCGMCPYDTSSYYNDASDVWGPGTKVIVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 313 - protein, 460 aa, synthetic construct
QVQLVQSGAEVRKPGSSVTISCKPVGGTFTNFAIHWVRQAPGQGLEWVGGRVPVVGIYKYGKKFHDRLRLYEDDPMKTVFLELRSLTSDDTGVYYCTRWRGCGMCPYDTSSYYNDASDVWGPGTKVIVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 314 - protein, 215 aa, synthetic construct
EIVLTQSPVTLSLSSGETGTLSCRASQNISSSWIAWYQQRRGQVPRLLISAASARAAGIPDRFTGRGSGTDFTLTITRLEPEDFGVYSCQYYGGSFFTFGPGTQVDVKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 315 - protein, 123 aa, synthetic construct
EVQLVESGGGLVKAGGSLILSCGVSNFRISAHTMNWVRRVPGGGLEWVASISTSSTYRDYADAVKGRFTVSRDDLEDFVYLQMHKMRVEDTAIYYCARKGSDRLSDNDPFDAWGPGTVVTVSP

SEQ ID NO: 316 - protein, 107 aa, synthetic construct
DVVMTQSPSTLSASVGDTITITCRASQSIETWLAWYQQKPGKAPKLLIYKASTLKTGVPSRFSGSGSGTEFTLTISGLQFDDFATYHCQHYAGYSATFGQGTRVEIK

SEQ ID NO: 317 - protein, 453 aa, synthetic construct
EVQLVESGGGLVKAGGSLILSCGVSNFRISAHTMNWVRRVPGGGLEWVASISTSSTYRDYADAVKGRFTVSRDDLEDFVYLQMHKMRVEDTAIYYCARKGSDRLSDNDPFDAWGPGTVVTVSPASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 318 - protein, 453 aa, synthetic construct
EVQLVESGGGLVKAGGSLILSCGVSNFRISAHTMNWVRRVPGGGLEWVASISTSSTYRDYADAVKGRFTVSRDDLEDFVYLQMHKMRVEDTAIYYCARKGSDRLSDNDPFDAWGPGTVVTVSPASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 319 - protein, 214 aa, synthetic construct
DVVMTQSPSTLSASVGDTITITCRASQSIETWLAWYQQKPGKAPKLLIYKASTLKTGVPSRFSGSGSGTEFTLTISGLQFDDFATYHCQHYAGYSATFGQGTRVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 320 - protein, 132 aa, synthetic construct
QMQLQESGPGLVKPGETLSLTCSVSGASISSSYWSWLRETPGKGLEWIGYTHHSGDTNYAPSLKSRVHLGLHPSKNQVSLSLTSVTAADTAVYYCARTLHGRRIYGVVAFNEFFTYFYWEVWGKGTQVTVSS

SEQ ID NO: 321 - protein, 105 aa, synthetic construct
SDISVAPGETVRISCGGESIGSRAVQWYQHRAGQAPKLIIYNNQDRPPGIPERFSGSPDIDFGTTATLTITNVEAGDEATYYCHIWDSRRPTNWVFGGGTTLTVL

SEQ ID NO: 322 - protein, 462 aa, synthetic construct
QMQLQESGPGLVKPGETLSLTCSVSGASISSSYWSWLRETPGKGLEWIGYTHHSGDTNYAPSLKSRVHLGLHPSKNQVSLSLTSVTAADTAVYYCARTLHGRRIYGVVAFNEFFTYFYWEVWGKGTQVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 323 - protein, 462 aa, synthetic construct
QMQLQESGPGLVKPGETLSLTCSVSGASISSSYWSWLRETPGKGLEWIGYTHHSGDTNYAPSLKSRVHLGLHPSKNQVSLSLTSVTAADTAVYYCARTLHGRRIYGVVAFNEFFTYFYWEVWGKGTQVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 324 - protein, 211 aa, synthetic construct
SDISVAPGETVRISCGGESIGSRAVQWYQHRAGQAPKLIIYNNQDRPPGIPERFSGSPDIDFGTTATLTITNVEAGDEATYYCHIWDSRRPTNWVFGGGTTLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 325 - protein, 132 aa, synthetic construct
QMQLQESGPGLVKPGETLSLTCSVSGASISSSYWSWLRETPGKGLEWIGYTHHSGDTNYAPSLKSRVHLGLHPSKNQVSLSLTSVTAADTAVYYCARTLHGRRIYGVVAFNEYYTYFYWPTWGKGTQVTVSS

SEQ ID NO: 326 - protein, 105 aa, synthetic construct
SDISVAPGETVRITCGGESIGSRAVQWYQHRPGQAPRLIIYNNQDRPPGIPERFSGSPDIDFGTTATLTISNVEAGDEATYYCHIWDSRRPTNWELGPGTTLTVL

SEQ ID NO: 327 - protein, 462 aa, synthetic construct
QMQLQESGPGLVKPGETLSLTCSVSGASISSSYWSWLRETPGKGLEWIGYTHHSGDTNYAPSLKSRVHLGLHPSKNQVSLSLTSVTAADTAVYYCARTLHGRRIYGVVAFNEYYTYFYWPTWGKGTQVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 328 - protein, 462 aa, synthetic construct
QMQLQESGPGLVKPGETLSLTCSVSGASISSSYWSWLRETPGKGLEWIGYTHHSGDTNYAPSLKSRVHLGLHPSKNQVSLSLTSVTAADTAVYYCARTLHGRRIYGVVAFNEYYTYFYWPTWGKGTQVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 329 - protein, 211 aa, synthetic construct
SDISVAPGETVRITCGGESIGSRAVQWYQHRPGQAPRLIIYNNQDRPPGIPERFSGSPDIDFGTTATLTISNVEAGDEATYYCHIWDSRRPTNWELGPGTTLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 330 - protein, 132 aa, synthetic construct
QMQLQESGPGLVKPGETLSLTCSVSGASISSSYWSWLRETPGKGLEWIGYTHHSGDTNYAPSLKSRVTIGLDPSKNQVSLSLTSVTAADTAVYYCARTLHGRRIYGVVAFNEYYTYFYWPTWGKGTQVTVSS

SEQ ID NO: 331 - protein, 462 aa, synthetic construct
QMQLQESGPGLVKPGETLSLTCSVSGASISSSYWSWLRETPGKGLEWIGYTHHSGDTNYAPSLKSRVTIGLDPSKNQVSLSLTSVTAADTAVYYCARTLHGRRIYGVVAFNEYYTYFYWPTWGKGTQVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 332 - protein, 462 aa, synthetic construct
QMQLQESGPGLVKPGETLSLTCSVSGASISSSYWSWLRETPGKGLEWIGYTHHSGDTNYAPSLKSRVTIGLDPSKNQVSLSLTSVTAADTAVYYCARTLHGRRIYGVVAFNEYYTYFYWPTWGKGTQVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 333 - protein, 132 aa, synthetic construct; identical to SEQ ID NO: 330

SEQ ID NO: 334 - protein, 110 aa, synthetic construct
QSVLTQPPSASGSPGQSVTISCTGTSSDIGASDYVSWYQQYPGEAPKVIIYDVTKRPSGVPDRFSGSKSGTTASLTVSGLQAEDEADYYCSSDAGRHTLLFGGGTKVTVL

SEQ ID NO: 335 - protein, 451 aa, synthetic construct
QVQLLESGPGLVRPSETLTLTCSVFNSRVSGYYYSWIRQPPGRGLEWIASTHFSLRPSRNPSLLSRVTTSIDTERYQVFLNMRSVTAADTAVYFCARGDASGWRADYFPHWGQGTLVVVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 336 - protein, 451 aa, synthetic construct
QVQLLESGPGLVRPSETLTLTCSVFNSRVSGYYYSWIRQPPGRGLEWIASTHFSLRPSRNPSLLSRVTTSIDTERYQVFLNMRSVTAADTAVYFCARGDASGWRADYFPHWGQGTLVVVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 337 - protein, 216 aa, synthetic construct
QSVLTQPPSASGSPGQSVTISCTGTSSDIGASDYVSWYQQYPGEAPKVIIYDVTKRPSGVPDRFSGSKSGTTASLTVSGLQAEDEADYYCSSDAGRHTLLFGGGTKVTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 338 - protein, 566 aa, synthetic construct
KKVVYGKCGDTVELTCTASQKKNIQFHWKNSNQIKILGNQGSFLTKGPSKLNDRVDSRRSLWDQGNFPLIIKNLKPEDSDTYICEVEDQKEEVQLVVVCGGGGSQVHLQESGPGLVKPSETLSLTCNVSGTLVRDNYWSWIRQPLGKQPEWIGYVHDSGDTNYNPSLKSRVHLSLDKSKNLVSLRLTGVTAADSAIYYCATTKHGRRIYGVVAFKEWFTYFYMDVWGKGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 339 - protein, 566 aa, synthetic construct
KKVVYGKCGDTVELTCTASQKKNIQFHWKNSNQIKILGNQGSFLTKGPSKLNDRVDSRRSLWDQGNFPLIIKNLKPEDSDTYICEVEDQKEEVQLVVVCGGGGSQLHLQESGPGLVKPPETLSLTCSVSGASINDAYWSWIRQSPGKRPEWVGYVHHSGDTNYNPSLKRRVTFSLDTAKNEVSLKLVDLTAADSATYFCARALHGKRIYGIVALGELFTYFYMDVWGKGTAVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 340 - protein, 571 aa, synthetic construct
KKVVYGKCGDTVELTCTASQKKNIQFHWKNSNQIKILGNQGSFLTKGPSKLNDRVDSRRSLWDQGNFPLIIKNLKPEDSDTYICEVEDQKEEVQLVVVCGGGGSQSQLQESGPRLVEASETLSLTCNVSGESTGACTYFWGWVRQAPGKGLEWIGSLSHCQSFWGSGWTFHNPSLKSRLTISLDTPKNQVFLKLTSLTAADTATYYCARFDGEVLVYNHWPKPAWVDLWGRGIPVTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 341 - protein, 571 aa, synthetic construct
KKVVYGKCGDTVELTCTASQKKNIQFHWKNSNQIKILGNQGSFLTKGPSKLNDRVDSRRSLWDQGNFPLIIKNLKPEDSDTYICEVEDQKEEVQLVVVCGGGGSQPQLQESGPGLVEASETLSLTCTVSGDSTAACDYFWGWVRQPPGKGLEWIGGLSHCAGYYNTGWTYHNPSLKSRLTISLDTPKNQVFLKLNSVTAADTAIYYCARFDGEVLVYHDWPKPAWVDLWGRGTLVTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 342 - protein, 569 aa, synthetic construct
KKVVYGKCGDTVELTCTASQKKNIQFHWKNSNQIKILGNQGSFLTKGPSKLNDRVDSRRSLWDQGNFPLIIKNLKPEDSDTYICEVEDQKEEVQLVVVCGGGGSQPQLQESGPGLVEASETLSLTCTVSGDSTGRCNYFWGWVRQPPGKGLEWIGSLSHCRSYYNTDWTYHNPSLKSRLTISLDTPKNQVFLRLTSVTAADTATYYCARFGGEVLVYRDWPKPAWVDLWGRGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 343 - protein, 563 aa, synthetic construct
KKVVYGKCGDTVELTCTASQKKNIQFHWKNSNQIKILGNQGSFLTKGPSKLNDRVDSRRSLWDQGNFPLIIKNLKPEDSDTYICEVEDQKEEVQLVVVCGGGGSQVQLQESGPGLVKPAETLSLTCSVSGESINTGHYYWGWVRQVPGKGLEWIGHIHYTTAVLHNPSLKSRLTIKIYTLRNQITLRLSNVTAADTAVYHCVRSGGDILYYYEWQKPHWFSPWGPGIHVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 344 - protein, 563 aa, synthetic construct
QVQLQESGPGLVKPAETLSLTCSVSGESINTGHYYWGWVRQVPGKGLEWIGHIHYTTAVLHNPSLKSRLTIKIYTLRNQITLRLSNVTAADTAVYHCVRSGGDILYYYEWQKPHWFSPWGPGIHVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGKGGGGSKKVVYGKCGDTVELTCTASQKKNIQFHWKNSNQIKILGNQGSFLTKGPSKLNDRVDSRRSLWDQGNFPLIIKNLKPEDSDTYICEVEDQKEEVQLVVVC

SEQ ID NO: 345 - protein, 320 aa, synthetic construct
KKVVYGKCGDTVELTCTASQKKNIQFHWKNSNQIKILGNQGSFLTKGPSKLNDRVDSRRSLWDQGNFPLIIKNLKPEDSDTYICEVEDQKEEVQLVVVCGGGGSQSALTQPPSASGSLGQSVTISCNGTSSDIGGWNFVSWYQQFPGRAPRLIIFEVNKRPSGVPGRFSGSKSGNSASLTVSGLQSDDEGQYFCSSLFGRWDVVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 346 - protein, 320 aa, synthetic construct
QSALTQPPSASGSLGQSVTISCNGTSSDIGGWNFVSWYQQFPGRAPRLIIFEVNKRPSGVPGRFSGSKSGNSASLTVSGLQSDDEGQYFCSSLFGRWDVVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECSGGGGSKKVVYGKCGDTVELTCTASQKKNIQFHWKNSNQIKILGNQGSFLTKGPSKLNDRVDSRRSLWDQGNFPLIIKNLKPEDSDTYICEVEDQKEEVQLVVVC

SEQ ID NO: 347 - protein, 563 aa, synthetic construct
KKVVYGKCGDTVELTCTASQKKNIQFHWKNSNQIKILGNQGSFLTKGPSKLNDRVDSRRSLWDQGNFPLIIKNLKPEDSDTYICEVEDQKEEVQLVVVCGGGGSQVQLQESGPGLVKPSETLSLTCTVSGDSINTGHHYWGWVRQVPGKGPEWIAHIHYNTAVLHNPALKSRVTISIFTLKNLITLSLSNVTAADTAVYFCVRSGGDILYYIEWQKPHWFYPWGPGILVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 348 - protein, 568 aa, synthetic construct
KKVVYGKCGDTVELTCTASQKKNIQFHWKNSNQIKILGNQGSFLTKGPSKLNDRVDSRRSLWDQGNFPLIIKNLKPEDSDTYICEVEDQKEEVQLVVVCGGGGSQLQLQESGPGLVKPSETLSLTCTVSGGSMRGTDWGENDFHYGWIRQSSAKGLEWIGSIHWRGRTTHYKTSFRSRATLSIDTSNNRFSLTFSFVTAADTAVYYCARHKYHDIFRVVPVAGWFDPWGQGLLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 349 - protein, 567 aa, synthetic construct
KKVVYGKCGDTVELTCTASQKKNIQFHWKNSNQIKILGNQGSFLTKGPSKLNDRVDSRRSLWDQGNFPLIIKNLKPEDSDTYICEVEDQKEEVQLVVVCGGGGSEVHLEESGPGLVRPSETLSLTCTASGGSIRGGEWGDSDYHWGWVRHSPEKGLEWIGSIHWRGTTHYNAPFRGRGRLSIDLSRNQFSLRLTSVTAEDTAVYYCVKHKYHDIVMVVPIAGWFDPWGQGLQVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 350 - protein, 565 aa, synthetic construct
KKVVYGKCGDTVELTCTASQKKNIQFHWKNSNQIKILGNQGSFLTKGPSKLNDRVDSRRSLWDQGNFPLIIKNLKPEDSDTYICEVEDQKEEVQLVVVCGGGGSEVQLVESGGGLVKPGGSLRLTCVASGFTFSDVWLNWVRQAPGKGLEWVGRIKSRTDGGTTDYAASVKGRFTISRDDSKNTLYLQMNSLKTEDTAVYSCTTDGFIMIRGVSEDYYYYYMDVWGKGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 351 - protein, 557 aa, synthetic construct
KKVVYGKCGDTVELTCTASQKKNIQFHWKNSNQIKILGNQGSFLTKGPSKLNDRVDSRRSLWDQGNFPLIIKNLKPEDSDTYICEVEDQKEEVQLVVVCGGGGSQVQLVQSGGGLVQPGGSLRLSCAAFGFNFSSYVMHWVRQAPGQGLEYLSAISSDGETTYHANSVKGRFTSSRDNSKNTLFLQMGSLRTEDVAVYYCARDRYYETSGSNAFDVWGQGTMVVVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 352 - protein, 564 aa, synthetic construct
KKVVYGKCGDTVELTCTASQKKNIQFHWKNSNQIKILGNQGSFLTKGPSKLNDRVDSRRSLWDQGNFPLIIKNLKPEDSDTYICEVEDQKEEVQLVVVCGGGGSQVQLVQSGAEVRKPGSSVTISCKPVGGTFTNFAIHWVRQAPGQGLEWVGGRVPVVGIYKYGKKFHDRLRLYEDDPMKTVFLELRSLTSDDTGVYYCTRWRGCGMCPYDTSSYYNDASDVWGPGTKVIVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 353 - protein, 557 aa, synthetic construct
KKVVYGKCGDTVELTCTASQKKNIQFHWKNSNQIKILGNQGSFLTKGPSKLNDRVDSRRSLWDQGNFPLIIKNLKPEDSDTYICEVEDQKEEVQLVVVCGGGGSEVQLVESGGGLVKAGGSLILSCGVSNFRISAHTMNWVRRVPGGGLEWVASISTSSTYRDYADAVKGRFTVSRDDLEDFVYLQMHKMRVEDTAIYYCARKGSDRLSDNDPFDAWGPGTVVTVSPASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 354 - protein, 566 aa, synthetic construct
KKVVYGKCGDTVELTCTASQKKNIQFHWKNSNQIKILGNQGSFLTKGPSKLNDRVDSRRSLWDQGNFPLIIKNLKPEDSDTYICEVEDQKEEVQLVVVCGGGGSQMQLQESGPGLVKPGETLSLTCSVSGASISSSYWSWLRETPGKGLEWIGYTHHSGDTNYAPSLKSRVHLGLHPSKNQVSLSLTSVTAADTAVYYCARTLHGRRIYGVVAFNEFFTYFYWEVWGKGTQVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 355 - protein, 566 aa, synthetic construct
KKVVYGKCGDTVELTCTASQKKNIQFHWKNSNQIKILGNQGSFLTKGPSKLNDRVDSRRSLWDQGNFPLIIKNLKPEDSDTYICEVEDQKEEVQLVVVCGGGGSQMQLQESGPGLVKPGETLSLTCSVSGASISSSYWSWLRETPGKGLEWIGYTHHSGDTNYAPSLKSRVHLGLHPSKNQVSLSLTSVTAADTAVYYCARTLHGRRIYGVVAFNEYYTYFYWPTWGKGTQVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 356 - protein, 566 aa, synthetic construct
KKVVYGKCGDTVELTCTASQKKNIQFHWKNSNQIKILGNQGSFLTKGPSKLNDRVDSRRSLWDQGNFPLIIKNLKPEDSDTYICEVEDQKEEVQLVVVCGGGGSQMQLQESGPGLVKPGETLSLTCSVSGASISSSYWSWLRETPGKGLEWIGYTHHSGDTNYAPSLKSRVTIGLDPSKNQVSLSLTSVTAADTAVYYCARTLHGRRIYGVVAFNEYYTYFYWPTWGKGTQVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 357 - protein, 555 aa, synthetic construct
KKVVYGKCGDTVELTCTASQKKNIQFHWKNSNQIKILGNQGSFLTKGPSKLNDRVDSRRSLWDQGNFPLIIKNLKPEDSDTYICEVEDQKEEVQLVVVCGGGGSQVQLLESGPGLVRPSETLTLTCSVFNSRVSGYYYSWIRQPPGRGLEWIASTHFSLRPSRNPSLLSRVTTSIDTERYQVFLNMRSVTAADTAVYFCARGDASGWRADYFPHWGQGTLVVVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 358 - protein, 320 aa, synthetic construct
KKVVYGKCGDTVELTCTASQKKNIQFHWKNSNQIKILGNQGSFLTKGPSKLNDRVDSRRSLWDQGNFPLIIKNLKPEDSDTYICEVEDQKEEVQLVVVCGGGGSQSVLTQPPSASGSPGQSVTISCTGTSSDIGASDYVSWYQQYPGEAPKVIIYDVTKRPSGVPDRFSGSKSGTTASLTVSGLQAEDEADYYCSSDAGRHTLLFGGGTKVTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 359 - protein, 555 aa, synthetic construct
QVQLLESGPGLVRPSETLTLTCSVFNSRVSGYYYSWIRQPPGRGLEWIASTHFSLRPSRNPSLLSRVTTSIDTERYQVFLNMRSVTAADTAVYFCARGDASGWRADYFPHWGQGTLVVVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGKGGGGSKKVVYGKCGDTVELTCTASQKKNIQFHWKNSNQIKILGNQGSFLTKGPSKLNDRVDSRRSLWDQGNFPLIIKNLKPEDSDTYICEVEDQKEEVQLVVVC

SEQ ID NO: 360 - protein, 320 aa, synthetic construct
QSVLTQPPSASGSPGQSVTISCTGTSSDIGASDYVSWYQQYPGEAPKVIIYDVTKRPSGVPDRFSGSKSGTTASLTVSGLQAEDEADYYCSSDAGRHTLLFGGGTKVTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECSGGGGSKKVVYGKCGDTVELTCTASQKKNIQFHWKNSNQIKILGNQGSFLTKGPSKLNDRVDSRRSLWDQGNFPLIIKNLKPEDSDTYICEVEDQKEEVQLVVVC

SEQ ID NO: 361 - protein, 35 aa, Human Immunodeficiency Virus 1
CTRPNNNTRKSIHIGPGRAFYTTGEIIGDIRQAHC

SEQ ID NO: 362 - protein, 561 aa, synthetic construct
KKVVYGKCGDTVELTCTASQKKNIQFHWKNSNQIKILGNQGSFLTKGPSKLNDRVDSRRSLWDQGNFPLIIKNLKPEDSDTYICEVEDQKEEVQLVVVCGGGGSQVQLVQSGAQMKNPGASVKVSCAPSGYTFTDFYIHWLRQAPGQGLQWMGWMNPQTGRTNTARNFQGRVTMTRDTSIGTAYMELRSLTSDDTAIYYCTTGGWISLYYDSSYYPNFDHWGQGTLLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 363 - protein, 849 aa, Human Immunodeficiency Virus 1
MKVMGTKKNYQHLWRWGIMLLGMLMMSSAAEQLWVTVYYGVPVWREANTTLFCASDAKAYDTEVHNVWATHACVPTDPNPQEVVMGNVTEDFNMWKNNMVEQMHEDIISLWDQSLKPCVKLTPLCVTLHCTNVTISSTNGSTANVTMREEMKNCSFNTTTVIRDKIQKEYALFYKLDIVPIEGKNTNTSYRLINCNTSVITQACPKVSFEPIPIHYCAPAGFAILKCNNKTFNGKGPCRNVSTVQCTHGIKPVVSTQLLLNGSLAEEDIIIRSENFTNNGKNIIVQLKEPVKINCTRPGNNTRRSINIGPGRAFYATGAIIGDIRKAHCNISTEQWNNTLTQIVDKLREQFGNKTIIFNQSSGGDPEVVMHTFNCGGEFFYCNSTQLFNSTWFNNGTSTWNSTADNITLPCRIKQVINMWQEVGKAMYAPPIRGQIDCSSNITGLILTRDGGSNSSQNETFRPGGGNMKDNWRSELYKYKVVKIEPLGIAPTRAKRRVVQREKRAVTLGAVFLGFLGAAGSTMGAASLTLTVQARLLLSGIVQQQSNLLRAIEAQQHMLQLTVWGIKQLQARVLAIERYLKDQQLLGIWGCSGKLICTTTVPWNTSWSNKSYDYIWNNMTWMQWEREIDNYTGFIYTLIEESQNQQEKNELELLELDKWASLWNWFNITNWLWYIKLFIMIIGGLVGLRIVCAVLSIVNRVRQGYSPLSFQTRLPNPRGPDRPEETEGEGGERDRDRSARLVNGFLAIIWDDLRSLCLFSYHRLRDLLLIVARVVEILGRRGWEILKYWWNLLKYWSQELKNSAVSLLNVTAIAVAEGTDRVIEIVQRAVRAILHIPTRIRQGFERALL

SEQ ID NO: 364 - protein, 475 aa, Human Immunodeficiency Virus 1
AEQLWVIVYYGVPVWREANTTLFCASDAKAYDTEVHNVWATHACVPTDPNPQEVVMGNVTEDFNMWKNNMVEQMHEDIISLWDQSLKPCVKLTPLCVTLHCTNVTISSTNGSTANVTMREEMKNCSFNTTTVIRDKIQKEYALFYKLDIVPIEGKNTNTSYRLINCNTSVITQACPKVSFEPIPIHYCAPAGFAILKCNNKTFNGKGPCRNVSTVQCTHGIKPVVSTQLLLNGSLAEEDIIIRSENFTNNGKNIIVQLKEPVKINCTRPGNNTRRSINIGPGRAFYATGAIIGDIRKAHCNISTEQWNNTLTQIVDKLREQFGNKTIIFNQSSGGDPEVVMHTFNCGGEFFYCNSTQLFNSTWFNNGTSTWNSTADNITLPCRIKQVINMWQEVGKAMYAPPIRGQIDCSSNITGLILTRDGGSNSSQNETFRPGGGNMKDNWRSELYKYKVVKIEPLGIAPTRAKRRVVQREKR

SEQ ID NO: 365 - protein, 105 aa, synthetic construct; identical to SEQ ID NO: 326

SEQ ID NO: 366 - protein, 211 aa, synthetic construct; identical to SEQ ID NO: 329

(19) United States
(12) Patent Application Publication
(10) Pub. No.: US 2025/0250328 A1
(43) Pub. Date: Aug. 7, 2025

(54) Antigen Binding Proteins

(71) Applicant: ViiV Healthcare UK (No.5) Limited, Stevenage (GB)

(72) Inventors: Eric ARNOULT, Branford, CT (US); Tiancen HU, Branford, CT (US); Mark R. KRYSTAL, Branford, CT (US); James SCHAWALDER, Durham, NC (US)

(73) Assignee: ViiV Healthcare UK (No.5) Limited, Stevenage (GB)

(21) Appl. No.: 19/086,720

(22) Filed: Mar. 21, 2025

Related U.S. Application Data
(63) Continuation of application No. 18/498,114, filed on Oct. 31, 2023.
(60) Provisional application No. 63/421,737, filed on Nov. 2, 2022.

(51) Int. Cl. C07K 16/10; A61K 39/00; A61P 31/18; C12N 15/63

(52) U.S. Cl. CPC C07K 16/1063; A61P 31/18; C12N 15/63; A61K 2039/505; C07K 2317/14

(57) ABSTRACT

Antigen binding proteins of the invention bind to Human Immunodeficiency Virus (HIV) envelope protein and are useful in treating and preventing HIV infection. In particular, the antigen binding proteins bind to two different epitopes on HIV envelope surface glycoprotein 120 (gp120): the V3 loop region and the CD4 binding site.

### CROSS-REFERENCE TO RELATED APPLICATIONS

This application is a Continuation of U.S. application Ser. No. 18/498,114, filed on Oct. 31, 2023, which claims the benefit under 35 U.S.C. § 119 (b) of U.S. Provisional Application No. 63/421,737, filed on Nov. 2, 2022; the contents of each of the above-identified applications are hereby incorporated by reference in their entireties.

### SEQUENCE LISTING

The instant application contains a Sequence Listing, which has been submitted electronically in XML format and is hereby incorporated by reference in its entirety. Said XML formatted copy, which was created on Oct. 5, 2023, is named “70263WO01.xml” and is 398,770 bytes in size.

### FIELD OF THE INVENTION

The invention is directed to an antigen binding protein that binds to the Human Immunodeficiency Virus (HIV) envelope and its use in treating or preventing HIV infection. The antigen binding protein of the invention binds to at least two different epitopes on the HIV envelope protein, in particular the V3 loop region (V3/glycan) and the CD4 binding site (CD4bs) of HIV envelope surface glycoprotein 120 (gp120).

### BACKGROUND TO THE INVENTION

HIV, the virus that over time may result in Acquired Immunodeficiency Syndrome (AIDS), continues to be a serious public health challenge and has claimed 40.1 million lives so far. HIV attacks the body's immune system, targeting CD4-positive white blood cells, and leaves those infected vulnerable to opportunistic infections such as tuberculosis and fungal infections, severe bacterial infections and some cancers. Globally, 38.4 million people were living with HIV at the end of 2021, with 1.5 million people becoming newly infected (WHO, Key Facts HIV, July 2022).

Whilst there is currently no cure for HIV infection, it can be treated with antiretroviral therapy (ART), which includes a number of different types of drugs that prevent the virus from multiplying (nucleoside reverse transcriptase inhibitors (NRTIs), non-nucleoside reverse transcriptase inhibitors (NNRTIs), protease inhibitors, entry inhibitors and integrase inhibitors), allowing the body's immune system to recover sufficiently for the infected patient to be asymptomatic. 75% of people living with HIV in 2021 received some form of ART. However, ART often requires taking medication every day for life and has the risk of serious and debilitating side effects. Further, increased use of ART has also been accompanied by the emergence of drug resistance, the levels of which have steadily increased in recent years.

Broadly neutralizing antibodies (bNAbs) could potentially provide longer-term HIV suppression, but individual bNAbs have only had limited success in previous studies. This is in part because antibody-resistant virus either already existed in the patient or emerged soon after treatment began (NIH Research Matters, 14 Jun. 2022). Combinations of bNAbs are currently being investigated in the presence or absence of ART (Nature, 606, 368-374, 2022).

Further treatment options are needed for HIV infection, in particular drugs that are long-acting and effective against a wide spectrum of HIV strains so that patients taking them are less susceptible to drug resistance.

### SUMMARY OF THE INVENTION

In a first aspect of the invention, an anti-HIV gp120-binding protein that binds to at least two different epitopes on human immunodeficiency virus (HIV) surface glycoprotein 120 (gp120) is provided.

In a second aspect of the invention, a bispecific anti-HIV gp120-binding protein comprising an anti-V3 bNAb and two copies of a CD4 domain is provided, wherein the C-terminus of one CD4 domain is attached directly or by a linker to the N-terminus of one of the anti-V3 bNAb heavy chains and the C-terminus of the other copy of the CD4 domain is attached directly or by a linker to the N-terminus of the other anti-V3 bNAb heavy chain.

In another aspect of the invention, a bispecific molecule that binds to human immunodeficiency virus (HIV)glycoprotein gp120 and human CD4 is provided, wherein the bispecific molecule comprises: (i) a first antigen-binding domain comprising an anti-V3 bNAb; (ii) a second antigen-binding domain comprising a CD4 domain, and (iii) a third antigen-binding domain comprising a CD4 domain.

In a third aspect of the invention, an anti-HIV gp120-binding protein having two identical heavy chains and two identical light chains is provided, comprising or consisting of: a heavy chain that is at least 95% identical to SEQ ID NO: 121 and a light chain that is at least 95% identical to SEQ ID NO:63.

In a fourth aspect of the invention, an anti-HIV gp120-binding protein consisting of two identical heavy chains of SEQ ID NO: 121 and two identical light chains of SEQ ID NO:63 is provided.

In a fifth aspect of the invention, an anti-HIV gp120-binding protein comprising or consisting of a sequence that is at least 95% identical to any one of SEQ ID NOs: 152-157 is provided.

In a sixth aspect of the invention, an anti-HIV gp120-binding protein consisting of SEQ ID NO: 155 is provided.

In further aspects of the invention, pharmaceutical compositions comprising anti-HIV gp120-binding proteins of the invention, methods of preventing HIV infection and methods of treating HIV infection with anti-HIV gp120-binding proteins of the invention, uses of anti-HIV gp120-binding proteins of the invention, methods of manufacturing anti-HIV gp120-binding proteins of the invention and kits comprising anti-HIV gp120-binding proteins of the invention are also provided.

### DESCRIPTION OF DRAWINGS/FIGURES

FIGS. 1A-1D show schematic designs of bispecific molecules of the invention. Human CD4 domains or variants thereof are fused, either directly or via linkers, to the N-termini of either the heavy chains (FIG. 1A), the light chains (FIG. 1B) or both chains (FIG. 1C) of anti-V3 bNAbs. Such designs facilitate concomitant binding of the human CD4 domain of the bispecific molecule and the V3 glycan binding domain of the bispecific molecule to HIV-1 gp120 and prevent HIV-1 virions from binding to and fusing with the cell membrane (FIG. 1D).

FIG. 2 shows IC50 values (nM) of soluble CD4 domains (SEQ ID NOs: 4-15) against a panel of HIV-1 envelopes in a PSV assay (ACTOne), together with the Tm for each soluble CD4 domain. The horizontal bars indicate geometric mean IC50.

FIGS. 3A-3B show that linker length between the CD4 domain and bNAb1 heavy chain N-terminus does not particularly affect anti-viral activity in a PSV assay (ACTOne) (FIG. 3A) but does change the PK of the resultant bispecific molecules in a humanized mouse model (Tg32-hFcRn strain) (FIG. 3B). Thermal stability of the CD4 domain also affects the PK of the bispecific molecules (FIG. 3B).

FIGS. 4A-4B show IC50 values (nM) of two bispecific molecules (SEQ ID NO: 121 and SEQ ID NO: 63; and SEQ ID NO: 102 and SEQ ID NO: 63) of the invention having a variant human CD4 domain (D1m-K8C-G99C, SEQ ID NO: 11; and D1m, SEQ ID NO:4 respectively) fused to each of the heavy chain N-termini of bNAb1 via a GGGGS (1xG4S) (SEQ ID NO:90) linker, and control molecules, against a panel of HIV-1 envelopes in a PSV assay (ACTOne) (FIG. 4A) and a different panel of HIV-1 envelopes from bNAb1-resistant strains in a PSV assay (ACTOne) (FIG. 4B). Each dot represents one HIV envelope. The horizontal bars indicate geometric mean IC50.

FIG. 5 shows IC50 values (nM) of single ORF versions of the most potent bispecific format (i.e. fusing CD4 D1 to the N-terminus of bNAb1 heavy chain), also referred to as scFv-Fc molecules (SEQ ID NOs: 152-157), in PSV assays (ACTOne cells). The horizontal bars indicate geometric mean IC50.

FIG. 6 shows IC50 values (nM) of two bispecific molecules (SEQ ID NO: 151 and SEQ ID NO: 89; and SEQ ID NO: 150 and SEQ ID NO: 89) of the invention having variant human CD4 domains (D1m, SEQ ID NO:4 and D1mD2, SEQ ID NO:2, respectively) fused to each of the heavy chain N-termini of bNAb6 (SEQ ID NO: 88 and 89) via a 4xG4S linker (SEQ ID NO:93), and control molecules, against a panel of HIV-1 envelopes in a PSV assay (ACTOne). Each dot represents one HIV envelope. The horizontal bars indicate geometric mean IC50.

FIG. 7 shows IC50 values (nM) of different bNAb1-derived bispecific formats and control molecules against a panel of HIV-1 envelopes in a PSV assay (ACTone).

### DETAILED DESCRIPTION OF THE INVENTION

### Definitions

“Affinity”, also referred to as “binding affinity”, is the strength of binding at a single interaction site, i.e., of one molecule, e.g., an antigen binding protein, to another molecule, e.g., its target antigen, at a single binding site. The binding affinity of an antigen binding protein to its target may be determined by equilibrium methods (e.g., enzyme-linked immunoabsorbent assay (ELISA) or radioimmunoassay (RIA)), or kinetics (e.g., BIACORE analysis).

“Alternative antibody formats” include alternative scaffolds in which one or more CDRs of the antigen binding protein can be arranged onto a suitable non-immunoglobulin protein scaffold or skeleton, such as an affibody, a SpA scaffold, an LDL receptor class A domain, an avimer (see, e.g., U.S. Patent Application Publication Nos. 2005/0053973, 2005/0089932, 2005/0164301) or an EGF domain.

“Antibody” is used herein to refer to a heterotetrameric glycoprotein with an approximate molecular weight of 150,000 daltons. An intact antibody is composed of two identical heavy chains (HCs) and two identical light chains (LCs) linked by covalent disulphide bonds. This H2L2 structure folds to form a ‘Y’ shape with three functional domains comprising two antigen-binding fragments, known as ‘Fab’ fragments (the ‘top’ of the ‘Y’), and a fragment crystallisable ‘Fc’ (the ‘bottom’ of the ‘Y’). The Fab fragment is composed of the variable domain at the amino-terminus, variable heavy (VH) or variable light (VL), and the constant domain at the carboxyl terminus, CH1 (heavy) and CL (light). The Fc fragment is composed of two domains formed by dimerization of paired CH2 and CH3 regions. The Fc may elicit effector functions by binding to receptors on immune cells or by binding C1q, the first component of the classical complement pathway. The five classes of antibodies IgM, IgA, IgG, IgE and IgD are defined by distinct heavy chain amino acid sequences, which are called μ, α, γ, ε and δ respectively; each heavy chain can pair with either a K or λ light chain. The majority of antibodies in the serum belong to the IgG class, there are four isotypes of human IgG (IgG1, IgG2, IgG3 and IgG4), the sequences of which differ mainly in their hinge region. In an embodiment, an anti-CD4bs antibody, as used herein, refers to an antibody that binds to a CD4 binding site

“Antigen binding antibody fragments” or “antigen binding fragments” or “antibody fragments” as used herein include Fab, F(ab′)₂, Fv, disulphide linked Fv, single chain Fv (scFv), disulphide-linked scFv, diabodies, TANDABS, etc. and modified versions of any of the foregoing (for a summary of alternative “antibody” formats see Holliger and Hudson, Nature Biotechnology, 23 (9), 1126-1136, 2005).

“Antigen binding protein” and “anti-gp120 binding protein” are used interchangeably herein and refer to antibodies and fragments thereof, alternative antibody formats, and other protein constructs, such as domains, that are capable of binding to HIV gp120. Envelope glycoprotein gp120 (or gp120) is a 120 kDa glycoprotein that is part of the outer layer of HIV. It presents itself as viral membrane spikes consisting of three molecules of gp120 linked together and anchored to the membrane by gp41 protein. Gp120 is essential for viral infection as it facilitates HIV entry into the host cell through its interaction with cell surface receptors. Gp120 is encoded by the HIV env gene. The env gene encodes a gene product of around 850 amino acids. The primary env product is the protein gp160, which gets cleaved into gp120 (about 480 amino acids) and gp41 (about 345 amino acids) in the endoplasmic reticulum by the cellular protease furin. The amino acid sequence of an exemplary gp160 from HIV clone WITO is provided below (SEQ ID NO: 363; the V3 loop is boldened and the potential N332 N-linked glycosylation site is boldened and underlined):

MKVMGTKKNYQHLWRWGIMLLGMLMMSSAAEQLWVTVYYGVPVWR
EANTTLFCASDAKAYDTEVHNVWATHACVPTDPNPQEVVMGNVTE
DFNMWKNNMVEQMHEDIISLWDQSLKPCVKLTPLCVTLHCTNVTI
SSTNGSTANVTMREEMKNCSFNTTTVIRDKIQKEYALFYKLDIVP
IEGKNTNTSYRLINCNTSVITQACPKVSFEPIPIHYCAPAGFAIL
KCNNKTFNGKGPCRNVSTVQCTHGIKPVVSTQLLLNGSLAEEDII
IRSENFTNNGKNIIVQLKEPVKINCTRPGNNTRRSINIGPGRAFY
ATGAIIGDIRKAHCNISTEQWNNTLTQIVDKLREQFGNKTIIFNQ
SSGGDPEVVMHTFNCGGEFFYCNSTQLFNSTWENNGTSTWNSTAD
NITLPCRIKQVINMWQEVGKAMYAPPIRGQIDCSSNITGLILTRD
GGSNSSQNETFRPGGGNMKDNWRSELYKYKVVKIEPLGIAPTRAK
RRVVQREKRAVTLGAVFLGFLGAAGSTMGAASLTLTVQARLLLSG
IVQQQSNLLRAIEAQQHMLQLTVWGIKQLQARVLAIERYLKDQQL
LGIWGCSGKLICTTTVPWNTSWSNKSYDYIWNNMTWMQWEREIDN
YTGFIYTLIEESQNQQEKNELELLELDKWASLWNWFNITNWLWYI
KLFIMIIGGLVGLRIVCAVLSIVNRVRQGYSPLSFQTRLPNPRGP
DRPEETEGEGGERDRDRSARLVNGFLAIIWDDLRSLCLFSYHRLR
DLLLIVARVVEILGRRGWEILKYWWNLLKYWSQELKNSAVSLLNV
TAIAVAEGTDRVIEIVQRAVRAILHIPTRIRQGFERALL

The amino acid of an exemplary gp120 is provided below (SEQ ID NO: 364; the V3 loop is boldened and the potential N332 N-linked glycosylation site is boldened and underlined):

AEQLWVIVYYGVPVWREANTTLFCASDAKAYDTEVHNVWATHACV
PTDPNPQEVVMGNVTEDFNMWKNNMVEQMHEDIISLWDQSLKPCV
KLTPLCVTLHCTNVTISSTNGSTANVTMREEMKNCSFNTTTVIRD
KIQKEYALFYKLDIVPIEGKNTNTSYRLINCNTSVITQACPKVSF
EPIPIHYCAPAGFAILKCNNKTFNGKGPCRNVSTVQCTHGIKPVV
STQLLLNGSLAEEDIIIRSENFTNNGKNIIVQLKEPVKINCTRPG
NNTRRSINIGPGRAFYATGAIIGDIRKAHCNISTEQWNNTLTQIV
DKLREQFGNKTIIFNQSSGGDPEVVMHTFNCGGEFFYCNSTQLFN
STWENNGTSTWNSTADNITLPCRIKQVINMWQEVGKAMYAPPIRG
QIDCSSNITGLILTRDGGSNSSQNETFRPGGGNMKDNWRSELYKY
KVVKIEPLGIAPTRAKRRVVQREKR

“Antigen binding site” and “paratope” are used interchangeably herein and refer to a particular site on an antigen binding protein that makes contact with and is capable of specifically binding to a site (i.e., epitope) on an antigen, e.g., HIV gp120. The antigen binding site may be formed by a single variable domain, or paired VH/VL domains as can be found on a standard antibody. Single-chain Fv (ScFv) domains can also provide antigen binding sites.

“Avidity” also referred to as functional affinity, is the cumulative strength of binding at multiple interaction sites, e.g., the sum total of the strength of binding of two molecules (or more) to one another at multiple sites, e.g., taking into account the valency of the interaction.

A “bispecific molecule” as used herein is an antigen binding protein that is capable of binding to two different epitopes on the same antigen, i.e., HIV gp120 protein. In particular, one epitope comprises part of or the whole of the V3 loop region of gp120 and the other epitope comprises part of or the whole of the CD4 binding site of gp120.

“Broadly neutralizing antibody” or “bNAb” as used herein, is meant an antibody that neutralizes more than one HIV-1 virus species (from diverse clades and different strains within a clade) in a neutralization assay. A broad neutralizing antibody may neutralize at least 2, 3, 4, 5, 6, 7, 8, 9 or more different strains of HIV-1, the strains belonging to the same or different clades.

“CD4 binding site” or “CD4-binding site” or “CD4bs” refers to a site on the HIV envelope protein gp120 that binds to CD4. (Cluster of differentiation factor 4). CD4 is a T-cell surface protein that serves as the primary receptor site for HIV during HIV infection. The CD4 binding site on gp120 is a highly conserved, discontinuous and conformational that comprises residues on either side of the HIV V4 loop (Curr HIV/AIDS Rep, 9 (1): 52-63, 2021) that binds to CD4.

A “CD4 domain” as used herein is a soluble recombinant form of human CD4 (Cluster of differentiation factor 4, a transmembrane glycoprotein found on T-cells), or a fragment thereof, that mimics the activity of native membrane-anchored human CD4 in its binding interactions with the HIV envelope protein. A CD4 domain of the present invention binds to the CD4-binding site of HIV gp120 and may block the ability of HIV gp120 to bind membrane-anchored CD4, e.g., on CD4+T cells. A CD4 domain of the invention may induce a structural rearrangement in gp120 upon binding, including a structural rearrangement of part or all of the V3 region of gp120. This structural rearrangement in gp120 results in a high affinity binding site for a chemokine coreceptor (CXCR4 and/or CCR5) being exposed. Native CD4 comprises four domains that are exposed on the extracellular surface of the cell, D1, D2, D3 and D4; a transmembrane domain; and a cytoplasmic tail domain. D1 and D3 resemble Ig variable domains and D2 and D4 resemble Ig constant domains. CD4 domains of the invention include one or more of domains D1 to D4 of CD4, or variants thereof. Examples of CD4 domains of the invention include wild-type D1 (SEQ ID NO:3); “mD1.22” (SEQ ID NO: 4), which is a variant of D1 of CD4 (Chen et al, JVI 88 (2): 1125-39, 2014); wild-type D1D2 (SEQ ID NO: 1); “mD1.22-D2” (SEQ ID NO:2), which is a variant of D1D2 (Fetzer et al., Journal of Virology, 92 (12), 2018); and further variants of mD1.22 (SEQ ID NOs: 5-21).

“CDRs” are defined as the complementarity determining region amino acid sequences of an antigen binding protein. These are the hypervariable regions of immunoglobulin heavy and light chains. There are three heavy chain and three light chain CDRs (or CDR regions) in the variable portion of an immunoglobulin. In one embodiment, the CDRs are defined based on the Kabat definition. In another embodiment, the CDRs are defined based on the Chothia definition. In a further embodiment, the Chothia definition is from Discovery Studio which uses the definitions from Chothia and Lesk, JMol Biol. 196 (4): 901-17 (1987) and Morea et al, Methods, 20:267-279 (2000). In another embodiment, the Chothia definition is based on the Chothia from Abysis definition. In a further embodiment, the CDRs are defined based on the IMGT definition. In another embodiment, the CDRs are defined based on the Honegger definition. In another embodiment, the CDRs are defined based on the contact definition. Thus, “CDRs” as used herein refers to all three heavy chain CDRs, all three light chain CDRs, all heavy and light chain CDRs, or at least two CDRs.

“Domain” refers to a folded polypeptide structure that retains its tertiary structure independent of the rest of the polypeptide. Generally, domains are responsible for discrete functional properties of polypeptides and in many cases may be added, removed or transferred to other polypeptides without loss of function of the remainder of the protein and/or of the domain.

“Effector Function” as used herein refers to one or more of antibody-mediated effects including antibody-dependent cell-mediated cytotoxicity (ADCC), antibody-mediated complement activation including complement-dependent cytotoxicity (CDC), complement-dependent cell-mediated phagocytosis (CDCP), antibody dependent complement-mediated cell lysis (ADCML), and Fc-mediated phagocytosis or antibody-dependent cellular phagocytosis (ADCP).

“Epitope” as used herein refers to the portion of an antigen (e.g., gp120) that makes contact with and is capable of specifically binding to a particular site (paratope) on an antigen binding protein. An epitope may be linear or conformational/discontinuous. A conformational/discontinuous epitope comprises amino acid residues that are separated by other sequences, i.e., it does not comprise a continuous sequence in the antigen's primary amino acid sequence, but instead relies on the tertiary folding of the polypeptide. Although the residues within a confirmational/discontinuous epitope may be from different regions of the polypeptide chain, they are in close proximity in the three-dimensional structure of the antigen.

In the case of multimeric antigens, a conformational or discontinuous epitope may include residues from different polypeptide chains. Particular residues comprised within an epitope can be determined through computer modelling programs or via three-dimensional structures obtained through methods known in the art, such as X-ray crystallography.

Epitope mapping can be carried out using various techniques known to persons skilled in the art as described in publications such as Methods in Molecular Biology ‘Epitope Mapping Protocols’, by Mike Schutkowski and Ulrich Reineke (volume 524, 2009) and Johan Rockberg and Johan Nilvebrant (volume 1785, 2018). Exemplary methods include peptide-based approaches such as pepscan whereby a series of overlapping peptides are screened for binding using techniques such as ELISA or by in vitro display of large libraries of peptides or protein mutants, e.g., on phage. Detailed epitope information can be determined by structural techniques including X-ray crystallography, solution nuclear magnetic resonance (NMR) spectroscopy and cryogenic-electron microscopy (cryo-EM). Mutagenesis, such as alanine scanning, is an effective approach whereby loss of binding analysis is used for epitope mapping. Another method is hydrogen/deuterium exchange (HDX) combined with proteolysis and liquid-chromatography mass spectrometry (LC-MS) analysis to characterize discontinuous or conformational epitopes.

“Half-life” or “t_{1/2}” refers to the time required for the serum concentration of an antigen binding protein to reach half of its original value. The serum half-life of proteins can be measured by pharmacokinetic studies according to the method described by Kim et al., 1994, Eur. J. of Immuno. 24:542-548. According to this method, radio-labelled protein is injected intravenously into mice and its plasma concentration is periodically measured as a function of time, for example, at about 3 minutes to about 72 hours after the injection. Other methods for pharmacokinetic analysis and determination of the half-life of a molecule will be familiar to those skilled in the art.

“HIV envelope protein” or “ENV” refers to a trimeric viral membrane-associated glycoprotein (gp) or ‘spike’. It is found on both the viral membrane and the cell membrane of infected host cells. The env gene encodes the gp160 polypeptide which forms a homotrimer and is cleaved into gp120 and gp41 polypeptides. Gp120 is a surface (SU)glycoprotein responsible for binding to receptor molecules and the transmembrane (TM)glycoprotein, gp41, mediates fusion of the viral membrane with the plasma cell membrane. Over half of the mass of the trimeric envelope ‘spike’ is an N-linked glycan shield that hides most amino acid-based epitopes on gp120. Binding of the cell surface receptor CD4 to HIV gp120 induces a structural rearrangement creating a high affinity binding site for a chemokine coreceptor (CXCR4 and/or CCR5), on gp120. Following gp120 binding to CXCR4 or CCR5 further conformational changes are triggered which results in gp120 disengaging from gp41, allowing for the fusion peptide of gp41 to be inserted into the cell membrane, which in turn triggers a sequence of structural changes resulting in membrane fusion (Dimitrov et al., Biochemistry 44 (37): 12471-12479, 2005).

“Human immunodeficiency virus (HIV)” has been characterized into two types: HIV-1 and HIV-2. HIV-1 is more virulent and more infective than HIV-2 and is the cause of the majority of HIV infections globally, whereas HIV-2 is limited to a much smaller number of people, mostly in

West Africa (Gilbert et al., Statistics in Medicine 22 (4): 573-593). Herein, when reference is made to “HIV” this is intended to mean “HIV-1”. HIV virions are spherical with viral glycoprotein “spikes”, the HIV envelope protein, protruding outwards. A conical capsid exists within the virion, enclosing a ribonucleoprotein complex comprising two copies of positive-sense single stranded RNA tightly bound to nucleocapsid proteins and enzymes needed for viral replication.

A “linker” is an amino acid sequence that links one domain in a polypeptide to another domain in a polypeptide. For example, a linker within the meaning of the invention includes an amino acid sequence that joins a CD4 domain to a bNAb heavy chain or a bNAb light chain. In an embodiment, the linker is not cleavable under intracellular conditions.

“Multi-specific antigen binding protein” or “MSABP” refers to an antigen binding protein that comprises at least two different antigen binding sites. Each of these antigen-binding sites is capable of binding to a different epitope, which may be present on the same antigen or different antigens. In an embodiment, the multi-specific antigen binding proteins of the invention are bispecific molecules capable of binding to two different epitopes on the HIV envelope protein. In particular, one epitope may comprise part of or the whole of the V3 loop region of gp120 and the other epitope may comprise part of or the whole of the CD4 binding site of gp120.

Symmetric formats of MSABPs combine multiple binding specificities in a single polypeptide chain or single HL pair including Fc-fusion proteins of fragment-based formats and formats whereby antibody fragments are fused to regular antibody molecules. Examples of symmetric formats may include DVD-Ig, TVD-Ig, CODV-Ig, (scFv) 4-Fc, IgG-(scFv) 2, Tetravalent DART-Fc, F(ab)₄CrossMab, IgG-HC-scFv, IgG-LC-scFv, mAb-dAb etc.

“Neutralizes” as used throughout the present specification means that the biological activity of HIV is reduced in the presence of an antigen binding protein as described herein in comparison to the biological activity of HIV in the absence of the antigen binding protein, in vitro or in vivo. For example, a neutralizing antigen binding protein of the invention may inhibit HIV entry into a target cell and reduce viral load in a patient infected with HIV.

“Percent identity” or “% identity” between a query amino acid sequence and a subject amino acid sequence is the “Identities” value, expressed as a percentage, that is calculated using a suitable algorithm (e.g., BLASTP, FASTA, Needleman-Wunsch, Smith-Waterman, LALIGN, or GenePAST/KERR) or software (e.g., DNASTAR Lasergene, GenomeQuest, EMBOSS needle or EMBOSS infoalign), over the entire length of the query sequence after a pair-wise global sequence alignment has been performed using a suitable algorithm (e.g., Needleman-Wunsch or GenePAST/KERR) or software (e.g. DNASTAR Lasergene or GenePAST/KERR). Importantly, a query amino acid sequence may be described by an amino acid sequence disclosed herein, in particular in one or more of the claims.

The query sequence may be 100% identical to the subject sequence, or it may include up to a certain integer number of amino acid alterations as compared to the subject sequence such that the % identity is less than 100%. For example, the query sequence is at least 50, 60, 70, 75, 80, 85, 90, 95, 96, 97, 98, or 99% identical to the subject sequence. In the case of amino acid sequences, such alterations include at least one amino acid residue deletion, substitution (including conservative and non-conservative substitutions), or insertion, wherein said alterations may occur at the amino- or carboxy-terminal positions of the query sequence or anywhere between those terminal positions, interspersed either individually among the amino acid residues in the query sequence or in one or more contiguous groups within the query sequence.

For antibody sequences, the % identity may be determined across the entire length of the query sequence, including the CDRs. Alternatively, the % identity may exclude one or more or all of the CDRs, for example all of the CDRs are 100% identical to the subject sequence and the % identity variation is in the remaining portion of the query sequence, e.g., the framework sequence, so that the CDR sequences are fixed and intact.

“Protein scaffold” as used herein includes, but is not limited to, an immunoglobulin (Ig) scaffold, for example an IgG scaffold, which may be a four chain or two chain antibody, or which may comprise only the Fc region of an antibody, or which may comprise one or more constant regions from an antibody, which constant regions may be of human origin.

The protein scaffold may be an Ig scaffold, for example an IgG, or IgA scaffold. The IgG scaffold may comprise some or all the domains of an intact antibody (i.e., CH1, CH2, CH3, VH, VL). The antigen binding protein may comprise an IgG scaffold selected from IgG1, IgG2, IgG3, IgG4 or IgG4PE. For example, the scaffold may be IgG1. The scaffold may consist of, or comprise, the Fc region of an antibody, or is a part thereof.

The protein scaffold may be a non-Ig scaffold. The protein scaffold may be a derivative of a scaffold selected from the group consisting of CTLA-4, lipocalin, Protein A derived molecules such as Z-domain of Protein A (Affibody, SpA), A-domain (Avimer/Maxibody); heat shock proteins such as GroEI and GroES; transferrin (trans-body); ankyrin repeat protein (DARPin); peptide aptamer; C-type lectin domain (Tetranectin); human γ-crystallin and human ubiquitin (affilins); PDZ domains; scorpion toxin kunitz type domains of human protease inhibitors; and fibronectin/adnectin; which has been subjected to protein engineering in order to obtain binding to an antigen, such as gp120.

“Single variable domain” refers to a folded polypeptide domain comprising sequences characteristic of antibody variable domains. It therefore includes complete antibody variable domains such as VH, VHH and VL and modified antibody variable domains, for example, in which one or more loops have been replaced by sequences that are not characteristic of antibody variable domains, or antibody variable domains that have been truncated or comprise N- or C-terminal extensions, as well as folded fragments of variable domains that retain at least the binding activity and specificity of the full-length domain. A single variable domain as defined herein is capable of binding an antigen or epitope independently of a different variable region or domain. A “domain antibody” or “DAB” may be considered the same as a human “single variable domain”. A single variable domain may be a human single variable domain, but also includes single variable domains from other species such as rodent (for example, as disclosed in WO 00/29004), nurse shark and Camelid VHHs Camelid VHHs are immunoglobulin single variable domain polypeptides that are derived from species including camel, llama, alpaca, dromedary, and guanaco, which produce heavy chain only antibodies naturally devoid of light chains. Such VHH domains may be humanised according to standard techniques available in the art, and such domains are considered to be “single variable domains”.

“Stabilizing mutation” refers to a change of an amino acid residue in a polypeptide sequence that increases the thermal thermostability of said polypeptide. Increased thermostability may be reflected in a melting temperature (Tₘ) increase of, for example, between 1 and 50° C. CD4 domains with stabilizing mutations include SEQ ID NOs: 5-21.

A “variant sequence” substantially retains the biological characteristics of the unmodified protein. In the case of an antibody sequence disclosed herein, the VH or VL (or HC or LC) sequence may be a variant sequence with up to 10 amino acid substitutions, additions or deletions. For example, the variant sequence may have up to 9, 8, 7, 6, 5, 4, 3, 2 or 1 amino acid substitution(s), addition(s) or deletion(s). The sequence variation may exclude one or more or all of the CDRs, for example the CDRs are the same as the VH or VL (or HC or LC) sequence and the variation is in the remaining portion of the VH or VL (or HC or LC) sequence, so that the CDR sequences are fixed and intact.

“V3 loop region”, “V3/glycan” or “V3” as used herein refers to the third variable region (V3) of HIV gp120. Comparison of predicted amino acid sequences from several different isolates has shown that sequence heterogeneity of gp120 is clustered in five variable regions (designated V1, V2, V3, V4, and V5.) The V3 region contains post-translational modifications, such as glycosylation, and is essential for viral infectivity. The V3 region, although only 35 amino acids long, exhibits considerable sequence variability. Additionally, variability in potential N-linked glycosylation sites allow for further variability in the variable regions of gp120. Together, the V3 region and the N-linked glycosylation sites within and adjacent to the region are understood to comprise the “V3 loop region,” “V3/glycan” or V3″ as used herein. For example, one site of glycosylation (e.g., oligomannose such as Man-5 to Man-9) is centered on amino acid residue N332 of gp120. Other sites of potential N-linked glycosylation within and adjacent to the V3 loop region include K295, N301, N386, N392 of gp120. The V3 loop is generally considered to be in the region between cysteine residues C296 and C331 of gp120, while some N-linked glycosylation sites are located directly adjacent to the V3 loop. The V3 loop comprises a highly conserved tetrapeptide sequence, GPGR (residues 312 to 315) (Ivanhoff et al., Virology, 187 (2) 1992). HIV-1 cellular entry depends on the interaction of the V3 loop region with an HIV co-receptor, commonly CCR5 or CXCR4. The V3 loop comprises: (i) the base (residues 296-299), (ii) the stem (residues 300-303 and 321-326), and (iii) the crown (residues 304-320) (Friedrich et al., Nature Communications 12, 6705 (2021)). A consensus sequence of the V3 region of gp120 (Milich et al., J Virol., 67 (9): 5623-5634 (1993)) is provided below:

CTRPNNNTRKSIHIGPGRAFYTTGEIIGDIRQAHC (SEQ ID NO: 361)

It is understood that the consensus sequence describes the highest frequency of residues emerging on each position of this region across multiple subtypes, but that the V3 loop region of a particular strain may exhibit sequence variability.

A “V3-bNAb” or “anti-V3 bNAb” is a bNAb that binds within the V3 loop region. A V3-bNAb may also be referred to herein as an anti-V3 antibody. A V3-bNAb may bind the N332 glycan in the V3 loop region and/or other N-linked glycosylation sites within and adjacent to the V3 loop region.

### STATEMENT OF THE INVENTION

An antigen binding protein of the invention binds to the Human Immunodeficiency Virus (HIV) envelope protein. In particular, the antigen binding protein binds to HIV envelope surface glycoprotein 120 (gp120) and is, therefore, also referred to herein as an anti-gp120 binding protein. The anti-gp120 binding protein of the invention binds to at least two different epitopes on gp120, including the V3 loop region (V3) and the CD4 binding site (CD4bs) of gp120.

Bispecific molecules of the invention that bind to the V3 loop region and the CD4bs of gp120 have been shown to effectively neutralize HIV and exhibit significantly better anti-viral activity than monospecific molecules that only bind to the V3 loop region or the CD4bs of gp120, and mixtures of these monospecific molecules. Without being bound by any particular theory, we postulate that the bispecific molecules of the invention bind the two different epitopes in the same or neighboring HIV envelope protein trimers at the same time, such that the bispecific molecules achieve stronger binding (increased avidity) to the HIV envelope proteins. This may be as a result of the high local concentration of the bispecific molecules' binding sites (paratopes) being “pre-positioned” around their target binding sites (epitopes) on the HIV envelope compared to their monospecific counterparts, which in turn leads to stronger anti-viral activity.

### Binding to the CD4 Binding Site (CD4bs) of HIV Gp120

The antigen binding protein of the invention comprises one or more paratopes that bind to the CD4bs of HIV gp120. Binding domains comprising such paratopes may be include by CD4 domains, as well as other anti-CD4bs domains, including those of anti-CD4bs antibodies and CD4bs-binding fragments thereof. Non-Ig constructs that bind to CD4bs are also part of the invention, such as single chain variable fragments (scFvs). In particular, non-Ig constructs such as scFv comprising one or more CDRs, preferably the three light chain CDRs or the three heavy chain CDRs, or a set of six CDRs of such anti-CD4bs antibodies are also part of the invention.

In an embodiment of the invention, the antigen binding protein of the invention comprises an anti-CD4bs antibody or CD4bs-binding fragment thereof, wherein such antibody or fragment thereof comprises a paratope that binds to the CD4bs of HIV gp120. In a further embodiment, the anti-CD4bs antibody is selected from the group consisting of: b12, HJ16, CH103-106, VRCO1-03, VRC-PG04, VRC-PG04b, VRC-CH30-34, 3BNC117, 3BNC60, NIH45-46, 12A12, 12A21, 8ANC131, 8ANC134, 1NC9, and 1B2530.

In an alternative or additional embodiment of the invention, a paratope that binds to the CD4bs of HIV gp120 is formed by a polypeptide domain that binds to the CD4bs of HIV gp120. In a more particular embodiment, the polypeptide domain is a CD4 domain.

### CD4 Domains

CD4 domains of the invention include SEQ ID NOs: 1-21.

In an embodiment of the invention, the CD4 domain is a CD4 D1 domain. In an embodiment, the CD4 domain is a human CD4 domain. CD4 D1 domains include human wild-type D1 (SEQ ID NO:3), mD1.22 (SEQ ID NO:4) also known as D1m, and further variants of mD1.22 (SEQ ID NOs: 5-21).

In an embodiment of the invention, the CD4 domain is a CD4 D1D2 domain. In an embodiment, the CD4 domain is a human CD4 D1D2 domain. CD4 D1D2 domains include human wild-type D1D2 (SEQ ID NO:1) and mD1.22-D2 (SEQ ID NO:2).

In an aspect of the invention, a stabilized CD4 domain is provided. In an embodiment of the invention, a stabilized CD4 D1 domain is provided. In an embodiment, the CD4 domain is thermally stable, i.e., thermostable. In an embodiment, the CD4 domain is a thermostable CD4 D1 domain.

In an embodiment of the invention, the CD4 domain comprises one or more stabilizing mutations. In an embodiment, the stabilizing mutations are in the CD4 D1 domain. In an embodiment, the CD4 D1 domain comprises one or more mutations selected from the group consisting of: K8C, K8I, K8V, T11C, E13C, K21C, Q25E, H27C, H27D, G38C, N52W, R58N, R58T, R58V, L61M, G65C, I70C, K72C, E87G, E91H, E91Q, and G99C. In an embodiment, the CD4 D1 domain comprises K8I. In an embodiment, the CD4 D1 domain comprises K8V. In an embodiment, the CD4 D1 domain comprises T1C and K72C. In an embodiment, the CD4 D1 domain comprises K8C and G99C.

CD4 domains of the invention comprising novel and inventive stabilizing mutations include SEQ ID NOS: 5-21.

Increased thermostability may be reflected in a melting temperature (Tm) increase of, for example, between 1 and 50° C.; in particular between 1 and 30° C.; in particular between 1 and 25° C., in particular between 1 and 21° C., more particularly between 5 and 21° C. The Tm increase is determined by measuring the Tm of the CD4 domain(s) comprising one or more stabilizing mutations and subtracting the Tm of the corresponding CD4 domain(s) without said mutation(s). For example, measuring the Tm of a stabilized CD4 D1 domain and subtracting the Tm of the wild-type CD4 D1 domain. In an embodiment, the Tm increase is about 8° C. In an embodiment, the Tm increase is about 9° C. In an embodiment, the Tm increase is about 12° C. In an embodiment, the Tm increase is about 21° C.

In an embodiment, the Tm of the CD4 domain is above 70° C. In an embodiment, the Tm of the CD4 domain is between 70° C. and 95° C. In an embodiment, the Tm of the CD4 domain is between 75° C. and 95° C. In an embodiment, the Tm of the CD4 domain is between 75° C. and 91° C. In an embodiment, the Tm of the CD4 domain is about 76° C., about 77° C., about 78° C., about 79° C., about 80° C., about 81° C., about 82° C., about 83° C., about 84° C., about 85° C., about 86° C., about 87° C., about 88° C., about 89° C., or about 90° C. In an embodiment, the Tm of the CD4 domain is about 90° C. In an embodiment, the Tm of the CD4 domain is about 89° C.

Tm may be determined by routine methods known in the art or as set out in the Examples. In an embodiment, Tm is determined using the Prometheus System (NanoTemper, München Germany).

### Binding to the V3 Loop Region of HIV Gp120

The antigen binding protein of the invention comprises one or more paratopes that bind to the V3 loop region of HIV gp120. Binding domains comprising such paratopes include an anti-V3 bNAb or a V3-binding fragment thereof, as well as a non-Ig construct that binds to V3.

An antigen binding protein of the invention may comprise heavy chain CDRs (CDRH1, CDRH2, and CDRH3) as set out in any row of Table 1. An antigen binding protein of the invention may comprise light chain CDRs (CDRL1, CDRL2, and CDRL3) as set out in any row of Table 1. An antigen binding protein of the invention may comprise a set of six CDRs (CDRH1, CDRH2, CDRH3, CDRL1, CDRL2 and CDRL3) as set out in any row of Table 1.

An antigen binding protein of the invention may comprise heavy chain CDRs (CDRH1, CDRH2, and CDRH3) of any one of PGT121-123, PGT125-131, PGT135-137, QA013.2, 10-1074, 10-1074LS, PGT121.414.LS and 2G12. An antigen binding protein of the invention may comprise light chain CDRs (CDRL1, CDRL2, and CDRL3) of any one of PGT121-123, PGT125-131, PGT135-137, QA013.2, 10-1074, 10-1074LS, PGT121.414.LS and 2G12. An antigen binding protein of the invention may comprise a set of six CDRs (CDRH1, CDRH2, CDRH3, CDRL1, CDRL2 and CDRL3) of any one of PGT121-123, PGT125-131, PGT135-137, QA013.2, 10-1074, 10-1074LS, PGT121.414.LS and 2G12.

An antigen binding protein of the invention may comprise a VH domain as set out in Table 2. An antigen binding protein of the invention may comprise a VL domain as set out in Table 2. An antigen binding protein of the invention may comprise a pair of variable domains (a VH and a VL) as set out in any row of Table 2.

Anti-V3 bNAbs

An antigen binding protein of the invention may comprise an anti-V3 bNAb or a V3-binding fragment thereof. An anti-V3 antibody includes an antibody comprising a set of CDRs (CDRH1, CDRH2, CDRH3, CDRL1, CDRL2 and CDRL3) as set out in any row of Table 1.

TABLE 1
SEQ ID NOs for the complementarity determining regions
(CDRs) of broadly neutralizing antibodies (bNAbs) 1-6

| | CDRH1 | CDRH2 | CDRH3 | CDRL1 | CDRL2 | CDRL3 |
|---|---|---|---|---|---|---|
| bNAb1 | 22 | 23 | 24 | 25 | 26 | 27 |
| bNAb2 | 28 | 29 | 30 | 31 | 32 | 33 |
| bNAb3 | 34 | 35 | 36 | 37 | 38 | 39 |
| bNAb4 | 40 | 41 | 42 | 43 | 44 | 45 |
| bNAb5 | 46 | 47 | 48 | 49 | 50 | 51 |
| bNAb6 | 52 | 53 | 54 | 55 | 56 | 57 |
| bNAb7 | 159 | 160 | 161 | 162 | 163 | 164 |
| bNAb8 | 165 | 166 | 167 | 168 | 169 | 170 |
| bNAb9 | 171 | 172 | 173 | 174 | 175 | 176 |
| bNAb10 | 177 | 178 | 179 | 180 | 181 | 182 |
| bNAb11 | 183 | 184 | 185 | 186 | 187 | 188 |
| bNAb12 | 189 | 190 | 191 | 192 | 193 | 194 |
| bNAb13 | 195 | 196 | 197 | 198 | 199 | 200 |
| bNAb14 | 201 | 202 | 203 | 204 | 205 | 206 |
| bNAb15 | 207 | 208 | 209 | 210 | 211 | 212 |
| bNAb16 | 213 | 214 | 215 | 216 | 217 | 218 |
| bNAb17 | 219 | 220 | 221 | 222 | 223 | 224 |
| bNAb18 | 225 | 226 | 227 | 228 | 229 | 230 |
| bNAb19 | 231 | 232 | 233 | 234 | 235 | 236 |
| bNAb20 | 237 | 238 | 239 | 240 | 241 | 242 |
| bNAb21 | 243 | 244 | 245 | 246 | 247 | 248 |
| bNAb23 | 249 | 250 | 251 | 252 | 253 | 254 |

In a particular embodiment, the anti-V3 antibody or V3-binding fragment thereof, comprises the CDRs of bNAb1. In an embodiment, the anti-V3 antibody or V3-binding fragment thereof, comprises a CDRH1 of SEQ ID NO:22, a CDRH2 of SEQ ID NO:23, a CDRH3 of SEQ ID NO: 24, a CDRL1 of SEQ ID NO:25, a CDRL2 of SEQ ID NO: 26 and a CDRL3 of SEQ ID NO:27.

An anti-V3 bNAb may be an antibody comprising a pair of variable domains (a VH and a VL) as set out in any row of Table 2.

An anti-V3 bNAb may be an antibody comprising a heavy chain (HC), with or without M428L/N434S (EU numbering) ‘LS’ mutations, and a light chain (LC) as set out in any row of Table 2. In an embodiment, the HC comprises LS.

TABLE 2
SEQ ID NOs for the variable regions (VH and VL)
and the heavy chains (HC) and light chains (LC)
of bNAbs 1-6. ‘LS’ refers to M428L/N434S
(EU numbering) mutations in the Fc portion of the bNAb.

| | VH | VL | HC without ‘LS’ | HC with ‘LS’ | LC |
|---|---|---|---|---|---|
| bNAb1 | 58 | 59 | 61 | 62 | 63 |
| bNAb1* | | 60* | | | 64* |
| bNAb2 | 65 | 66 | 67 | 68 | 69 |
| bNAb3 | 70 | 71 | 72 | 73 | 74 |
| bNAb4 | 75 | 76 | 77 | 78 | 79 |
| bNAb5 | 80 | 81 | 82 | 83 | 84 |
| bNAb6 | 85 | 86 | 87 | 88 | 89 |
| bNAb7 | 255 | 256 | 257 | 258 | 259 |
| bNAb8 | 260 | 261 | 262 | 263 | 264 |
| bNAb9 | 265 | 266 | 267 | 268 | 269 |
| bNAb10 | 270 | 271 | 272 | 273 | 274 |
| bNAb11 | 275 | 276 | 277 | 278 | 279 |
| bNAb12 | 280 | 281 | 282 | 283 | 284 |
| bNAb13 | 285 | 286 | 287 | 288 | 289 |
| bNAb14 | 290 | 291 | 292 | 293 | 294 |
| bNAb15 | 295 | 296 | 297 | 298 | 299 |
| bNAb16 | 300 | 301 | 302 | 303 | 304 |
| bNAb17 | 305 | 306 | 307 | 308 | 309 |
| bNAb18 | 310 | 311 | 312 | 313 | 314 |
| bNAb19 | 315 | 316 | 317 | 318 | 319 |
| bNAb20 | 320 | 321 | 322 | 323 | 324 |
| bNAb21 | 325 | 326 | 327 | 328 | 329 |
| bNAb22^{#} | 330 | | 331 | 332 | |
| bNAb 23 | 334 | 335 | 336 | 337 | 338 |

*a light chain variant with F32Y in the VL and LC^{#}a heavy chain variant with {~TC to inserts definition}

In an embodiment, the anti-V3 antibody or V3-binding fragment thereof, comprises a VH domain of SEQ ID NO:58 and a VL domain of SEQ ID NO:59 or 60. In an embodiment, the anti-V3 antibody or V3-binding fragment thereof, comprises a VH domain of SEQ ID NO:58 and a VL domain of SEQ ID NO:59. In an embodiment, the anti-V3 antibody or V3-binding fragment thereof, comprises a VH domain of SEQ ID NO: 65 and a VL domain of SEQ ID NO: 66. In an embodiment, the anti-V3 antibody or V3-binding fragment thereof, comprises a VH domain of SEQ ID NO:70 and a VL domain of SEQ ID NO:71. In an embodiment, the anti-V3 antibody or V3-binding fragment thereof, comprises a VH domain of SEQ ID NO:75 and a VL domain of SEQ ID NO:76. In an embodiment, the anti-V3 antibody or V3-binding fragment thereof, comprises a VH domain of SEQ ID NO:80 and a VL domain of SEQ ID NO:81. In an embodiment, the anti-V3 antibody or V3-binding fragment thereof, comprises a VH domain of SEQ ID NO: 85 and a VL domain of SEQ ID NO:86. In an embodiment, the anti-V3 antibody or V3-binding fragment thereof, comprises a VH domain of SEQ ID NO:225 and a VL domain of SEQ ID NO:226. In an embodiment, the anti-V3 antibody or V3-binding fragment thereof, comprises a VH domain of SEQ ID NO:260 and a VL domain of SEQ ID NO:261. In an embodiment, the anti-V3 antibody or V3-binding fragment thereof, comprises a VH domain of SEQ ID NO: 265 and a VL domain of SEQ ID NO:266. In an embodiment, the anti-V3 antibody or V3-binding fragment thereof, comprises a VH domain of SEQ ID NO:270 and a VL domain of SEQ ID NO: 271. In an embodiment, the anti-V3 antibody or V3-binding fragment thereof, comprises a VH domain of SEQ ID NO: 275 and a VL domain of SEQ ID NO:276. In an embodiment, the anti-V3 antibody or V3-binding fragment thereof, comprises a VH domain of SEQ ID NO: 280 and a VL domain of SEQ ID NO:281. In an embodiment, the anti-V3 antibody or V3-binding fragment thereof, comprises a VH domain of SEQ ID NO:285 and a VL domain of SEQ ID NO:286. In an embodiment, the anti-V3 antibody or V3-binding fragment thereof, comprises a VH domain of SEQ ID NO: 290 and a VL domain of SEQ ID NO:291. In an embodiment, the anti-V3 antibody or V3-binding fragment thereof, comprises a VH domain of SEQ ID NO:295 and a VL domain of SEQ ID NO: 296. In an embodiment, the anti-V3 antibody or V3-binding fragment thereof, comprises a VH domain of SEQ ID NO: 300 and a VL domain of SEQ ID NO:301. In an embodiment, the anti-V3 antibody or V3-binding fragment thereof, comprises a VH domain of SEQ ID NO: 305 and a VL domain of SEQ ID NO:306. In an embodiment, the anti-V3 antibody or V3-binding fragment thereof, comprises a VH domain of SEQ ID NO:310 and a VL domain of SEQ ID NO:311. In an embodiment, the anti-V3 antibody or V3-binding fragment thereof, comprises a VH domain of SEQ ID NO: 320 and a VL domain of SEQ ID NO:321. In an embodiment, the anti-V3 antibody or V3-binding fragment thereof, comprises a VH domain of SEQ ID NO: 225 or SEQ ID NO: 330 and a VL domain of SEQ ID NO:326. In an embodiment, the anti-V3 antibody or V3-binding fragment thereof, comprises a VH domain of SEQ ID NO:334 and a VL domain of SEQ ID NO:335.

In an embodiment, the anti-V3 antibody comprises a HC of SEQ ID NO:61 or 62 and a LC of SEQ ID NO:63 or 64. In an embodiment, the anti-V3 antibody comprises a HC of SEQ ID NO:62 and a LC of SEQ ID NO: 63. In an embodiment, the anti-V3 antibody comprises a HC of SEQ ID NO:67 or 68 and a LC of SEQ ID NO:69. In an embodiment, the anti-V3 antibody comprises a HC of SEQ ID NO: 72 or 73 and a LC of SEQ ID NO:74. In an embodiment, the anti-V3 antibody comprises a HC of SEQ ID NO:77 or 78 and a LC of SEQ ID NO:79. In an embodiment, the anti-V3 antibody comprises a HC of SEQ ID NO: 82 or 83 and a LC of SEQ ID NO: 84. In an embodiment, the anti-V3 antibody comprises a HC of SEQ ID NO: 87 or 88 and a LC of SEQ ID NO: 89 In an embodiment, the anti-V3 antibody comprises a HC of SEQ ID NO: 257 or 258 and a LC of SEQ ID NO:259. In an embodiment, the anti-V3 antibody comprises a HC of SEQ ID NO: 262 or 263 and a LC of SEQ ID NO:264. In an embodiment, the anti-V3 antibody comprises a HC of SEQ ID NO: 267 or 268 and a LC of SEQ ID NO: 269. In an embodiment, the anti-V3 antibody comprises a HC of SEQ ID NO:272 or 273 and a LC of SEQ ID NO: 274. In an embodiment, the anti-V3 antibody comprises a HC of SEQ ID NO:277 or 278 and a LC of SEQ ID NO:279. In an embodiment, the anti-V3 antibody comprises a HC of SEQ ID NO: 282 or 283 and a LC of SEQ ID NO:284. In an embodiment, the anti-V3 antibody comprises a HC of SEQ ID NO: 292 or 293 and a LC of SEQ ID NO:294. In an embodiment, the anti-V3 antibody comprises a HC of SEQ ID NO: 297 or 298 and a LC of SEQ ID NO:299. In an embodiment, the anti-V3 antibody comprises a HC of SEQ ID NO:302 or 303 and a LC of SEQ ID NO:304. In an embodiment, the anti-V3 antibody comprises a HC of SEQ ID NO:307 or 308 and a LC of SEQ ID NO: 309. In an embodiment, the anti-V3 antibody comprises a HC of SEQ ID NO: 312 or 313 and a LC of SEQ ID NO: 314. In an embodiment, the anti-V3 antibody comprises a HC of SEQ ID NO: 317 or 318 and a LC of SEQ ID NO:319. In an embodiment, the anti-V3 antibody comprises a HC of SEQ ID NO: 322 or 323 and a LC of SEQ ID NO:324. In an embodiment, the anti-V3 antibody comprises a HC of SEQ ID NO: 327, 328, 331 or 332 and a LC of SEQ ID NO:329. In an embodiment, the anti-V3 antibody comprises a HC of SEQ ID NO:336 or 337 and a LC of SEQ ID NO:329.

Anti-V3 bNAbs known in the art include PGT121-123, PGT125-131, PGT135-137, DH270.6, QA013.2, 10-1074, 10-1074LS, PGT121.414.LS and 2G12, 438-B11, 447-52D, BG18, DH270.6, ePGT121v1, ePGT121v2, ePGT121v3, EPTC112, and F425-B4e8. In an embodiment, the anti-V3 antibody or V3-binding fragment thereof, comprises the six CDRs of any one of PGT121-123, PGT125-131, PGT135-137, DH270.6, QA013.2, 10-1074, 2G12, 438-B11, 447-52D, BG18, DH270.6, ePGT121v1, ePGT121v2, ePGT121v3, EPTC112, and F425-B4e8. In an embodiment, the anti-V3 antibody or V3-binding fragment thereof, comprises the VH domain and the VL domain any one of PGT121-123, PGT125-131, PGT135-137, DH270.6, QA013.2, 10-1074, 2G12, 438-B11, 447-52D, BG18, DH270.6, ePGT121v1, ePGT121v2, ePGT121v3, EPTC112, and F425-B4e8.

An antigen binding protein of the invention may comprise an anti-V3 scFv of any one of the aforementioned anti-V3 bNAbs. In an embodiment, the scFv comprises a VH and VL pair as set out in Table 2. In an embodiment, the scFv comprises a VH and VL pair of any one of PGT121-123, PGT125-131, PGT135-137, QA013.2, 10-1074, 10-1074LS, PGT121.414.LS and 2G12. In an embodiment, the C-terminus of the VH domain is attached directly or via a linker to the N-terminus of the VL domain. In an embodiment, the C-terminus of the VL domain is attached directly or via a linker to the N-terminus of the VH domain. In an embodiment, the scFv comprises a VH domain of SEQ ID NO: 58 and a VL domain of SEQ ID NO: 59. In an embodiment, the scFv comprises a VH domain of SEQ ID NO: 65 and a VL domain of SEQ ID NO:66. In an embodiment, the scFv comprises a VH domain of SEQ ID NO: 70 and a VL domain of SEQ ID NO:71. In an embodiment, the scFv comprises a VH domain of SEQ ID NO: 75 and a VL domain of SEQ ID NO:76. In an embodiment, the scFv comprises a VH domain of SEQ ID NO:80 and a VL domain of SEQ ID NO:81. In an embodiment, the scFv comprises a VH domain of SEQ ID NO: 85 and a VL domain of SEQ ID NO: 86.

An anti-V3 scFv may be fused to an Fc domain. In an embodiment, the scFv is fused to a human Fc domain directly or via a linker (scFv-Fc).

### Linkers

Examples of suitable linkers include amino acid sequences that are from 1 amino acid to 150 amino acids in length. In particular, from 1 to 140 amino acids, from 1 to 130 amino acids, from 1 to 120 amino acids, from 1 to 110 amino acids, from 1 to 100 amino acids, from 1 to 90 amino acids, from 1 to 80 amino acids, from 1 to 70 amino acids, from 1 to 60 amino acids, from 1 to 50 amino acids, from 1 to 40 amino acids, from 1 to 30 amino acids, from 1 to 20 amino acids, from 1 to 10 amino acids, from 5 to 30 amino acids.

In an embodiment, the linker is an amino acid sequence from 5 to 30 amino acids in length. In an embodiment, the linker is an amino acid sequence as set forth in any one of SEQ ID NOs: 90 to 95. In an embodiment, the linker is an amino acid sequence as set forth in SEQ ID NO:90. In an embodiment, the linker is a multimer of the amino acid sequence as set forth in SEQ ID NO:90. In an embodiment, the linker is [SEQ ID NO: 90]ⁿ, wherein n is an integer from 1 to 6. In an embodiment, the linker is an amino acid sequence as set forth in SEQ ID NO:91. In an embodiment, the linker is an amino acid sequence as set forth in SEQ ID NO: 92. In an embodiment, the linker is an amino acid sequence as set forth in SEQ ID NO:93. In an embodiment, the linker is an amino acid sequence as set forth in SEQ ID NO:94. In an embodiment, the linker is an amino acid sequence as set forth in SEQ ID NO:95.

Any of the aforementioned linkers may be incorporated into an antigen binding protein of the invention. In particular, any of the aforementioned linkers may be used to join a domain within the antigen binding protein to another domain within the antigen binding protein. In particular, any of the aforementioned linkers may be used to join a domain within the antigen binding protein that binds to the CD4-binding site of HIV gp120 to another domain within the antigen binding protein that binds to the V3 loop region of HIV gp120. Further, any of the aforementioned linkers may be used to join a CD4 domain as disclosed herein to a bNAb as disclosed herein. In an embodiment, the linker is an amino acid sequence as set forth in any one of SEQ ID NOs: 90 to 95. In an embodiment, the linker is an amino acid sequence as set forth in SEQ ID NO:90.

In an embodiment, a linker is used to join the C-terminus of a CD4 domain to the N-terminus of a bNAb heavy chain variable domain. In an embodiment, a linker is used to join the C-terminus of a CD4 domain to the N-terminus of a bNAb light chain variable domain. In an embodiment, a linker is used to join the C-terminus of a CD4 domain to the N-terminus of a bNAb heavy chain variable domain and a linker is used to join the C-terminus of a CD4 domain to the N-terminus of a bNAb light chain variable domain. In an embodiment, a linker is used to join the C-terminus of a CD4 domain to the N-terminus of a bNAb heavy chain variable domain and an identical linker is used to join the C-terminus of a CD4 domain to the N-terminus of a bNAb light chain variable domain. In an embodiment, the linker is an amino acid sequence as set forth in any one of SEQ ID NOs: 90 to 95. In an embodiment, the linker is an amino acid sequence as set forth in SEQ ID NO: 90.

In an embodiment, a linker is used to join the N-terminus of a CD4 domain to the C-terminus of a bNAb heavy chain. In an embodiment, a linker is used to join the N-terminus of a CD4 domain to the C-terminus of a bNAb heavy chain variable domain. In an embodiment, a linker is used to join the N-terminus of a CD4 domain to the C-terminus of a bNAb light chain. In an embodiment, a linker is used to join the N-terminus of a CD4 domain to the C-terminus of a bNAb light chain variable domain. In an embodiment, a linker is used to join the N-terminus of a CD4 domain to the C-terminus of an Fc domain. In an embodiment, the linker is an amino acid sequence as set forth in any one of SEQ ID NOs: 90 to 95. In an embodiment, the linker is an amino acid sequence as set forth in SEQ ID NO:90.

In an embodiment of the invention, the domain of the antigen binding protein that binds to the CD4-binding site of HIV gp120 is joined directly to another domain within the antigen binding protein that binds to the V3 loop region of HIV gp120, i.e., a linker is not used. In an embodiment, a CD4 domain as disclosed herein is joined directly to a bNAb as disclosed herein.

Any of the aforementioned linkers may be used to join a VH and VL pair as disclosed herein to form a scFv. In an embodiment, the linker between the VH domain and the VL domain of the scFv is selected from the group consisting of SEQ ID NOs: 90-95. In a particular embodiment, the linker between the VH domain and the VL domain of the scFv is SEQ ID NO:93.

Any of the aforementioned linkers may be used to join a scFv as disclosed herein to an Fc domain. In an embodiment, the scFv is fused to a human Fc via a linker selected from the group consisting of SEQ ID NO:90-95. In an embodiment, the scFv is fused to a human Fc via a linker of SEQ ID NO:91.

### Bispecific Molecules

A bispecific molecule of the invention comprises one or more paratopes that bind to the CD4bs of HIV gp120 and one or more paratopes that bind to the V3 loop region of HIV gp120.

Paratopes that bind to the CD4bs of HIV gp120 may be formed by CD4 domains disclosed herein, as well as other CD4bs-binding domains disclosed herein, including those of anti-CD4bs antibodies and CD4bs-binding fragments thereof, and non-Ig constructs that bind to CD4bs.

Paratopes that bind to the V3 loop region of HIV gp120 may be formed by anti-V3 antibodies and V3-binding fragments thereof disclosed herein, as well as non-Ig constructs that bind to V3 disclosed herein.

In an embodiment, the bispecific molecule comprises a paratope that binds to the CD4bs of HIV gp120 that is formed by an anti-CD4bs antibody or CD4bs-binding fragment thereof and a paratope that binds to the V3 loop region of HIV gp120 that is formed by an anti-V3 antibody or a V3-binding fragment thereof.

In an embodiment, the bispecific molecule comprises an anti-CD4bs antibody or CD4bs-binding fragment thereof and an anti-V3 antibody or a V3-binding fragment thereof.

In an embodiment, the bispecific molecule comprises a CD4 domain and a paratope that binds to the V3 loop region of HIV gp120 that is formed by an anti-V3 antibody or a V3-binding fragment thereof.

In an embodiment, the bispecific molecule comprises a CD4 domain and an anti-V3 antibody or a V3-binding fragment thereof.

In an embodiment, the bispecific molecule comprises a CD4 domain of any one of SEQ ID NOs: 1-21 and an anti-V3 antibody or a V3-binding fragment thereof.

In an embodiment, the bispecific molecule comprises a CD4 domain of SEQ ID NO: 11 and an anti-V3 antibody or a V3-binding fragment thereof.

In an embodiment, the bispecific molecule comprises a CD4 domain and an anti-V3 antibody, selected from the group consisting of: PGT121-123, PGT125-131, PGT135-137, QA013.2, 10-1074, 10-1074LS, PGT121.414.LS and 2G12, or a V3-binding fragment thereof.

In an embodiment, the bispecific molecule comprises a CD4 domain of any one of SEQ ID NOs: 1-21 and an anti-V3 antibody selected from the group consisting of: PGT121-123, PGT125-131, PGT135-137, QA013.2, 10-1074, 10-1074LS, PGT121.414.LS and 2G12, or a V3-binding fragment thereof.

In an embodiment, the bispecific molecule comprises a CD4 domain of SEQ ID NO: 11 and an anti-V3 antibody selected from the group consisting of: PGT121-123, PGT125-131, PGT135-137, QA013.2, 10-1074, 10-1074LS, PGT121.414.LS and 2G12, or a V3-binding fragment thereof. In an embodiment, the bispecific molecule comprises a CD4 domain and an anti-V3 antibody comprising a set of CDRs (CDRH1, CDRH2, CDRH3, CDRL1, CDRL2 and CDRL3) as set out in any row of Table 1, or a V3-binding fragment thereof.

In an embodiment, the bispecific molecule comprises a CD4 domain of any one of SEQ ID NOs: 1-21 and an anti-V3 antibody comprising a set of CDRs (CDRH1, CDRH2, CDRH3, CDRL1, CDRL2 and CDRL3) as set out in any row of Table 1, or a V3-binding fragment thereof.

In an embodiment, the bispecific molecule comprises a CD4 domain of SEQ ID NO: 11 and an anti-V3 antibody comprising a set of CDRs (CDRH1, CDRH2, CDRH3, CDRL1, CDRL2 and CDRL3) as set out in any row of Table 1 or a V3-binding fragment thereof.

In an embodiment, the bispecific molecule comprises a CD4 domain of SEQ ID NO: 11 and an anti-V3 antibody comprising a CDRH1 of SEQ ID NO:22, a CDRH2 of SEQ ID NO:23, a CDRH3 of SEQ ID NO:24, a CDRL1 of SEQ ID NO:25, a CDRL2 of SEQ ID NO: 26 and a CDRL3 of SEQ ID NO: 27, or a V3-binding fragment thereof.

In an embodiment, the bispecific molecule comprises a CD4 domain and an anti-V3 antibody comprising a pair of variable domains (a VH and a VL) as set out in any row of Table 2, or a V3-binding fragment thereof.

In an embodiment, the bispecific molecule comprises a CD4 domain of any one of SEQ ID NOs: 1-21 and an anti-V3 antibody comprising a pair of variable domains (a VH and a VL) as set out in any row of Table 2, or a V3-binding fragment thereof.

In an embodiment, the bispecific molecule comprises a CD4 domain of SEQ ID NO: 11 and an anti-V3 antibody comprising a pair of variable domains (a VH and a VL) as set out in any row of Table 2, or a V3-binding fragment thereof.

In an embodiment, the bispecific molecule comprises a CD4 domain and an anti-V3 antibody or a V3-binding fragment thereof comprising a VH domain of SEQ ID NO:58 and a VL domain of SEQ ID NO: 59 or 60.

In an embodiment, the bispecific molecule comprises a CD4 domain of any one of SEQ ID NOs: 1-21 and an anti-V3 antibody comprising a VH domain of SEQ ID NO:58 and a VL domain of SEQ ID NO:59 or 60.

In an embodiment, the bispecific molecule comprises a CD4 domain of SEQ ID NO: 11 and an anti-V3 antibody comprising a VH domain of SEQ ID NO:58 and a VL domain of SEQ ID NO:59 or 60.

In an embodiment, the bispecific molecule comprises a CD4 domain of any one of SEQ ID NOs: 1-21 and an anti-V3 antibody comprising a VH domain of SEQ ID NO:65 and a VL domain of SEQ ID NO:66.

In an embodiment, the bispecific molecule comprises a CD4 domain of SEQ ID NO: 11 and an anti-V3 antibody comprising a VH domain of SEQ ID NO:65 and a VL domain of SEQ ID NO:66. In an embodiment, the bispecific molecule comprises a CD4 domain of any one of SEQ ID NOs: 1-21 and an anti-V3 antibody comprising a VH domain of SEQ ID NO:70 and a VL domain of SEQ ID NO:71.

In an embodiment, the bispecific molecule comprises a CD4 domain of SEQ ID NO: 11 and an anti-V3 antibody comprising a VH domain of SEQ ID NO:70 and a VL domain of SEQ ID NO:71. In an embodiment, the bispecific molecule comprises a CD4 domain of any one of SEQ ID NOs: 1-21 and an anti-V3 antibody comprising a VH domain of SEQ ID NO:75 and a VL domain of SEQ ID NO:76.

In an embodiment, the bispecific molecule comprises a CD4 domain of SEQ ID NO: 11 and an anti-V3 antibody comprising a VH domain of SEQ ID NO:75 and a VL domain of SEQ ID NO:76. In an embodiment, the bispecific molecule comprises a CD4 domain of any one of SEQ ID NOs: 1-21 and an anti-V3 antibody comprising a VH domain of SEQ ID NO:80 and a VL domain of SEQ ID NO:81.

In an embodiment, the bispecific molecule comprises a CD4 domain of SEQ ID NO: 11 and an anti-V3 antibody comprising a VH domain of SEQ ID NO: 80 and a VL domain of SEQ ID NO:81.

In an embodiment, the bispecific molecule comprises a CD4 domain of any one of SEQ ID NOs: 1-21 and an anti-V3 antibody comprising a VH domain of SEQ ID NO:85 and a VL domain of SEQ ID NO:86.

In an embodiment, the bispecific molecule comprises a CD4 domain of SEQ ID NO: 11 and an anti-V3 antibody comprising a VH domain of SEQ ID NO:85 and a VL domain of SEQ ID NO:86.

An anti-V3 antibody as described above may be an antibody comprising a heavy chain (HC), with or without M428L/N434S (EU numbering) ‘LS’ mutations. In an embodiment, the HC comprises LS.

In an embodiment, the bispecific molecule comprises a CD4 domain of any one of SEQ ID NOs: 1-21 and an anti-V3 antibody comprising a HC of SEQ ID NO:61 or 62 and a LC of SEQ ID NO: 63 or 64.

In an embodiment, the bispecific molecule comprises a CD4 domain of SEQ ID NO: 11 and an anti-V3 antibody comprising a HC of SEQ ID NO:61 or 62 and a LC of SEQ ID NO: 63 or 64.

In an embodiment, the bispecific molecule comprises a CD4 domain of any one of SEQ ID NOs: 1-21 and an anti-V3 antibody comprising a HC of SEQ ID NO:62 and a LC of SEQ ID NO:63.

In an embodiment, the bispecific molecule comprises a CD4 domain of SEQ ID NO: 11 and an anti-V3 antibody comprising a HC of SEQ ID NO:62 and a LC of SEQ ID NO:63.

In an embodiment, the bispecific molecule comprises a CD4 domain of any one of SEQ ID NOs: 1-21 and an anti-V3 antibody comprising a HC of SEQ ID NO:67 or 68 and a LC of SEQ ID NO: 69.

In an embodiment, the bispecific molecule comprises a CD4 domain of SEQ ID NO: 11 and an anti-V3 antibody comprising a HC of SEQ ID NO:67 or 68 and a LC of SEQ ID NO:69.

In an embodiment, the bispecific molecule comprises a CD4 domain of any one of SEQ ID NOs: 1-21 and an anti-V3 antibody comprising a HC of SEQ ID NO: 72 or 73 and a LC of SEQ ID NO: 74.

In an embodiment, the bispecific molecule comprises a CD4 domain of SEQ ID NO: 11 and an anti-V3 antibody comprising a HC of SEQ ID NO:72 or 73 and a LC of SEQ ID NO:74.

In an embodiment, the bispecific molecule comprises a CD4 domain of any one of SEQ ID NOs: 1-21 and an anti-V3 antibody comprising a HC of SEQ ID NO:77 or 78 and a LC of SEQ ID NO: 79.

In an embodiment, the bispecific molecule comprises a CD4 domain of SEQ ID NO: 11 and an anti-V3 antibody comprising a HC of SEQ ID NO:77 or 78 and a LC of SEQ ID NO:79.

In an embodiment, the bispecific molecule comprises a CD4 domain of any one of SEQ ID NOs: 1-21 and an anti-V3 antibody comprising a HC of SEQ ID NO: 82 or 83 and a LC of SEQ ID NO: 84.

In an embodiment, the bispecific molecule comprises a CD4 domain of SEQ ID NO:11 and an anti-V3 antibody comprising a HC of SEQ ID NO:82 or 83 and a LC of SEQ ID NO:84.

In an embodiment, the bispecific molecule comprises a CD4 domain of any one of SEQ ID NOs: 1-21 and an anti-V3 antibody comprising a HC of SEQ ID NO:87 or 88 and a LC of SEQ ID NO: 89

In an embodiment, the bispecific molecule comprises a CD4 domain of SEQ ID NO:11 and an anti-V3 antibody comprising a HC of SEQ ID NO: 87 or 88 and a LC of SEQ ID NO:89.

In an embodiment, the bispecific molecule comprises an anti-V3 bNAb as disclosed herein and two copies of a CD4 domain as disclosed herein, wherein the C-terminus of one CD4 domain is attached directly or by a linker to the N-terminus of one of the anti-V3 bNAb heavy chains and the C-terminus of the other copy of the CD4 domain is attached directly or by a linker to the N-terminus of the other anti-V3 bNAb heavy chain.

In an embodiment, the bispecific molecule comprises an anti-V3 bNAb as disclosed herein and two copies of a CD4 domain as disclosed herein, wherein the C-terminus of one CD4 domain is attached directly or by a linker to the N-terminus of one of the anti-V3 bNAb light chains and the C-terminus of the other copy of the CD4 domain is attached directly or by a linker to the N-terminus of the other anti-V3 bNAb light chain.

In an embodiment, the bispecific molecule comprises an anti-V3 bNAb as disclosed herein and four copies of a CD4 domain as disclosed herein, wherein the C-terminus of the first CD4 domain is attached directly or by a linker to the N-terminus of one of the anti-V3 bNAb heavy chains, the C-terminus of the second CD4 domain is attached directly or by a linker to the N-terminus of the other anti-V3 bNAb heavy chains, the third CD4 domain is attached directly or by a linker to the N-terminus of one of the anti-V3 bNAb light chains, and the fourth CD4 domain is attached directly or by a linker to the N-terminus of the other anti-V3 bNAb light chains.

In an embodiment, the bispecific molecule comprises an anti-V3 bNAb as disclosed herein and two copies of a CD4 domain as disclosed herein, wherein the N-terminus of the first CD4 domain is attached directly or by a linker to the C-terminus of one of the anti-V3 bNAb heavy chains and the N-terminus of the other copy of the CD4 domain is attached directly or by a linker to the C-terminus of the other anti-V3 bNAb heavy chain.

In an embodiment, the bispecific molecule comprises an anti-V3 bNAb as disclosed herein and two copies of a CD4 domain of SEQ ID NO:11, wherein the C-terminus of one CD4 domain is attached by a linker of SEQ ID NO: 90 to the N-terminus of one of the anti-V3 bNAb heavy chains and the C-terminus of the other copy of the CD4 domain is attached by a linker of SEQ ID NO: 90 to the N-terminus of the other anti-V3 bNAb heavy chain.

In an embodiment, the bispecific molecule comprises an anti-V3 bNAb as disclosed herein and two copies of a CD4 domain of SEQ ID NO:11, wherein the C-terminus of one CD4 domain is attached by a linker of SEQ ID NO: 90 to the N-terminus of one of the anti-V3 bNAb light chains and the C-terminus of the other copy of the CD4 domain is attached by a linker of SEQ ID NO: 90 to the N-terminus of the other anti-V3 bNAb light chain.

In an embodiment, the bispecific molecule comprises an anti-V3 bNAb as disclosed herein and four copies of a CD4 domain of SEQ ID NO: 11, wherein the C-terminus of the first CD4 domain is attached by a linker of SEQ ID NO: 90 to the N-terminus of one of the anti-V3 bNAb heavy chains, the C-terminus of the second CD4 domain is attached by a linker of SEQ ID NO: 90 to the N-terminus of the other anti-V3 bNAb heavy chains, the third CD4 domain is by a linker of SEQ ID NO: 90 to the N-terminus of one of the anti-V3 bNAb light chains, and the fourth CD4 domain is attached by a linker of SEQ ID NO: 90 to the N-terminus of the other anti-V3 bNAb light chains.

In an embodiment, the bispecific molecule comprises an anti-V3 bNAb as disclosed herein and two copies of a CD4 domain of SEQ ID NO: 11, wherein the N-terminus of the first CD4 domain is attached by a linker of SEQ ID NO: 90 to the C-terminus of one of the anti-V3 bNAb heavy chains and the N-terminus of the other copy of the CD4 domain is attached by a linker of SEQ ID NO: 90 to the C-terminus of the other anti-V3 bNAb heavy chain.

In an embodiment, the bispecific molecule comprises an anti-V3 bNAb comprising a VH of SEQ ID NO: 58 and a VL of SEQ ID NO:59, and two copies of a CD4 domain of SEQ ID NO: 11, wherein the C-terminus of one CD4 domain is attached by a linker of SEQ ID NO: 90 to the N-terminus of one of the anti-V3 bNAb heavy chains and the C-terminus of the other copy of the CD4 domain is attached by a linker of SEQ ID NO: 90 to the N-terminus of the other anti-V3 bNAb heavy chain.

In an embodiment, the bispecific molecule an anti-V3 bNAb comprising a VH of SEQ ID NO: 58 and a VL of SEQ ID NO:59, and two copies of a CD4 domain of SEQ ID NO: 11, wherein the C-terminus of one CD4 domain is attached by a linker of SEQ ID NO: 90 to the N-terminus of one of the anti-V3 bNAb light chains and the C-terminus of the other copy of the CD4 domain is attached by a linker of SEQ ID NO: 90 to the N-terminus of the other anti-V3 bNAb light chain.

In an embodiment, the bispecific molecule comprises an anti-V3 bNAb comprising a VH of SEQ ID NO: 58 and a VL of SEQ ID NO:59, and four copies of a CD4 domain of SEQ ID NO: 11, wherein the C-terminus of the first CD4 domain is attached by a linker of SEQ ID NO: 90 to the N-terminus of one of the anti-V3 bNAb heavy chains, the C-terminus of the second CD4 domain is attached by a linker of SEQ ID NO: 90 to the N-terminus of the other anti-V3 bNAb heavy chains, the third CD4 domain is by a linker of SEQ ID NO: 90 to the N-terminus of one of the anti-V3 bNAb light chains, and the fourth CD4 domain is attached by a linker of SEQ ID NO: 90 to the N-terminus of the other anti-V3 bNAb light chains.

In an embodiment, the bispecific molecule comprises an anti-V3 bNAb comprising a VH of SEQ ID NO:58 and a VL of SEQ ID NO:59, and two copies of a CD4 domain of SEQ ID NO:11, wherein the N-terminus of the first CD4 domain is attached by a linker of SEQ ID NO: 90 to the C-terminus of one of the anti-V3 bNAb heavy chains and the N-terminus of the other copy of the CD4 domain is attached by a linker of SEQ ID NO: 90 to the C-terminus of the other anti-V3 bNAb heavy chain.

In an embodiment, the bispecific molecule comprises a HC of any one of SEQ ID NOs: 96-107, 116, 117 or 119-135; and a LC of SEQ ID NO:63.

In an embodiment, the bispecific molecule comprises a HC of SEQ ID NO:62; and a LC of any one of SEQ ID NOs: 108-115 and 118.

In an embodiment, the bispecific molecule comprises a HC of SEQ ID NO:68; and a LC of SEQ ID NO: 142 or 143.

In an embodiment, the bispecific molecule comprises a HC of any one of SEQ ID NOs: 136-141; and a LC of SEQ ID NO:69.

In an embodiment, the bispecific molecule comprises a HC of SEQ ID NO: 144 or 145; and a LC of SEQ ID NO:74.

In an embodiment, the bispecific molecule comprises a HC of SEQ ID NO:146 or 147; and a LC of SEQ ID NO:79.

In an embodiment, the bispecific molecule comprises a HC of SEQ ID NO:148 or 149; and a LC of SEQ ID NO:84.

In an embodiment, the bispecific molecule comprises a HC of SEQ ID NO: 150 or 151; and a LC of SEQ ID NO:89.

In an embodiment, the bispecific molecule consists of two heavy chains and two light chains, wherein the heavy chain is at least 95% identical to SEQ ID NO: 121 and the light chain that is at least 95% identical to SEQ ID NO:63.

In an embodiment, the bispecific molecule consists of two heavy chains and two light chains, wherein the heavy chain is at least 96% identical to SEQ ID NO: 121 and the light chain that is at least 96% identical to SEQ ID NO:63.

In an embodiment, the bispecific molecule consists of two heavy chains and two light chains, wherein the heavy chain is at least 97% identical to SEQ ID NO: 121 and the light chain that is at least 97% identical to SEQ ID NO:63.

In an embodiment, the bispecific molecule consists of two heavy chains and two light chains, wherein the heavy chain is at least 98% identical to SEQ ID NO: 121 and the light chain that is at least 98% identical to SEQ ID NO:63.

In an embodiment, the bispecific molecule consists of two heavy chains and two light chains, wherein the heavy chain is at least 99% identical to SEQ ID NO:121 and the light chain that is at least 99% identical to SEQ ID NO:63.

In an embodiment, the bispecific molecule consists of two heavy chains of SEQ ID NO: 121 and two light chains of SEQ ID NO:63.

An antigen binding protein of the invention may comprise an anti-V3 scFv of any one of the aforementioned anti-V3 bNAbs. In an embodiment, the scFv comprises a VH and VL pair as set out in Table 2. In an embodiment, the scFv comprises a VH and VL pair of any one of PGT121-123, PGT125-131, PGT135-137, QA013.2, 10-1074, 10-1074LS, PGT121.414.LS and 2G12. In an embodiment, the C-terminus of the VH domain is attached directly or via a linker to the N-terminus of the VL domain. In an embodiment, the C-terminus of the VL domain is attached directly or via a linker to the N-terminus of the VH domain. In an embodiment, the linker between the VH domain and the VL domain of the scFv is selected from the group consisting of SEQ ID NOs: 90-95. In an embodiment, the linker between the VH domain and the VL domain of the scFv is SEQ ID NO:93.

In an embodiment, the scFv comprises a VH domain of SEQ ID NO:58 and a VL domain of SEQ ID NO: 59. In an embodiment, the scFv comprises a VH domain of SEQ ID NO:65 and a VL domain of SEQ ID NO:66. In an embodiment, the scFv comprises a VH domain of SEQ ID NO:70 and a VL domain of SEQ ID NO:71. In an embodiment, the scFv comprises a VH domain of SEQ ID NO: 75 and a VL domain of SEQ ID NO:76. In an embodiment, the scFv comprises a VH domain of SEQ ID NO: 80 and a VL domain of SEQ ID NO: 81. In an embodiment, the scFv comprises a VH domain of SEQ ID NO: 85 and a VL domain of SEQ ID NO: 86. In an embodiment, a linker of SEQ ID NO: 93 joins the VH domain and the VL domain of the scFv. In an embodiment, a linker of SEQ ID NO: 93 joins the C terminal of the VH domain to the N terminal of the VL domain to form the scFv. In an embodiment, a linker of SEQ ID NO:93 joins the C terminal of the VL domain to the N terminal of the VH domain to form the scFv.

An anti-V3 scFv may be fused to an Fc domain. In an embodiment, the scFv is fused to a human Fc domain directly or via a linker (scFv-Fc). In an embodiment, the C-terminus of the scFv is fused to the N-terminus of a human Fc domain via a linker selected from the group consisting of SEQ ID NOs: 90-95. In an embodiment, the N-terminus of the scFv is fused to the C-terminus of a human Fc domain via a linker selected from the group consisting of SEQ ID NOs: 90-95. In an embodiment, the scFv is fused to a human Fc domain via a linker of SEQ ID NO:91. In an embodiment, the Fc domain comprises a half-life extending mutation. In an embodiment, the half-life extending mutation is LS.

A scFv-Fc may be fused directly or via a linker to a CD4 domain. In an embodiment, the scFv-Fc is fused via a linker selected from the group consisting of SEQ ID NOs: 90-95 to a CD4 domain.

In an embodiment, the antigen binding protein comprises or consists of: (1) a scFv comprising a VH and VL pair as set out in any row of Table 2, wherein the VH and VL domain are joined to form a scFV via a linker selected from the group consisting of SEQ ID NOs: 90-95; (2) a CD4 domain selected from the group consisting of SEQ ID NOs: 1-21; and (3) and Fc domain comprising LS half-life extending mutations; wherein (1), (2) and (3) are joined together in any order directly or via a linker, and wherein each linker is selected from the group consisting of SEQ ID NOs: 90-95.

In an embodiment, the antigen binding protein comprises or consists of: (1) a scFv comprising a VH and VL pair as set out in any row of Table 2, wherein the VH and VL domain are joined to form a scFV via a linker of SEQ ID NO:93; (2) a CD4 domain selected from the group consisting of SEQ ID NOs: 1-21; and (3) and Fc domain comprising LS half-life extending mutations; wherein (1), (2) and (3) are joined together in any order via a linker between each domain, and wherein the linker is SEQ ID NOs: 90.

In an embodiment, the bispecific molecule of the invention comprises a sequence that is at least 95%, 96%, 97%, 98%, 99% or 100% identical to any one of SEQ ID NOs: 152-157. In an embodiment, an antigen binding protein of the invention consists of a sequence that is at least 95%, 96%, 97%, 98%, 99% or 100% identical to any one of SEQ ID NOs: 152-157. In an embodiment, an antigen binding protein of the invention comprises or consists of SEQ ID NO: 152. In an embodiment, an antigen binding protein of the invention comprises or consists of SEQ ID NO:153. In an embodiment, an antigen binding protein of the invention comprises or consists of SEQ ID NO: 154. In an embodiment, an antigen binding protein of the invention comprises or consists of SEQ ID NO: 155. In an embodiment, an antigen binding protein of the invention comprises or consists of SEQ ID NO: 156. In an embodiment, an antigen binding protein of the invention comprises or consists of SEQ ID NO: 157.

### Production Methods

Antigen binding proteins may be prepared by any of a number of conventional techniques. For example, antigen binding proteins may be purified from cells that naturally express them (e.g., an antibody can be purified from a hybridoma that produces it), or produced in recombinant expression systems.

A number of different expression systems and purification regimes can be used to generate the antigen binding proteins of the invention. Generally, host cells are transformed with a recombinant expression vector encoding the desired antigen binding protein. The expression vector may be maintained by the host as a separate genetic element or integrated into the host chromosome depending on the expression system. A wide range of host cells can be employed, including Prokaryotes (including Gram-negative or Gram-positive bacteria, for example Escherichia coli, Bacilli sp., Pseudomonas sp., Corynebacterium sp.), Eukaryotes including yeast (for example Saccharomyces cerevisiae, Pichia pastoris), fungi (for example Aspergillus sp.), or higher Eukaryotes including insect cells and cell lines of mammalian origin (for example, CHO, NS0, PER.C6, HEK293, HeLa).

The host cell may be an isolated host cell. The host cell is usually not part of a multicellular organism (e.g., plant or animal). The host cell may be a non-human host cell.

Appropriate cloning and expression vectors for use with bacterial, fungal, yeast, and mammalian host cells are known in the art.

The cells can be cultured under conditions that promote expression of the antigen binding protein using a variety of equipment such as shake flasks, spinner flasks, and bioreactors. The polypeptide(s) is (are) recovered by conventional protein purification procedures. Protein purification procedures typically consist of a series of unit operations comprised of various filtration and chromatographic processes developed to selectively concentrate and isolate the antigen binding protein. The purified antigen binding protein may be formulated in a pharmaceutically acceptable composition.

### Fc Modifications

Fc engineering methods can be applied to modify the functional or pharmacokinetics properties of an antigen binding protein, in particular an antibody. Effector function may be altered by making mutations in the Fc region that increase or decrease binding to C1q or Fcγ receptors and modify CDC or ADCC activity respectively. Modifications to the glycosylation pattern of an antibody can also be made to change the effector function.

The interaction between the Fc region of an antigen binding protein or antibody and various Fc receptors (FcR), including FcγRI(CD64), FcγRII(CD32), FcγRIII(CD16), FcRn, C1q, and type II Fc receptors is believed to mediate the effector functions of the antigen binding protein or antibody. Significant biological effects can be a consequence of effector functionality. Usually, the ability to mediate effector function requires binding of the antigen binding protein or antibody to an antigen and not all antigen binding proteins or antibodies will mediate every effector function.

Effector function can be assessed in a number of ways including, for example, evaluating ADCC effector function of antibody coated to target cells mediated by Natural Killer (NK) cells via FcγRIII, or monocytes/macrophages via FcγRI, or evaluating CDC effector function of antibody coated to target cells mediated by complement cascade via Clq. For example, an antigen binding protein of the present invention can be assessed for ADCC effector function in a Natural Killer cell assay. Examples of such assays can be found in Shields et al, 2001, The Journal of Biological Chemistry, Vol. 276, p. 6591-6604; Chappel et al, 1993, The Journal of Biological Chemistry, Vol 268, p. 25124-25131; Lazar et al, 2006, PNAS, 103; 4005-4010.

Examples of assays to determine CDC function include those described in J Imm Meth, 1995, 184:29-38.

The effects of mutations on effector functions (e.g., FcRn binding, FcγRs and Clq binding, CDC, ADCML, ADCC, ADCP) can be assessed, e.g., as described in Grevys et al., J Immunol. 2015 Jun. 1; 194 (11): 5497-5508, or Tam et al., Antibodies 2017, 6 (3); Monnet et al., 2014 mAbs, 6:2, 422-436.

Throughout this specification, amino acid residues in Fc regions, in antibody sequences or full-length antigen binding protein sequences, are numbered according to the EU index numbering convention.

The long half-life of IgG antibodies is reported to be dependent on their binding to FcRn. Therefore, substitutions that increase the binding affinity of IgG to FcRn at pH 6.0 while maintaining the pH dependence of the interaction with target, by engineering the constant region, have been extensively studied (Ghetie et al., Nature Biotech. 15:637-640, 1997; Hinton et al., JBC 279:6213-6216, 2004; Dall'Acqua et al., 10 J Immunol 117:1129-1138, 2006). The in-vivo half-life of antigen binding proteins of the present invention may be altered by modification of a heavy chain constant domain or an FcRn binding domain therein.

In adult mammals, FcRn, plays a key role in maintaining serum antibody levels by acting as a protective receptor that binds and salvages antibodies of the IgG isotype from degradation. IgG molecules are endocytosed by endothelial cells and, if they bind to FcRn, are recycled out of the cells back into circulation. In contrast, IgG molecules that enter the cells and do not bind to FcRn and are targeted to the lysosomal pathway where they are degraded.

FcRn is believed to be involved in both antibody clearance and the transcytosis across tissues (see Junghans R. P (1997) Immunol.Res 16. 29-57 and Ghetie et al (2000) Annu. Rev. Immunol. 18, 739-766). Human IgG1 residues determined to interact directly with human FcRn include Ile253, Ser254, Lys288, Thr307, Gln311, Asn434 and His435. Mutations at any of these positions may enable increased serum half-life and/or altered effector properties of antigen binding proteins of the invention.

Antigen binding proteins of the present invention may have amino acid modifications that increase the affinity of the constant domain or fragment thereof for FcRn. Increasing the half-life (i.e., serum half-life) of therapeutic and diagnostic IgG antibodies and other bioactive molecules has many benefits including reducing the amount and/or frequency of dosing of these molecules. In one embodiment, an antigen binding protein of the invention comprises all or a portion (an FcRn binding portion) of an IgG constant domain having one or more of the following amino acid modifications.

For example, with reference to IgG1, M252Y/S254T/T256E (commonly referred to as “YTE” mutations) and M428L/N434S (commonly referred to as “LS” mutations) increase FcRn binding at pH 6.0 (Wang et al. 2018). In an embodiment, an antigen binding protein of the invention comprises an Fc domain with the LS mutations. In an embodiment, an antigen binding protein of the invention comprises a bNAb in which the LS mutations are present in both of the heavy chain Fc domains.

Half-life and FcRn binding can also be extended by introducing H433K and N434F mutations (commonly referred to as “HN” or “NHance” mutations) (with reference to IgG1) (WO2006/130834).

Additionally, various publications describe methods for obtaining physiologically active molecules with modified half-lives, either by introducing an FcRn-binding polypeptide into the molecules (WO97/43316, U.S. Pat. Nos. 5,869,046, 5,747,035, WO96/32478 and WO91/14438) or by fusing the molecules with antibodies whose FcRn-binding affinities are preserved, but affinities for other Fc receptors have been greatly reduced (WO99/43713), or fusing with FcRn binding domains of antibodies (WO00/09560, U.S. Pat. No. 4,703,039).

### Post-Translational Modifications

The skilled person will appreciate that, upon production of an antigen binding protein, such as a bispecific molecule of the invention in a host cell, post-translational modifications may occur. For example, this may include the cleavage of certain leader sequences, the addition of various sugar moieties in various glycosylation patterns, non-enzymatic glycation, deamidation, oxidation, disulfide bond scrambling and other cysteine variants such as free sulfhydryls, racemized disulfides, thioethers and trisulfide bonds, isomerisation, C-terminal lysine clipping, and N-terminal glutamine cyclisation. The present invention encompasses the use of antigen binding proteins that have been subjected to, or have undergone, one or more post-translational modifications. Thus an antigen binding protein of the invention includes an “antigen binding protein” as defined earlier that has undergone a post-translational modification such as described herein.

Glycation is a post-translational non-enzymatic chemical reaction between a reducing sugar, such as glucose, and a free amine group in the protein, and is typically observed at the epsilon amine of lysine side chains or at the N-Terminus of the protein. Glycation can occur during production and storage only in the presence of reducing sugars.

Deamidation can occur during production and storage, is an enzymatic reaction primarily converting asparagine (N) to iso-aspartic acid (iso-aspartate) and aspartic acid (aspartate) (D) at approximately 3:1 ratio. This deamidation reaction is therefore related to isomerization of aspartate (D) to iso-aspartate. The deamidation of asparagine and the isomerisation of aspartate, both involve the intermediate succinimide. To a much lesser degree, deamidation can occur with glutamine residues in a similar manner. Deamidation can occur in a CDR, in a Fab (non-CDR region), or in the Fc region.

Oxidation can occur during production and storage (i.e., in the presence of oxidizing conditions) and results in a covalent modification of a protein, induced either directly by reactive oxygen species or indirectly by reaction with secondary by-products of oxidative stress. Oxidation happens primarily with methionine residues, but may occur at tryptophan and free cysteine residues. Oxidation can occur in a CDR, in a Fab (non-CDR) region, or in the Fc region.

Disulfide bond scrambling can occur during production and basic storage conditions. Under certain circumstances, disulfide bonds can break or form incorrectly, resulting in unpaired cysteine residues (—SH). These free (unpaired) sulfhydryls (—SH) can promote shuffling.

The formation of a thioether and racemization of a disulphide bond can occur under basic conditions, in production or storage, through a beta elimination of disulphide bridges back to cysteine residues via a dehydroalanine and persulfide intermediate. Subsequent crosslinking of dehydroalanine and cysteine results in the formation of a thioether bond or the free cysteine residues can reform a disulphide bond with a mixture of D- and L-cysteine.

Trisulfides result from insertion of a sulfur atom into a disulphide bond (Cys-S—S-S-Cys) and are formed due to the presence of hydrogen sulphide in production cell culture.

N-terminal glutamine (Q) and glutamate (glutamic acid) (E) in the heavy chain and/or light chain is likely to form pyroglutamate (pGlu) via cyclization. Most pGlu formation happens in the production bioreactor, but it can be formed non-enzymatically, depending on pH and temperature of processing and storage conditions. Cyclization of N-terminal Q or E is commonly observed in natural human antibodies.

C-terminal lysine clipping is an enzymatic reaction catalyzed by carboxypeptidases, and is commonly observed in recombinant and natural human antibodies. Variants of this process include removal of lysine from one or both heavy chains due to cellular enzymes from the recombinant host cell. Upon administration to the human subject/patient is likely to result in the removal of any remaining C-terminal lysines.

### Pharmaceutical Compositions

Antigen binding proteins as described herein may be incorporated into pharmaceutical compositions for use in the treatment or prevention of HIV infection. In one embodiment, the pharmaceutical composition comprises an antigen binding protein in combination with one or more pharmaceutically acceptable carriers and/or excipients.

Such compositions comprise a pharmaceutically acceptable carrier as known and called for by acceptable pharmaceutical practice.

Pharmaceutical compositions may be administered by injection or continuous infusion (examples include, but are not limited to, intravenous, intraperitoneal, intradermal, subcutaneous, intramuscular, intraocular, and intraportal). In one embodiment, the composition is suitable for intravenous administration. In one embodiment, the composition is suitable for subcutaneous administration.

Pharmaceutical compositions may be suitable for topical administration (which includes, but is not limited to, epicutaneous, inhaled, intranasal or ocular administration) or enteral administration (which includes, but is not limited to, oral, vaginal, or rectal administration).

The pharmaceutical composition may be included in a kit containing the antigen binding protein together with other medicaments, for example dolutegravir or cabotegravir, and/or with instructions for use. For convenience, the kit may comprise the reagents in predetermined amounts with instructions for use. The kit may also include devices used for administration of the pharmaceutical composition.

The terms “individual”, “subject” and “patient” are used herein interchangeably. In one embodiment, the subject is a human.

The antigen binding proteins described herein may be used in methods of treatment or prevention of HIV infection and AIDs. The antigen binding proteins described herein may be used in the manufacture of medicaments for the treatment or prevention of HIV infection and AIDs. The antigen binding proteins described may be used in an effective amount for therapeutic, prophylactic or preventative treatment. A therapeutically effective amount of the antigen binding protein described herein is an amount effective to ameliorate or reduce one or more symptoms of HIV infection. A prophylactically effective amount of the antigen binding protein described herein is an amount effective to prevent one or more symptoms of HIV infection.

### Combinations

Antigen binding proteins of the present invention may be employed alone or in combination with other therapeutic agents, or a prodrug thereof. Combination therapies according to the present invention thus comprise the administration of an antigen binding protein and the administration of at least one other agent which may be useful in the treatment or prevention of HIV infection and/or AIDS. An antigen binding protein of the present invention and the other therapeutic agent may be formulated and administered together in a single pharmaceutical composition or may be formulated and administered separately. When formulated and administered separately, administration may occur simultaneously or sequentially in any order.

Antigen binding proteins as described herein may be combined with, for example, one or more of an antiretroviral agent, an anti-infective agent, an immunomodulator, and other HIV entry inhibitors.

Antiretroviral agents include Nucleoside Reverse Transcriptase Inhibitors (NRTIs), Non-Nucleoside Reverse Transcriptase Inhibitors (NNRTIs), Nucleoside Reverse Transcriptase Translocation Inhibitors (NRTTIs), Protease Inhibitors (PIS), Entry Inhibitors (EI), Integrase Strand Transfer Inhibitors (INSTI), Maturation Inhibitors (MIs), and Capsid Inhibitors (CIs).

NRTIs may include, but are not limited to: abacavir, adefovir, adefovir dipivoxil, alovudine, amdoxovir, apricitabine, calanolide A, censavudine, didanosine, elvucitabine, emtricitabine, fozivudine, lamivudine, racivir, stampidine, stavudine, tenofovir disoproxil fumerate, tenofovir alafenamide, todoxil, zalcitabine, and zidovudine.

NNRTIs may include, but are not limited to, HBY 097 (Hoechst/Bayer), capravirine, delaviridine, doravirine, efavirenz, etravirine, immunocal, lersivirine, loviride, nevirapine, oltipraz, and rilpivirine.

NRTTIs include, but are not limited to, islatravir.

PIs may include, but are not limited to, amprenavir, atazanavir, brecanavir, cobicistat, darunavir, fosamprenavir, indinavir, lasinavir, lopinavir, palinavir, nelfinavir, ritonavir, saquinavir, and tipranavir.

EIs are discussed in DRUGS OF THE FUTURE 1999, 24 (12), 1355-1362; CELL, Vol. 9, 243-246, Oct. 29, 1999; and DRUG DISCOVERY TODAY, Vol. 5, No. 5, May 2000, pp. 183-194; and Meanwell et al., Current Opinion in Drug Discovery & Development (2003), 6 (4), 451-461. In particular, the antigen binding proteins of the invention can be utilized in combination with attachment inhibitors, fusion inhibitors, and chemokine receptor antagonists aimed at either the CCR5 or CXCR4 coreceptor. HIV attachment inhibitors are also set forth in U.S. Pat. Nos. 7,354,924 and 7,745,625. EIs may include, but are not limited to, cenicriviroc, enfuvirtide, fostemsavir, ibalizumab, leronlimab, maraviroc, vicriviroc and VIR-576.

INSTIs may include, but are not limited to, bictegravir, cabotegravir, dolutegravir, elvitegravir, and raltegravir. In an embodiment, the INSTI is dolutegravir or cabotegravir. In an embodiment, the INSTI is cabotegravir.

Maturation inhibitors may include, but are not limited to, bevirimat, BMS-955176, GSK3640254, GSK3739937, PA-344 and PA-457. It will be understood that GSK3640254 is a compound as described in Dicker I, Jeffrey J L, Protack T, et al., Antimicrob Agents Chemother. 2022; 66 (1). GSK3739937, also known as VH3739937, is the compound of clinical trial NCT04493684.

Capsid inhibitors may include, but are not limited to, GSK4004280, GSK4011499, and lencapavir.

Anti-infective agents include, but are not limited to, clindamycin with primaquine, daunorubicin, fluconazole, intraconazole, nystatin pastille, ornidyl eflornithine, megestrol acetate, pentamidine isethionate, piritrexim, trimethoprim, trimetrexate, recombinant human erythropoietin, recombinant human growth hormone, spiramycin, testosterone and total enteral nutrition,

Immunomodulators include, but are not limited to, acemannan, alpha-2-interferon, AS-101, bropirimine, CL246,738, FP-21399, gamma interferon, granulocyte macrophage colony stimulating factor, HIV core particle immunostimulant, interleukin-2, immune globulin, IMREG-1, IMREG-2, imuthiol diethyl dithio carbamate, methionine enkephalin, MTP-PE muramyl tripeptide, remune, recombinant soluble human CD4, rCD4-IgG hybrids, SK&F106528, thymopentin, and tumour necrosis factor (TNF).

The antigen binding proteins of the present invention may also be used in combination with agents that induce HIV expression, such as latency reversing agents. Several latency reversing agents include, but are not limited to, the following: histone deacetylase inhibitors (e.g., vorinostat, panobinostat, romidepin), histone crotonyl transferase inhibitors (sodium corotonate), protein kinase C agonists (e.g., bryostatin, ingenol B), disulfiram, TLR7 agonists (e.g., GS-9620), and bromodomain inhibitors (e.g., JQ1, iBET151).

The antigen binding proteins of the present invention may also be used in combination with other agents that induce HIV expression, such as agents for clearance therapy. Several examples of agents for clearance therapy, or of immunological combinations for clearance, include, but are not limited to, the following: bNAbs, CD4-Ig, eCD4-Ig, and dual-affinity re-targeting (DART) proteins.

Antigen binding proteins of the invention may be used in combination with broadly neutralizing HIV-1 antibodies, including 1NC9, 1B2530, 2F5, 2G12, 3NBC60, 3BNC117, 4E10, 8ANC131, 8ANC134, 10-1074, 10-1074LS, 10E8, 12A12, 12A21, b12, CAP206-CH12, CH01-04, CH103-106, elipovimab (formerly known as GS-9722), HJ16, M66.6, N6LS (also known as VRC-HIVMAB091-00-AB and the compound of clinical trial NCT03538626), NIH45-46, PG9, PG16, PGT121-123, PGT125-131, PGT135-137, PGT141-145, PGT121.414.LS, PGT151 2G12, QA013.2, VRC01-03, VRC-PG04, VRC-PG04b, VRC-CH30-34.

Other agents that may be combined with antigen binding proteins of the invention include BIT225, GSK4000422/VH4000422, and S-648414 (the compound of clinical trial NCT04147715).

The scope of combinations of compounds of this invention with HIV agents is not limited to those mentioned above but includes in principle any combination with any pharmaceutical composition useful for the treatment and/or prevention of HIV infection and/or AIDS.

The invention is illustrated by the following clauses:
1. An anti-HIV gp120-binding protein that binds to at least two different epitopes on human immunodeficiency virus (HIV) surface glycoprotein 120 (gp120).
2. The anti-HIV gp120-binding protein of clause 1, wherein one of the at least two epitopes comprises one or more amino acid residues of the CD4-binding site of HIV gp120.
3. The anti-HIV gp120-binding protein of clause 1 or clause 2, wherein one of the at least two epitopes comprises one or more amino acid residues of the V3 loop region (V3) of HIV gp120.
4. The anti-HIV gp120-binding protein of clause 3, wherein one of the at least two epitopes comprises a cluster of mannose glycans centered on N332 of HIV gp120.
5. The anti-HIV gp120-binding protein of any one of the preceding clauses that binds to the CD4-binding site of HIV gp120 and the V3 loop region of HIV gp120.
6. The anti-HIV gp120-binding protein of any one of the preceding clauses, comprising a CD4-domain.
7. The anti-HIV gp120-binding protein of clause 6, wherein the CD4 domain is a CD4 D1 domain or CD4 D1D2 domain.
8. The anti-HIV gp120-binding protein of clause 6 or clause 7, wherein the CD4 domain comprises one or more stabilizing mutations.
9. The anti-HIV gp120-binding protein of clause 8, wherein the CD4 domain has a Tm of between 70° C. and 95° C.
10. The anti-HIV gp120-binding protein of any one of clauses 6 to 9, wherein the CD4 D1 domain comprises one or more mutations selected from the group consisting of: K8C, K8I, K8V, T11C, E13C, K21C, Q25E, H27C, H27D, G38C, N52W, R58N, R58T, R58V, L61M, G65C, I70C, K72C, E87G, E91H, E91Q, and G99C.
11. The anti-HIV gp120-binding protein of clause 9, wherein the CD4 domain has a Tm of about 90° C.
12. The anti-HIV gp120-binding protein of clause 10 or clause 11, wherein the CD4 domain comprises K8C and G99C.
13. The anti-HIV gp120-binding protein of clause 6, comprising any one of SEQ ID NOs: 1-21.
14. The anti-HIV gp120-binding protein of any one of clauses 6 to 13, comprising SEQ ID NO: 11.
15. The anti-HIV gp120-binding protein of any one of clauses 3 to 14, comprising a set of 6 CDRs (CDRH1, CDRH2, CDRH3, CDRL1, CDRL2 and CDRL3) as set forth in any row of Table 1.
16. The anti-HIV gp120-binding protein of clause 15, comprising a CDRH1 of SEQ ID NO:22, a CDRH2 of SEQ ID NO:23, a CDRH3 of SEQ ID NO:24, a CDRL1 of SEQ ID NO: 25, a CDRL2 of SEQ ID NO:26 and a CDRL3 of SEQ ID NO:27.
17. The anti-HIV gp120-binding protein of any one of the preceding clauses, comprising an immunoglobulin (Ig) scaffold.
18. The anti-HIV gp120-binding protein of clause 15, clause 16 or clause 17, comprising a heavy chain variable (VH) domain and a light chain variable (VL) domain pair as set forth in any row of Table 2.
19. The anti-HIV gp120-binding protein of clause 18, comprising a VH domain of SEQ ID NO: 58 and a VL domain of SEQ ID NO:59 or SEQ ID NO:60.
20. The anti-HIV gp120-binding protein of any one of clauses 17 to 19, comprising an Fc domain.
21. The anti-HIV gp120-binding protein of clause 20, wherein the Fc domain comprises a mutation that increases the half-life of the anti-HIV gp120-binding protein compared to the same anti-HIV gp120-binding protein without said mutation.
22. The anti-HIV gp120-binding protein of clause 21, wherein the Fc domain comprises any one of the following sets of mutations (EU numbering):
   M428L and N434S (LS);
   L309D, Q311H and N434S (DHS);
   M252Y, S254T and T256E (YTE); and
   H433K and N434F(HN).
23. The anti-HIV gp120-binding protein of clause 22, wherein the Fc domain comprises LS.
24. The anti-HIV gp120-binding protein of any one of the preceding clauses comprising a broadly neutralizing antibody (bNAb).
25. The anti-HIV gp120-binding protein of clause 24, wherein the bNAb is an anti-V3 bNAb.
26. The anti-HIV gp120-binding protein of clause 25, wherein the anti-V3 bNAb comprises a heavy chain (HC) and a light chain (LC) pair as set forth in any row of Table 2.
27. The anti-HIV gp120-binding protein of clause 26, wherein the HC comprises SEQ ID NO: 61 or SEQ ID NO: 62 and the LC comprises SEQ ID NO: 63 or SEQ ID NO:64.
28. The anti-HIV gp120-binding protein of clause 27, wherein the HC comprises SEQ ID NO: 62 and the LC comprises SEQ ID NO: 63.
29. The anti-HIV gp120-binding protein of any one of clauses 17 to 19, comprising an anti-V3 bNAb scFv.
30. The anti-HIV gp120-binding protein of clause 29, wherein the scFv comprises a VH domain of SEQ ID NO:58 and a VL domain of SEQ ID NO:59.
31. The anti-HIV gp120-binding protein of clause 29 or clause 30, wherein the C-terminus of the VH domain is fused directly or via a linker to the N-terminus of the VL domain.
32. The anti-HIV gp120-binding protein of clause 29 or clause 30, wherein the C-terminus of the VL domain is fused directly or via a linker to the N-terminus of the VH domain.
33. The anti-HIV gp120-binding protein of clause 31 or clause 32, wherein the linker between the VH domain and the VL domain of the scFv is selected from the group consisting of SEQ ID NOs: 90-95.
34. The anti-HIV gp120-binding protein of clause 33, wherein the linker between the VH domain and the VL domain of the scFv is SEQ ID NO: 93.
35. The anti-HIV gp120-binding protein of any one of clauses 29 to 34, wherein the scFv is fused to a human Fc domain directly or via a linker (scFv-Fc).
36. The anti-HIV gp120-binding protein of clause 35, wherein the scFv is fused to a human Fc via a linker selected from the group consisting of SEQ ID NO:90-95.
37. The anti-HIV gp120-binding protein of clause 35, wherein the scFv is fused to a human Fc via a linker of SEQ ID NO:91.
38. The anti-HIV gp120-binding protein of any one of clauses 35 to 37, wherein the Fc domain is as defined in any one of claims 21 to 23.
39. The anti-HIV gp120-binding protein of any one of clauses 35 to 38, comprising any one of SEQ ID NOs: 152-157.
40. A bispecific anti-HIV gp120-binding protein comprising an anti-V3 bNAb and two copies of a CD4 domain, wherein the C-terminus of one CD4 domain is attached directly or by a linker to the N-terminus of one of the anti-V3 bNAb heavy chains and the C-terminus of the other copy of the CD4 domain is attached directly or by a linker to the N-terminus of the other anti-V3 bNAb heavy chain.
41. The bispecific protein according to clause 40, wherein each CD4 domain is attached via a linker to each of the heavy chains.
42. The bispecific protein according to clause 41, wherein the linker is selected from the group consisting of SEQ ID NOs: 90 to 95.
43. The bispecific protein according to clause 42, wherein the linker is SEQ ID NO:90.
44. The bispecific protein according to any one of clauses 40 to 43, wherein the CD4 domain is selected from the group consisting of SEQ ID NO: 1-21.
45. The bispecific protein according to clause 44, wherein the CD4 domain is SEQ ID NO: 11.
46. The bispecific protein according to any one of clauses 40 to 45, wherein the anti-V3 bNAb is selected from the group consisting of bNAb1, bNAb1*, bNAb2, bNAb3, bNAb4, bNAb5 and bNAb6 as set forth in Table 2.
47. The bispecific protein as claimed in claim 46, wherein the anti-V3 bNAb is bNAb1.
48. The bispecific protein as claimed in any one of claims 40 to 47, wherein the anti-V3 bNAb Fc comprises LS.
49. An anti-HIV gp120-binding protein having two identical heavy chains and two identical light chains, comprising or consisting of:
   a heavy chain that is at least 95% identical to SEQ ID NO:121 and
   a light chain that is at least 95% identical to SEQ ID NO:63.
50. An anti-HIV gp120-binding protein consisting of two heavy chains of SEQ ID NO:121 and two light chains of SEQ ID NO:63.
51. An anti-HIV gp120-binding protein comprising or consisting of a sequence that is at least 95% identical to any one of SEQ ID NOs: 152-157.
52. An anti-HIV gp120-binding protein consisting of SEQ ID NO: 155.
53. A pharmaceutical composition comprising the anti-HIV gp120-binding protein as defined in any one of the preceding clauses and a pharmaceutically acceptable excipient.
54. A method of treating or preventing an HIV infection in a human comprising administering to the human an anti-HIV gp120-binding protein according to any one of clauses 1 to 52, or a pharmaceutical composition according to clause 53, whereby viral load in the human is decreased.
55. An anti-HIV gp120-binding protein according to any one of clauses 1 to 52, or a pharmaceutical composition according to clause 53, for use in treating or preventing an HIV infection in a human.
56. Use of an anti-HIV gp120-binding protein according to any one of clauses 1 to 52, or a pharmaceutical composition according to clause 53, in the manufacture of a medicament for treating or preventing an HIV infection in a human.
57. A kit comprising in separate containers: an anti-HIV gp120-binding protein according to any one of clauses 1 to 52 and an anti-viral drug that inhibits cellular entry, replication, or transcription of HIV in a human.
58. The kit according to clause 57, wherein the antiviral drug is selected from the group consisting of: Nucleoside Reverse Transcriptase Inhibitors (NRTIs), Non-Nucleoside Reverse Transcriptase Inhibitors (NNRTIs), Protease Inhibitors (PIS), Entry Inhibitors, Integrase Strand Transfer Inhibitors (INSTI), Maturation Inhibitors (MIS), Capsid Inhibitors (CIs) and Nucleoside Reverse Transcriptase Translocation Inhibitors (NRTTIs)
59. The kit according to clause 58, wherein the antiviral drug is an INSTI.
60. The kit according to clause 59, wherein the INSTI is dolutegravir or cabotegravir.
61. A nucleic acid sequence that encodes an anti-HIV gp120-binding protein according to any one of clauses 1 to 52.
62. An expression vector that comprises the nucleic acid sequence of clause 61.
63. A host cell that comprises the nucleic acid sequence of clause 61 or the expression vector of clause 62.
64. A host cell that comprises two expression vectors:
   a first expression vector comprising a nucleic acid sequence encoding a heavy chain of SEQ ID NO: 121; and
   a second expression vector comprising a nucleic acid sequence encoding a light chain of SEQ ID NO:63.
65. A method of producing an anti-HIV gp120-binding protein, comprising culturing the host cell as defined in clauses 63 or 64 under conditions suitable for expression of said nucleic acid sequence or vector, whereby an anti-HIV gp120-binding protein is produced.
66. A soluble CD4 domain having a Tm above 70° C.
67. A soluble CD4 domain comprising one or more stabilizing mutations selected from the group consisting of K8C, K8I, K8V, T11C, E13C, K21C, Q25E, H27C, H27D, G38C, N52W, R58N, R58T, R58V, L61M, G65C, I70C, K72C, E87G, E91H, E91Q, and G99C.
68. The soluble CD4 domain of clause 66 or clause 67 having a Tm of between 70° C. and 95° C.
69. The soluble CD4 domain of clause 68 having a Tm of about 90° C.
70. The soluble CD4 domain of any one of clauses 66 to 69, comprising K8C and G99C.
71. The soluble CD4 domain of any one of clauses 66 to 68, comprising K8I.
72. The soluble CD4 domain of any one of clauses 66 to 68, comprising K8V.
73. The soluble CD4 domain of any one of clauses 66 to 68, comprising T11C and K72C
74. The soluble CD4 domain of any one of clauses 66 to 68, comprising any one of SEQ ID NOs: 5-21.
75. The soluble CD4 domain of clause 74, comprising SEQ ID NO: 11.
76. The soluble CD4 domain of any one of clauses 66 to 75, wherein the CD4 domain is fused directly or via a linker to a human Fc domain.
77. The soluble CD4 domain of clause 76, wherein the Fc domain comprises LS.
78. The soluble CD4 domain of clause 76 or clause 77, wherein the linker is selected from the group consisting of SEQ ID NOs: 90-95.

### Examples

### Example 1—Antigen Binding Protein Production

Plasmids encoding the antigen binding proteins of the invention were expressed in EXPI293 or FREESTYLE 293-F cells using the manufacturer's standard protocol (ThermoFisher Scientific, Waltham, MA). The expressed medium was harvested by centrifugation (4000 rpm for 10 min) and the antigen binding proteins were purified by filtration through a 0.22 μm filter (Millipore Sigma, Burlington, MA) and fast protein liquid chromatography (FPLC) (ÄKTATM Pure, Cytiva, Marlborough MA). The medium was then passed through a Mabselect SuRe column (Cytiva, Marlborough MA) to capture the antigen binding proteins and the column was washed sequentially with phosphate-buffered saline (PBS) before elution.

The antigen binding proteins were then exchanged into a final buffer by using dialysis, a desalting column and preparative size exclusion column (SEC). The purity of the antigen binding proteins was evaluated by using sodium dodecyl sulphate-polyacrylamide gel electrophoresis (SDS-PAGE) and on a size exclusion column on a high-performance liquid chromatography system (SEC-HPLC).

Antigen binding protein concentrations were determined by measuring absorbance at 280 nm wavelength (A280) on a NanoDrop machine (ThermoFisher Scientific, Waltham, MA), and their molecular mass was measured by using liquid chromatography-mass spectrometry (LC-MS) to confirm their identity.

The endotoxin level in the final purified products was measured on an ENDOSAFE system (Charles River Labs, Wilmington MA) to make sure it was sufficiently low (usually <1 EU (Endotoxin Unit)/mg of protein) for downstream anti-viral studies.

### Example 2—Anti-Viral Activity

The anti-viral activity of the antigen binding proteins was measured in a pseudotyped virus (PSV) assay. Pseudotyped HIV-1 virus (PSV) contains deletions in the genome that make it unable to produce infectious virions, but it can be used to measure the activity of cell entry inhibitors (i.e., molecules that prevent the binding of HIV-1 virions to the target cell membrane and/or prevent entry of HIV-1 into target cells), which include the antigen binding proteins of the invention.

PSV was produced in HEK-293T cells (ATCC, Manassas VA) by co-transfecting expression plasmids encoding the HIV-1 gp160 envelope gene and an HIV-1 backbone plasmid using TRANSIT-2020 transfection reagent (Mirus Bio, Madison WI). A panel of HIV-1 PSVs expressing different gp160 envelope trimers was generated to evaluate the effectiveness of the antigen binding proteins of the invention against a wide spectrum of HIV-1 strains.

i. ACTOne Cells

The genome of PSV used in this assay contains a luciferase gene that is expressed once the virus enters cells. Accordingly, the luminescence signal (after adding a substrate of luciferase) can be used to determine the level of viral infection.

The 50% tissue culture infectious dose (TCID) of a single thawed aliquot of each batch of PSV was determined in ACTOne cells. The ACTOne cell-line used in this assay was derived in-house from a genetically engineered 293T cell clone that expresses CD4, CXCR4, and CCR5. Cells were maintained in growth medium composed of Dulbecco's modified Eagle's medium (DMEM, Life Technologies) at 37° C. in a humidified 5% CO2-95% air environment. Cell monolayers were split by treatment with Trypsin-EDTA (0.05%).

To run the anti-viral assay, ACTOne cells were detached by treating the cell culture flask with trypsin (trypsinization) and resuspended in growth medium containing 2% of DMSO to a density of 2.5×10⁵ cells/ml. One hundred μl of such cells was added to 10 μl of antigen binding protein pre-loaded in a 96-well plate. Ninety μl of PSV was then added to each well. The assay plates were incubated at 37° C. in a humidified incubator at 5% CO2 level. Plates were developed after 72 hours of incubation by adding 50 μl of BRIGHTGLO luciferase reagent (Promega, Madison WI) to each well, and transferring the plates to an ENVISION multilabel plate reader (PerkinElmer, Waltham MA) to measure the luminescence and determine the level of virus that had infected the cells. The higher the luminescence signal, the higher the level of infection.

Raw data were analyzed using an in-house template in an IDBS system to calculate half-maximal inhibitory concentration (IC50) values which reflects the activity of the antigen binding proteins of the invention at inhibiting viral entry (the smaller the number is, the more active the molecule is).

ii. TZM.bl cells

Alternatively, the PSV assay was carried out using a luciferase-based assay in a TZM.bl cell line. The TZM-bl cell line is derived from a Hela cell clone that was engineered to express CD4, CCR5 and CXCR4 and to contain integrated reporter genes for firefly luciferase and E. coli β-galactosidase under the control of an HIV-1 long terminal repeat (Wei et al., Antimicrobial agents and chemotherapy 46:1896-905 (2002)) permitting sensitive and accurate measurements of infection.

The detailed materials and methodology have been described elsewhere (Mentefiori, Curr. Protoc. Immunol., 2005, Chapter 12; Seaman et al., Journal of Virology, February 2010, 84 (3), p. 1439-1452). In brief, the assay measures the reduction in luciferase reporter gene expression in TZM.bl cells following a single round of virus infection.

Five-fold serial dilutions of the antigen binding proteins of the invention, from 50 μg/ml to 3.2 ng/ml, were performed in duplicate in 10% DMEM growth medium (100 μ/well). An amount of 200 TCID50 (50% tissue culture infectious dose) of virus was added to each well in a volume of 50 μl, and the plates were incubated for 1 h at 37° C.

TZM.bl cells were then added (1×10⁴/well in a 100-μl volume) in 10% D-MEM growth medium containing DEAE-dextran (Sigma, St. Louis, MO) at a final concentration of 11 μg/ml. Assay controls included TZM.bl cells alone (cell control) and TZM.bl cells with virus (virus control).

Following a 48-hour incubation at 37° C., 150 μl of assay medium was removed from each well and 100 μl of BRIGHTGLO luciferase reagent (Promega, Madison, WI) was added. The cells were allowed to lyse for 2 min, and then 150 μl of the cell lysate was transferred to a 96-well black solid plate, and luminescence was measured using a Victor 3 luminometer (Perkin Elmer).

The 50% and 80% inhibitory concentration (IC50 and IC80) values were calculated as the serum dilution that caused a 50% and 80% reduction respectively, in relative luminescence units (RLU) compared to the level in the virus control wells after subtraction of cell control RLU. All data were analyzed with 5-parameter curve fitting using neutralizing antibody analysis software provided by the CAVD Vaccine Immunology Statistical Center.

### Example 3—Stability of Soluble CD4 Domains

All soluble human CD4 domains tested contain a set of “base” mutations in human CD4 domain 1 (D1) over the wild-type sequence (SEQ ID NO:3) that enable the folding of human CD4 D1 on its own. Soluble CD4 D1 with this set of mutations is known as mD1.22 (Chen et al., J Virol. 2014 Jan; 88 (2): 1125-39) and the mutations therein consist of: L5Y, S23N, A55V, I76P, L96V, and F98V (SEQ ID NO:4, also referred to as D1m herein).

To achieve better developability and pharmacokinetics, further mutations were introduced into mD1.22 (SEQ ID NO:4) to enhance its thermal stability. The additional stabilizing mutations were designed based on several methodologies: 1) computational simulation by using Free Energy Perturbation (FEP+, Schrodinger, New York, NY USA); 2) computational simulation by using disulfide-bond scan in Molecular Operating Environment program (MOE, Chemical Computing Group, Montreal Canada); and 3) panning a library of human CD4 D1 with each residue mutated, one by one, to the other 19 types of amino acids (site saturation mutagenesis, TWIST BioScience, San Francisco, CA USA) using phage display under thermally challenging conditions (i.e., incubating the phage at room temperature, 70° C., and 80° C., then selecting the CD4 domain variants that can still bind to recombinant HIV-1 gp120 (CN54 strain, Acro Biosystems, Beijing China)).

The best performing variants (SEQ ID NOs: 5-21) were fused with 6×His tag at their C-termini, expressed and purified from mammalian cells using methods as described in Example 1, except that purification was via a Ni-NTA resin (Cytiva, Marlborough MA) instead of Mabselect SuRe column, with standard protocol from the vendor.

These purified CD4 D1 variants (with C-terminal 6×His tag) were then evaluated to determine their melting temperature (Tm, using Prometheus System, NanoTemper, München Germany), which indicates thermal stability, as well as their anti-viral activity against HIV-1 pseudotyped virus (see Example 2 above for methods using ACTOne cells).

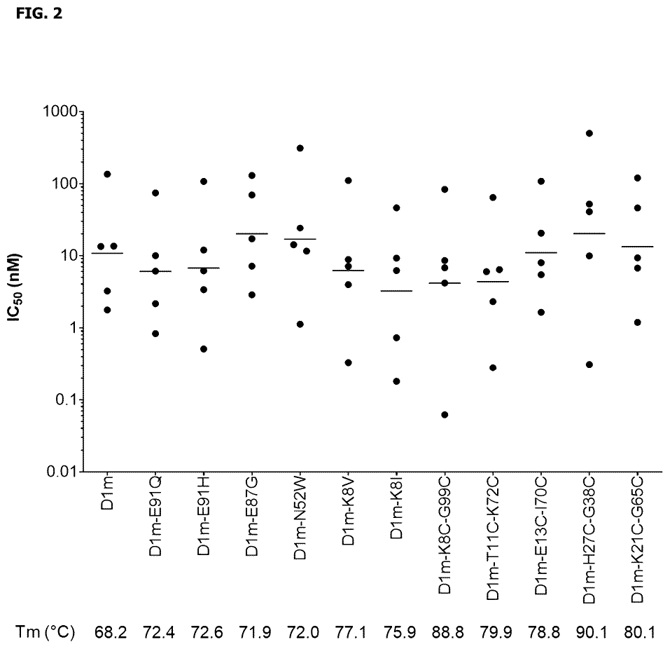

As shown in Table 3 below and in FIG. 2, several CD4 D1 variants (SEQ ID NO: 5-15) showed dramatically improved thermal stability over the “baseline” or “control” CD4 D1 (D1m, SEQ ID NO: 4), while maintaining similar anti-viral activity.

TABLE 3
Melting temperature of soluble CD4 domains

| CD4 Domain | SEQ ID NO | Tm (° C.) |
|---|---|---|
| D1m | 4 | 68.2 |
| D1m-E91Q | 6 | 72.4 |
| D1m-E91H | 7 | 72.6 |
| D1m-E87G | 8 | 71.9 |
| D1m-N52W | 9 | 72 |
| D1m-K8V | 5 | 77.1 |
| D1m-K8I | 10 | 75.9 |
| D1m-K8C-G99C | 11 | 88.8 |
| D1m-T11C-K72C | 12 | 79.9 |
| D1m-E13C-I70C | 13 | 78.8 |
| D1m-H27C-G38C | 14 | 90.1 |
| D1m-K21C-G65C | 15 | 80.1 |

### Example 4—Antigen Binding Protein Format and Linker Length

The fusion position of the CD4 domain in the anti-V3 bNAbs (e.g., whether to fuse the CD4 domain to the light chain or heavy chain or both, whether to fuse the CD4 domain to the N-terminus or C-terminus of these chains, or whether to fuse the CD4 domain in the middle of the heavy chain (in between CH1 and CH2 domains)) has an effect on the anti-viral potency of the resulting bispecific as shown in Table 4.1 and Table 5 below.

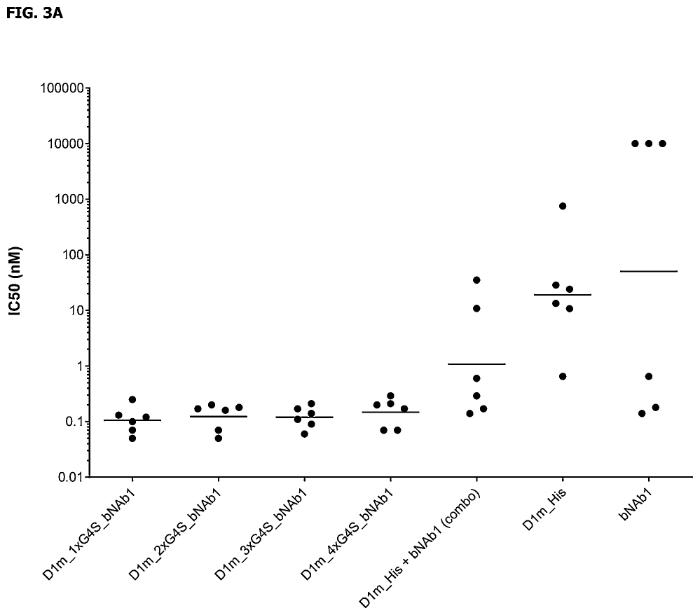

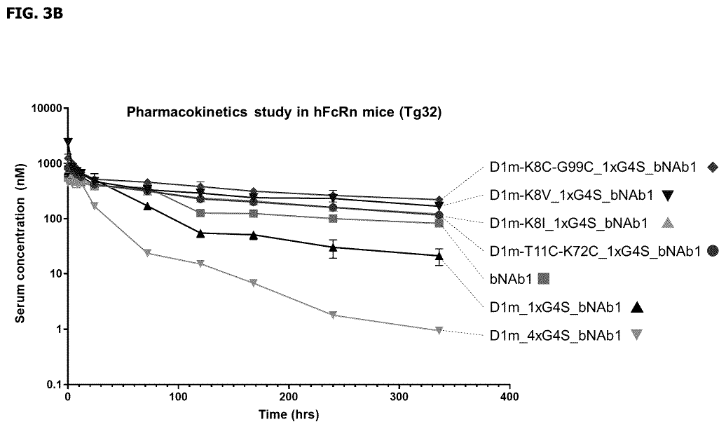

We observed that the most potent bispecific molecule resulted from fusing CD4 D1 to the N-terminus of the heavy chain of bNAb1 (molecule 1 in Table 4.1, which neutralized 6 envelopes with IC50<160 pM and 1 envelope with IC50 about 3 nM in PSV assay). In this bispecific format, the linker length between the CD4 domain and bNAb1 heavy chain N-terminus does not particularly affect anti-viral activity (FIG. 3A), but changes the pharmacokinetics (PK) of the resultant bispecific molecules dramatically (FIG. 3B).

As shown in FIG. 3B, the shorter-linker bispecific (D1m_1xG4S_bNAb1, SEQ ID NOs: 102 and 63) showed much better PK (longer half-life and lower clearance rate) than the longer-linker bispecific (D1m_4xG4S_bNAb1, SEQ ID NOs: 105 and 63)) in a humanized mouse model (Tg32 strain where human neonatal Fc receptor (hFcRn) replaced the corresponding mouse gene (mFcRn), The Jackson Laboratory, Bar Harbor, Maine USA).

TABLE 4.1
(see also FIG. 7) IC50 (nM) of different bNAb1-derived bispecific formats and control molecules
against a panel of HIV-1 envelopes in a PSV assay (ACTOne cells)

| | bNAb1-Derived Molecules | | | | | | | | | | |
|---|---|---|---|---|---|---|---|---|---|---|---|
| Envelope | 1 | 2 | 3 | 4 | 5 | 6 | 7 | 8 | 9 | 10 | 11 |
| 42368 | 0.16 | 0.91 | 1.49 | 4.90 | 1.24 | 15.73 | 5.60 | 1.44 | 4.32 | 1.58 | >500 |
| CC1/85 | 0.04 | 0.05 | 0.49 | 0.01 | 0.01 | 0.03 | 0.04 | 0.07 | 7.02 | 1.98 | 0.03 |
| NL4-3 | 0.05 | 0.17 | 0.17 | 0.10 | 0.14 | 0.52 | 0.45 | 0.22 | 0.31 | 0.31 | 131.20 |
| T278.50 | 0.12 | 0.12 | 1.29 | 0.04 | 0.02 | 0.06 | 0.13 | 0.11 | 3.99 | 1.43 | 0.17 |
| X2088.C9 | 0.07 | 0.86 | 0.72 | 13.28 | 1.50 | 67.68 | 18.25 | 5.25 | 17.49 | 9.20 | 402.35 |
| ZM106.9 | 0.05 | 0.18 | 0.38 | 0.04 | 0.03 | 0.05 | 0.07 | 0.09 | 26.02 | 15.14 | 0.07 |
| CAP45.2.00.G3 | 3.73 | 2.68 | 2.53 | 13.89 | 2.13 | 20.44 | 102.16 | 20.41 | 131.43 | 20.48 | >500 |

Table 4.1 Molecule Key:
1 = D1m-K8C-G99C_1xG4S_bNAb1 (SEQ ID NOs: 121 + 63)
2 = D1m-K8C-G99C_1xG4S_bNAb1-lc (SEQ ID NOs: 62 + 115)
3 = D1m-1xG4S_bNAb1-BothChains (SEQ ID NOs: 102 + 109)
4 = bNAb1-mid_1xG4S_D1m-K8C-G99C (SEQ ID NOs: 116 + 63)
5 = bNAb1-HC_1xG4S_D1m-K8C-G99C (SEQ ID NOs: 117 + 63)
6 = bNAb1-LC_1xG4S_D1m-K8C-G99C (SEQ ID NOs: 62 + 118)
7 = D1m-His (SEQ ID NO: 4*) + bNAb1(SEQ ID NOs: 62 + 63) (combo)
8 = D1m-K8C-G99C_Fc (SEQ ID NO: 158) + bNAb1 (SEQ ID NOs: 62 + 63) (combo)
9 = D1m-His (SEQ ID NO: 4*)
10 = D1m-K8C-G99C_Fc (SEQ ID NO: 158)
11 = bNAb1 (SEQ ID NOs: 62 + 63)
* plus a 6xHis tag (six C-terminal histidine residues)

TABLE 4.2
IC50 (nM) of a bNAb1-derived bispecific against entry inhibitor
resistant envelopes in a PSV assay (ACTOne cells)

| | | bNAb1-derived |
|---|---|---|
| Type | Envelope | bispecific |
| 10E8-insensitive | KER2008.12 | 0.05 |
| | T266-60 | 0.06 |
| | ZM106.9 | 0.05 |
| | X2088.c9 | 0.07 |
| | MB539.2B7 | 0.04 |
| | JR2 with W680R | 0.03 |
| | and K683Q | |
| N6-insensitive | BL01 | 0.07 |
| | T278-50 | 0.12 |
| | 6471_V1_C16 | 0.06 |
| | CH0219_E4 | 0.08 |
| Temsavir- and Ibalizumab- | 21-116102 | 0.36 |
| insensitive | 21-116108 | 0.01 |
| Maraviroc-insensitive | MP5.7 | 0.13 |
| | MP11.38 | 0.05 |
| | MP35.2 | 0.13 |
| | MP49.20 | 0.02 |
| | MP53.36 | 0.04 |

Table 4.2 Molecule Key:
1 = D1m-K8C-G99C_1xG4S_bNAb1 (SEQ ID NOs: 121 + 63)

TABLE 5
IC50 (nM) of different bNAb2-, bNAb3- and bNAb4-derived bispecific formats and
control molecules against a panel of HIV-1 envelopes in PSV assay (ACTOne cells)

| | | | | | | | | bNAb3-Derived | | |
|---|---|---|---|---|---|---|---|---|---|---|
| | bNAb2-Derived Molecules | | | | | | | Molecules | | |
| Envelope | 1 | 2 | 3 | 4 | 5 | 6 | 7 | 8 | 9 | 10 |
| 42368 | 1.79 | 0.75 | 1.24 | 269.20 | 4.59 | 2.03 | >500 | 0.37 | 0.17 | 5.01 |
| CC1/85 | 0.64 | 0.46 | 0.33 | 107.46 | 9.44 | 1.98 | >500 | 0.29 | 0.07 | 4.07 |
| NL4-3 | 0.44 | 0.26 | 0.42 | 8.44 | 0.80 | 0.22 | 150.32 | 0.08 | 0.07 | 0.11 |
| T278.50 | 0.16 | 0.10 | 0.09 | 0.48 | 0.30 | 0.14 | 0.43 | 0.02 | 0.03 | 0.02 |
| X2088.C9 | 0.06 | 0.05 | 0.05 | 0.07 | 0.02 | 0.09 | 0.09 | 0.06 | 0.03 | 0.05 |
| ZM106.9 | 0.05 | 0.02 | 0.02 | 0.04 | 0.01 | 0.02 | 0.03 | 0.03 | 0.03 | 0.01 |
| CAP45.2.00.G3 | 0.10 | 0.10 | 0.11 | 2.49 | 12.51 | 3.63 | 36.53 | 1.69 | 0.25 | 96.75 |
| HIV-2-AID | 14.16 | 19.13 | 20.28 | 136.88 | >500 | * | >500 | * | * | * |
| HIV-2-ATM88 | 1.34 | 1.22 | 2.28 | 2.06 | 70.58 | * | >500 | * | * | * |
| HIV-2-HCC-01 | 35.94 | 17.47 | 24.36 | >500 | >500 | * | >500 | * | * | * |

| | bNAb3-Derived | | | | | | | |
|---|---|---|---|---|---|---|---|---|
| | Molecules | | bNAb4-Derived Molecules | | | | CD4 Controls | |
| Envelope | 11 | 12 | 13 | 14 | 15 | 16 | 17 | 18 |
| 42368 | 2.12 | >500 | 0.50 | 8.78 | 3.42 | >500 | 4.32 | 1.58 |
| CC1/85 | 1.15 | 38.33 | 0.10 | 1.39 | 0.77 | 2.36 | 7.02 | 1.98 |
| NL4-3 | 0.06 | 0.15 | 0.19 | 0.86 | 0.48 | 327.61 | 0.31 | 0.31 |
| T278.50 | 0.02 | 0.01 | 0.13 | 0.23 | 0.41 | 0.47 | 3.99 | 1.43 |
| X2088.C9 | 0.03 | 0.03 | 0.03 | 0.01 | 0.04 | 0.02 | 17.49 | 9.20 |
| ZM106.9 | 0.03 | 0.03 | 0.03 | 0.02 | 0.03 | 0.03 | 26.02 | 15.14 |
| CAP45.2.00.G3 | 61.81 | >500 | 13.18 | 469.16 | 42.98 | >500 | 131.43 | 20.48 |
| HIV-2-AID | * | * | * | * | * | * | 103.54 | 24.35 |
| HIV-2-ATM88 | * | * | * | * | * | * | 3.93 | 2.72 |
| HIV-2-HCC-01 | * | * | * | * | * | * | >500 | >300 |

* not tested
Table 5 Molecule Key:
1 = D1m-K8C-G99C_2xG4S_bNAb2 (SEQ ID NOs: 137 + 69)
2 = D1m-K8C-G99C_3xG4S_bNAb2 (SEQ ID NOs: 138 + 69)
3 = D1m-K8C-G99C_4xG4S_bNAb2 (SEQ ID NOs: 139 + 69)
4 = bNAb2-LC_1xG4S_D1m-K8C-G99C (SEQ ID NOs: 68 + 143)
5 = bNAb2 (SEQ ID NOs: 68 + 69) + D1m_His (SEQ ID NO: 4*) (combo)
6 = bNAb2 (SEQ ID NOs: 68 + 69) + D1m-K8C-G99C_Fc (SEQ ID NO: 158) (combo)
7 = bNAb2 (SEQ ID NOs: 68 + 69)
8 = D1m-K8C-G99C_1xG4S_bNAb3 (SEQ ID NOs: 144 + 74)
9 = D1m-K8C-G99C_4xG4S_bNAb3 (SEQ ID NOs: 145 + 74)
10 = bNAb3 (SEQ ID NOs: 73 + 74) + D1m_His (SEQ ID NO: 4*) (combo)
11 = bNAb3 (SEQ ID NOs: 73 + 74) + D1m-K8C-G99C_Fc (SEQ ID NO: 158) (combo)
12 = bNAb3 (SEQ ID NOs: 73 + 74)
13 = D1m-K8C-G99C_4xG4S_bNAb4 (SEQ ID NOs: 147 + 79)
14 = bNAb4 (SEQ ID NOs: 78 + 79) + D1m_His (SEQ ID NO: 4*) (combo)
15 = bNAb4 (SEQ ID NOs: 78 + 79) + D1m-K8C-G99C_Fc (SEQ ID NO: 158) (combo)
16 = bNAb4 (SEQ ID NOs: 78 + 79)
17 = D1m_His (SEQ ID NO: 4*)
18 = D1m-K8C-G99C_Fc (SEQ ID NO: 158)
* plus a 6xHis tag (six C-terminal histidine residues)

Thermal stabilization of CD4 D1 (see Example 3 above) further enhanced the PK of the bispecific molecules (D1m-K8C-G99C_1xG4S_bNAb1, SEQ ID NOs: 121 and 63; D1m-T11C-K72C_1xG4S_bNAb1, SEQ ID NOs: 122 and 63; D1m-K8I_1xG4S_bNAb1, SEQ ID NOs: 119 and 63; and D1m-K8V_1xG4S_bNAb1, SEQ ID NOs: 120 and 63) as shown in Table 6 below.

TABLE 6
The effect of (1) linker length between the CD4 domain and
bNAb1 heavy chain, and (2) thermal stability of CD4 D1,
on the PK of bispecific molecules in hFcRn mice (Tg32)

| | Tm of | | |
|---|---|---|---|
| Molecule | CD4 D1 | Half-life | Clearance |
| (SEQ ID NOs) | (° C.) | (days) | (ml/day/kg) |
| bNAb1 (62 + 63) | — | 5.8 | 21.6 |
| D1m_4xG4S_bNAb1 (105 + 63) | 68.2 | 2.2 | 144 |
| D1m_1xG4S_bNAb1 (102 + 63) | 68.2 | 4.1 | 31.7 |
| D1m-K8C-G99C_1xG4S_bNAb1 | 88.8 | 10.2 | 7.4 |
| (121 + 63) | | | |
| D1m-T11C- | 79.9 | 8.9 | 12.9 |
| K72C_1xG4S_bNAb1 (122 + 63) | | | |
| D1m-K8I_1xG4S_bNAb1 | 75.9 | 9.3 | 12 |
| (119 + 63) | | | |
| D1m-K8V_1xG4S_bNAb1 | 77.1 | 7.3 | 12.8 |
| (120 + 63) | | | |

Accordingly, the best molecules for further development contain shorter linker lengths between the CD4 domain and bNAb (1xG4S) and contain a thermally stable CD4 domain(s).

### Example 5—Anti-Viral Activity of bNAb1-Derived Bispecific Molecules

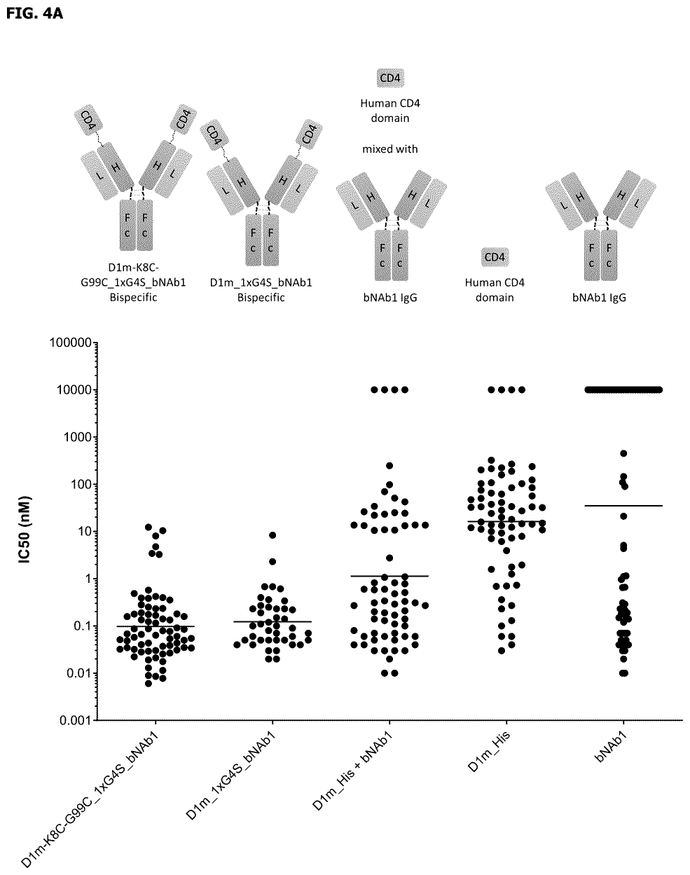

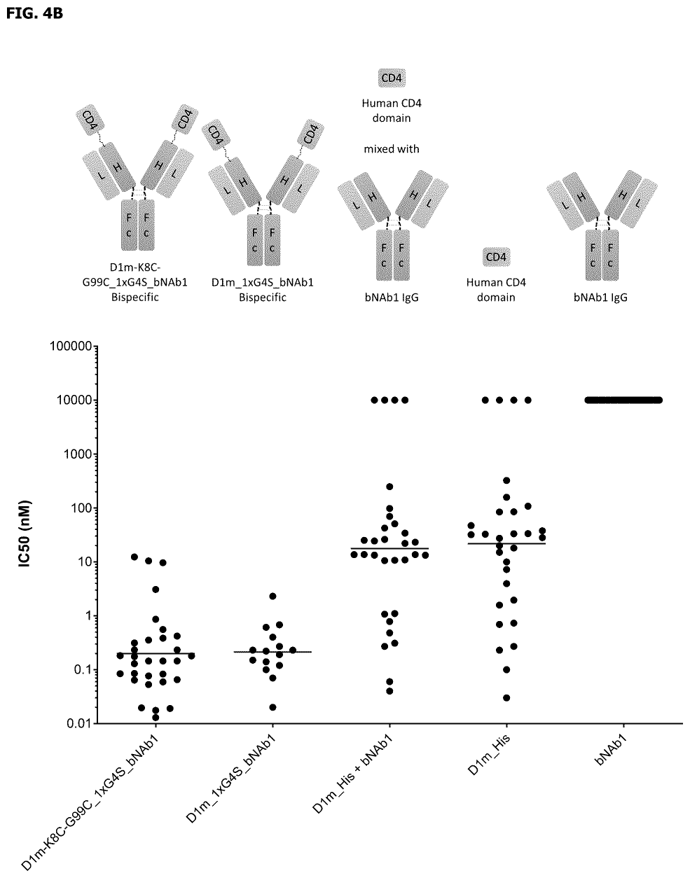

As shown in FIG. 4A, bispecific molecules (D1m_1xG4S_bNAb1 bispecific having SEQ ID NOs: 102 and 63; and D1m-K8C-G99C_1xG4S_bNAb1 bispecific having SEQ ID NOs: 121 and 63) with human CD4 domains (CD4 D1m, SEQ ID NO:4, and a K8C and G99C variant thereof, SEQ ID NO: 11, respectively) fused to the N-termini of each of the heavy chains of bNAb1 (SEQ ID NOs: 62 and 63) via a GGGGS linker (SEQ ID NOs: 90) showed dramatically and consistently higher activity (fully neutralized all envelopes tested with geometric mean IC50˜0.1 nM) than the two individual components (human CD4 domain and bNAb1) alone and their mixture (did not neutralize all envelopes tested, geometric mean IC50 much higher than the bispecific molecules). This clearly shows that the fusion strategy provides strong anti-viral synergy.

Interestingly, soluble CD4 has been considered to have negative synergy with bNAb1 (Ivan et al., Plos Biol. 17 (1), January 2019), based on the result that mixing soluble CD4 with bNAb1 can weaken its anti-viral activity. We found that when soluble CD4 is fused with bNAb1 instead of being mixed, the anti-viral activity is enhanced dramatically.

FIG. 4B shows the anti-viral activity of the bispecific molecules (SEQ ID NOs: 102 and 63; and SEQ ID NOs: 121 and 63) and control molecules against a panel of PSV strains insensitive to bNAb1. It can be seen that that the bispecific molecules (fully neutralized all envelopes tested with geometric IC50˜0.2 nM) are much more potent than the individual components alone and their mixture (did not neutralize all envelopes tested, geometric mean IC50>10 nM), indicating strong anti-viral synergy. Such synergy is most obvious against the strains that are insensitive to both CD4 and bNAb1, where only the bispecific molecules showed complete inhibition of viral entry with good activity, while neither soluble CD4 domain nor bNAb1 nor their mixture exhibited significant activity.

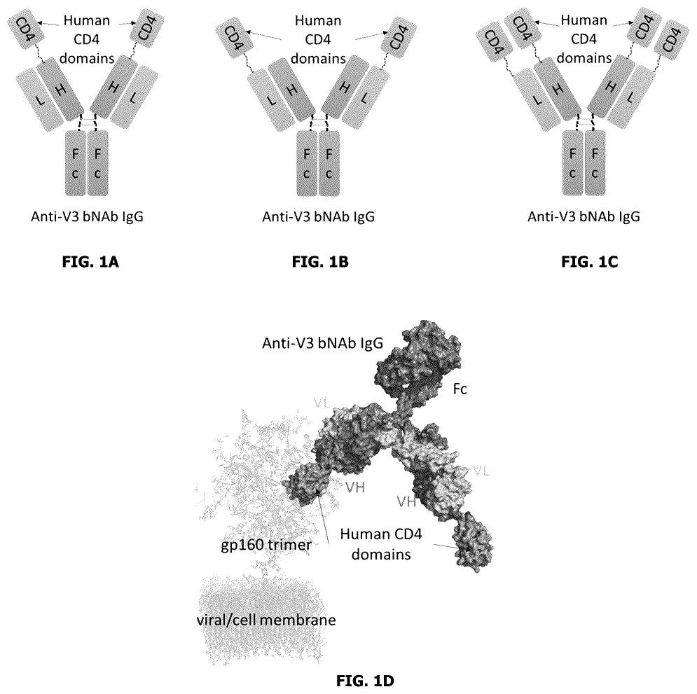

As shown in Table 4.1 above, although all of the bispecific molecules tested are able to inhibit viral entry, the most consistent and potent bispecific molecule is when the CD4 domain is fused to the N-termini of each of the heavy chains of bNAb1, which correlates well with the structure-based design (FIG. 1D).

Furthermore, as shown in Table 4.2 above, when tested in PSV assay against the HIV-1 envelopes insensitive to several entry inhibitors (10E8, N6, Temsavir, Ibalizumab, and Maraviroc), the bispecific molecule (SEQ ID NOs: 121 and 63) fully neutralized all these envelopes with IC50s<400 pM.

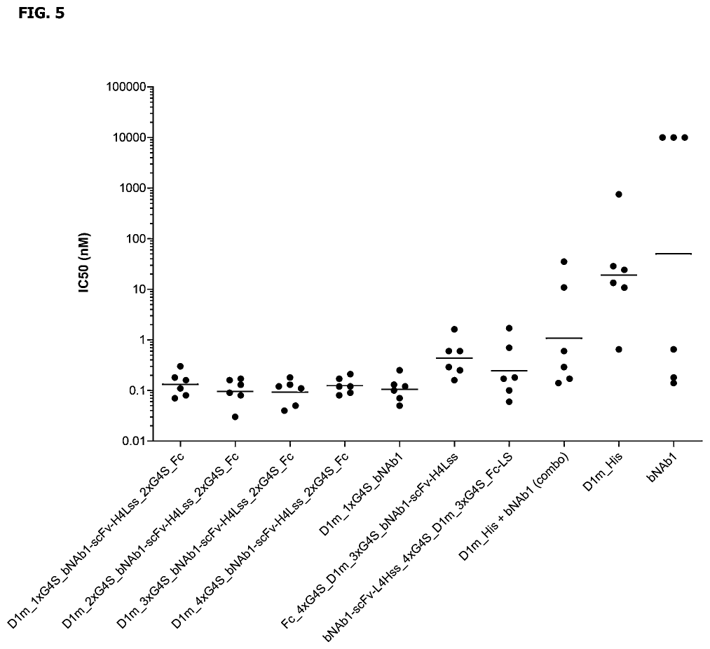

In addition, the most potent bispecific format (i.e. fusing CD4 D1 to the N-terminus of bNAb1 heavy chain) was converted to a single open-reading-frame (ORF) version, by replacing the Fab arm with a scFv fragment of bNAb1. As shown in FIG. 5, several such single-ORF molecules, also referred to as scFv-Fc molecules (SEQ ID NOs: 152-157), showed equivalent potency as the leading bispecific format in PSV assays (ACTOne cells).

Given that these single-ORF molecules are each encoded by a single <2 kb gene and contain an Fc domain for an increased half-life, they can be readily delivered by gene therapy vehicles, such as adeno-associated virus (AAV), enabling them to be constantly secreted into circulation at a therapeutic concentration. Such a strategy would result in an ‘ultra-long’ acting therapy against HIV-1.

### Example 6—Anti-Viral Activity of bNAb6-Derived Bispecific Molecules

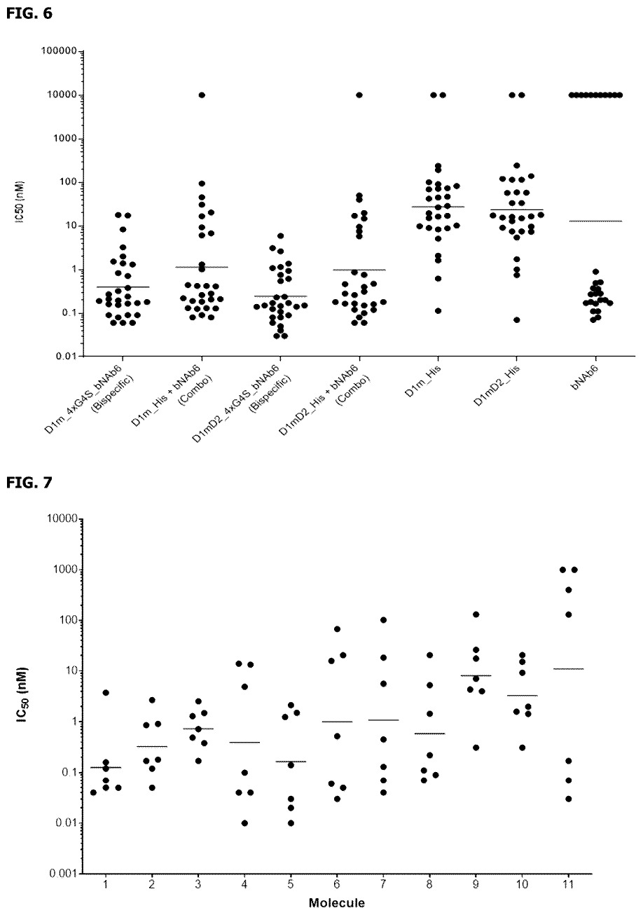

FIG. 6 and Table 7 below show the anti-viral activity of bNAb6-derived bispecific molecules and control molecules.

The plot of IC50 values (FIG. 6) from PSV assays (ACTOne) clearly shows that when CD4 domain 1 (D1m, SEQ ID NO:4) or domains 1 and 2 (D1mD2, SEQ ID NO:2) is fused to the N-termini of the bNAb6 (SEQ ID NOs: 88 and 89) heavy chain, the resultant molecule is much more active than a simple mixture of soluble CD4 domain and bNAb6 antibody, indicating strong synergy.

Table 7 shows the anti-viral activity of the bispecific molecules (D1m_4xG4S_bNAb6, SEQ ID NOs: 151 and SEQ ID NOs: 89; D1mD2_4xG4S_bNAb6, SEQ ID NO: 150 and SEQ ID NO: 89; and D1m-K8C-G99C_1xG4S_bNAb6 (SEQ ID NOs: 362+89)) and control molecules against HIV-1 strains resistant to bNAb6 antibody. As can be seen, the bNAb6-derived bispecific molecules are much more potent than the mixtures against double-resistant or insensitive strains.

TABLE 7
IC50 (nM) of bNAb6-derived bispecific formats and control molecules
against a panel of HIV-1 envelopes in PSV assay (ACTOne cells)

| | bNAb6-Derived Molecules | | | | | | |
|---|---|---|---|---|---|---|---|
| Envelope | 1 | 2 | 3 | 4 | 5 | 6 | 7 |
| CAP45.2.00.G3 | 17.79 | >500 | 5.90 | >500 | 131.43 | >500 | >500 |
| CH119 | 8.30 | 93.62 | 2.60 | 49.76 | 90.66 | 119.94 | >500 |
| BJOX2000 | 17.27 | 45.23 | 3.10 | 16.96 | 70.87 | 33.40 | >500 |
| X1632_S2_B10 | 1.38 | 20.26 | 0.75 | 19.70 | 16.25 | 14.94 | >500 |
| TRO11 | 1.99 | 16.55 | 1.14 | 14.64 | 68.43 | 17.27 | >500 |
| T278.50 | 0.71 | 9.26 | 1.08 | 9.51 | 3.99 | 16.02 | >500 |
| LAI | 1.52 | 6.76 | 0.93 | 7.46 | 8.79 | 5.41 | >500 |
| 42368 | 1.30 | 6.05 | 0.54 | 5.75 | 4.32 | 7.29 | >500 |
| NL4-3 | 0.17 | 1.31 | 0.11 | 0.75 | 0.31 | 0.74 | >500 |
| HXB2 | 0.06 | 0.08 | 0.03 | 0.06 | 0.11 | 0.07 | >500 |

| | bNAb6- | | | |
|---|---|---|---|---|
| | Derived | | | |
| | Molecules | | | |
| Envelope | 8 | 9 | 7 | 10 |
| X2088.C9 | 0.17 | 0.15 | 0.15 | 9.20 |
| ZM106.9 | 0.10 | 0.10 | 0.08 | 15.14 |
| 3637_V5_C3 | 103.00 | 328.50 | >500 | >500 |
| 3468_V1_C12 | 20.85 | 47.70 | >500 | 64.15 |
| Q461_E2 | 4.00 | 12.70 | >500 | 11.76 |
| 3326_V4_C3 | 72.10 | 483.00 | >500 | >500 |
| 42368 | 0.92 | 2.13 | >500 | 1.58 |
| NL4-3 | 0.27 | * | >500 | 0.31 |
| T278.50 | 1.33 | * | >500 | 1.43 |
| CAP45.2.00.G3 | 9.64 | * | >500 | 20.48 |
| CC1/85 | 0.42 | * | * | 1.98 |
| HIV-2-HCC-01 | 175.65 | * | * | >300 |

* not tested
Table 7 Molecule Key:
1 = D1m_4xG4S_bNAb6 (SEQ ID NOs: 151 + 89)
2 = D1m_His (SEQ ID NO: 4*) + bNAb6 (SEQ ID NOs: 88 + 89)
3 = D1mD2_4xG4S_bNAb6 (SEQ ID NOs: 150 + 89)
4 = D1mD2_His (SEQ ID NO: 2*) + bNAb6 (SEQ ID NOs: 88 + 89)
5 = D1m_His (SEQ ID NO: 4*)
6 = D1mD2_His (SEQ ID NO: 2*)
7 = bNAb6 (SEQ ID NOs: 88 + 89)
8 = D1m-K8C-G99C_1xG4S_bNAb6 (SEQ ID NOs: 362 + 89)
9 = bNAb6 (SEQ ID NOs: 88 + 89) + D1m-K8C-G99C_Fc (SEQ ID NO: 158) (combo)
10 = D1m-K8C-G99C_Fc (SEQ ID NO: 158)
* plus a 6xHis tag (six C-terminal histidine residues)

### Example 7—Anti-Viral Activity of Further Anti-V3 bNAb-Derived Bispecific Molecules

Additional bispecific molecules comprising CD4 domains fused to other anti-V3 bNAbs (bNAb2, bNAb3, bNAb4,) were tested, with the results shown in Table 5 above and Tables 8-23 below, a similar synergistic anti-viral activity was observed against one or more envelopes.

Accordingly, the strategy of fusing a soluble CD4 domain to anti-V3 loop bNAb can be generally applied to enhance the potency and spectrum of these bNAbs.

### CONCLUSIONS

On the free HIV-1 virus, the V3 loop of gp120 is in its native “closed” state. The V3 loop is known to adopt various conformations (from different structures in Protein Databank), indicating its flexibility. During HIV-1 infection, the binding of gp120 to cell surface CD4 triggers conformational changes of the V3 loop to “open” itself to bind co-receptors such as CXCR4 or CCR5.

Anti-V3 bNAbs mainly recognizes a pattern of glycans on the V3 loop of gp120, along with the backbone atoms of a few amino acid residues in the V3 loop (Krumm et al., Retrovirology 13 (8), 2016). Such “plasticity” of the V3 loop may facilitate the binding of anti-V3 loop bNAbs to this loop when it is “opened” by CD4 binding.

We hypothesize that when soluble CD4 and anti-V3 bNAbs are simply mixed together, the conformational change of the V3 loop triggered by soluble CD4 may be too transient for the anti-V3 bNAb to capture, therefore no synergistic activity is observed. But, in the context of bispecific molecules, when CD4 binds to the CD4 binding site (CD4bs) on gp120, the anti-V3 bNAb is at such high local concentration that it can immediately capture the exposed V3 loop glycans; this in turn could stabilize the binding of soluble CD4 to gp120 and form a positive feedback loop.

TABLE 8
IC50 (nM) of different bNAb5-derived bispecific
formats and control molecules against a panel
of HIV-1 envelopes in PSV assay (ACTOne cells)

| | | | | CD4 |
|---|---|---|---|---|
| | bNAb5-Derived Molecules | | | Controls |
| Envelope | Bispecific | Combo | mAb | CD4 |
| 42368 | 1.11 | 2.35 | >500 | 1.58 |
| CC1/85 | 0.67 | 2.53 | >500 | 1.98 |
| NL4-3 | 0.26 | 0.26 | 2.62 | 0.31 |
| T278.50 | 3.21 | 1.59 | 96.06 | 1.43 |
| X2088.C9 | 0.54 | 0.14 | 0.17 | 9.20 |
| ZM106.9 | 0.94 | 1.09 | 2.04 | 15.14 |
| CAP45.2.00.G3 | 44.41 | 63.82 | >500 | 20.48 |
| 25710 | 0.51 | 0.49 | 8.10 | 1.32 |
| TRO11 | 0.91 | 0.58 | 0.39 | 146.64 |
| 398F1 | 4.85 | 1.35 | 1.83 | 90.47 |
| CNE8 | 134.25 | 240.63 | 51.37 | 346.64 |
| X2278 | 0.60 | 5.46 | 9.53 | 44.56 |
| BJOX2000 | 1.11 | 1.86 | 126.82 | 2.34 |
| X1632 | 2.32 | 2.38 | >500 | 1.82 |
| CE1176 | 1.04 | 2.59 | 57.07 | 4.22 |
| 246F3 | 7.18 | 5.27 | 40.90 | 4.28 |
| CH119 | 2.63 | 3.22 | 63.54 | 3.25 |
| CE0217 | 2.42 | 2.19 | 409.83 | 2.53 |
| CNE55 | 20.92 | 36.59 | 3300.62 | 41.82 |
| JR-CSF | 0.31 | 0.45 | 0.64 | 5.47 |
| JRFL | 0.42 | 1.16 | 58.23 | 1.12 |

Table 8 Molecule Key:
Bispecific = D1m-K8C-G99C_1xG4S_bNAb5 (SEQ ID NOs: 148 + 84)
Combo = bNAb5 (SEQ ID NOs:83 + 84) + D1m-K8C-G99C_Fc (SEQ ID NO: 158) (combo)
mAb = bNAb5 (SEQ ID NOs:83 + 84)
CD4 = D1m-K8C-G99C_Fc (SEQ ID NO: 158)

TABLE 9
IC50 (nM) of different bNAb7-derived bispecific
formats and control molecules against a panel
of HIV-1 envelopes in PSV assay (ACTOne cells)

| | | | | CD4 |
|---|---|---|---|---|
| | bNAb7-Derived Molecules | | | Controls |
| Envelope | Bispecific | Combo | mAb | CD4 |
| 42368 | 1.38 | 2.40 | >500 | 1.58 |
| X2088.C9 | 0.09 | 0.19 | 0.22 | 9.20 |
| ZM106.9 | 0.03 | 0.03 | 0.04 | 15.14 |
| 3326_V4_C3 | 17.63 | >500 | >500 | >500 |
| 3637_V5_C3 | 260.71 | 388.10 | >500 | >500 |
| 3468_V1_C12 | 2.50 | 32.37 | >500 | 64.15 |
| 620345_C1 | 4.03 | 5.83 | >500 | 99.28 |
| 0260.v5.c36 | 0.16 | 0.22 | 0.75 | 119.29 |
| TH976_17 | 70.02 | 35.04 | >500 | 39.05 |
| Q461_E2 | 14.61 | 20.92 | >500 | 11.76 |
| 928_28 | 0.43 | 0.57 | 32.73 | 0.77 |
| YU-2 | 0.07 | 0.16 | 0.58 | 0.20 |
| 6471_V1_C16 | 0.59 | 0.50 | >500 | 5.07 |
| 93UG065 | 50.99 | 40.15 | >500 | 142.61 |
| CC1/85 | 0.21 | * | * | 1.98 |
| NL4-3 | 0.33 | * | * | 0.31 |
| T278.50 | 1.79 | * | * | 1.43 |
| CAP45.2.00.G3 | 0.22 | * | * | 20.48 |
| HIV-2-HCC-01 | 183.57 | * | * | >300 |

* not tested
Table 9 Molecule Key:
Bispecific = D1m-K8C-G99C_1xG4S_bNAb7 (SEQ ID NO: 338 + 259)
Combo = bNAb7 (SEQ ID NOs: 258 + 259) + D1m-K8C-G99C_Fc (SEQ ID NO: 158) (combo)
mAb = bNAb7 (SEQ ID NOs: 258 + 259)
CD4 = D1m-K8C-G99C_Fc (SEQ ID NO: 158)

TABLE 10
IC50 (nM) of different bNAb8-derived bispecific
formats and control molecules against a panel
of HIV-1 envelopes in PSV assay (ACTOne cells)

| | | | | CD4 |
|---|---|---|---|---|
| | bNAb8-Derived Molecules | | | Controls |
| Envelope | Bispecific | Combo | mAb | CD4 |
| X2088.C9 | 0.11 | 0.22 | 0.23 | 9.20 |
| ZM106.9 | 0.02 | 0.03 | 0.03 | 15.14 |
| 3637_V5_C3 | 104.00 | 305.50 | >500 | >500 |
| 3468_V1_C12 | 0.06 | 0.81 | 1.70 | 64.15 |
| Q461_E2 | 7.06 | 6.59 | >500 | 11.76 |
| 3326_V4_C3 | 75.70 | 367.00 | >500 | >500 |
| 42368 | 0.97 | 1.35 | >500 | 1.58 |
| CC1/85 | 0.14 | * | * | 1.98 |
| NL4-3 | 0.14 | * | * | 0.31 |
| T278.50 | 1.20 | * | * | 1.43 |
| CAP45.2.00.G3 | 2.72 | * | * | 20.48 |
| HIV-2-HCC-01 | 33.29 | * | * | >300 |

* not tested
Table 10 Molecule Key:
Bispecific = D1m-K8C-G99C_1xG4S_bNAb8 (SEQ ID Nos: 339 + 264)
Combo = bNAb8 (SEQ ID Nos: 263 + 264) + D1m-K8C-G99C_Fc (SEQ ID NO: 158) (combo)
mAb = bNAb8 (SEQ ID Nos: 263 + 264)
CD4 = D1m-K8C-G99C_Fc (SEQ ID NO: 158)

TABLE 11
IC50 (nM) of different bNAb9-derived bispecific
formats and control molecules against a panel
of HIV-1 envelopes in PSV assay (ACTOne cells)

| | | | | CD4 |
|---|---|---|---|---|
| | bNAb9-Derived Molecules | | | Controls |
| Envelope | Bispecific | Combo | mAb | CD4 |
| 42368 | 0.15 | 1.35 | >500 | 1.58 |
| X2088.C9 | 0.18 | 9.50 | >500 | 9.20 |
| ZM106.9 | 0.08 | 16.28 | >500 | 15.14 |
| 3326_V4_C3 | 3.87 | >500 | >500 | >500 |
| 3637_V5_C3 | 6.74 | >500 | >500 | >500 |
| 3468_V1_C12 | 1.51 | 23.22 | >500 | 64.15 |
| 620345_C1 | 1.02 | 3.25 | >500 | 99.28 |
| 0260.v5.c36 | 0.19 | 0.14 | 0.26 | 119.29 |
| TH976_17 | 20.42 | 27.24 | >500 | 39.05 |
| Q461_E2 | 0.55 | 18.54 | >500 | 11.76 |
| 928_28 | 0.20 | 0.42 | >500 | 0.77 |
| YU-2 | 0.12 | 0.09 | 0.36 | 0.20 |
| 6471_V1_C16 | 0.11 | 1.17 | >500 | 5.07 |
| 93UG065 | 0.18 | 31.44 | >500 | 142.61 |
| CC1/85 | 0.03 | * | * | 1.98 |
| NL4-3 | 0.07 | * | * | 0.31 |
| T278.50 | 0.14 | * | * | 1.43 |
| CAP45.2.00.G3 | 1.03 | * | * | 20.48 |
| HIV-2-HCC-01 | 1.68 | * | * | >300 |

* not tested
Table 11 Molecule Key:
Bispecific = D1m-K8C-G99C_1xG4S_bNAb9 (SEQ ID NOs: 340 + 269)
Combo = bNAb9 (SEQ ID NOs: 268 + 269) + D1m-K8C-G99C_Fc (SEQ ID NO: 158) (combo)
mAb = bNAb9 (SEQ ID NOs: 268 + 269)
CD4 = D1m-K8C-G99C_Fc (SEQ ID NO: 158)

TABLE 12
IC50 (nM) of different bNAb10-derived bispecific
formats and control molecules against a panel
of HIV-1 envelopes in PSV assay (ACTOne cells)

| | | | | CD4 |
|---|---|---|---|---|
| | bNAb10-Derived Molecules | | | Controls |
| Envelope | Bispecific | Combo | mAb | CD4 |
| 42368 | 0.17 | 2.29 | 189.62 | 1.58 |
| X2088.C9 | 0.17 | 9.94 | 233.05 | 9.20 |
| ZM106.9 | 0.09 | 24.96 | 71.31 | 15.14 |
| 3326_V4_C3 | 14.28 | >500 | >500 | >500 |
| 3637_V5_C3 | 0.36 | 439.54 | >500 | >500 |
| 3468_V1_C12 | 0.21 | 38.64 | 258.28 | 64.15 |
| 620345_C1 | 0.33 | 0.85 | >500 | 99.28 |
| 0260.v5.c36 | 0.17 | 0.29 | 0.48 | 119.29 |
| TH976_17 | 18.49 | 31.63 | >500 | 39.05 |
| Q461_E2 | 0.41 | 15.03 | >500 | 11.76 |
| 928_28 | 0.17 | 0.42 | 320.41 | 0.77 |
| YU-2 | 0.12 | 0.23 | 0.81 | 0.20 |
| 6471_V1_C16 | 0.12 | 1.23 | 91.84 | 5.07 |
| 93UG065 | 0.13 | 0.12 | 0.21 | 142.61 |
| CC1/85 | 0.04 | * | * | 1.98 |
| NL4-3 | 0.08 | * | * | 0.31 |
| T278.50 | 0.16 | * | * | 1.43 |
| CAP45.2.00.G3 | 4.55 | * | * | 20.48 |
| HIV-2-HCC-01 | 0.95 | * | * | >300 |

* not tested
Table 12 Molecule Key:
Bispecific = D1m-K8C-G99C_1xG4S_bNAb10 (SEQ ID Nos: 341 + 274)
Combo = bNAb10 (SEQ ID Nos: 273 + 274) + D1m-K8C-G99C_Fc (SEQ ID NO: 158) (combo)
mAb = bNAb10 (SEQ ID Nos: 273 + 274)
CD4 = D1m-K8C-G99C_Fc (SEQ ID NO: 158)

TABLE 13
IC50 (nM) of different bNAb11-derived bispecific
formats and control molecules against a panel
of HIV-1 envelopes in PSV assay (ACTOne cells)

| | | | | CD4 |
|---|---|---|---|---|
| | bNAb11-Derived Molecules | | | Controls |
| Envelope | Bispecific | Combo | mAb | CD4 |
| 42368 | 0.48 | 2.65 | >500 | 1.58 |
| X2088.C9 | 0.15 | 7.06 | 95.12 | 9.20 |
| ZM106.9 | 0.07 | 14.74 | 73.68 | 15.14 |
| 3326_V4_C3 | 51.71 | 146.99 | >500 | >500 |
| 3637_V5_C3 | 6.73 | 410.00 | >500 | >500 |
| 3468_V1_C12 | 1.80 | 25.99 | >500 | 64.15 |
| 620345_C1 | 1.27 | 10.97 | 438.41 | 99.28 |
| 0260.v5.c36 | 0.20 | 0.42 | 0.57 | 119.29 |
| TH976_17 | 17.37 | 55.43 | >500 | 39.05 |
| Q461_E2 | 0.51 | 12.32 | >500 | 11.76 |
| 928_28 | 0.27 | 0.46 | >500 | 0.77 |
| YU-2 | 0.10 | 0.26 | 0.76 | 0.20 |
| 6471_V1_C16 | 0.25 | 0.71 | 253.40 | 5.07 |
| 93UG065 | 0.22 | 24.72 | 89.50 | 142.61 |
| CC1/85 | 0.09 | * | * | 1.98 |
| NL4-3 | 0.16 | * | * | 0.31 |
| T278.50 | 0.26 | * | * | 1.43 |
| CAP45.2.00.G3 | 5.82 | * | * | 20.48 |
| HIV-2-HCC-01 | 8.69 | * | * | >300 |

* not tested
Table 13 Molecule Key:
Bispecific = D1m-K8C-G99C_1xG4S_bNAb11 (SEQ ID NOs: 342 + 279)
Combo = bNAb11 (SEQ ID NOs: 278 + 279) + D1m-K8C-G99C_Fc (SEQ ID NO: 158) (combo)
mAb = bNAb11 (SEQ ID NOs: 278 + 279)
CD4 = D1m-K8C-G99C_Fc (SEQ ID NO: 158)

TABLE 14
IC50 (nM) of different bNAb12-derived bispecific formats and control molecules
against a panel of HIV-1 envelopes in PSV assay (ACTOne cells)

| | | | | | | | CD4 |
|---|---|---|---|---|---|---|---|
| | bNAb12-Derived Molecules | | | | | | Controls |
| Envelope | Bispecific1 | Bispecific2 | Bispecific3 | Bispecific4 | Combo | mAb | CD4 |
| X2088.C9 | 0.59 | 13.09 | 4.43 | >500 | 7.37 | >500 | 9.20 |
| ZM106.9 | 0.13 | 0.13 | 0.27 | 3.37 | 1.55 | 1.85 | 15.14 |
| 3637_V5_C3 | 9.45 | 130.00 | 62.45 | >500 | 474.07 | >500 | >500 |
| 3468_V1_C12 | 0.82 | 2.94 | 4.41 | 103.85 | 16.30 | 343.72 | 64.15 |
| Q461_E2 | 0.26 | 0.3 | 0.5 | 4.5 | 5.16 | 41.90 | 11.76 |
| 3326_V4_C3 | 9.63 | 63.69 | 81.80 | >500 | 266.93 | >500 | >500 |
| 42368 | 0.15 | 6.2 | 1.3 | 167.0 | 1.84 | 344.45 | 1.58 |
| 620345_C1 | 0.33 | * | * | * | 2.13 | 179.12 | 99.28 |
| 0260.v5.c36 | 0.44 | * | * | * | 0.50 | 5.19 | 119.29 |
| TH976_17 | 29.83 | * | * | * | 36.40 | >500 | 39.05 |
| 928_28 | 0.46 | * | * | * | 0.48 | 482.30 | 0.77 |
| YU-2 | 0.21 | * | * | * | 0.29 | 77.41 | 0.20 |
| 6471_V1_C16 | 0.26 | * | * | * | 0.59 | >500 | 5.07 |
| 93UG065 | 0.42 | * | * | * | 0.86 | 3.17 | 142.61 |
| CC1/85 | 0.12 | * | * | * | * | * | 1.98 |
| NL4-3 | 0.14 | * | * | * | * | * | 0.31 |
| T278.50 | 0.19 | * | * | * | * | * | 1.43 |
| CAP45.2.00.G3 | 1.29 | * | * | * | * | * | 20.48 |
| HIV-2-HCC-01 | 4.04 | * | * | * | * | * | >300 |

* not tested
Table 14 Molecule Key:
Bispecific1 = D1m-K8C-G99C_1xG4S_bNAb12 (SEQ ID NOs: 343 + 284)
Bispecific2 = bNAb12_HC_1xG4S_D1m-K8C-G99C (SEQ ID NOs: 344 + 284)
Bispecific3 = D1m-K8C-G99C_1xG4S_LC-bNAb12 (SEQ ID NOs: 283 + 345)
Bispecific4 = bNAb12_LC_1xG4S_D1m-K8C-G99C (SEQ ID NOs: 283 + 346)
Combo = bNAb12 (SEQ ID NOs: 283 + 284) + D1m-K8C-G99C_Fc (SEQ ID NO: 158) (combo)
mAb = bNAb12 (SEQ ID NOs: 283 + 284)
CD4 = D1m-K8C-G99C_Fc (SEQ ID NO: 158)

TABLE 15
IC50 (nM) of different bNAb13-derived bispecific
formats and control molecules against a panel
of HIV-1 envelopes in PSV assay (ACTOne cells)

| | bNAb13-Derived Molecules | | | CD4 Controls |
|---|---|---|---|---|
| Envelope | Bispecific | Combo | mAb | CD4 |
| 42368 | 0.24 | 1.52 | >500 | 1.58 |
| X2088.C9 | 2.05 | 5.18 | >500 | 9.20 |
| ZM106.9 | 0.28 | 3.04 | 22.43 | 15.14 |
| 3326_V4_C3 | 52.07 | 191.00 | >500 | >500 |
| 3637_V5_C3 | 37.30 | 266.00 | >500 | >500 |
| 3468_V1_C12 | 4.25 | 25.00 | >500 | 64.15 |
| Q461_E2 | 0.50 | 4.14 | >500 | 11.76 |
| CC1/85 | 0.19 | * | * | 1.98 |
| 620345_C1 | 3.58 | * | >500 | 99.28 |
| 0260.v5.c36 | 0.36 | * | 18.10 | 119.29 |
| TH976_17 | 37.26 | * | >500 | 39.05 |
| 928_28 | 0.79 | * | 201.39 | 0.77 |
| YU-2 | 0.17 | * | 48.30 | 0.20 |
| 6471_V1_C16 | 0.16 | * | 133.35 | 5.07 |
| 93UG065 | 0.58 | * | 46.41 | 142.61 |
| NL4-3 | 0.21 | * | * | 0.31 |
| T278.50 | 0.34 | * | * | 1.43 |
| CAP45.2.00.G3 | 2.26 | * | * | 20.48 |
| HIV-2-HCC-01 | 11.13 | * | * | >300 |

* not tested
Table 15 Molecule Key:
Bispecific = D1m-K8C-G99C_1xG4S_bNAb13 (SEQ ID NOs: 347 + 289)
Combo = bNAb13 (SEQ ID NOs: 288 + 289) + D1m-K8C-G99C_Fc (SEQ ID NO: 158) (combo)
mAb = bNAb13 (SEQ ID NOs: 288 + 289)
CD4 = D1m-K8C-G99C_Fc (SEQ ID NO: 158)

TABLE 16
IC50 (nM) of different bNAb14-derived bispecific
formats and control molecules against a panel
of HIV-1 envelopes in PSV assay (ACTOne cells)

| | bNAb14-Derived Molecules | | | CD4 Controls |
|---|---|---|---|---|
| Envelope | Bispecific | Combo | mAb | CD4 |
| X2088.C9 | 1.26 | 1.73 | 2.79 | 9.20 |
| ZM106.9 | 0.62 | 1.08 | 1.26 | 15.14 |
| 3637_V5_C3 | 22.85 | 84.20 | >500 | >500 |
| 3468_V1_C12 | 17.35 | 35.05 | >500 | 64.15 |
| Q461_E2 | 2.77 | 11.40 | >500 | 11.76 |
| 3326_V4_C3 | 196.50 | >500 | >500 | >500 |
| 42368 | 1.15 | 1.84 | >500 | 1.58 |
| CC1/85 | 1.74 | * | * | 1.98 |
| NL4-3 | 0.29 | * | * | 0.31 |
| T278.50 | 6.81 | * | * | 1.43 |
| CAP45.2.00.G3 | 2.85 | * | * | 20.48 |
| HIV-2-HCC-01 | 98.75 | * | * | >300 |

* not tested
Table 16 Molecule Key:
Bispecific = D1m-K8C-G99C_1xG4S_bNAb14 (SEQ ID NOs: 348 + 294)
Combo = bNAb14 (SEQ ID NOs: 293 + 294) + D1m-K8C-G99C_Fc (SEQ ID NO: 158) (combo)
mAb = bNAb14 (SEQ ID NOs: 293 + 294)
CD4 = D1m-K8C-G99C_Fc (SEQ ID NO: 158)

TABLE 17
IC50 (nM) of different bNAb15-derived bispecific
formats and control molecules against a panel
of HIV-1 envelopes in PSV assay (ACTOne cells)

| | bNAb15-Derived Molecules | | | CD4 Controls |
|---|---|---|---|---|
| Envelope | Bispecific | Combo | mAb | CD4 |
| X2088.C9 | 1.19 | 0.95 | 2.58 | 9.20 |
| ZM106.9 | 0.68 | 0.40 | 1.34 | 15.14 |
| 3637_V5_C3 | 25.45 | 5.33 | 5.00 | >500 |
| 3468_V1_C12 | 22.00 | 35.80 | >500 | 64.15 |
| Q461_E2 | 2.81 | 5.98 | >500 | 11.76 |
| 3326_V4_C3 | 142.00 | >500 | >500 | >500 |
| 42368 | 1.11 | 1.55 | >500 | 1.58 |
| CC1/85 | 1.65 | * | * | 1.98 |
| NL4-3 | 0.36 | * | * | 0.31 |
| T278.50 | 7.66 | * | * | 1.43 |
| CAP45.2.00.G3 | 4.48 | * | * | 20.48 |
| HIV-2-HCC-01 | 142.16 | * | * | >300 |

* not tested
Table 17 Molecule Key:
Bispecific = D1m-K8C-G99C_1xG4S_bNAb15 (SEQ ID NOs: 349 + 299)
Combo = bNAb15 (SEQ ID NOs: 298 + 299) + D1m-K8C-G99C_Fc (SEQ ID NO: 158) (combo)
mAb = bNAb15 (SEQ ID NOs: 298 + 299)
CD4 = D1m-K8C-G99C_Fc (SEQ ID NO: 158)

TABLE 18
IC50 (nM) of different bNAb16-derived bispecific
formats and control molecules against a panel
of HIV-1 envelopes in PSV assay (ACTOne cells)

| | bNAb16-Derived Molecules | | | CD4 Controls |
|---|---|---|---|---|
| Envelope | Bispecific | Combo | mAb | CD4 |
| X2088.C9 | 0.25 | 2.02 | 289.00 | 9.20 |
| ZM106.9 | 2.42 | 11.63 | >500 | 15.14 |
| 3637_V5_C3 | 19.15 | 129.50 | >500 | >500 |
| 3468_V1_C12 | 11.21 | 26.75 | >500 | 64.15 |
| Q461_E2 | 2.03 | 6.06 | >500 | 11.76 |
| 3326_V4_C3 | 31.75 | 148.50 | >500 | >500 |
| 42368 | 0.69 | 1.92 | >500 | 1.58 |
| CC1/85 | 0.56 | * | * | 1.98 |
| NL4-3 | 0.14 | * | * | 0.31 |
| T278.50 | 2.29 | * | * | 1.43 |
| CAP45.2.00.G3 | 2.08 | * | * | 20.48 |
| HIV-2-HCC-01 | 237.46 | * | * | >300 |

* not tested
Table 18 Molecule Key:
Bispecific = D1m-K8C-G99C_1xG4S_bNAb16 (SEQ ID NOs: 350 +304)
Combo = bNAb16 (SEQ ID NOs: 303 + 304) + D1m-K8C-G99C_Fc (SEQ ID NO: 158) (combo)
mAb = bNAb16 (SEQ ID NOs: 303 + 304)
CD4 = D1m-K8C-G99C_Fc (SEQ ID NO: 158)

TABLE 19
IC50 (nM) of different bNAb17-derived bispecific
formats and control molecules against a panel
of HIV-1 envelopes in PSV assay (ACTOne cells)

| | bNAb17-Derived Molecules | | | CD4 Controls |
|---|---|---|---|---|
| Envelope | Bispecific | Combo | mAb | CD4 |
| X2088.C9 | 0.84 | 2.44 | >500 | 9.20 |
| ZM106.9 | 8.48 | 9.13 | >500 | 15.14 |
| 3637_V5_C3 | 45.75 | 97.90 | >500 | >500 |
| 3468_V1_C12 | 39.60 | 27.85 | >500 | 64.15 |
| Q461_E2 | 6.70 | 6.03 | >500 | 11.76 |
| 3326_V4_C3 | 287.50 | 171.50 | >500 | >500 |
| 42368 | 2.14 | 1.50 | >500 | 1.58 |
| CC1/85 | 0.91 | * | * | 1.98 |
| NL4-3 | 0.43 | * | * | 0.31 |
| T278.50 | 4.25 | * | * | 1.43 |
| CAP45.2.00.G3 | 1.39 | * | * | 20.48 |
| HIV-2-HCC-01 | 300.00 | * | * | >300 |

* not tested
Table 19 Molecule Key:
Bispecific = D1m-K8C-G99C_1xG4S_bNAb17 (SEQ ID NOs: 351 + 309)
Combo = bNAb17 (SEQ ID NOs: 308 + 309) + D1m-K8C-G99C_Fc (SEQ ID NO: 158) (combo)
mAb = bNAb17 (SEQ ID NOs: 308 + 309)
CD4 = D1m-K8C-G99C_Fc (SEQ ID NO: 158)

TABLE 20
IC50 (nM) of different bNAb18-derived bispecific
formats and control molecules against a panel
of HIV-1 envelopes in PSV assay (ACTOne cells)

| | bNAb18-Derived Molecules | | | CD4 Controls |
|---|---|---|---|---|
| Envelope | Bispecific | Combo | mAb | CD4 |
| X2088.C9 | 0.63 | 0.26 | 0.20 | 9.20 |
| ZM106.9 | 0.25 | 0.08 | 0.11 | 15.14 |
| 3637_V5_C3 | 102.85 | 377.00 | >500 | >500 |
| 3468_V1_C12 | 26.60 | 38.60 | >500 | 64.15 |
| Q461_E2 | 3.16 | 9.98 | >500 | 11.76 |
| 3326_V4_C3 | 157.00 | 369.50 | >500 | >500 |
| 42368 | 0.94 | 2.18 | >500 | 1.58 |
| CC1/85 | 2.06 | * | * | 1.98 |
| NL4-3 | 0.33 | * | * | 0.31 |
| T278.50 | 5.83 | * | * | 1.43 |
| CAP45.2.00.G3 | 20.89 | * | * | 20.48 |
| HIV-2-HCC-01 | 93.74 | * | * | >300 |

* not tested
Table 20 Molecule Key:
Bispecific = D1m-K8C-G99C_1xG4S_bNAb18 (SEQ ID NOs: 352 + 314)
Combo = bNAb18 (SEQ ID NOs: 313 + 314) + D1m-K8C-G99C_Fc (SEQ ID NO: 158) (combo)
mAb = bNAb18 (SEQ ID NOs: 313 + 314)
CD4 = D1m-K8C-G99C_Fc (SEQ ID NO: 158)

TABLE 21
IC50 (nM) of different bNAb19-derived bispecific
formats and control molecules against a panel
of HIV-1 envelopes in PSV assay (ACTOne cells)

| | bNAb19-Derived Molecules | | | CD4 Controls |
|---|---|---|---|---|
| Envelope | Bispecific | Combo | mAb | CD4 |
| X2088.C9 | 1.63 | 4.40 | >300 | 9.20 |
| ZM106.9 | 3.91 | 8.27 | >300 | 15.14 |
| 3637_V5_C3 | 16.50 | 168.00 | >300 | >500 |
| 3468_V1_C12 | 4.56 | 24.54 | >300 | 64.15 |
| Q461_E2 | 3.18 | 4.49 | >300 | 11.76 |
| 3326_V4_C3 | 130.00 | 241.67 | >300 | >500 |
| 42368 | 1.16 | 1.10 | >300 | 1.58 |
| CC1/85 | 0.52 | * | * | 1.98 |
| NL4-3 | 0.23 | * | * | 0.31 |
| T278.50 | 0.79 | * | * | 1.43 |
| CAP45.2.00.G3 | 5.54 | * | * | 20.48 |
| HIV-2-HCC-01 | 6.54 | * | * | >300 |

* not tested
Table 21 Molecule Key:
Bispecific = D1m-K8C-G99C_1xG4S_bNAb19 (SEQ ID NOs: 353 + 319)
Combo = bNAb19 (SEQ ID NOs: 318 + 319) + D1m-K8C-G99C_Fc (SEQ ID NO: 158) (combo)
mAb = bNAb19 (SEQ ID NOs: 318 + 319)
CD4 = D1m-K8C-G99C_Fc (SEQ ID NO: 158)

TABLE 22
IC50 (nM) of different bNAb20/21/22-derived bispecific formats and control
molecules against a panel of HIV-1 envelopes in PSV assay (ACTOne cells)

| | bNAb20- | | | bNAb21- | | | bNAb22- | | | CD4 |
|---|---|---|---|---|---|---|---|---|---|---|
| | Derived Molecules | | | Derived Molecules | | | Derived Molecules | | | Controls |
| Envelope | 1 | 2 | 3 | 4 | 5 | 6 | 7 | 8 | 9 | 10 |
| X2088.C9 | 0.03 | 0.04 | 0.04 | 0.02 | 0.02 | 0.02 | 0.04 | 0.03 | 0.04 | 9.20 |
| ZM106.9 | 0.01 | 0.02 | 0.02 | 0.01 | 0.01 | 0.01 | 0.01 | 0.01 | 0.01 | 15.14 |
| 3637_V5_C3 | 6.2 | 21.9 | 61.6 | 4.7 | 117.0 | 326.0 | 6.8 | 115.0 | 484.0 | >500 |
| 3468_V1_C12 | 0.01 | 0.02 | 0.02 | 0.01 | 0.01 | 0.01 | 0.01 | 0.02 | 0.01 | 64.15 |
| Q461_E2 | 0.9 | 5.9 | 72.1 | 0.4 | 10.0 | 186.0 | 2.0 | 7.1 | >500 | 11.76 |
| 3326_V4_C3 | 1.3 | 24.1 | 24.0 | 0.3 | 14.5 | 12.5 | 0.6 | 6.1 | 16.0 | >500 |
| 42368 | 0.5 | 3.7 | 59.4 | 0.2 | 1.9 | 128.0 | 0.7 | 1.2 | 391.0 | 1.58 |

Table 22 Molecule Key:
1 = D1m-K8C-G99C_1xG4S_bNAb20 (SEQ ID NOs: 354 + 324)
2 = bNAb20 (SEQ ID NOs: 323 + 324) + D1m-K8C-G99C_Fc (SEQ ID NO: 158) (combo)
3 = bNAb20 (SEQ ID NOs: 323 + 324)
4 = D1m-K8C-G99C_1xG4S_bNAb21 (SEQ ID NOs: 355 + 329)
5 = bNAb21 (SEQ ID NOs: 328 + 329) + D1m-K8C-G99C_Fc (SEQ ID NO: 158) (combo)
6 = bNAb21 (SEQ ID NOs: 328 + 329)
7 = D1m-K8C-G99C_1xG4S_bNAb22 (SEQ ID NOs: 356 + 366)
8 = bNAb22 (SEQ ID NOs: 332 + 366) + D1m-K8C-G99C_Fc (SEQ ID NO: 158) (combo)
9 = bNAb22 (SEQ ID NOs: 332 + 366)
10 = D1m-K8C-G99C_Fc (SEQ ID NO: 158)

TABLE 23
IC50 (nM) of different bNAb23-derived
bispecific formats and control molecules against a panel
of HIV-1 envelopes in PSV assay (ACTOne cells)

| | | | | | | CD4 |
|---|---|---|---|---|---|---|
| | bNAb23-Derived Molecules | | | | | Controls |
| Envelope | 1 | 2 | 3 | 4 | 5 | 6 |
| X2088.C9 | 6.05 | 0.09 | 12.05 | 5.89 | >500 | 9.20 |
| ZM106.9 | 3.96 | 0.12 | 6.06 | 15.06 | 17.4 | 15.14 |
| 3637_V5_C3 | 129.50 | 2.26 | >500 | 294.00 | >500 | >500 |
| 3468_V1_C12 | 62.10 | 6.17 | 384.00 | 53.05 | >500 | 64.15 |
| Q461_E2 | 4.29 | 0.80 | 116.00 | 7.11 | >500 | 11.76 |
| 3326_V4_C3 | 44.90 | 3.94 | 108.00 | 173.00 | 15.3 | >500 |
| 42368 | 0.62 | 0.30 | 10.70 | 1.57 | >500 | 1.58 |

Table 23 Molecule Key:
1 = D1m-K8C-G99C_1xG4S_bNAb23 (SEQ ID NOs: 357 + 337)
2 = D1m-K8C-G99C_1xG4S_bNAb23-LC (SEQ ID NOs: 336 + 358)
3 = bNAb23-HC_1xG4S_D1m-K8C-G99C (SEQ ID NOs: 359 + 337)
4 = bNAb23 (SEQ ID NOs: 336 + 337) + D1m-K8C-G99C_Fc (SEQ ID NO: 158) (combo)
5 = bNAb23 (SEQ ID NOs: 336 + 337)
6 = D1m-K8C-G99C_Fc (SEQ ID NO: 158)

### Example 8—Broad Spectrum Anti-Viral Activity of a bNAb1-Derived Bispecific Molecule (SEQ ID NO:121 and SEQ ID NO:63)

A selected bispecific molecule derived from bNAb1 having two heavy chains (SEQ ID NO: 121) and two light chains (SEQ ID NO: 63) was independently tested against an external panel of pseudo-typed HIV-1 virus containing 119 HIV-1 envelopes and 1 control envelope in a PSV assay (TMZ.bl—see Example 3 above), to further evaluate its breadth and potency. As shown in Table 24, the bispecific molecule completely and potently inhibited all of the 119 HIV-1 envelopes in this assay.

TABLE 24
IC50 and IC80 values (μg/ml) of bNAb1-derived
bispecific molecule (SEQ ID NO: 121 and SEQ ID
NO: 63) against 119 HIV-1 envelopes in PSV assay

| Virus ID | Clade* | IC50 | IC80 | MPI |
|---|---|---|---|---|
| 6535.3 | B | 0.007 | 0.021 | 100 |
| QH0692.42 | B | 0.020 | 0.066 | 100 |
| SC422661.8 | B | 0.013 | 0.032 | 100 |
| PVO.4 | B | 0.010 | 0.037 | 100 |
| TRO.11 | B | 0.011 | 0.026 | 100 |
| AC10.0.29 | B | 0.006 | 0.022 | 100 |
| RHPA4259.7 | B | 0.007 | 0.021 | 100 |
| THRO4156.18 | B | 0.027 | 0.095 | 100 |
| REJO4541.67 | B | 0.022 | 0.054 | 100 |
| TRJO4551.58 | B | 0.006 | 0.022 | 100 |
| WITO4160.33 | B | 0.024 | 0.099 | 100 |
| CAAN5342.A2 | B | 0.023 | 0.085 | 100 |
| WEAU_d15_410_787 | B (T/F) | 0.009 | 0.033 | 100 |
| 1006_11_C3_1601 | B (T/F) | 0.005 | 0.014 | 100 |
| 1054_07_TC4_1499 | B (T/F) | 0.014 | 0.041 | 100 |
| 1056_10_TA11_1826 | B (T/F) | 0.009 | 0.026 | 100 |
| 1012_11_TC21_3257 | B (T/F) | 0.004 | 0.015 | 100 |
| 6240_08_TA5_4622 | B (T/F) | 0.019 | 0.054 | 100 |
| 6244_13_B5_4576 | B (T/F) | 0.011 | 0.039 | 100 |
| 62357_14_D3_4589 | B (T/F) | 0.021 | 0.060 | 100 |
| SC05_8C11_2344 | B (T/F) | 0.016 | 0.045 | 100 |
| Du156.12 | C | 0.005 | 0.018 | 100 |
| Du172.17 | C | 0.010 | 0.029 | 100 |
| Du422.1 | C | 0.011 | 0.044 | 100 |
| ZM197M.PB7 | C | 1.148 | 5.807 | 100 |
| ZM214M.PL15 | C | 0.020 | 0.069 | 100 |
| ZM233M.PB6 | C | 0.046 | 0.171 | 100 |
| ZM249M.PL1 | C | 0.006 | 0.021 | 100 |
| ZM53M.PB12 | C | 0.275 | 0.908 | 100 |
| ZM109F.PB4 | C | 0.036 | 0.132 | 100 |
| ZM135M.PL10a | C | 0.030 | 0.119 | 100 |
| CAP45.2.00.G3 | C | 0.260 | 2.976 | 100 |
| CAP210.2.00.E8 | C | 0.023 | 0.102 | 100 |
| HIV-001428-2.42 | C | 0.002 | 0.004 | 100 |
| HIV-0013095-2.11 | C | 0.016 | 0.042 | 100 |
| HIV-16055-2.3 | C | 0.044 | 0.159 | 100 |
| HIV-16845-2.22 | C | 0.042 | 0.147 | 100 |
| Ce1086_B2 | C (T/F) | 0.085 | 0.297 | 100 |
| Ce0393_C3 | C (T/F) | 0.008 | 0.022 | 100 |
| Ce1176_A3 | C (T/F) | 0.010 | 0.028 | 100 |
| Ce2010_F5 | C (T/F) | 0.206 | 0.693 | 100 |
| Ce0682_E4 | C (T/F) | 0.080 | 1.232 | 100 |
| Ce1172_H1 | C (T/F) | 0.005 | 0.015 | 100 |
| Ce2060_G9 | C (T/F) | 0.016 | 0.059 | 100 |
| Ce703010054_2A2 | C (T/F) | 0.342 | 1.030 | 100 |
| BF1266.431a | C (T/F) | 0.106 | 1.283 | 100 |
| 246F C1G | C (T/F) | 0.009 | 0.031 | 100 |
| 249M B10 | C (T/F) | 0.627 | 5.222 | 99 |
| ZM247v1(Rev-) | C (T/F) | 0.004 | 0.014 | 100 |
| 7030102001E5(Rev-) | C (T/F) | 0.010 | 0.025 | 100 |
| 1394C9G1(Rev-) | C (T/F) | 0.008 | 0.028 | 100 |
| Ce704809221_1B3 | C (T/F) | 0.014 | 0.060 | 100 |
| CNE19 | BC | 0.054 | 0.270 | 100 |
| CNE20 | BC | 0.001 | 0.004 | 100 |
| CNE21 | BC | 0.004 | 0.013 | 100 |
| CNE17 | BC | 0.023 | 0.073 | 100 |
| CNE30 | BC | 0.026 | 0.074 | 100 |
| CNE52 | BC | 0.018 | 0.043 | 100 |
| CNE53 | BC | 0.005 | 0.013 | 100 |
| CNE58 | BC | 0.008 | 0.022 | 100 |
| MS208.A1 | A | 1.457 | 6.230 | 100 |
| Q23.17 | A | 0.010 | 0.025 | 100 |
| Q461.e2 | A | 0.025 | 0.086 | 100 |
| Q769.d22 | A | 0.046 | 0.122 | 100 |
| Q259.d2.17 | A | 0.167 | 0.459 | 100 |
| Q842.d12 | A | 0.006 | 0.015 | 100 |
| 0260.v5.c36 | A | 0.031 | 0.111 | 100 |
| 3415.v1.c1 | A | 0.010 | 0.028 | 100 |
| 3365.v2.c20 | A | 0.011 | 0.036 | 100 |
| 191955_A11 | A (T/F) | 0.032 | 0.115 | 100 |
| 191084 B7-19 | A (T/F) | 0.009 | 0.027 | 100 |
| 9004SS_A3_4 | A (T/F) | 0.008 | 0.023 | 100 |
| T257-31 | CRF02_AG | 0.022 | 0.064 | 100 |
| 928-28 | CRF02_AG | 0.029 | 0.086 | 100 |
| 263-8 | CRF02_AG | 0.011 | 0.043 | 100 |
| T250-4 | CRF02_AG | 0.005 | 0.012 | 100 |
| T251-18 | CRF02_AG | 0.023 | 0.064 | 100 |
| T278-50 | CRF02_AG | 0.010 | 0.027 | 100 |
| T255-34 | CRF02_AG | 0.025 | 0.091 | 100 |
| 211-9 | CRF02_AG | 0.025 | 0.085 | 100 |
| 235-47 | CRF02_AG | 0.170 | 0.800 | 100 |
| 620345.c01 | CRF01_AE | 0.247 | 2.737 | 100 |
| CNE8 | CRF01_AE | 0.007 | 0.021 | 100 |
| C1080.c03 | CRF01_AE | 0.014 | 0.027 | 100 |
| R2184.c04 | CRF01_AE | 0.006 | 0.022 | 100 |
| R1166.c01 | CRF01_AE | 0.252 | 1.001 | 100 |
| R3265.c06 | CRF01_AE | 0.065 | 0.254 | 100 |
| C2101.c01 | CRF01_AE | 0.005 | 0.016 | 100 |
| C3347.c11 | CRF01_AE | 0.006 | 0.015 | 100 |
| C4118.c09 | CRF01_AE | 0.512 | 4.356 | 100 |
| CNE5 | CRF01_AE | 0.014 | 0.038 | 100 |
| BJOX009000.02.4 | CRF01_AE | 0.006 | 0.020 | 100 |
| BJOX015000.11.5 | CRF01_AE (T/F) | 0.013 | 0.035 | 100 |
| BJOX010000.06.2 | CRF01_AE (T/F) | 0.035 | 0.114 | 100 |
| BJOX025000.01.1 | CRF01_AE (T/F) | 0.035 | 0.140 | 100 |
| BJOX028000.10.3 | CRF01_AE (T/F) | 0.004 | 0.010 | 100 |
| X1193_c1 | G | 0.015 | 0.034 | 100 |
| P0402_c2_11 | G | 0.005 | 0.019 | 100 |
| X1254_c3 | G | 0.014 | 0.040 | 100 |
| X2088_c9 | G | 0.017 | 0.048 | 100 |
| X2131_C1_B5 | G | 0.014 | 0.039 | 100 |
| P1981_C5_3 | G | 0.014 | 0.038 | 100 |
| X1632_S2_B10 | G | 0.027 | 0.097 | 100 |
| 3016.v5.c45 | D | 0.041 | 0.117 | 100 |
| A07412M1.vrc12 | D | 0.009 | 0.034 | 100 |
| 231965.c01 | D | 0.023 | 0.068 | 100 |
| 231966.c02 | D | 0.006 | 0.018 | 100 |
| 6405.v4.c34 | D | 0.029 | 0.082 | 100 |
| 3817.v2.c59 | CD | 0.025 | 0.063 | 100 |
| 6480.v4.c25 | CD | 0.008 | 0.024 | 100 |
| 6952.v1.c20 | CD | 0.037 | 0.109 | 100 |
| 6811.v7.c18 | CD | 0.004 | 0.014 | 100 |
| 89-F1_2_25 | CD | 0.035 | 0.110 | 100 |
| 3301.v1.c24 | AC | 0.005 | 0.018 | 100 |
| 6041.v3.c23 | AC | 0.109 | 0.553 | 100 |
| 6540.v4.c1 | AC | 0.002 | 0.012 | 100 |
| 6545.v4.c1 | AC | 0.011 | 0.031 | 100 |
| 0815.v3.c3 | ACD | 0.003 | 0.008 | 100 |
| 3103.v3.c10 | ACD | 0.012 | 0.027 | 100 |
| MuLV | Negative | >50 | >50 | 0 |
| | Control | | | |

### Example 9—Activity of bNAb1-Derived Bispecific Molecules Against Laboratory and Clinical Isolates of HIV-1 in Replicating Virus Assays

### Method

### Clinical and Laboratory Isolates

All clinical and laboratory isolates were originally obtained from the NIH AIDS Reagent Program (currently NIH HIV Reagent Program, hivreagentprogram.org/). The proviral clone of NL₄₋₃ (obtained from NIH) was used to make the replicating reporter virus NLRepRluc, in which a section of the nef gene from the proviral clone of NL₄₋₃ was replaced with the Renilla luciferase gene. Virus was produced through transfection of HEK293T cells using Lipofectamine Plus (Invitrogen, Carlsbad, CA), according to the manufacturer's instructions. The replication-competent virus was harvested 3 days after transfection of HEK 293T cells with the modified pNLRepRluc proviral clone and titrated in MT-2 cells using luciferase activity as a biomarker.

Clinical isolates were initially propagated in human PBMC cells. T-tropic laboratory virus strains IIIB, NL₄₋₃, HXB2, LAI, MN and RF viruses were propagated in MT-2 cells, while M-tropic laboratory strains Bal and JR-FL were propagated in PM1 cells. Titers of virus stocks were determined in PBMC using a virus infectivity assay with a p24 antigen endpoint (p24 ELISA kit; PerkinElmer Life Sciences). All those viruses were further titered in MT2 or CCR5-B6 cells before experiments by using luciferase enzyme activity as an endpoint for 50% tissue culture infectious dose (TCID₅₀) determination.

### Cells

MT-2 cells were obtained from the American Type Culture Collection (ATCC) and were propagated in RPMI 1640 medium supplemented with 10% heat-inactivated fetal bovine serum (FBS), 100 units/ml of penicillin G, 100 μg/ml of streptomycin, 10 mM HEPES buffer pH 7.55 and 2-mM L-glutamine. HEK293T cells were obtained from the ATCC and propagated in DMEM media supplemented with 10% heat-inactivated FBS. The ACTOne cells were originally derived from HEK293T cells and express CD4, CCR5 and CXCR4. They are grown in DMEM media supplemented with 10% heat-inactivated FBS, 100 U/ml of penicillin G, 100 μg/ml of streptomycin, 5 μg/ml blasticidin, 200 μg/ml G418 and 1.5 μg/ml puromycin. CCR5-B6 cells were generated in-house at ViiV Branford CT USA. To generate CCR5-B6 cells, human CCR5 lentiviral particles were used to infect the MT4-B6 cells (obtained from Bristol-Myers Squibb) that has an integrated copy of the LTR-fire-fly luciferase reporter (backbone: plenti-P2A-Puro, RC223291L3V, Origene) and stable cells were selected by using G418 (0.6 mg/ml) and puromycin (2 μg/ml). The CCR5-B6 cells express firefly luciferase from an HIV-1 LTR promoter after infection with HIV. They are grown in RPMI 1640 supplemented with 10% heat inactivated fetal bovine serum (FBS), 10 mM HEPES buffer pH 7.55, 2 mM L-glutamine, 100 units/ml penicillin G, and 100 μg/ml streptomycin, 2 μg/ml puromycin, 0.6 mg/ml G418.

### Replicating Virus Assay

### Assay Using NLRepRluc Virus

The NLRepRluc was used to infect MT-2 cells at a multiplicity of 0.01 for 1 hour before adding the proteins to the 96-well plates. Antibodies were serially diluted four-fold and 11 concentrations were plated in triplicate. After 4 days of incubation, cells were processed and quantitated for virus growth by the amount of expressed luciferase. Luciferase was quantitated using the ENDUREN substrate from Promega (Madison, WI) according to the manufacturer's instructions. Luciferase activity was measured immediately on an ENVISION multilabel plate reader (PerkinElmer, Waltham MA). EC₅₀ values were calculated by comparing the amount of luciferase produced in the presence of antigen binding protein compared to wells where no antigen binding protein (DMSO control) was added. A 5-parameter sigmoidal equation was used to fit the resulting signal vs. concentration curves, and the concentration of each antigen binding protein that produced 50% maximal inhibition (EC₅₀) was determined. The results of three independent experiments were averaged and plotted, with error bars corresponding to 1 standard deviation.

### Replicating Virus Assay Using Laboratory Strains and Clinical Isolates

Replicating laboratory strains and clinical isolates were prepared as described above. MT2 cells or CCR5-B6 cells were resuspended in corresponding media and distributed to 96-well assay plates (26,000 cells/well in 100 uL; Corning, Tewksbury, MA) containing serial dilutions of inhibitors in DMSO (5 or 3-fold dilutions, columns 1-10). The blank controls were wells containing DMSO (column 11,12). Replicating whole viruses of laboratory strains or clinic isolates were diluted in RPMI-1640 culture medium based on 50% tissue culture infectious dose (TCID₅₀) determination such that undiluted stock virus was added to the first well, and 100 μl of viral culture medium were loaded to the wells already containing compounds and cells (total 200 μl/well), resulting in a final concentration of DMSO of 1%. Plates were incubated at 37° C. and 5% CO₂ for approximately 4 days. After that, Renilla luciferase activity was measured (Enduren reagent, Promega Corp., Madison, WI) on an EnVision Multilabel pate reader (Perkin Elmer, Inc., Waltham MA). The 50% effective concentration (EC₅₀) was calculated by using the exponential form of the median effect equation where (Fa)=1/[1+(ED₅₀/drug conc.)].

### Results

Table 25.1 shows that the bNAb1-derived bispecific molecules are consistently about 10-fold more active than the mixture of the component parts (bNAb1 and CD4 domain), again indicating a clear anti-viral synergy result from fusing these component binding domains.

TABLE 25.1
EC50 (nM) values of bNAb1-derived bispecific and control
molecules against NL4-3 in a replicating virus assay

| Molecules (SEQ ID NO) | EC50 (nM) |
|---|---|
| D1m_1xG4S_bNAb1 (102 + 63) | 0.03 |
| D1m_2xG4S_bNAb1 (103 + 63) | 0.03 |
| D1m_3xG4S_bNAb1 (104 + 63) | 0.02 |
| D1m_4xG4S_bNAb1 (105 + 63) | 0.03 |
| D1m_His (4*) + bNAb1 (62 + 63) | 0.22 |
| D1m_His (4*) | 0.23 |
| bNAb1 (62 + 63) | >500 |

*plus a 6xHis tag (six C-terminal histidine residues)

A bNAb1-derived bispecific molecule (D1m-K8C-G99C_1xG4S_bNAb1, SEQ ID NOs: 121+63) was tested against a panel of 13 clinical and 8 laboratory HIV-1 isolates in a replicating virus assay using MT-2 and CCR5-B6 cells. As shown in Table 25.2, this molecule neutralized all strains, with EC50s less than 1 nM (geometric mean EC50 being 0.11 nM against clinical isolates and 0.26 nM against lab strains), demonstrating again its strong potency and breadth of activity.

TABLE 25.2
EC₅₀ (nM) of a bNAb1-derived bispecific against
a panel of HIV-1 clinical isolates and laboratory
strains in a replicating virus assay

| | | |
|---|---|---|
| | | bNAb1-Derived |
| Isolates | Clade | Bispecific |

Clinical isolates

| | | |
|---|---|---|
| 93US141 | B | 0.04 |
| 93US144 | B | 0.07 |
| ASM34 | B | 0.15 |
| BK132 | B | 0.19 |
| BZ167 | B | 0.05 |
| CC1/85 | B | 0.2 |
| CM237 | B | 0.03 |
| ETH2220 | C | 0.08 |
| I-2496 | A | 0.15 |
| SE364 | C | 0.2 |
| UG268 | C | 0.06 |
| UG270 | D | 0.42 |
| US4 | B | 0.39 |

Laboratory strains

| | |
|---|---|
| HXB2 | 0.04 |
| BaL | 0.83 |
| IIIB/H9 | 0.56 |
| IIIB/HOS | 0.56 |
| JR-FL | 0.04 |
| LAI | 0.39 |
| MIN | 0.15 |
| NL4-3 | 0.39 |
| RF | 0.56 |

Table 25.2 Molecule Key:
1 = D1m-K8C-G99C_1xG4S_bNAb1 (SEQ ID NOs: 121 + 63)

### Example 10—In Vitro Resistance Barrier

To assess the resistance barrier of the bispecific molecules, we examined the relative rates at which HIV NL4-3 virus can escape inhibition by a panel of antibodies versus a DMSO control.

MT2 cells (2.0×10⁵/well in RPMI 1640+50 mg/ml penicillin and streptomycin+10 mM HEPES buffer pH 7.55+2 mM L-glutamine. +0.2% DMSO) were pre-infected at an MOI (multiplicity of infection) of 0.005 for 2.5 hours and then pelleted to remove unbound virus particles. One mL of infected cells was added to each well of a 24 well plate. Replicates of antibody dilutions at the concentrations of 20-, 30-, or 40-fold of IC50 values were then added (1 mL of a 2X stock) to achieve 2 mL assay volume.

Every 3-4 days images of the wells were captured and 1 mL of each well is removed and replaced by fresh preparations of each condition. This process was continued until there was viral breakthrough (observed cytopathic effect (CPE)>80%) or until there was a confirmed elimination of infected cells (via challenged elimination). When either condition was achieved, the sample was collected as pellet and supernatant (via centrifugation) and stored at −80° C. until genotypic analysis was performed to confirm the presence of resistance mutations. The days from infection to breakthrough were used to estimate the resistance barrier of a certain molecule. The longer it takes the virus to develop CPE, which indicates resistance, the higher the resistance barrier is.

Table 26 shows that, in this experimental setting, the bNAb1-derived bispecific molecule (SEQ ID NO:105 and SEQ ID NO:63) exhibited a much higher resistance barrier than the soluble CD4 domain or bNAb1 alone at all concentrations. This again indicates synergy between soluble CD4 domains and bNAb1 when fused together.

TABLE 26

| | | | Average days |
|---|---|---|---|
| Group | Molecule | Conc. (nM) | before CPE |
| 20× EC50 | D1m_4xG4S_bNAb1 | 4.2 | 16 |
| | D1m_His | 8.4 | 4 |
| | bNAb1 | 18 | 4 |
| 30× EC50 | D1m_4xG4S_bNAb1 | 6.3 | 54 |
| | D1m_His | 12.5 | 5 |
| | bNAb1 | 27 | 4 |
| 40× EC50 | D1m_4xG4S_bNAb1 | 8.4 | 62 |
| | D1m_His | 16.7 | 7 |
| | bNAb1 | 36 | 4 |

Table 26 Molecule Key:
D1m_4xG4S_bNAb1 = SEQ ID NO: 105 and 63
D1m_His = SEQ ID NO: 4 plus a 6xHis tag (six C-terminal histidine residues)
bNAb1 = SEQ ID NOs: 62 and 63

### Example 11—Anti-HIV-2 Activity of a bNAb1-Derived Bispecific Molecule (SEQ ID NO: 121 and SEQ ID NO:63) in Pseudotyped and Replicating Virus Assays

HIV-2 differs from HIV-1 in that it originates from the transmission of simian immunodeficiency virus (SIV) from sooty mangabeys (SIVsmm) to human (Gao et al, J Virol. 1994; 68 (11): 7433-7447) whilst 20 HIV-1 stems from the transmission from chimpanzees and western gorillas (HIV-1 group M and O, respectively). HIV-2 can also cause AIDS but is far less pathogenic and wide-spread than HIV-1 (de Silva et al, Trends Microbiol. 2008; 16 (12): 588-595; Da Silva et al, AIDS. 2008; 22 (10): 1195-1202). Though also using CD4 for infection (Sattentau et al, AIDS. 1988; 2 (2): 101-105), HIV-2 shares only 40% identity in the gp160 amino acid sequence with HIV-1, and is therefore less sensitive or 25 insensitive to HIV-1 envelope-directed bnAbs (Kong et al, JVI 2012; 86 (2): 947-960). The bNAb1-derived bispecific molecule (SEQ ID NO: 121 and SEQ ID NO: 63) showed strong potency and anti-viral synergy against the 2 HIV-2 Env-pseudotypes tested in PSV assays and the laboratory NIHZ strain examined in a replicating virus assay (Table 27), illustrating its exceptional breadth of anti-HIV activity and excellent synergy.

TABLE 27
IC50 (nM) of a bNAb1-derived bispecific and
control molecules against HIV-2 strains

| | | bNAb1-derived molecules | | | |
|---|---|---|---|---|---|
| Strain | Assay | 1 | 2 | 3 | 4 |
| HIV-2-ATM88 | PSV | 0.07 | 3.93 | >500 | 10.53 |
| HIV-2-HCC-01 | PSV | 0.78 | >500 | >500 | >500 |
| HIV-2 NIHZ | Replicating | 2.68 | * | * | * |
| | virus | | | | |

* not tested
Table 27 Molecule Key:
1 = D1m-K8C-G99C_1xG4S_bNAb1 (SEQ ID NOs: 121 + 63)
2 = D1m_His (SEQ ID NO: 4*)
3 = bNAb1 (SEQ ID NOs: 62 + 63)
4 = D1m_His (SEQ ID NO: 4*) + bNAb1(SEQ ID NOs: 62 + 63) (combo)
* plus a 6xHis tag (six C-terminal histidine residues)

SEQUENCE LISTING

| SEQ ID | | |
|---|---|---|
| NO | Name/Identifier | Description |
| 1 | D1D2 | Human CD4 D1D2 (wild type) |
| 2 | D1mD2 | Human CD4 mD1.22-D2 (D1 mutations |
| | | S23N, A55V, and L96V) |
| 3 | D1 | Human CD4 D1 (wild type) |
| 4 | D1m | Human CD4 mD1.22 (NCBI accession code: |
| | | QHY83614.1; D1 mutations L5Y, S23N, |
| | | A55V, I76P, L96V and F98V) |
| 5 | D1m-K8V | Human CD4 mD1.22 + K8V |
| 6 | D1m-E91Q | Human CD4 mD1.22 + E91Q |
| 7 | D1m-E91H | Human CD4 mD1.22 + E91H |
| 8 | D1m-E87G | Human CD4 mD1.22 + E87G |
| 9 | D1m-N52W | Human CD4 mD1.22 + N52W |
| 10 | D1m-K8I | Human CD4 mD1.22 + K8I |
| 11 | D1m-K8C-G99C | Human CD4 mD1.22 + K8C, G99C |
| 12 | D1m-T11C-K72C | Human CD4 mD1.22 + T11C, K72C |
| 13 | D1m-E13C-I70C | Human CD4 mD1.22 + E13C, I70C |
| 14 | D1m-H27C-G38C | Human CD4 mD1.22 + H27C, G38C |
| 15 | D1m-K21C-G65C | Human CD4 mD1.22 + K21C, G65C |
| 16 | D1m-Q25E | Human CD4 mD1.22 + Q25E |
| 17 | D1m-H27D | Human CD4 mD1.22 + H27D |
| 18 | D1m-R58V | Human CD4 mD1.22 + R58V |
| 19 | D1m-R58N | Human CD4 mD1.22 + R58N |
| 20 | D1m-R58T | Human CD4 mD1.22 + R58T |
| 21 | D1m-L61M | Human CD4 mD1.22 + L61M |
| 22 | bNAb1 CDRH1 | CDRH1 of bNAb1 |
| 23 | bNAb1 CDRH2 | CDRH2 of bNAb1 |
| 24 | bNAb1 CDRH3 | CDRH3 of bNAb1 |
| 25 | bNAb1 CDRL1 | CDRL1 of bNAb1 |
| 26 | bNAb1 CDRL2 | CDRL2 of bNAb1 |
| 27 | bNAb1 CDRL3 | CDRL3 of bNAb1 |
| 28 | bNAb2 CDRH1 | CDRH1 of bNAb2 |
| 29 | bNAb2 CDRH2 | CDRH2 of bNAb2 |
| 30 | bNAb2 CDRH3 | CDRH3 of bNAb2 |
| 31 | bNAb2 CDRL1 | CDRL1 of bNAb2 |
| 32 | bNAb2 CDRL2 | CDRL2 of bNAb2 |
| 33 | bNAb2 CDRL3 | CDRL3 of bNAb2 |
| 34 | bNAb3 CDRH1 | CDRH1 of bNAb3 |
| 35 | bNAb3 CDRH2 | CDRH2 of bNAb3 |
| 36 | bNAb3 CDRH3 | CDRH3 of bNAb3 |
| 37 | bNAb3 CDRL1 | CDRL1 of bNAb3 |
| 38 | bNAb3 CDRL2 | CDRL2 of bNAb3 |
| 39 | bNAb3 CDRL3 | CDRL3 of bNAb3 |
| 40 | bNAb4 CDRH1 | CDRH1 of bNAb4 |
| 41 | bNAb4 CDRH2 | CDRH2 of bNAb4 |
| 42 | bNAb4 CDRH3 | CDRH3 of bNAb4 |
| 43 | bNAb4 CDRL1 | CDRL1 of bNAb4 |
| 44 | bNAb4 CDRL2 | CDRL2 of bNAb4 |
| 45 | bNAb4 CDRL3 | CDRL3 of bNAb4 |
| 46 | bNAb5 CDRH1 | CDRH1 of bNAb5 |
| 47 | bNAb5 CDRH2 | CDRH2 of bNAb5 |
| 48 | bNAb5 CDRH3 | CDRH3 of bNAb5 |
| 49 | bNAb5 CDRL1 | CDRL1 of bNAb5 |
| 50 | bNAb5 CDRL2 | CDRL2 of bNAb5 |
| 51 | bNAb5 CDRL3 | CDRL3 of bNAb5 |
| 52 | bNAb6 CDRH1 | CDRH1 of bNAb6 |
| 53 | bNAb6 CDRH2 | CDRH2 of bNAb6 |
| 54 | bNAb6 CDRH3 | CDRH3 of bNAb6 |
| 55 | bNAb6 CDRL1 | CDRL1 of bNAb6 |
| 56 | bNAb6 CDRL2 | CDRL2 of bNAb6 |
| 57 | bNAb6 CDRL3 | CDRL3 of bNAb6 |
| 58 | bNAb1 VH | Heavy chain variable region of bNAb1 |
| 59 | bNAb1 VL | Light chain variable region of bNAb1 |
| 60 | bNAb1* VL | Light chain variable region of bNAb1 with |
| | | F32Y mutation |
| 61 | bnAb1 HC | Full heavy chain of bNAb1 |
| 62 | bNAb1 HC + LS | Full heavy chain of bNAb1 with |
| | | M428L/N434S mutations |
| 63 | bNAb1 LC | Full light chain of bNAb1 |
| 64 | bNAb1* LC | Full light chain of bNAb1 with F32Y |
| | | mutation |
| 65 | bNAb2 VH | Heavy chain variable region of bNAb2 |
| 66 | bNAb2 VL | Light chain variable region of bNAb2 |
| 67 | bNAb2 HC | Full heavy chain of bNAb2 |
| 68 | bNAb2 HC + LS | Full heavy chain of bNAb2 with |
| | | M428L/N434S mutations |
| 69 | bNAb2 LC | Full light chain of bNAb2 |
| 70 | bNAb3 VH | Heavy chain variable region of bNAb3 |
| 71 | bNAb3 VL | Light chain variable region of bNAb3 |
| 72 | bNAb3 HC | Full heavy chain of bNAb3 |
| 73 | bNAb3 HC + LS | Full heavy chain of bNAb3 with |
| | | M428L/N434S mutations |
| 74 | bNAb3 LC | Full light chain of bNAb3 |
| 75 | bNAb4 VH | Heavy chain variable region of bNAb4 |
| 76 | bNAb4 VL | Light chain variable region of bNAb4 |
| 77 | bNAb4 HC | Full heavy chain of bNAb4 |
| 78 | bNAb4 HC + LS | Full heavy chain of bNAb4 with |
| | | M428L/N434S mutations |
| 79 | bNAb4 LC | Full light chain of bNAb4 |
| 80 | bNAb5 VH | Heavy chain variable region of bNAb5 |
| 81 | bNAb5 VL | Light chain variable region of bNAb5 |
| 82 | bNAb5 HC | Full heavy chain of bNAb5 |
| 83 | bNAb5 HC + LS | Full heavy chain of bNAb5 with |
| | | M428L/N434S mutations |
| 84 | bNAb5 LC | Full light chain of bNAb5 |
| 85 | bNAb6 VH | Heavy chain variable region of bNAb6 |
| 86 | bNAb6 VL | Light chain variable region of bNAb6 |
| 87 | bNAb6 HC | Full heavy chain of bNAb6 |
| 88 | bNAb6 HC + LS | Full heavy chain of bNAb6 with |
| | | M428L/N434S mutations |
| 89 | bNAb6 LC | Full light chain of bNAb6 |
| 90 | 1xG4S | Linker |
| 91 | 2xG4S | Linker |
| 92 | 3xG4S | Linker |
| 93 | 4xG4S | Linker |
| 94 | 5xG4S | Linker |
| 95 | 6xG4S | Linker |
| 96 | D1mD2_0xG4S_bNAb1-HC | Human CD4 mD1.22-D2 fused to the N- |
| | | terminus of bNAb1 heavy chain with no |
| | | linker, Fc includes M428L/N434S |
| 97 | D1mD2_1xG4S_bNAb1-HC | Human CD4 mD1.22-D2 fused to the N- |
| | | terminus of bNAb1 heavy chain with 1xG4S |
| | | linker, Fc includes M428L/N434S |
| 98 | D1mD2_2xG4S_bNAb1-HC | Human CD4 mD1.22-D2 fused to the N- |
| | | terminus of bNAb1 heavy chain with 2xG4S |
| | | linker, Fc includes M428L/N434S |
| 99 | D1mD2_3xG4S_bNAb1-HC | Human CD4 mD1.22-D2 fused to the N- |
| | | terminus of bNAb1 heavy chain with 3xG4S |
| | | linker, Fc includes M428L/N434S |
| 100 | D1mD2_4xG4S_bNAb1-HC | Human CD4 mD1.22-D2 fused to the N- |
| | | terminus of bNAb1 heavy chain with 4xG4S |
| | | linker, Fc includes M428L/N434S |
| 101 | D1m_0xG4S_bNAb1-HC | Human CD4 mD1.22 fused to the N- |
| | | terminus of bNAb1 heavy chain with no |
| | | linker, Fc includes M428L/N434S |
| 102 | D1m_1xG4S_bNAb1-HC | Human CD4 mD1.22 fused to the N- |
| | | terminus of bNAb1 heavy chain with 1xG4S |
| | | linker, Fc includes M428L/N434S |
| 103 | D1m_2xG4S_bNAb1-HC | Human CD4 mD1.22 fused to the N- |
| | | terminus of bNAb1 heavy chain with 2xG4S |
| | | linker, Fc includes M428L/N434S |
| 104 | D1m_3xG4S_bNAb1-HC | Human CD4 mD1.22 fused to the N- |
| | | terminus of bNAb1 heavy chain with 3xG4S |
| | | linker, Fc includes M428L/N434S |
| 105 | D1m_4xG4S_bNAb1-HC | Human CD4 mD1.22 fused to the N- |
| | | terminus of bNAb1 heavy chain with 4xG4S |
| | | linker, Fc includes M428L/N434S |
| 106 | D1m_5xG4S_bNAb1-HC | Human CD4 mD1.22 fused to the N- |
| | | terminus of bNAb1 heavy chain with 5xG4S |
| | | linker, Fc includes M428L/N434S |
| 107 | D1m_6xG4S_bNAb1-HC | Human CD4 mD1.22 fused to the N- |
| | | terminus of bNAb1 heavy chain with 6xG4S |
| | | linker, Fc includes M428L/N434S |
| 108 | D1m_0xG4S_bNAb1-LC | Human CD4 mD1.22 fused to the N- |
| | | terminus of bNAb1 light chain with no linker |
| 109 | D1m_1xG4S_bNAb1-LC | Human CD4 mD1.22 fused to the N- |
| | | terminus of bNAb1 light chain with 1xG4S |
| | | linker |
| 110 | D1m_2xG4S_bNAb1-LC | Human CD4 mD1.22 fused to the N- |
| | | terminus of bNAb1 light chain with 2xG4S |
| | | linker |
| 111 | D1m_3xG4S_bNAb1-LC | Human CD4 mD1.22 fused to the N- |
| | | terminus of bNAb1 light chain with 3xG4S |
| | | linker |
| 112 | D1m_4xG4S_bNAb1-LC | Human CD4 mD1.22 fused to the N- |
| | | terminus of bNAb1 light chain with 4xG4S |
| | | linker |
| 113 | D1m_5xG4S_bNAb1-LC | Human CD4 mD1.22 fused to the N- |
| | | terminus of bNAb1 light chain with 5xG4S |
| | | linker |
| 114 | D1m_6xG4S_bNAb1-LC | Human CD4 mD1.22 fused to the N- |
| | | terminus of bNAb1 light chain with 6xG4S |
| | | linker |
| 115 | D1m-K8C-G99C_1xG4S_bNAb1-LC | Human CD4 mD1.22 + K8C + G99C fused |
| | | to the N-terminus of bNAb1 light chain with |
| | | 1xG4S linker |
| 116 | bNAb1-HC-mid_1xG4S_D1m-K8C- | Human CD4 mD1.22 + K8C + G99C fused in |
| | G99C | between the CH1 domain and hinge of |
| | | bNAb1 heavy chain with 1xG4S linker, Fc |
| | | includes M428L/N434S |
| 117 | bNAb1-HC_1xG4S_D1m-K8C-G99C | Human CD4 mD1.22 + K8C + G99C fused |
| | | to the C-terminus of bNAb1 heavy chain |
| | | with 1xG4S linker, Fc includes M428L/N434S |
| 118 | bNAb1-LC_1xG4S_D1m-K8C-G99C | Human CD4 mD1.22 + K8C + G99C fused |
| | | to the C-terminus of bNAb1 light chain with |
| | | 1xG4S linker |
| 119 | D1m-K8I_1xG4S_bNAb1-HC | Human CD4 mD1.22 + K8I fused to the N- |
| | | terminus of bNAb1 heavy chain with 1xG4S |
| | | linker, Fc includes M428L/N434S |
| 120 | D1m-K8V_1xG4S_bNAb1-HC | Human CD4 mD1.22 + K8V fused to the N- |
| | | terminus of bNAb1 heavy chain with 1xG4S |
| | | linker, Fc includes M428L/N434S |
| 121 | D1m-K8C-G99C_1xG4S_bNAb1-HC | Human CD4 mD1.22 + K8C + G99C fused |
| | | to the N-terminus of bNAb1 heavy chain |
| | | with 1xG4S linker, Fc includes M428L/N434S |
| 122 | D1m-T11C-K72C_1xG4S_bNAb1- | Human CD4 mD1.22 + T11C + K72C fused |
| | HC | to the N-terminus of bNAb1 heavy chain |
| | | with 1xG4S linker, Fc includes M428L/N434S |
| 123 | D1m-E91Q_1xG4S_bNAb1-HC | Human CD4 mD1.22 + E91Q fused to the |
| | | N-terminus of bNAb1 heavy chain with |
| | | 1xG4S linker, Fc includes M428L/N434S |
| 124 | D1m-E91H_1xG4S_bNAb1-HC | Human CD4 mD1.22 + E91H fused to the |
| | | N-terminus of bNAb1 heavy chain with |
| | | 1xG4S linker, Fc includes M428L/N434S |
| 125 | D1m-Q25E_1xG4S_bNAb1-HC | Human CD4 mD1.22 + Q25E fused to the |
| | | N-terminus of bNAb1 heavy chain with |
| | | 1xG4S linker, Fc includes M428L/N434S |
| 126 | D1m-H27D_1xG4S_bNAb1-HC | Human CD4 mD1.22 + H27D fused to the |
| | | N-terminus of bNAb1 heavy chain with |
| | | 1xG4S linker, Fc includes M428L/N434S |
| 127 | D1m-R58V_1xG4S_bNAb1-HC | Human CD4 mD1.22 + R58V fused to the |
| | | N-terminus of bNAb1 heavy chain with |
| | | 1xG4S linker, Fc includes M428L/N434S |
| 128 | D1m-R58N_1xG4S_bNAb1-HC | Human CD4 mD1.22 + R58N fused to the |
| | | N-terminus of bNAb1 heavy chain with |
| | | 1xG4S linker, Fc includes M428L/N434S |
| 129 | D1m-R58T_1xG4S_bNAb1-HC | Human CD4 mD1.22 + R58T fused to the N- |
| | | terminus of bNAb1 heavy chain with 1xG4S |
| | | linker, Fc includes M428L/N434S |
| 130 | D1m-L61M_1xG4S_bNAb1-HC | Human CD4 mD1.22 + L61M fused to the |
| | | N-terminus of bNAb1 heavy chain with |
| | | 1xG4S linker, Fc includes M428L/N434S |
| 131 | D1m-E13C-I70C_1xG4S_bNAb1-HC | Human CD4 mD1.22 + E13C + I70C fused |
| | | to the N-terminus of bNAb1 heavy chain |
| | | with 1xG4S linker, Fc includes M428L/N434S |
| 132 | D1m-H27C-G38C_1xG4S_bNAb1- | Human CD4 mD1.22 + H27C + G38C fused |
| | HC | to the N-terminus of bNAb1 heavy chain |
| | | with 1xG4S linker, Fc includes M428L/N434S |
| 133 | D1m-K21C-G65C_1xG4S_bNAb1- | Human CD4 mD1.22 + K21C + G65C fused |
| | HC | to the N-terminus of bNAb1 heavy chain |
| | | with 1xG4S linker, Fc includes M428L/N434S |
| 134 | D1m-E87G_1xG4S_bNAb1-HC | Human CD4 mD1.22 + E87G fused to the |
| | | N-terminus of bNAb1 heavy chain with |
| | | 1xG4S linker, Fc includes M428L/N434S |
| 135 | D1m-N52W_1xG4S_bNAb1-HC | Human CD4 mD1.22 + N52W fused to the |
| | | N-terminus of bNAb1 heavy chain with |
| | | 1xG4S linker, Fc includes M428L/N434S |
| 136 | D1m-K8C-G99C_1xG4S_bNAb2-HC | Human CD4 mD1.22 + K8C + G99C fused |
| | | to the N-terminus of bNAb2 heavy chain |
| | | with 1xG4S linker, Fc includes M428L/N434S |
| 137 | D1m-K8C-G99C_2xG4S_bNAb2-HC | Human CD4 mD1.22 + K8C + G99C fused |
| | | to the N-terminus of bNAb2 heavy chain |
| | | with 2xG4S linker, Fc includes M428L/N434S |
| 138 | D1m-K8C-G99C_3xG4S_bNAb2-HC | Human CD4 mD1.22 + K8C + G99C fused |
| | | to the N-terminus of bNAb2 heavy chain |
| | | with 3xG4S linker, Fc includes M428L/N434S |
| 139 | D1m-K8C-G99C_4xG4S_bNAb2-HC | Human CD4 mD1.22 + K8C + G99C fused |
| | | to the N-terminus of bNAb2 heavy chain |
| | | with 4xG4S linker, Fc includes M428L/N434S |
| 140 | bNAb2-HC-mid_1xG4S_D1m-K8C- | Human CD4 mD1.22 + K8C + G99C fused in |
| | G99C | between the CH1 domain and hinge of |
| | | bNAb2 heavy chain with 1xG4S linkers, Fc |
| | | includes M428L/N434S |
| 141 | bNAb2-HC_1xG4S_D1m-K8C-G99C | Human CD4 mD1.22 + K8C + G99C fused |
| | | to the C-terminus of bNAb2 heavy chain |
| | | with 1xG4S linker, Fc includes M428L/N434S |
| 142 | D1m-K8C-G99C_1xG4S_bNAb2-LC | Human CD4 mD1.22 + K8C + G99C fused |
| | | to the N-terminus of bNAb2 light chain with |
| | | 1xG4S linker |
| 143 | bNAb2-LC_1xG4S_D1m-K8C-G99C | Human CD4 mD1.22 + K8C + G99C fused |
| | | to the C-terminus of bNAb2 light chain with |
| | | 1xG4S linker |
| 144 | D1m-K8C-G99C_1xG4S_bNAb3-HC | Human CD4 mD1.22 + K8C + G99C fused |
| | | to the N-terminus of bNAb3 heavy chain |
| | | with 1xG4S linker, Fc includes M428L/N434S |
| 145 | D1m-K8C-G99C_4xG4S_bNAb3-HC | Human CD4 mD1.22 + K8C + G99C fused |
| | | to the N-terminus of bNAb3 heavy chain |
| | | with 4xG4S linker, Fc includes M428L/N434S |
| 146 | D1m-K8C-G99C_1xG4S_bNAb4-HC | Human CD4 mD1.22 + K8C + G99C fused |
| | | to the N-terminus of bNAb4 heavy chain |
| | | with 1xG4S linker, Fc includes M428L/N434S |
| 147 | D1m-K8C-G99C_4xG4S_bNAb4-HC | Human CD4 mD1.22 + K8C + G99C fused |
| | | to the N-terminus of bNAb4 heavy chain |
| | | with 4xG4S linker, Fc includes M428L/N434S |
| 148 | D1m-K8C-G99C_1xG4S_bNAb5-HC | Human CD4 mD1.22 + K8C + G99C fused |
| | | to the N-terminus of bNAb5 heavy chain |
| | | with 1xG4S linker, Fc includes M428L/N434S |
| 149 | D1m-K8C-G99C_4xG4S_bNAb5-HC | Human CD4 mD1.22 + K8C + G99C fused |
| | | to the N-terminus of bNAb5 heavy chain |
| | | with 4xG4S linker, Fc includes M428L/N434S |
| 150 | D1mD2_4xG4S_bNAb6-HC | Human CD4 mD1.22-D2 fused to the N- |
| | | terminus of bNAb6 heavy chain with 4xG4S |
| | | linker, Fc includes M428L/N434S |
| 151 | D1m_4xG4S_bNAb6-HC | Human CD4 mD1.22 fused to the N- |
| | | terminus of bNAb6 heavy chain with 4xG4S |
| | | linker, Fc includes M428L/N434S |
| 152 | D1m_4xG4S_bNAb1-scFv-H4Lss | Human CD4 mD1.22 fused, via 4xG4S |
| | 2xG4S_Fc | linker, to the N-terminus of bNAb1 scFv |
| | | (VH-4xG4S-VL, with stabilizing disulfide |
| | | bond G44C/HC - G100C/LC), which is fused, |
| | | via 2xG4S linker, to the N-terminus of |
| | | human Fc fragment including M428L/N434S |
| 153 | D1m_3xG4S_bNAb1-scFv-H4Lss_ | Human CD4 mD1.22 fused, via 3xG4S |
| | 2xG4S_Fc | linker, to the N-terminus of bNAb1 scFv |
| | | (VH-4xG4S-VL, with stabilizing disulfide |
| | | bond G44C/HC - G100C/LC), which is fused, |
| | | via 2xG4S linker, to the N-terminus of |
| | | human Fc fragment including M428L/N434S |
| 154 | D1m_2xG4S_bNAb1-scFv-H4Lss_ | Human CD4 mD1.22 fused, via 2xG4S |
| | 2xG4S_Fc | linker, to the N-terminus of bNAb1 scFv |
| | | (VH-4xG4S-VL, with stabilizing disulfide |
| | | bond G44C/HC - G100C/LC), which is fused, |
| | | via 2xG4S linker, to the N-terminus of |
| | | human Fc fragment including M428L/N434S |
| 155 | D1m_1xG4S_bNAb1-scFv-H4Lss_ | Human CD4 mD1.22 fused, via 1xG4S |
| | 2xG4S_Fc | linker, to the N-terminus of bNAb1 scFv |
| | | (VH-4xG4S-VL, with stabilizing disulfide |
| | | bond G44C/HC - G100C/LC), which is fused, |
| | | via 2xG4S linker, to the N-terminus of |
| | | human Fc fragment including M428L/N434S |
| 156 | Fc_4xG4S_D1m_3xG4S_bNAb1- | Human CD4 mD1.22 fused, via 3xG4S |
| | scFv-H4Lss | linker, to the N-terminus of bNAb1 scFv |
| | | (VH-4xG4S-VL, with stabilizing disulfide |
| | | bond G44C/HC - G100C/LC), which is fused, |
| | | via 3xG4S linker, to the C-terminus of |
| | | human Fc fragment including M428L/N434S |
| 157 | bNAb1-scFv-L4Hss_4xG4S_D1m_ | bNAb1 scFv (VL-4xG4S-VH, with stabilizing |
| | 3xG4S_Fc | disulfide bond G44C/HC - G100C/LC) fused, |
| | | via 4xG4S linker, to the N-terminus of |
| | | human CD4 mD1.22, which is fused, via |
| | | 3xG4S linker, to the N-terminus of human |
| | | Fc fragment including M428L/N434S |
| 158 | D1m-K8C-G99C_Fc | Human CD4 mD1.22 + K8C, G99C with C- |
| | | terminal Fc tag including M428L/N434S |
| 159 | bNAb7 CDRH1 | CDRH1 of bNAb7 |
| 160 | bNAb7 CDRH2 | CDRH2 of bNAb7 |
| 161 | bNAb7 CDRH3 | CDRH3 of bNAb7 |
| 162 | bNAb7 CDRL1 | CDRL1 of bNAb7 |
| 163 | bNAb7 CDRL2 | CDRL2 of bNAb7 |
| 164 | bNAb7 CDRL3 | CDRL3 of bNAb7 |
| 165 | bNAb8 CDRH1 | CDRH1 of bNAb8 |
| 166 | bNAb8 CDRH2 | CDRH2 of bNAb8 |
| 167 | bNAb8 CDRH3 | CDRH3 of bNAb8 |
| 168 | bNAb8 CDRL1 | CDRL1 of bNAb8 |
| 169 | bNAb8 CDRL2 | CDRL2 of bNAb8 |
| 170 | bNAb8 CDRL3 | CDRL3 of bNAb8 |
| 171 | bNAb9 CDRH1 | CDRH1 of bNAb9 |
| 172 | bNAb9 CDRH2 | CDRH2 of bNAb9 |
| 173 | bNAb9 CDRH3 | CDRH3 of bNAb9 |
| 174 | bNAb9 CDRL1 | CDRL1 of bNAb9 |
| 175 | bNAb9 CDRL2 | CDRL2 of bNAb9 |
| 176 | bNAb9 CDRL3 | CDRL3 of bNAb9 |
| 177 | bNAb10 CDRH1 | CDRH1 of bNAb10 |
| 178 | bNAb10 CDRH2 | CDRH2 of bNAb10 |
| 179 | bNAb10 CDRH3 | CDRH3 of bNAb10 |
| 180 | bNAb10 CDRL1 | CDRL1 of bNAb10 |
| 181 | bNAb10 CDRL2 | CDRL2 of bNAb10 |
| 182 | bNAb10 CDRL3 | CDRL3 of bNAb10 |
| 183 | bNAb11 CDRH1 | CDRH1 of bNAb11 |
| 184 | bNAb11 CDRH2 | CDRH2 of bNAb11 |
| 185 | bNAb11 CDRH3 | CDRH3 of bNAb11 |
| 186 | bNAb11 CDRL1 | CDRL1 of bNAb11 |
| 187 | bNAb11 CDRL2 | CDRL2 of bNAb11 |
| 188 | bNAb11 CDRL3 | CDRL3 of bNAb11 |
| 189 | bNAb12 CDRH1 | CDRH1 of bNAb12 |
| 190 | bNAb12 CDRH2 | CDRH2 of bNAb12 |
| 191 | bNAb12 CDRH3 | CDRH3 of bNAb12 |
| 192 | bNAb12 CDRL1 | CDRL1 of bNAb12 |
| 193 | bNAb12 CDRL2 | CDRL2 of bNAb12 |
| 194 | bNAb12 CDRL3 | CDRL3 of bNAb12 |
| 195 | bNAb13 CDRH1 | CDRH1 of bNAb13 |
| 196 | bNAb13 CDRH2 | CDRH2 of bNAb13 |
| 197 | bNAb13 CDRH3 | CDRH3 of bNAb13 |
| 198 | bNAb13 CDRL1 | CDRL1 of bNAb13 |
| 199 | bNAb13 CDRL2 | CDRL2 of bNAb13 |
| 200 | bNAb13 CDRL3 | CDRL3 of bNAb13 |
| 201 | bNAb14 CDRH1 | CDRH1 of bNAb14 |
| 202 | bNAb14 CDRH2 | CDRH2 of bNAb14 |
| 203 | bNAb14 CDRH3 | CDRH3 of bNAb14 |
| 204 | bNAb14 CDRL1 | CDRL1 of bNAb14 |
| 205 | bNAb14 CDRL2 | CDRL2 of bNAb14 |
| 206 | bNAb14 CDRL3 | CDRL3 of bNAb14 |
| 207 | bNAb15 CDRH1 | CDRH1 of bNAb15 |
| 208 | bNAb15 CDRH2 | CDRH2 of bNAb15 |
| 209 | bNAb15 CDRH3 | CDRH3 of bNAb15 |
| 210 | bNAb15 CDRL1 | CDRL1 of bNAb15 |
| 211 | bNAb15 CDRL2 | CDRL2 of bNAb15 |
| 212 | bNAb15 CDRL3 | CDRL3 of bNAb15 |
| 213 | bNAb16 CDRH1 | CDRH1 of bNAb16 |
| 214 | bNAb16 CDRH2 | CDRH2 of bNAb16 |
| 215 | bNAb16 CDRH3 | CDRH3 of bNAb16 |
| 216 | bNAb16 CDRL1 | CDRL1 of bNAb16 |
| 217 | bNAb16 CDRL2 | CDRL2 of bNAb16 |
| 218 | bNAb16 CDRL3 | CDRL3 of bNAb16 |
| 219 | bNAb17 CDRH1 | CDRH1 of bNAb17 |
| 220 | bNAb17 CDRH2 | CDRH2 of bNAb17 |
| 212 | bNAb17 CDRH3 | CDRH3 of bNAb17 |
| 222 | bNAb17 CDRL1 | CDRL1 of bNAb17 |
| 223 | bNAb17 CDRL2 | CDRL2 of bNAb17 |
| 224 | bNAb17 CDRL3 | CDRL3 of bNAb17 |
| 225 | bNAb18 CDRH1 | CDRH1 of bNAb18 |
| 226 | bNAb18 CDRH2 | CDRH2 of bNAb18 |
| 227 | bNAb18 CDRH3 | CDRH3 of bNAb18 |
| 228 | bNAb18 CDRL1 | CDRL1 of bNAb18 |
| 229 | bNAb18 CDRL2 | CDRL2 of bNAb18 |
| 230 | bNAb18 CDRL3 | CDRL3 of bNAb18 |
| 231 | bNAb19 CDRH1 | CDRH1 of bNAb19 |
| 232 | bNAb19 CDRH2 | CDRH2 of bNAb19 |
| 233 | bNAb19 CDRH3 | CDRH3 of bNAb19 |
| 234 | bNAb19 CDRL1 | CDRL1 of bNAb19 |
| 235 | bNAb19 CDRL2 | CDRL2 of bNAb19 |
| 236 | bNAb19 CDRL3 | CDRL3 of bNAb19 |
| 237 | bNAb20 CDRH1 | CDRH1 of bNAb20 |
| 238 | bNAb20 CDRH2 | CDRH2 of bNAb20 |
| 239 | bNAb20 CDRH3 | CDRH3 of bNAb20 |
| 240 | bNAb20 CDRL1 | CDRL1 of bNAb20 |
| 241 | bNAb20 CDRL2 | CDRL2 of bNAb20 |
| 242 | bNAb20 CDRL3 | CDRL3 of bNAb20 |
| 243 | bNAb21 and bNAb22 CDRH1 | CDRH1 of bNAb21 and bNAb22 |
| 244 | bNAb21 and bNAb22 CDRH2 | CDRH2 of bNAb21 and bNAb22 |
| 245 | bNAb21 and bNAb22 CDRH3 | CDRH3 of bNAb21 and bNAb22 |
| 246 | bNAb21 and bNAb22 CDRL1 | CDRL1 of bNAb21 and bNAb22 |
| 247 | bNAb21 and bNAb22 CDRL2 | CDRL2 of bNAb21 and bNAb22 |
| 248 | bNAb21 and bNAb22 CDRL3 | CDRL3 of bNAb21 and bNAb22 |
| 249 | bNAb23 CDRH1 | CDRH1 of bNAb23 |
| 250 | bNAb23 CDRH2 | CDRH2 of bNAb23 |
| 251 | bNAb23 CDRH3 | CDRH3 of bNAb23 |
| 252 | bNAb23 CDRL1 | CDRL1 of bNAb23 |
| 253 | bNAb23 CDRL2 | CDRL2 of bNAb23 |
| 254 | bNAb23 CDRL3 | CDRL3 of bNAb23 |
| 255 | bNAb7 VH | Heavy chain variable region of bNAb7 |
| 256 | bNAb7 VL | Light chain variable region of bNAb7 |
| 257 | bNAb7 HC | Full heavy chain of bNAb7 |
| 258 | bNAb7 HC + LS | Full heavy chain of bNAb7 with |
| | | M428L/N434S mutations |
| 259 | bNAb7 LC | Full light chain of bNAb7 |
| 260 | bNAb8 VH | Heavy chain variable region of bNAb8 |
| 261 | bNAb8 VL | Light chain variable region of bNAb8 |
| 262 | bNAb8 HC | Full heavy chain of bNAb8 |
| 263 | bNAb8 HC + LS | Full heavy chain of bNAb8 with |
| | | M428L/N434S mutations |
| 264 | bNAb8 LC | Full light chain of bNAb8 |
| 265 | bNAb9 VH | Heavy chain variable region of bNAb9 |
| 266 | bNAb9 VL | Light chain variable region of bNAb9 |
| 267 | bNAb9 HC | Full heavy chain of bNAb9 |
| 268 | bNAb9 HC + LS | Full heavy chain of bNAb9 with |
| | | M428L/N434S mutations |
| 269 | bNAb9 LC | Full light chain of bNAb9 |
| 270 | bNAb10 VH | Heavy chain variable region of bNAb10 |
| 271 | bNAb10 VL | Light chain variable region of bNAb10 |
| 272 | bNAb10 HC | Full heavy chain of bNAb10 |
| 273 | bNAb10 HC + LS | Full heavy chain of bNAb10 with |
| | | M428L/N434S mutations |
| 274 | bNAb10 LC | Full light chain of bNAb10 |
| 275 | bNAb11 VH | Heavy chain variable region of bNAb11 |
| 276 | bNAb11 VL | Light chain variable region of bNAb11 |
| 277 | bNAb11 HC | Full heavy chain of bNAb11 |
| 278 | bNAb11 HC + LS | Full heavy chain of bNAb11 with |
| | | M428L/N434S mutations |
| 279 | bNAb11 LC | Full light chain of bNAb11 |
| 280 | bNAb12 VH | Heavy chain variable region of bNAb12 |
| 281 | bNAb12 VL | Light chain variable region of bNAb12 |
| 282 | bNAb12 HC | Full heavy chain of bNAb12 |
| 283 | bNAb12 HC + LS | Full heavy chain of bNAb12 with |
| | | M428L/N434S mutations |
| 284 | bNAb12 LC | Full light chain of bNAb12 |
| 285 | bNAb13 VH | Heavy chain variable region of bNAb13 |
| 286 | bNAb13 VL | Light chain variable region of bNAb13 |
| 287 | bNAb13 HC | Full heavy chain of bNAb13 |
| 288 | bNAb13 HC + LS | Full heavy chain of bNAb13 with |
| | | M428L/N434S mutations |
| 289 | bNAb13 LC | Full light chain of bNAb13 |
| 290 | bNAb14 VH | Heavy chain variable region of bNAb13 |
| 291 | bNAb14 VL | Light chain variable region of bNAb14 |
| 292 | bNAb14 HC | Full heavy chain of bNAb14 |
| 293 | bNAb14 HC + LS | Full heavy chain of bNAb14 with |
| | | M428L/N434S mutations |
| 294 | bNAb14 LC | Full light chain of bNAb14 |
| 295 | bNAb15 VH | Heavy chain variable region of bNAb15 |
| 296 | bNAb15 VL | Light chain variable region of bNAb15 |
| 297 | bNAb15 HC | Full heavy chain of bNAb15 |
| 298 | bNAb15 HC + LS | Full heavy chain of bNAb15 with |
| | | M428L/N434S mutations |
| 299 | bNAb15 LC | Full light chain of bNAb15 |
| 300 | bNAb16 VH | Heavy chain variable region of bNAb16 |
| 301 | bNAb16 VL | Light chain variable region of bNAb16 |
| 302 | bNAb16 HC | Full heavy chain of bNAb16 |
| 303 | bNAb16 HC + LS | Full heavy chain of bNAb16 with |
| | | M428L/N434S mutations |
| 304 | bNAb16 LC | Full light chain of bNAb16 |
| 305 | bNAb17 VH | Heavy chain variable region of bNAb17 |
| 306 | bNAb17 VL | Light chain variable region of bNAb17 |
| 307 | bNAb17 HC | Full heavy chain of bNAb17 |
| 308 | bNAb17 HC + LS | Full heavy chain of bNAb17 with |
| | | M428L/N434S mutations |
| 309 | bNAb17 LC | Full light chain of bNAb17 |
| 310 | bNAb18 VH | Heavy chain variable region of bNAb17 |
| 311 | bNAb18 VL | Light chain variable region of bNAb17 |
| 312 | bNAb18 HC | Full heavy chain of bNAb17 |
| 313 | bNAb18 HC + LS | Full heavy chain of bNAb17 with |
| | | M428L/N434S mutations |
| 314 | bNAb18 LC | Full light chain of bNAb18 |
| 315 | bNAb19 VH | Heavy chain variable region of bNAb19 |
| 316 | bNAb19 VL | Light chain variable region of bNAb19 |
| 317 | bNAb19 HC | Full heavy chain of bNAb19 |
| 318 | bNAb19 HC + LS | Full heavy chain of bNAb19 with |
| | | M428L/N434S mutations |
| 319 | bNAb19 LC | Full light chain of bNAb19 |
| 320 | bNAb20 VH | Heavy chain variable region of bNAb20 |
| 321 | bNAb20 VL | Light chain variable region of bNAb20 |
| 322 | bNAb20 HC | Full heavy chain of bNAb20 |
| 323 | bNAb20 HC + LS | Full heavy chain of bNAb20 with |
| | | M428L/N434S mutations |
| 324 | bNAb20 LC | Full light chain of bNAb20 |
| 325 | bNAb21 VH | Heavy chain variable region of bNAb21 |
| 326 | bNAb21 VL | Light chain variable region of bNAb21 |
| 327 | bNAb21 HC | Full heavy chain of bNAb21 |
| 328 | bNAb21 HC + LS | Full heavy chain of bNAb21 with |
| | | M428L/N434S mutations |
| 329 | bNAb21 LC | Full light chain of bNAb21 |
| 330 | bNAb22 VH | Full heavy chain of bNAb22 |
| 331 | bNAb22 HC | Full heavy chain of bNAb22 with |
| | | M428L/N434S mutations |
| 332 | bNAb22 HV + LS | Full light chain of bNAb22 |
| 333 | bNAb23 VH | Heavy chain variable region of bNAb23 |
| 334 | bNAb23 VL | Light chain variable region of bNAb23 |
| 335 | bNAb23 HC | Full heavy chain of bNAb23 |
| 336 | bNAb23 HC + LS | Full heavy chain of bNAb23 with |
| | | M428L/N434S mutations |
| 337 | bNAb23 LC | Full light chain of bNAb23 |
| 338 | D1m-K8C-G99C_1xG4S_bNAb7-HC | Human CD4 mD1.22 + K8C + G99C fused |
| | | to the N-terminus of bNAb7 heavy chain |
| | | with 1xG4S linker, Fc includes M428L/N434S |
| 339 | D1m-K8C-G99C_1xG4S_bNAb8-HC | Human CD4 mD1.22 + K8C + G99C fused |
| | | to the N-terminus of bNAb8 heavy chain |
| | | with 1xG4S linker, Fc includes M428L/N434S |
| 340 | D1m-K8C-G99C_1xG4S_bNAb9-HC | Human CD4 mD1.22 + K8C + G99C fused |
| | | to the N-terminus of bNAb9 heavy chain |
| | | with 1xG4S linker, Fc includes M428L/N434S |
| 341 | D1m-K8C-G99C_1xG4S_bNAb10- | Human CD4 mD1.22 + K8C + G99C fused |
| | HC | to the N-terminus of bNAb10 heavy chain |
| | | with 1xG4S linker, Fc includes M428L/N434S |
| 342 | D1m-K8C-G99C_1xG4S_bNAb11- | Human CD4 mD1.22 + K8C + G99C fused |
| | HC | to the N-terminus of bNAb11 heavy chain |
| | | with 1xG4S linker, Fc includes M428L/N434S |
| 343 | D1m-K8C-G99C_1xG4S_bNAb12- | Human CD4 mD1.22 + K8C + G99C fused |
| | HC | to the N-terminus of bNAb12 heavy chain |
| | | with 1xG4S linker, Fc includes M428L/N434S |
| 344 | bNAb12_HC_1xG4S_D1m-K8C- | Human CD4 mD1.22 + K8C + G99C fused |
| | G99C | to the C-terminus of bNAb12 heavy chain |
| | | with 1xG4S linker, Fc includes M428L/N434S |
| 345 | D1m-K8C-G99C_1xG4S_LC-bNAb12 | Human CD4 mD1.22 + K8C + G99C fused |
| | | to the N-terminus of bNAb12 light chain |
| | | with 1xG4S linker |
| 346 | bNAb12_LC_1xG4S_D1m-K8C- | Human CD4 mD1.22 + K8C + G99C fused |
| | G99C-HC | to the C-terminus of bNAb12 light chain |
| | | with 1xG4S linker |
| 347 | D1m-K8C-G99C_1xG4S_bNAb13- | Human CD4 mD1.22 + K8C + G99C fused |
| | HC | to the N-terminus of bNAb13 heavy chain |
| | | with 1xG4S linker, Fc includes M428L/N434S |
| 348 | D1m-K8C-G99C_1xG4S_bNAb14- | Human CD4 mD1.22 + K8C + G99C fused |
| | HC | to the N-terminus of bNAb14 heavy chain |
| | | with 1xG4S linker, Fc includes M428L/N434S |
| 349 | D1m-K8C-G99C_1xG4S_bNAb15- | Human CD4 mD1.22 + K8C + G99C fused |
| | HC | to the N-terminus of bNAb15 heavy chain |
| | | with 1xG4S linker, Fc includes M428L/N434S |
| 350 | D1m-K8C-G99C_1xG4S_bNAb16- | Human CD4 mD1.22 + K8C + G99C fused |
| | HC | to the N-terminus of bNAb16 heavy chain |
| | | with 1xG4S linker, Fc includes M428L/N434S |
| 351 | D1m-K8C-G99C_1xG4S_bNAb17- | Human CD4 mD1.22 + K8C + G99C fused |
| | HC | to the N-terminus of bNAb17 heavy chain |
| | | with 1xG4S linker, Fc includes M428L/N434S |
| 352 | D1m-K8C-G99C_1xG4S_bNAb18- | Human CD4 mD1.22 + K8C + G99C fused |
| | HC | to the N-terminus of bNAb18 heavy chain |
| | | with 1xG4S linker, Fc includes M428L/N434S |
| 353 | D1m-K8C-G99C_1xG4S_bNAb19- | Human CD4 mD1.22 + K8C + G99C fused |
| | HC | to the N-terminus of bNAb19 heavy chain |
| | | with 1xG4S linker, Fc includes M428L/N434S |
| 354 | D1m-K8C-G99C_1xG4S_bNAb20- | Human CD4 mD1.22 + K8C + G99C fused |
| | HC | to the N-terminus of bNAb20 heavy chain |
| | | with 1xG4S linker, Fc includes M428L/N434S |
| 355 | D1m-K8C-G99C_1xG4S_bNAb21- | Human CD4 mD1.22 + K8C + G99C fused |
| | HC | to the N-terminus of bNAb21 heavy chain |
| | | with 1xG4S linker, Fc includes M428L/N434S |
| 356 | D1m-K8C-G99C_1xG4S_bNAb22- | Human CD4 mD1.22 + K8C + G99C fused |
| | HC | to the N-terminus of bNAb22 heavy chain |
| | | with 1xG4S linker, Fc includes M428L/N434S |
| 357 | D1m-K8C-G99C_1xG4S_bNAb23- | Human CD4 mD1.22 + K8C + G99C fused |
| | HC | to the N-terminus of bNAb23 heavy chain |
| | | with 1xG4S linker, Fc includes M428L/N434S |
| 358 | D1m-K8C-G99C_1xG4S_bNAb23-LC | Human CD4 mD1.22 + K8C + G99C fused |
| | | to the N-terminus of bNAb23 light chain |
| | | with 1xG4S linker |
| 359 | bNAb23-HC_1xG4S_D1m-K8C- | Human CD4 mD1.22 + K8C + G99C fused |
| | G99C | to the C-terminus of bNAb23 heavy chain |
| | | with 1xG4S linker, Fc includes M428L/N434S |
| 360 | bNAb23_LC_1xG4S_D1m-K8C- | Human CD4 mD1.22 + K8C + G99C fused |
| | G99C | to the C-terminus of bNAb23 light chain |
| | | with 1xG4S linker |
| 361 | V3 loop | V3 loop consensus sequence |
| 362 | D1m-K8C-G99C_1xG4S_bNAb6-HC | Human CD4 mD1.22 + K8C + G99C fused |
| | | to the N-terminus of bNAb6 heavy chain |
| | | with 1xG4S linker, Fc includes M428L/N434S |
| 363 | Exemplary gp160 | Exemplary gp160 sequence |
| 364 | Exemplary gp120 | Exemplary gp120 sequence |
| 365 | bNAb22 VL | Light chain variable region of bNAb22 |
| 366 | bNAb22 LC | Full light chain of bNAb22 |

SEQ ID NO: 1
KKVVLGKKGDTVELTCTASQKKSIQFHWKNSNQIKILGNQGSFLT
KGPSKLNDRADSRRSLWDQGNFPLIIKNLKIEDSDTYICEVEDQK
EEVQLLVFGLTANSDTHLLQGQSLTLTLESPPGSSPSVQCRSPRG
KNIQGGKTLSVSQLELQDSGTWTCTVLQNQKKVEFKIDIVVLA
SEQ ID NO: 2
KKVVLGKKGDTVELTCTASQKKNIQFHWKNSNQIKILGNQGSFLT
KGPSKLNDRVDSRRSLWDQGNFPLIIKNLKIEDSDTYICEVEDQK
EEVQLVVFGLTANSDTHLLQGQSLTLTLESPPGSSPSVQCRSPRG
KNIQGGKTLSVSQLELQDSGTWTCTVLQNQKKVEFKIDIVVLAF
SEQ ID NO: 3
KKVVLGKKGDTVELTCTASQKKSIQFHWKNSNQIKILGNQGSFLT
KGPSKLNDRADSRRSLWDQGNFPLIIKNLKIEDSDTYICEVEDQK
EEVQLLVFG
SEQ ID NO: 4
KKVVYGKKGDTVELTCTASQKKNIQFHWKNSNQIKILGNQGSFLT
KGPSKLNDRVDSRRSLWDQGNFPLIIKNLKPEDSDTYICEVEDQK
EEVQLVVVG
SEQ ID NO: 5
KKVVYGKVGDTVELTCTASQKKNIQFHWKNSNQIKILGNQGSFLT
KGPSKLNDRVDSRRSLWDQGNFPLIIKNLKPEDSDTYICEVEDQK
EEVQLVVVG
SEQ ID NO: 6
KKVVYGKKGDTVELTCTASQKKNIQFHWKNSNQIKILGNQGSFLT
KGPSKLNDRVDSRRSLWDQGNFPLIIKNLKPEDSDTYICEVEDQK
QEVQLVVVG
SEQ ID NO: 7
KKVVYGKKGDTVELTCTASQKKNIQFHWKNSNQIKILGNQGSFLT
KGPSKLNDRVDSRRSLWDQGNFPLIIKNLKPEDSDTYICEVEDQK
HEVQLVVVG
SEQ ID NO: 8
KKVVYGKKGDTVELTCTASQKKNIQFHWKNSNQIKILGNQGSFLT
KGPSKLNDRVDSRRSLWDQGNFPLIIKNLKPEDSDTYICEVGDQK
EEVQLVVVG
SEQ ID NO: 9
KKVVYGKKGDTVELTCTASQKKNIQFHWKNSNQIKILGNQGSFLT
KGPSKLWDRVDSRRSLWDQGNFPLIIKNLKPEDSDTYICEVEDQK
EEVQLVVVG
SEQ ID NO: 10
KKVVYGKIGDTVELTCTASQKKNIQFHWKNSNQIKILGNQGSFLT
KGPSKLNDRVDSRRSLWDQGNFPLIIKNLKPEDSDTYICEVEDQK
EEVQLVVVG
SEQ ID NO: 11
KKVVYGKCGDTVELTCTASQKKNIQFHWKNSNQIKILGNQGSFLT
KGPSKLNDRVDSRRSLWDQGNFPLIIKNLKPEDSDTYICEVEDQK
EEVQLVVVC
SEQ ID NO: 12
KKVVYGKKGDCVELTCTASQKKNIQFHWKNSNQIKILGNQGSFLT
KGPSKLNDRVDSRRSLWDQGNFPLIICNLKPEDSDTYICEVEDQK
EEVQLVVVG
SEQ ID NO: 13
KKVVYGKKGDTVCLTCTASQKKNIQFHWKNSNQIKILGNQGSFLT
KGPSKLNDRVDSRRSLWDQGNFPLCIKNLKPEDSDTYICEVEDQK
EEVQLVVVG
SEQ ID NO: 14
KKVVYGKKGDTVELTCTASQKKNIQFCWKNSNQIKILCNQGSFLT
KGPSKLNDRVDSRRSLWDQGNFPLIIKNLKPEDSDTYICEVEDQK
EEVQLVVVG
SEQ ID NO: 15
KKVVYGKKGDTVELTCTASQCKNIQFHWKNSNQIKILGNQGSFLT
KGPSKLNDRVDSRRSLWDQCNFPLIIKNLKPEDSDTYICEVEDQK
EEVQLVVVG
SEQ ID NO: 16
KKVVYGKKGDTVELTCTASQKKNIEFHWKNSNQIKILGNQGSFLT
KGPSKLNDRVDSRRSLWDQGNFPLIIKNLKPEDSDTYICEVEDQK
EEVQLVVVG
SEQ ID NO: 17
KKVVYGKKGDTVELTCTASQKKNIQFDWKNSNQIKILGNQGSFLT
KGPSKLNDRVDSRRSLWDQGNFPLIIKNLKPEDSDTYICEVEDQK
EEVQLVVVG
SEQ ID NO: 18
KKVVYGKKGDTVELTCTASQKKNIQFHWKNSNQIKILGNQGSFLT
KGPSKLNDRVDSVRSLWDQGNFPLIIKNLKPEDSDTYICEVEDQK
EEVQLVVVG
SEQ ID NO: 19
KKVVYGKKGDTVELTCTASQKKNIQFHWKNSNQIKILGNQGSFLT
KGPSKLNDRVDSNRSLWDQGNFPLIIKNLKPEDSDTYICEVEDQK
EEVQLVVVG
SEQ ID NO: 20
KKVVYGKKGDTVELTCTASQKKNIQFHWKNSNQIKILGNQGSFLT
KGPSKLNDRVDSTRSLWDQGNFPLIIKNLKPEDSDTYICEVEDQK
EEVQLVVVG
SEQ ID NO: 21
KKVVYGKKGDTVELTCTASQKKNIQFHWKNSNQIKILGNQGSFLT
KGPSKLNDRVDSRRSMWDQGNFPLIIKNLKPEDSDTYICEVEDQK
EEVQLVVVG
SEQ ID NO: 22
ACNSFWG
SEQ ID NO: 23
SLSHCASYWNRGWTYHNPSLKS
SEQ ID NO: 24
FGGEVLRYTDWPKPAWVDL
SEQ ID NO: 25
TGTSNNFVS
SEQ ID NO: 26
DVNKRPS
SEQ ID NO: 27
GSLVGNWDVI
SEQ ID NO: 28
DSYWS
SEQ ID NO: 29
YVHKSGDTNYSPSLKS
SEQ ID NO: 30
TLHGRRIYGIVAFNEWFTYFYMDV
SEQ ID NO: 31
GEKSLGSRAVQ
SEQ ID NO: 32
NNQDRPS
SEQ ID NO: 33
HIWDSRVPTKWV
SEQ ID NO: 34
SDHSWT
SEQ ID NO: 35
DIHYNGATTYNPSLRS
SEQ ID NO: 36
NAIRIYGVVALGEWFHYGMDV
SEQ ID NO: 37
SGAPLTSRFTY
SEQ ID NO: 38
RSSQRSS
SEQ ID NO: 39
QSSDTSDSYKM
SEQ ID NO: 40
NYYWT
SEQ ID NO: 41
YISDRESATYNPSLNS
SEQ ID NO: 42
ARRGQRIYGVVSFGEFFYYYSMDV
SEQ ID NO: 43
GRQALGSRAVQ
SEQ ID NO: 44
NNQDRPS
SEQ ID NO: 45
HMWDSRSGFSWS
SEQ ID NO: 46
GGEWGDKDYHWG
SEQ ID NO: 47
SIHWRGTTHYKESLRR
SEQ ID NO: 48
HRHHDVFMLVPIAGWFDV
SEQ ID NO: 49
RASQNINKNLA
SEQ ID NO: 50
ETYSKIA
SEQ ID NO: 51
QQYEEWPRT
SEQ ID NO: 52
DFYIH
SEQ ID NO: 53
WMNPQTGRTNTARNFQG
SEQ ID NO: 54
GGWISLYYDSSYYPNFDH
SEQ ID NO: 55
TGTKYDVGSHDLVS
SEQ ID NO: 56
EVNKRPS
SEQ ID NO: 57
CSFGGSATVV
SEQ ID NO: 58
QPQLQESGPTLVEASETLSLTCAVSGDSTAACNSFWGWVRQPPGK
GLEWVGSLSHCASYWNRGWTYHNPSLKSRLTLALDTPKNLVFLKL
NSVTAADTATYYCARFGGEVLRYTDWPKPAWVDLWGRGTLVTVSS
SEQ ID NO: 59
QSALTQPPSASGSPGQSITISCTGTSNNFVSWYQQHAGKAPKLVI
YDVNKRPSGVPDRFSGSKSGNTASLTVSGLQTDDEAVYYCGSLVG
NWDVIFGGGTKLTVL
SEQ ID NO: 60
QSALTQPPSASGSPGQSITISCTGTSNNYVSWYQQHAGKAPKLVI
YDVNKRPSGVPDRFSGSKSGNTASLTVSGLQTDDEAVYYCGSLVG
NWDVIFGGGTKLTVL
SEQ ID NO: 61
QPQLQESGPTLVEASETLSLTCAVSGDSTAACNSFWGWVRQPPGK
GLEWVGSLSHCASYWNRGWTYHNPSLKSRLTLALDTPKNLVFLKL
NSVTAADTATYYCARFGGEVLRYTDWPKPAWVDLWGRGTLVTVSS
ASTKGPSVFPLAPSSKSTSGGTAALGCLVKDYFPEPVTVSWNSGA
LTSGVHTFPAVLQSSGLYSLSSVVTVPSSSLGTQTYICNVNHKPS
NTKVDKKVEPKSCDKTHTCPPCPAPELLGGPSVFLFPPKPKDTLM
ISRTPEVTCVVVDVSHEDPEVKFNWYVDGVEVHNAKTKPREEQYN
STYRVVSVLTVLHQDWLNGKEYKCKVSNKALPAPIEKTISKAKGQ
PREPQVYTLPPSRDELTKNQVSLTCLVKGFYPSDIAVEWESNGQP
ENNYKTTPPVLDSDGSFFLYSKLTVDKSRWQQGNVFSCSVMHEAL
HNHYTQKSLSLSPGK
SEQ ID NO: 62
QPQLQESGPTLVEASETLSLTCAVSGDSTAACNSFWGWVRQPPGK
GLEWVGSLSHCASYWNRGWTYHNPSLKSRLTLALDTPKNLVFLKL
NSVTAADTATYYCARFGGEVLRYTDWPKPAWVDLWGRGTLVTVSS
ASTKGPSVFPLAPSSKSTSGGTAALGCLVKDYFPEPVTVSWNSGA
LTSGVHTFPAVLQSSGLYSLSSWVTVPSSSLGTQTYICNVNHKPS
NTKVDKKVEPKSCDKTHTCPPCPAPELLGGPSVFLFPPKPKDTLM
ISRTPEVTCVVVDVSHEDPEVKFNWYVDGVEVHNAKTKPREEQYN
STYRVVSVLTVLHQDWLNGKEYKCKVSNKALPAPIEKTISKAKGQ
PREPQVYTLPPSRDELTKNQVSLTCLVKGFYPSDIAVEWESNGQP
ENNYKTTPPVLDSDGSFFLYSKLTVDKSRWQQGNVFSCSVLHEAL
HSHYTQKSLSLSPGK
SEQ ID NO: 63
QSALTQPPSASGSPGQSITISCTGTSNNFVSWYQQHAGKAPKLVI
YDVNKRPSGVPDRFSGSKSGNTASLTVSGLQTDDEAVYYCGSLVG
NWDVIFGGGTKLTVLGQPKAAPSVTLFPPSSEELQANKATLVCLI
SDFYPGAVTVAWKADSSPVKAGVETTTPSKQSNNKYAASSYLSLT
PEQWKSHRSYSCQVTHEGSTVEKTVAPTECS
SEQ ID NO: 64
QSALTQPPSASGSPGQSITISCTGTSNNYVSWYQQHAGKAPKLVI
YDVNKRPSGVPDRFSGSKSGNTASLTVSGLQTDDEAVYYCGSLVG
NWDVIFGGGTKLTVLGQPKAAPSVTLFPPSSEELQANKATLVCLI
SDFYPGAVTVAWKADSSPVKAGVETTTPSKQSNNKYAASSYLSLT
PEQWKSHRSYSCQVTHEGSTVEKTVAPTECS
SEQ ID NO: 65
QMQLQESGPGLVKPSETLSLTCSVSGASISDSYWSWIRRSPGKGL
EWIGYVHKSGDTNYSPSLKSRVNLSLDTSKNQVSLSLVAATAADS
GKYYCARTLHGRRIYGIVAFNEWFTYFYMDVWGNGTQVTVSS
SEQ ID NO: 66
SDISVAPGETARISCGEKSLGSRAVQWYQHRAGQAPSLIIYNNQD
RPSGIPERFSGSPDSPFGTTATLTITSVEAGDEADYYCHIWDSRV
PTKWVFGGGTTLTVL
SEQ ID NO: 67
QMQLQESGPGLVKPSETLSLTCSVSGASISDSYWSWIRRSPGKGL
EWIGYVHKSGDTNYSPSLKSRVNLSLDTSKNQVSLSLVAATAADS
GKYYCARTLHGRRIYGIVAFNEWFTYFYMDVWGNGTQVTVSSAST
KGPSVFPLAPSSKSTSGGTAALGCLVKDYFPEPVTVSWNSGALTS
GVHTFPAVLQSSGLYSLSSVVTVPSSSLGTQTYICNVNHKPSNTK
VDKRVEPKSCDKTHTCPPCPAPELLGGPSVFLFPPKPKDTLMISR
TPEVTCVVVDVSHEDPEVKFNWYVDGVEVHNAKTKPREEQYNSTY
RVVSVLTVLHQDWLNGKEYKCKVSNKALPAPIEKTISKAKGQPRE
PQVYTLPPSRDELTKNQVSLTCLVKGFYPSDIAVEWESNGQPENN
YKTTPPVLDSDGSFFLYSKLTVDKSRWQQGNVFSCSVMHEALHNH
YTQKSLSLSPGK
SEQ ID NO: 68
QMQLQESGPGLVKPSETLSLTCSVSGASISDSYWSWIRRSPGKGL
EWIGYVHKSGDTNYSPSLKSRVNLSLDTSKNQVSLSLVAATAADS
GKYYCARTLHGRRIYGIVAFNEWFTYFYMDVWGNGTQVTVSSAST
KGPSVFPLAPSSKSTSGGTAALGCLVKDYFPEPVTVSWNSGALTS
GVHTFPAVLQSSGLYSLSSVVTVPSSSLGTQTYICNVNHKPSNTK
VDKRVEPKSCDKTHTCPPCPAPELLGGPSVFLFPPKPKDTLMISR
TPEVTCVVVDVSHEDPEVKFNWYVDGVEVHNAKTKPREEQYNSTY
RVVSVLTVLHQDWLNGKEYKCKVSNKALPAPIEKTISKAKGQPRE
PQVYTLPPSRDELTKNQVSLTCLVKGFYPSDIAVEWESNGQPENN
YKTTPPVLDSDGSFFLYSKLTVDKSRWQQGNVFSCSVLHEALHSH
YTQKSLSLSPGK
SEQ ID NO: 69
SDISVAPGETARISCGEKSLGSRAVQWYQHRAGQAPSLIIYNNQD
RPSGIPERFSGSPDSPFGTTATLTITSVEAGDEADYYCHIWDSRV
PTKWVFGGGTTLTVLGQPKAAPSVTLFPPSSEELQANKATLVCLI
SDFYPGAVTVAWKADSSPVKAGVETTTPSKQSNNKYAASSYLSLT
PEQWKSHKSYSCQVTHEGSTVEKTVAPTECS
SEQ ID NO: 70
QVQLRESGPGLVKPSETLSLSCTVSNDSRPSDHSWTWVRQSPGKA
LEWIGDIHYNGATTYNPSLRSRVRIELDQSIPRFSLKMTSMTAAD
TGMYYCARNAIRIYGVVALGEWFHYGMDVWGQGTAVTVSS
SEQ ID NO: 71
SSELTQPPSVSVSPGQTARITCSGAPLTSRFTYWYRQKPGQAPVL
IISRSSQRSSGWSGRFSASWSGTTVTLTIRGVQADDEADYYCQSS
DTSDSYKMFGGGTKLTVL
SEQ ID NO: 72
QVQLRESGPGLVKPSETLSLSCTVSNDSRPSDHSWTWVRQSPGKA
LEWIGDIHYNGATTYNPSLRSRVRIELDQSIPRFSLKMTSMTAAD
TGMYYCARNAIRIYGVVALGEWFHYGMDVWGQGTAVTVSSASTKG
PSVFPLAPSSKSTSGGTAALGCLVKDYFPEPVTVSWNSGALTSGV
HTFPAVLQSSGLYSLSSVVTVPSSSLGTQTYICNVNHKPSNTKVD
KRVEPKSCDKTHTCPPCPAPELLGGPSVFLFPPKPKDTLMISRTP
EVTCVVVDVSHEDPEVKFNWYVDGVEVHNAKTKPREEQYNSTYRV
VSVLTVLHQDWLNGKEYKCKVSNKALPAPIEKTISKAKGQPREPQ
VYTLPPSRDELTKNQVSLTCLVKGFYPSDIAVEWESNGQPENNYK
TTPPVLDSDGSFFLYSKLTVDKSRWQQGNVFSCSVMHEALHNHYT
QKSLSLSPGK
SEQ ID NO: 73
QVQLRESGPGLVKPSETLSLSCTVSNDSRPSDHSWTWVRQSPGKA
LEWIGDIHYNGATTYNPSLRSRVRIELDQSIPRFSLKMTSMTAAD
TGMYYCARNAIRIYGVVALGEWFHYGMDVWGQGTAVTVSSASTKG
PSVFPLAPSSKSTSGGTAALGCLVKDYFPEPVTVSWNSGALTSGV
HTFPAVLQSSGLYSLSSVVTVPSSSLGTQTYICNVNHKPSNTKVD
KRVEPKSCDKTHTCPPCPAPELLGGPSVFLFPPKPKDTLMISRTP
EVTCVVVDVSHEDPEVKFNWYVDGVEVHNAKTKPREEQYNSTYRV
VSVLTVLHQDWLNGKEYKCKVSNKALPAPIEKTISKAKGQPREPQ
VYTLPPSRDELTKNQVSLTCLVKGFYPSDIAVEWESNGQPENNYK
TTPPVLDSDGSFFLYSKLTVDKSRWQQGNVFSCSVLHEALHSHYT
QKSLSLSPGK
SEQ ID NO: 74
SSELTQPPSVSVSPGQTARITCSGAPLTSRFTYWYRQKPGQAPVL
IISRSSQRSSGWSGRFSASWSGTTVTLTIRGVQADDEADYYCQSS
DTSDSYKMFGGGTKLTVLGQPAAAPSVTLFPPSSEELQANKATLV
CLISDFYPGAVTVAWKADSSPVKAGVETTTPSKQSNNKYAASSYL
SLTPEQWKSHKSYSCQVTHEGSTVEKTVAPTEC
SEQ ID NO: 75
QVQLQESGPGLVKPSETLSVTCSVSGDSMNNYYWTWIRQSPGKGL
EWIGYISDRESATYNPSLNSRVVISRDTSKNQLSLKLNSVTPADT
AVYYCATARRGQRIYGVVSFGEFFYYYSMDVWGKGTTVTVSS
SEQ ID NO: 76
SYVRPLSVALGETARISCGRQALGSRAVQWYQHRPGQAPILLIYN
NQDRPSGIPERFSGTPDINFGTRATLTISGVEAGDEADYYCHMWD
SRSGFSWSFGGATRLTVL
SEQ ID NO: 77
QVQLQESGPGLVKPSETLSVTCSVSGDSMNNYYWTWIRQSPGKGL
EWIGYISDRESATYNPSLNSRVVISRDTSKNQLSLKLNSVTPADT
AVYYCATARRGQRIYGVVSFGEFFYYYSMDVWGKGTTVTVSSAST
KGPSVFPLAPSSKSTSGGTAALGCLVKDYFPEPVTVSWNSGALTS
GVHTFPAVLQSSGLYSLSSVVTVPSSSLGTQTYICNVNHKPSNTK
VDKRVEPKSCDKTHTCPPCPAPELLGGPSVFLFPPKPKDTLMISR
TPEVTCVVVDVSHEDPEVKFNWYVDGVEVHNAKTKPREEQYNSTY
RVVSVLTVLHQDWLNGKEYKCKVSNKALPAPIEKTISKAKGQPRE
PQVYTLPPSRDELTKNQVSLTCLVKGFYPSDIAVEWESNGQPENN
YKTTPPVLDSDGSFFLYSKLTVDKSRWQQGNVFSCSVMHEALHNH
YTQKSLSLSPGK
SEQ ID NO: 78
QVQLQESGPGLVKPSETLSVTCSVSGDSMNNYYWTWIRQSPGKGL
EWIGYISDRESATYNPSLNSRVVISRDTSKNQLSLKLNSVTPADT
AVYYCATARRGQRIYGVVSFGEFFYYYSMDVWGKGTTVTVSSAST
KGPSVFPLAPSSKSTSGGTAALGCLVKDYFPEPVTVSWNSGALTS
GVHTFPAVLQSSGLYSLSSVVTVPSSSLGTQTYICNVNHKPSNTK
VDKRVEPKSCDKTHTCPPCPAPELLGGPSVFLFPPKPKDTLMISR
TPEVTCVVVDVSHEDPEVKFNWYVDGVEVHNAKTKPREEQYNSTY
RVVSVLTVLHQDWLNGKEYKCKVSNKALPAPIEKTISKAKGQPRE
PQVYTLPPSRDELTKNQVSLTCLVKGFYPSDIAVEWESNGQPENN
YKTTPPVLDSDGSFFLYSKLTVDKSRWQQGNVFSCSVLHEALHSH
YTQKSLSLSPGK
SEQ ID NO: 79
SYVRPLSVALGETARISCGRQALGSRAVQWYQHRPGQAPILLIYN
NQDRPSGIPERFSGTPDINFGTRATLTISGVEAGDEADYYCHMWD
SRSGFSWSFGGATRLTVLGQPKAAPSVTLFPPSSEELQANKATLV
CLISDFYPGAVTVAWKADSSPVKAGVETTTPSKQSNNKYAASSYL
SLTPEQWKSHRSYSCQVTHEGSTVEKTVAPTECS
SEQ ID NO: 80
QLQMQESGPGLVKPSETLSLSCTVSGDSIRGGEWGDKDYHWGWVR
HSAGKGLEWIGSIHWRGTTHYKESLRRRVSMSIDTSRNWFSLRLA
SVTAADTAVYFCARHRHHDVFMLVPIAGWFDVWGPGVQVTVSS
SEQ ID NO: 81
EIVMTQSPDTLSVSPGETVTLSCRASQNINKNLAWYQYKPGQSPR
LVIFETYSKIAAFPARFVASGSGTEFTLTINNMQSEDVAVYYCQQ
YEEWPRTFGQGTKVDIK
SEQ ID NO: 82
QLQMQESGPGLVKPSETLSLSCTVSGDSIRGGEWGDKDYHWGWVR
HSAGKGLEWIGSIHWRGTTHYKESLRRRVSMSIDTSRNWFSLRLA
SVTAADTAVYFCARHRHHDVFMLVPIAGWFDVWGPGVQVTVSSAS
TKGPSVFPLAPSSKSTSGGTAALGCLVKDYFPEPVTVSWNSGALT
SGVHTFPAVLQSSGLYSLSSVVTVPSSSLGTQTYICNVNHKPSNT
KVDKRVEPKSCDKTHTCPPCPAPELLGGPSVFLFPPKPKDTLMIS
RTPEVTCVVVDVSHEDPEVKFNWYVDGVEVHNAKTKPREEQYNST
YRVVSVLTVLHQDWLNGKEYKCKVSNKALPAPIEKTISKAKGQPR
EPQVYTLPPSRDELTKNQVSLTCLVKGFYPSDIAVEWESNGQPEN
NYKTTPPVLDSDGSFFLYSKLTVDKSRWQQGNVFSCSVMHEALHN
HYTQKSLSLSPGK
SEQ ID NO: 83
QLQMQESGPGLVKPSETLSLSCTVSGDSIRGGEWGDKDYHWGWVR
HSAGKGLEWIGSIHWRGTTHYKESLRRRVSMSIDTSRNWFSLRLA
SVTAADTAVYFCARHRHHDVFMLVPIAGWFDVWGPGVQVTVSSAS
TKGPSVFPLAPSSKSTSGGTAALGCLVKDYFPEPVTVSWNSGALT
SGVHTFPAVLQSSGLYSLSSVVTVPSSSLGTQTYICNVNHKPSNT
KVDKRVEPKSCDKTHTCPPCPAPELLGGPSVFLFPPKPKDTLMIS
RTPEVTCVVVDVSHEDPEVKFNWYVDGVEVHNAKTKPREEQYNST
YRVVSVLTVLHQDWLNGKEYKCKVSNKALPAPIEKTISKAKGQPR
EPQVYTLPPSRDELTKNQVSLTCLVKGFYPSDIAVEWESNGQPEN
NYKTTPPVLDSDGSFFLYSKLTVDKSRWQQGNVFSCSVLHEALHS
HYTQKSLSLSPGK
SEQ ID NO: 84
EIVMTQSPDTLSVSPGETVTLSCRASQNINKNLAWYQYKPGQSPR
LVIFETYSKIAAFPARFVASGSGTEFTLTINNMQSEDVAVYYCQQ
YEEWPRTFGQGTKVDIKRTVAAPSVFIFPPSDEQLKSGTASVVCL
LNNFYPREAKVQWKVDNALQSGNSQESVTEQDSKDSTYSLSSTLT
LSKADYEKHKVYACEVTHQGLSSPVTKSFNRGEC
SEQ ID NO: 85
QVQLVQSGAQMKNPGASVKVSCAPSGYTFTDFYIHWLRQAPGQGL
QWMGWMNPQTGRTNTARNFQGRVTMTRDTSIGTAYMELRSLTSDD
TAIYYCTTGGWISLYYDSSYYPNFDHWGQGTLLTVSS
SEQ ID NO: 86
QSALTQPASVSGSPGQSITISCTGTKYDVGSHDLVSWYQQYPGKV
PKYMIYEVNKRPSGVSNRFSGSKSGNTASLTISGLRAEDEADYYC
CSFGGSATVVCGGGTKVTVL
SEQ ID NO: 87
QVQLVQSGAQMKNPGASVKVSCAPSGYTFTDFYIHWLRQAPGQGL
QWMGWMNPQTGRTNTARNFQGRVTMTRDTSIGTAYMELRSLTSDD
TAIYYCTTGGWISLYYDSSYYPNFDHWGQGTLLTVSSASTKGPSV
FPLAPSSKSTSGGTAALGCLVKDYFPEPVTVSWNSGALTSGVHTF
PAVLQSSGLYSLSSVVTVPSSSLGTQTYICNVNHKPSNTKVDKKV
EPKSCDKTHTCPPCPAPELLGGPSVFLFPPKPKDTLMISRTPEVT
CVVDVSHEDPEVKFNWYVDGVEVHNAKTKPREEQYNSTYRVVSVL
TVLHQDWLNGKEYKCKVSNKALPAPIEKTISKAKGQPREPQVYTL
PPSRDELTKNQVSLTCLVKGFYPSDIAVEWESNGQPENNYKTTPP
VLDSDGSFFLYSKLTVDKSRWQQGNVFSCSVMHEALHNHYTQKSL
SLSPGK
SEQ ID NO: 88
QVQLVQSGAQMKNPGASVKVSCAPSGYTFTDFYIHWLRQAPGQGL
QWMGWMNPQTGRTNTARNFQGRVTMTRDTSIGTAYMELRSLTSDD
TAIYYCTTGGWISLYYDSSYYPNFDHWGQGTLLTVSSASTKGPSV
FPLAPSSKSTSGGTAALGCLVKDYFPEPVTVSWNSGALTSGVHTF
PAVLQSSGLYSLSSVVTVPSSSLGTQTYICNVNHKPSNTKVDKKV
EPKSCDKTHTCPPCPAPELLGGPSVFLFPPKPKDTLMISRTPEVT
CVVVDVSHEDPEVKFNWYVDGVEVHNAKTKPREEQYNSTYRVVSV
LTVLHQDWLNGKEYKCKVSNKALPAPIEKTISKAKGQPREPQVYT
LPPSRDELTKNQVSLTCLVKGFYPSDIAVEWESNGQPENNYKTTP
PVLDSDGSFFLYSKLTVDKSRWQQGNVFSCSVLHEALHSHYTQKS
LSLSPGK
SEQ ID NO: 89
QSALTQPASVSGSPGQSITISCTGTKYDVGSHDLVSWYQQYPGKV
PKYMIYEVNKRPSGVSNRFSGSKSGNTASLTISGLRAEDEADYYC
CSFGGSATVVCGGGTKVTVLGQPKAAPSVTLFPPSSEELQANKAT
LVCLISDFYPGAVTVAWKADSSPVKAGVETTTPSKQSNNKYAASS
YLSLTPEQWKSHRSYSCQVTHEGSTVEKTVAPTECS
SEQ ID NO: 90
GGGGS
SEQ ID NO: 91
GGGGSGGGGS
SEQ ID NO: 92
GGGGSGGGGSGGGGS
SEQ ID NO: 93
GGGGSGGGGSGGGGSGGGGS
SEQ ID NO: 94
GGGGSGGGGSGGGGSGGGGSGGGGS
SEQ ID NO: 95
GGGGSGGGGSGGGGSGGGGSGGGGSGGGGS
SEQ ID NO: 96
KKVVLGKKGDTVELTCTASQKKNIQFHWKNSNQIKILGNQGSFLT
KGPSKLNDRVDSRRSLWDQGNFPLIIKNLKIEDSDTYICEVEDQK
EEVQLVVFGLTANSDTHLLQGQSLTLTLESPPGSSPSVQCRSPRG
KNIQGGKTLSVSQLELQDSGTWTCTVLQNQKKVEFKIDIVVLAFQ
PQLQESGPTLVEASETLSLTCAVSGDSTAACNSFWGWVRQPPGKG
LEWVGSLSHCASYWNRGWTYHNPSLKSRLTLALDTPKNLVFLKLN
SVTAADTATYYCARFGGEVLRYTDWPKPAWVDLWGRGTLVTVSSA
STKGPSVFPLAPSSKSTSGGTAALGCLVKDYFPEPVTVSWNSGAL
TSGVHTFPAVLQSSGLYSLSSVVTVPSSSLGTQTYICNVNHKPSN
TKVDKKVEPKSCDKTHTCPPCPAPELLGGPSVFLFPPKPKDTLMI
SRTPEVTCVVVDVSHEDPEVKFNWYVDGVEVHNAKTKPREEQYNS
TYRVVSVLTVLHQDWLNGKEYKCKVSNKALPAPIEKTISKAKGQP
REPQVYTLPPSRDELTKNQVSLTCLVKGFYPSDIAVEWESNGQPE
NNYKTTPPVLDSDGSFFLYSKLTVDKSRWQQGNVFSCSVLHEALH
SHYTQKSLSLSPGK
SEQ ID NO: 97
KKVVLGKKGDTVELTCTASQKKNIQFHWKNSNQIKILGNQGSFLT
KGPSKLNDRVDSRRSLWDQGNFPLIIKNLKIEDSDTYICEVEDQK
EEVQLVVFGLTANSDTHLLQGQSLTLTLESPPGSSPSVQCRSPRG
KNIQGGKTLSVSQLELQDSGTWTCTVLQNQKKVEFKIDIVVLAFG
GGGSQPQLQESGPTLVEASETLSLTCAVSGDSTAACNSFWGWVRQ
PPGKGLEWVGSLSHCASYWNRGWTYHNPSLKSRLTLALDTPKNLV
FLKLNSVTAADTATYYCARFGGEVLRYTDWPKPAWVDLWGRGTLV
TVSSASTKGPSVFPLAPSSKSTSGGTAALGCLVKDYFPEPVTVSW
NSGALTSGVHTFPAVLQSSGLYSLSSVVTVPSSSLGTQTYICNVN
HKPSNTKVDKKVEPKSCDKTHTCPPCPAPELLGGPSVFLFPPKPK
DTLMISRTPEVTCVVVDVSHEDPEVKFNWYVDGVEVHNAKTKPRE
EQYNSTYRVVSVLTVLHQDWLNGKEYKCKVSNKALPAPIEKTISK
AKGQPREPQVYTLPPSRDELTKNQVSLTCLVKGFYPSDIAVEWES
NGQPENNYKTTPPVLDSDGSFFLYSKLTVDKSRWQQGNVFSCSVL
HEALHSHYTQKSLSLSPGK
SEQ ID NO: 98
KKVVLGKKGDTVELTCTASQKKNIQFHWKNSNQIKILGNQGSFLT
KGPSKLNDRVDSRRSLWDQGNFPLIIKNLKIEDSDTYICEVEDQK
EEVQLVVFGLTANSDTHLLQGQSLTLTLESPPGSSPSVQCRSPRG
KNIQGGKTLSVSQLELQDSGTWTCTVLQNQKKVEFKIDIVVLAFG
GGGSGGGGSQPQLQESGPTLVEASETLSLTCAVSGDSTAACNSFW
GWVRQPPGKGLEWVGSLSHCASYWNRGWTYHNPSLKSRLTLALDT
PKNLVFLKLNSVTAADTATYYCARFGGEVLRYTDWPKPAWVDLWG
RGTLVTVSSASTKGPSVFPLAPSSKSTSGGTAALGCLVKDYFPEP
VTVSWNSGALTSGVHTFPAVLQSSGLYSLSSVVTVPSSSLGTQTY
ICNVNHKPSNTKVDKKVEPKSCDKTHTCPPCPAPELLGGPSVFLF
PPKPKDTLMISRTPEVTCVVVDVSHEDPEVKFNWYVDGVEVHNAK
TKPREEQYNSTYRVVSVLTVLHQDWLNGKEYKCKVSNKALPAPIE
KTISKAKGQPREPQVYTLPPSRDELTKNQVSLTCLVKGFYPSDIA
VEWESNGQPENNYKTTPPVLDSDGSFFLYSKLTVDKSRWQQGNVF
SCSVLHEALHSHYTQKSLSLSPGK
SEQ ID NO: 99
KKVVLGKKGDTVELTCTASQKKNIQFHWKNSNQIKILGNQGSFLT
KGPSKLNDRVDSRRSLWDQGNFPLIIKNLKIEDSDTYICEVEDQK
EEVQLVVFGLTANSDTHLLQGQSLTLTLESPPGSSPSVQCRSPRG
KNIQGGKTLSVSQLELQDSGTWTCTVLQNQKKVEFKIDIVVLAFG
GGGSGGGGSGGGGSQPQLQESGPTLVEASETLSLTCAVSGDSTAA
CNSFWGWVRQPPGKGLEWVGSLSHCASYWNRGWTYHNPSLKSRLT
LALDTPKNLVFLKLNSVTAADTATYYCARFGGEVLRYTDWPKPAW
VDLWGRGTLVTVSSASTKGPSVFPLAPSSKSTSGGTAALGCLVKD
YFPEPVTVSWNSGALTSGVHTFPAVLQSSGLYSLSSVVTVPSSSL
GTQTYICNVNHKPSNTKVDKKVEPKSCDKTHTCPPCPAPELLGGP
SVFLFPPKPKDTLMISRTPEVTCVVVDVSHEDPEVKFNWYVDGVE
VHNAKTKPREEQYNSTYRVVSVLTVLHQDWLNGKEYKCKVSNKAL
PAPIEKTISKAKGQPREPQVYTLPPSRDELTKNQVSLTCLVKGFY
PSDIAVEWESNGQPENNYKTTPPVLDSDGSFFLYSKLTVDKSRWQ
QGNVFSCSVLHEALHSHYTQKSLSLSPGK
SEQ ID NO: 100
KKVVLGKKGDTVELTCTASQKKNIQFHWKNSNQIKILGNQGSFLT
KGPSKLNDRVDSRRSLWDQGNFPLIIKNLKIEDSDTYICEVEDQK
EEVQLVVFGLTANSDTHLLQGQSLTLTLESPPGSSPSVQCRSPRG
KNIQGGKTLSVSQLELQDSGTWTCTVLQNQKKVEFKIDIVVLAFG
GGGSGGGGSGGGGSGGGGSQPQLQESGPTLVEASETLSLTCAVSG
DSTAACNSFWGWVRQPPGKGLEWVGSLSHCASYWNRGWTYHNPSL
KSRLTLALDTPKNLVFLKLNSVTAADTATYYCARFGGEVLRYTDW
PKPAWVDLWGRGTLVTVSSASTKGPSVFPLAPSSKSTSGGTAALG
CLVKDYFPEPVTVSWNSGALTSGVHTFPAVLQSSGLYSLSSVVTV
PSSSLGTQTYICNVNHKPSNTKVDKKVEPKSCDKTHTCPPCPAPE
LLGGPSVFLFPPKPKDTLMISRTPEVTCVVVDVSHEDPEVKFNWY
VDGVEVHNAKTKPREEQYNSTYRVVSVLTVLHQDWLNGKEYKCKV
SNKALPAPIEKTISKAKGQPREPQVYTLPPSRDELTKNQVSLTCL
VKGFYPSDIAVEWESNGQPENNYKTTPPVLDSDGSFFLYSKLTVD
KSRWQQGNVFSCSVLHEALHSHYTQKSLSLSPGK
SEQ ID NO: 101
KKVVYGKKGDTVELTCTASQKKNIQFHWKNSNQIKILGNQGSFLT
KGPSKLNDRVDSRRSLWDQGNFPLIIKNLKPEDSDTYICEVEDQK
EEVQLVVVGQPQLQESGPTLVEASETLSLTCAVSGDSTAACNSFW
GWVRQPPGKGLEWVGSLSHCASYWNRGWTYHNPSLKSRLTLALDT
PKNLVFLKLNSVTAADTATYYCARFGGEVLRYTDWPKPAWVDLWG
RGTLVTVSSASTKGPSVFPLAPSSKSTSGGTAALGCLVKDYFPEP
VTVSWNSGALTSGVHTFPAVLQSSGLYSLSSVVTVPSSSLGTQTY
ICNVNHKPSNTKVDKKVEPKSCDKTHTCPPCPAPELLGGPSVFLF
PPKPKDTLMISRTPEVTCVVVDVSHEDPEVKFNWYVDGVEVHNAK
TKPREEQYNSTYRVVSVLTVLHQDWLNGKEYKCKVSNKALPAPIE
KTISKAKGQPREPQVYTLPPSRDELTKNQVSLTCLVKGFYPSDIA
VEWESNGQPENNYKTTPPVLDSDGSFFLYSKLTVDKSRWQQGNVF
SCSVLHEALHSHYTQKSLSLSPGK
SEQ ID NO: 102
KKVVYGKKGDTVELTCTASQKKNIQFHWKNSNQIKILGNQGSFLT
KGPSKLNDRVDSRRSLWDQGNFPLIIKNLKPEDSDTYICEVEDQK
EEVQLVVVGGGGGSQPQLQESGPTLVEASETLSLTCAVSGDSTAA
CNSFWGWVRQPPGKGLEWVGSLSHCASYWNRGWTYHNPSLKSRLT
LALDTPKNLVFLKLNSVTAADTATYYCARFGGEVLRYTDWPKPAW
VDLWGRGTLVTVSSASTKGPSVFPLAPSSKSTSGGTAALGCLVKD
YFPEPVTVSWNSGALTSGVHTFPAVLQSSGLYSLSSVVTVPSSSL
GTQTYICNVNHKPSNTKVDKKVEPKSCDKTHTCPPCPAPELLGGP
SVFLFPPKPKDTLMISRTPEVTCVVVDVSHEDPEVKFNWYVDGVE
VHNAKTKPREEQYNSTYRVVSVLTVLHQDWLNGKEYKCKVSNKAL
PAPIEKTISKAKGQPREPQVYTLPPSRDELTKNQVSLTCLVKGFY
PSDIAVEWESNGQPENNYKTTPPVLDSDGSFFLYSKLTVDKSRWQ
QGNVFSCSVLHEALHSHYTQKSLSLSPGK
SEQ ID NO: 103
KKVVYGKKGDTVELTCTASQKKNIQFHWKNSNQIKILGNQGSFLT
KGPSKLNDRVDSRRSLWDQGNFPLIIKNLKPEDSDTYICEVEDQK
EEVQLVVVGGGGGSGGGGSQPQLQESGPTLVEASETLSLTCAVSG
DSTAACNSFWGWVRQPPGKGLEWVGSLSHCASYWNRGWTYHNPSL
KSRLTLALDTPKNLVFLKLNSVTAADTATYYCARFGGEVLRYTDW
PKPAWVDLWGRGTLVTVSSASTKGPSVFPLAPSSKSTSGGTAALG
CLVKDYFPEPVTVSWNSGALTSGVHTFPAVLQSSGLYSLSSVVTV
PSSSLGTQTYICNVNHKPSNTKVDKKVEPKSCDKTHTCPPCPAPE
LLGGPSVFLFPPKPKDTLMISRTPEVTCVVVDVSHEDPEVKFNWY
VDGVEVHNAKTKPREEQYNSTYRVVSVLTVLHQDWLNGKEYKCKV
SNKALPAPIEKTISKAKGQPREPQVYTLPPSRDELTKNQVSLTCL
VKGFYPSDIAVEWESNGQPENNYKTTPPVLDSDGSFFLYSKLTVD
KSRWQQGNVFSCSVLHEALHSHYTQKSLSLSPGK
SEQ ID NO: 104
KKVVYGKKGDTVELTCTASQKKNIQFHWKNSNQIKILGNQGSFLT
KGPSKLNDRVDSRRSLWDQGNFPLIIKNLKPEDSDTYICEVEDQK
EEVQLVVVGGGGGSGGGGSGGGGSQPQLQESGPTLVEASETLSLT
CAVSGDSTAACNSFWGWVRQPPGKGLEWVGSLSHCASYWNRGWTY
HNPSLKSRLTLALDTPKNLVFLKLNSVTAADTATYYCARFGGEVL
RYTDWPKPAWVDLWGRGTLVTVSSASTKGPSVFPLAPSSKSTSGG
TAALGCLVKDYFPEPVTVSWNSGALTSGVHTFPAVLQSSGLYSLS
SVVTVPSSSLGTQTYICNVNHKPSNTKVDKKVEPKSCDKTHTCPP
CPAPELLGGPSVFLFPPKPKDTLMISRTPEVTCVVVDVSHEDPEV
KFNWYVDGVEVHNAKTKPREEQYNSTYRVVSVLTVLHQDWLNGKE
YKCKVSNKALPAPIEKTISKAKGQPREPQVYTLPPSRDELTKNQV
SLTCLVKGFYPSDIAVEWESNGQPENNYKTTPPVLDSDGSFFLYS
KLTVDKSRWQQGNVFSCSVLHEALHSHYTQKSLSLSPGK
SEQ ID NO: 105
KKVVYGKKGDTVELTCTASQKKNIQFHWKNSNQIKILGNQGSFLT
KGPSKLNDRVDSRRSLWDQGNFPLIIKNLKPEDSDTYICEVEDQK
EEVQLVVVGGGGGSGGGGSGGGGSGGGGSQPQLQESGPTLVEASE
TLSLTCAVSGDSTAACNSFWGWVRQPPGKGLEWVGSLSHCASYWN
RGWTYHNPSLKSRLTLALDTPKNLVFLKLNSVTAADTATYYCARF
GGEVLRYTDWPKPAWVDLWGRGTLVTVSSASTKGPSVFPLAPSSK
STSGGTAALGCLVKDYFPEPVTVSWNSGALTSGVHTFPAVLQSSG
LYSLSSVVTVPSSSLGTQTYICNVNHKPSNTKVDKKVEPKSCDKT
HTCPPCPAPELLGGPSVFLFPPKPKDTLMISRTPEVTCVVVDVSH
EDPEVKFNWYVDGVEVHNAKTKPREEQYNSTYRVVSVLTVLHQDW
LNGKEYKCKVSNKALPAPIEKTISKAKGQPREPQVYTLPPSRDEL
TKNQVSLTCLVKGFYPSDIAVEWESNGQPENNYKTTPPVLDSDGS
FFLYSKLTVDKSRWQQGNVFSCSVLHEALHSHYTQKSLSLSPGK
SEQ ID NO: 106
KKVVYGKKGDTVELTCTASQKKNIQFHWKNSNQIKILGNQGSFLT
KGPSKLNDRVDSRRSLWDQGNFPLIIKNLKPEDSDTYICEVEDQK
EEVQLVVVGGGGGSGGGGSGGGGSGGGGSGGGGSQPQLQESGPTL
VEASETLSLTCAVSGDSTAACNSFWGWVRQPPGKGLEWVGSLSHC
ASYWNRGWTYHNPSLKSRLTLALDTPKNLVFLKLNSVTAADTATY
YCARFGGEVLRYTDWPKPAWVDLWGRGTLVTVSSASTKGPSVFPL
APSSKSTSGGTAALGCLVKDYFPEPVTVSWNSGALTSGVHTFPAV
LQSSGLYSLSSVVTVPSSSLGTQTYICNVNHKPSNTKVDKKVEPK
SCDKTHTCPPCPAPELLGGPSVFLEPPKPKDTLMISRTPEVTCVV
DVSHEDPEVKFNWYVDGVEVHNAKTKPREEQYNSTYRVVSVLTVL
HQDWLNGKEYKCKVSNKALPAPIEKTISKAKGQPREPQVYTLPPS
RDELTKNQVSLTCLVKGFYPSDIAVEWESNGQPENNYKTTPPVLD
SDGSFFLYSKLTVDKSRWQQGNVFSCSVLHEALHSHYTQKSLSLS
PGK
SEQ ID NO: 107
KKVVYGKKGDTVELTCTASQKKNIQFHWKNSNQIKILGNQGSFLT
KGPSKLNDRVDSRRSLWDQGNFPLIIKNLKPEDSDTYICEVEDQK
EEVQLVVVGGGGGSGGGGSGGGGSGGGGSGGGGSGGGGSQPQLQE
SGPTLVEASETLSLTCAVSGDSTAACNSFWGWVRQPPGKGLEWVG
SLSHCASYWNRGWTYHNPSLKSRLTLALDTPKNLVFLKLNSVTAA
DTATYYCARFGGEVLRYTDWPKPAWVDLWGRGTLVTVSSASTKGP
SVFPLAPSSKSTSGGTAALGCLVKDYFPEPVTVSWNSGALTSGVH
TFPAVLQSSGLYSLSSVVTVPSSSLGTQTYICNVNHKPSNTKVDK
KVEPKSCDKTHTCPPCPAPELLGGPSVFLFPPKPKDTLMISRTPE
VTCVVVDVSHEDPEVKFNWYVDGVEVHNAKTKPREEQYNSTYRVV
SVLTVLHQDWLNGKEYKCKVSNKALPAPIEKTISKAKGQPREPQV
YTLPPSRDELTKNQVSLTCLVKGFYPSDIAVEWESNGQPENNYKT
TPPVLDSDGSFFLYSKLTVDKSRWQQGNVFSCSVLHEALHSHYTQ
KSLSLSPGK
SEQ ID NO: 108
KKVVYGKKGDTVELTCTASQKKNIQFHWKNSNQIKILGNQGSFLT
KGPSKLNDRVDSRRSLWDQGNFPLIIKNLKPEDSDTYICEVEDQK
EEVQLVVVGQSALTQPPSASGSPGQSITISCTGTSNNFVSWYQQH
AGKAPKLVIYDVNKRPSGVPDRFSGSKSGNTASLTVSGLQTDDEA
VYYCGSLVGNWDVIFGGGTKLTVLGQPKAAPSVTLFPPSSEELQA
NKATLVCLISDFYPGAVTVAWKADSSPVKAGVETTTPSKQSNNKY
AASSYLSLTPEQWKSHRSYSCQVTHEGSTVEKTVAPTECS
SEQ ID NO: 109
KKVVYGKKGDTVELTCTASQKKNIQFHWKNSNQIKILGNQGSFLT
KGPSKLNDRVDSRRSLWDQGNFPLIIKNLKPEDSDTYICEVEDQK
EEVQLVVVGGGGGSQSALTQPPSASGSPGQSITISCTGTSNNFVS
WYQQHAGKAPKLVIYDVNKRPSGVPDRFSGSKSGNTASLTVSGLQ
TDDEAVYYCGSLVGNWDVIFGGGTKLTVLGQPKAAPSVTLFPPSS
EELQANKATLVCLISDFYPGAVTVAWKADSSPVKAGVETTTPSKQ
SNNKYAASSYLSLTPEQWKSHRSYSCQVTHEGSTVEKTVAPTECS
SEQ ID NO: 110
KKVVYGKKGDTVELTCTASQKKNIQFHWKNSNQIKILGNQGSFLT
KGPSKLNDRVDSRRSLWDQGNFPLIIKNLKPEDSDTYICEVEDQK
EEVQLVVVGGGGGSGGGGSQSALTQPPSASGSPGQSITISCTGTS
NNFVSWYQQHAGKAPKLVIYDVNKRPSGVPDRFSGSKSGNTASLT
VSGLQTDDEAVYYCGSLVGNWDVIFGGGTKLTVLGQPKAAPSVTL
FPPSSEELQANKATLVCLISDFYPGAVTVAWKADSSPVKAGVETT
TPSKQSNNKYAASSYLSLTPEQWKSHRSYSCQVTHEGSTVEKTVA
PTECS
SEQ ID NO: 111
KKVVYGKKGDTVELTCTASQKKNIQFHWKNSNQIKILGNQGSFLT
KGPSKLNDRVDSRRSLWDQGNFPLIIKNLKPEDSDTYICEVEDQK
EEVQLVVVGGGGGSGGGGSGGGGSQSALTQPPSASGSPGQSITIS
CTGTSNNFVSWYQQHAGKAPKLVIYDVNKRPSGVPDRFSGSKSGN
TASLTVSGLQTDDEAVYYCGSLVGNWDVIFGGGTKLTVLGQPKAA
PSVTLFPPSSEELQANKATLVCLISDFYPGAVTVAWKADSSPVKA
GVETTTPSKQSNNKYAASSYLSLTPEQWKSHRSYSCQVTHEGSTV
EKTVAPTECS
SEQ ID NO: 112
KKVVYGKKGDTVELTCTASQKKNIQFHWKNSNQIKILGNQGSFLT
KGPSKLNDRVDSRRSLWDQGNFPLIIKNLKPEDSDTYICEVEDQK
EEVQLVVVGGGGGSGGGGSGGGGSGGGGSQSALTQPPSASGSPGQ
SITISCTGTSNNFVSWYQQHAGKAPKLVIYDVNKRPSGVPDRFSG
SKSGNTASLTVSGLQTDDEAVYYCGSLVGNWDVIFGGGTKLTVLG
QPKAAPSVTLFPPSSEELQANKATLVCLISDFYPGAVTVAWKADS
SPVKAGVETTTPSKQSNNKYAASSYLSLTPEQWKSHRSYSCQVTH
EGSTVEKTVAPTECS
SEQ ID NO: 113
KKVVYGKKGDTVELTCTASQKKNIQFHWKNSNQIKILGNQGSFLT
KGPSKLNDRVDSRRSLWDQGNFPLIIKNLKPEDSDTYICEVEDQK
EEVQLVVVGGGGGSGGGGSGGGGSGGGGSGGGGSQSALTQPPSAS
GSPGQSITISCTGTSNNFVSWYQQHAGKAPKLVIYDVNKRPSGVP
DRFSGSKSGNTASLTVSGLQTDDEAVYYCGSLVGNWDVIFGGGTK
LTVLGQPKAAPSVTLFPPSSEELQANKATLVCLISDFYPGAVTVA
WKADSSPVKAGVETTTPSKQSNNKYAASSYLSLTPEQWKSHRSYS
CQVTHEGSTVEKTVAPTECS
SEQ ID NO: 114
KKVVYGKKGDTVELTCTASQKKNIQFHWKNSNQIKILGNQGSFLT
KGPSKLNDRVDSRRSLWDQGNFPLIIKNLKPEDSDTYICEVEDQK
EEVQLVVVGGGGGSGGGGSGGGGSGGGGSGGGGSGGGGSQSALTQ
PPSASGSPGQSITISCTGTSNNFVSWYQQHAGKAPKLVIYDVNKR
PSGVPDRFSGSKSGNTASLTVSGLQTDDEAVYYCGSLVGNWDVIF
GGGTKLTVLGQPKAAPSVTLFPPSSEELQANKATLVCLISDFYPG
AVTVAWKADSSPVKAGVETTTPSKQSNNKYAASSYLSLTPEQWKS
HRSYSCQVTHEGSTVEKTVAPTECS
SEQ ID NO: 115
KKVVYGKCGDTVELTCTASQKKNIQFHWKNSNQIKILGNQGSFLT
KGPSKLNDRVDSRRSLWDQGNFPLIIKNLKPEDSDTYICEVEDQK
EEVQLVVVCGGGGSQSALTQPPSASGSPGQSITISCTGTSNNFVS
WYQQHAGKAPKLVIYDVNKRPSGVPDRFSGSKSGNTASLTVSGLQ
TDDEAVYYCGSLVGNWDVIFGGGTKLTVLGQPKAAPSVTLFPPSS
EELQANKATLVCLISDFYPGAVTVAWKADSSPVKAGVETTTPSKQ
SNNKYAASSYLSLTPEQWKSHRSYSCQVTHEGSTVEKTVAPTECS
SEQ ID NO: 116
QPQLQESGPTLVEASETLSLTCAVSGDSTAACNSFWGWVRQPPGK
GLEWVGSLSHCASYWNRGWTYHNPSLKSRLTLALDTPKNLVFLKL
NSVTAADTATYYCARFGGEVLRYTDWPKPAWVDLWGRGTLVTVSS
ASTKGPSVFPLAPSSKSTSGGTAALGCLVKDYFPEPVTVSWNSGA
LTSGVHTFPAVLQSSGLYSLSSVVTVPSSSLGTQTYICNVNHKPS
NTKVDKKVEPKSCGGGGSKKVVYGKCGDTVELTCTASQKKNIQFH
WKNSNQIKILGNQGSFLTKGPSKLNDRVDSRRSLWDQGNFPLIIK
NLKPEDSDTYICEVEDQKEEVQLVVVCGGGGSDKTHTCPPCPAPE
LLGGPSVFLFPPKPKDTLMISRTPEVTCVVVDVSHEDPEVKFNWY
VDGVEVHNAKTKPREEQYNSTYRVVSVLTVLHQDWLNGKEYKCKV
SNKALPAPIEKTISKAKGQPREPQVYTLPPSRDELTKNQVSLTCL
VKGFYPSDIAVEWESNGQPENNYKTTPPVLDSDGSFFLYSKLTVD
KSRWQQGNVFSCSVLHEALHSHYTQKSLSLSPGK
SEQ ID NO: 117
QPQLQESGPTLVEASETLSLTCAVSGDSTAACNSFWGWVRQPPGK
GLEWVGSLSHCASYWNRGWTYHNPSLKSRLTLALDTPKNLVFLKL
NSVTAADTATYYCARFGGEVLRYTDWPKPAWVDLWGRGTLVTVSS
ASTKGPSVFPLAPSSKSTSGGTAALGCLVKDYFPEPVTVSWNSGA
LTSGVHTFPAVLQSSGLYSLSSVVTVPSSSLGTQTYICNVNHKPS
NTKVDKKVEPKSCDKTHTCPPCPAPELLGGPSVFLFPPKPKDTLM
ISRTPEVTCVVVDVSHEDPEVKFNWYVDGVEVHNAKTKPREEQYN
STYRVVSVLTVLHQDWLNGKEYKCKVSNKALPAPIEKTISKAKGQ
PREPQVYTLPPSRDELTKNQVSLTCLVKGFYPSDIAVEWESNGQP
ENNYKTTPPVLDSDGSFFLYSKLTVDKSRWQQGNVFSCSVLHEAL
HSHYTQKSLSLSPGGGGGSKKVVYGKCGDTVELTCTASQKKNIQF
HWKNSNQIKILGNQGSFLTKGPSKLNDRVDSRRSLWDQGNFPLII
KNLKPEDSDTYICEVEDQKEEVQLVVVC
SEQ ID NO: 118
QSALTQPPSASGSPGQSITISCTGTSNNFVSWYQQHAGKAPKLVI
YDVNKRPSGVPDRFSGSKSGNTASLTVSGLQTDDEAVYYCGSLVG
NWDVIFGGGTKLTVLGQPKAAPSVTLFPPSSEELQANKATLVCLI
SDFYPGAVTVAWKADSSPVKAGVETTTPSKQSNNKYAASSYLSLT
PEQWKSHRSYSCQVTHEGSTVEKTVAPTECSGGGGSKKVVYGKCG
DTVELTCTASQKKNIQFHWKNSNQIKILGNQGSFLTKGPSKLNDR
VDSRRSLWDQGNFPLIIKNLKPEDSDTYICEVEDQKEEVQLVVVC
SEQ ID NO: 119
KKVVYGKIGDTVELTCTASQKKNIQFHWKNSNQIKILGNQGSFLT
KGPSKLNDRVDSRRSLWDQGNFPLIIKNLKPEDSDTYICEVEDQK
EEVQLVVVGGGGGSQPQLQESGPTLVEASETLSLTCAVSGDSTAA
CNSFWGWVRQPPGKGLEWVGSLSHCASYWNRGWTYHNPSLKSRLT
LALDTPKNLVFLKLNSVTAADTATYYCARFGGEVLRYTDWPKPAW
VDLWGRGTLVTVSSASTKGPSVFPLAPSSKSTSGGTAALGCLVKD
YFPEPVTVSWNSGALTSGVHTFPAVLQSSGLYSLSSVVTVPSSSL
GTQTYICNVNHKPSNTKVDKKVEPKSCDKTHTCPPCPAPELLGGP
SVFLFPPKPKDTLMISRTPEVTCVVVDVSHEDPEVKFNWYVDGVE
VHNAKTKPREEQYNSTYRVVSVLTVLHQDWLNGKEYKCKVSNKAL
PAPIEKTISKAKGQPREPQVYTLPPSRDELTKNQVSLTCLVKGFY
PSDIAVEWESNGQPENNYKTTPPVLDSDGSFFLYSKLTVDKSRWQ
QGNVFSCSVLHEALHSHYTQKSLSLSPGK
SEQ ID NO: 120
KKVVYGKVGDTVELTCTASQKKNIQFHWKNSNQIKILGNQGSFLT
KGPSKLNDRVDSRRSLWDQGNFPLIIKNLKPEDSDTYICEVEDQK
EEVQLVVVGGGGGSQPQLQESGPTLVEASETLSLTCAVSGDSTAA
CNSFWGWVRQPPGKGLEWVGSLSHCASYWNRGWTYHNPSLKSRLT
LALDTPKNLVFLKLNSVTAADTATYYCARFGGEVLRYTDWPKPAW
VDLWGRGTLVTVSSASTKGPSVFPLAPSSKSTSGGTAALGCLVKD
YFPEPVTVSWNSGALTSGVHTFPAVLQSSGLYSLSSWVTVPSSSL
GTQTYICNVNHKPSNTKVDKKVEPKSCDKTHTCPPCPAPELLGGP
SVFLFPPKPKDTLMISRTPEVTCVVVDVSHEDPEVKFNWYVDGVE
VHNAKTKPREEQYNSTYRVVSVLTVLHQDWLNGKEYKCKVSNKAL
PAPIEKTISKAKGQPREPQVYTLPPSRDELTKNQVSLTCLVKGFY
PSDIAVEWESNGQPENNYKTTPPVLDSDGSFFLYSKLTVDKSRWQ
QGNVFSCSVLHEALHSHYTQKSLSLSPGK
SEQ ID NO: 121
KKVVYGKCGDTVELTCTASQKKNIQFHWKNSNQIKILGNQGSFLT
KGPSKLNDRVDSRRSLWDQGNFPLIIKNLKPEDSDTYICEVEDQK
EEVQLVVVCGGGGSQPQLQESGPTLVEASETLSLTCAVSGDSTAA
CNSFWGWVRQPPGKGLEWVGSLSHCASYWNRGWTYHNPSLKSRLT
LALDTPKNLVFLKLNSVTAADTATYYCARFGGEVLRYTDWPKPAW
VDLWGRGTLVTVSSASTKGPSVFPLAPSSKSTSGGTAALGCLVKD
YFPEPVTVSWNSGALTSGVHTFPAVLQSSGLYSLSSWVTVPSSSL
GTQTYICNVNHKPSNTKVDKKVEPKSCDKTHTCPPCPAPELLGGP
SVFLFPPKPKDTLMISRTPEVTCVVVDVSHEDPEVKFNWYVDGVE
VHNAKTKPREEQYNSTYRVVSVLTVLHQDWLNGKEYKCKVSNKAL
PAPIEKTISKAKGQPREPQVYTLPPSRDELTKNQVSLTCLVKGFY
PSDIAVEWESNGQPENNYKTTPPVLDSDGSFFLYSKLTVDKSRWQ
QGNVFSCSVLHEALHSHYTQKSLSLSPGK
SEQ ID NO: 122
KKVVYGKKGDCVELTCTASQKKNIQFHWKNSNQIKILGNQGSFLT
KGPSKLNDRVDSRRSLWDQGNFPLIICNLKPEDSDTYICEVEDQK
EEVQLVVVGGGGGSQPQLQESGPTLVEASETLSLTCAVSGDSTAA
CNSFWGWVRQPPGKGLEWVGSLSHCASYWNRGWTYHNPSLKSRLT
LALDTPKNLVFLKLNSVTAADTATYYCARFGGEVLRYTDWPKPAW
VDLWGRGTLVTVSSASTKGPSVFPLAPSSKSTSGGTAALGCLVKD
YFPEPVTVSWNSGALTSGVHTFPAVLQSSGLYSLSSVVTVPSSSL
GTQTYICNVNHKPSNTKVDKKVEPKSCDKTHTCPPCPAPELLGGP
SVFLFPPKPKDTLMISRTPEVTCVVVDVSHEDPEVKFNWYVDGVE
VHNAKTKPREEQYNSTYRVVSVLTVLHQDWLNGKEYKCKVSNKAL
PAPIEKTISKAKGQPREPQVYTLPPSRDELTKNQVSLTCLVKGFY
PSDIAVEWESNGQPENNYKTTPPVLDSDGSFFLYSKLTVDKSRWQ
QGNVFSCSVLHEALHSHYTQKSLSLSPGK
SEQ ID NO: 123
KKVVYGKKGDTVELTCTASQKKNIQFHWKNSNQIKILGNQGSFLT
KGPSKLNDRVDSRRSLWDQGNFPLIIKNLKPEDSDTYICEVEDQK
QEVQLVVVGGGGGSQPQLQESGPTLVEASETLSLTCAVSGDSTAA
CNSFWGWVRQPPGKGLEWVGSLSHCASYWNRGWTYHNPSLKSRLT
LALDTPKNLVFLKLNSVTAADTATYYCARFGGEVLRYTDWPKPAW
VDLWGRGTLVTVSSASTKGPSVFPLAPSSKSTSGGTAALGCLVKD
YFPEPVTVSWNSGALTSGVHTFPAVLQSSGLYSLSSVVTVPSSSL
GTQTYICNVNHKPSNTKVDKKVEPKSCDKTHTCPPCPAPELLGGP
SVFLFPPKPKDTLMISRTPEVTCVVVDVSHEDPEVKFNWYVDGVE
VHNAKTKPREEQYNSTYRVVSVLTVLHQDWLNGKEYKCKVSNKAL
PAPIEKTISKAKGQPREPQVYTLPPSRDELTKNQVSLTCLVKGFY
PSDIAVEWESNGQPENNYKTTPPVLDSDGSFFLYSKLTVDKSRWQ
QGNVFSCSVLHEALHSHYTQKSLSLSPGK
SEQ ID NO: 124
KKVVYGKKGDTVELTCTASQKKNIQFHWKNSNQIKILGNQGSFLT
KGPSKLNDRVDSRRSLWDQGNFPLIIKNLKPEDSDTYICEVEDQK
HEVQLVVVGGGGGSQPQLQESGPTLVEASETLSLTCAVSGDSTAA
CNSFWGWVRQPPGKGLEWVGSLSHCASYWNRGWTYHNPSLKSRLT
LALDTPKNLVFLKLNSVTAADTATYYCARFGGEVLRYTDWPKPAW
VDLWGRGTLVTVSSASTKGPSVFPLAPSSKSTSGGTAALGCLVKD
YFPEPVTVSWNSGALTSGVHTFPAVLQSSGLYSLSSVVTVPSSSL
GTQTYICNVNHKPSNTKVDKKVEPKSCDKTHTCPPCPAPELLGGP
SVFLFPPKPKDTLMISRTPEVTCVVVDVSHEDPEVKFNWYVDGVE
VHNAKTKPREEQYNSTYRVVSVLTVLHQDWLNGKEYKCKVSNKAL
PAPIEKTISKAKGQPREPQVYTLPPSRDELTKNQVSLTCLVKGFY
PSDIAVEWESNGQPENNYKTTPPVLDSDGSFFLYSKLTVDKSRWQ
QGNVFSCSVLHEALHSHYTQKSLSLSPGK
SEQ ID NO: 125
KKVVYGKKGDTVELTCTASQKKNIEFHWKNSNQIKILGNQGSFLT
KGPSKLNDRVDSRRSLWDQGNFPLIIKNLKPEDSDTYICEVEDQK
EEVQLVVVGGGGGSQPQLQESGPTLVEASETLSLTCAVSGDSTAA
CNSFWGWVRQPPGKGLEWVGSLSHCASYWNRGWTYHNPSLKSRLT
LALDTPKNLVFLKLNSVTAADTATYYCARFGGEVLRYTDWPKPAW
VDLWGRGTLVTVSSASTKGPSVFPLAPSSKSTSGGTAALGCLVKD
YFPEPVTVSWNSGALTSGVHTFPAVLQSSGLYSLSSVVTVPSSSL
GTQTYICNVNHKPSNTKVDKKVEPKSCDKTHTCPPCPAPELLGGP
SVFLFPPKPKDTLMISRTPEVTCVVVDVSHEDPEVKFNWYVDGVE
VHNAKTKPREEQYNSTYRVVSVLTVLHQDWLNGKEYKCKVSNKAL
PAPIEKTISKAKGQPREPQVYTLPPSRDELTKNQVSLTCLVKGFY
PSDIAVEWESNGQPENNYKTTPPVLDSDGSFFLYSKLTVDKSRWQ
QGNVFSCSVLHEALHSHYTQKSLSLSPGK
SEQ ID NO: 126
KKVVYGKKGDTVELTCTASQKKNIQFDWKNSNQIKILGNQGSFLT
KGPSKLNDRVDSRRSLWDQGNFPLIIKNLKPEDSDTYICEVEDQK
EEVQLVVVGGGGGSQPQLQESGPTLVEASETLSLTCAVSGDSTAA
CNSFWGWVRQPPGKGLEWVGSLSHCASYWNRGWTYHNPSLKSRLT
LALDTPKNLVFLKLNSVTAADTATYYCARFGGEVLRYTDWPKPAW
VDLWGRGTLVTVSSASTKGPSVFPLAPSSKSTSGGTAALGCLVKD
YFPEPVTVSWNSGALTSGVHTFPAVLQSSGLYSLSSVVTVPSSSL
GTQTYICNVNHKPSNTKVDKKVEPKSCDKTHTCPPCPAPELLGGP
SVFLFPPKPKDTLMISRTPEVTCVVVDVSHEDPEVKFNWYVDGVE
VHNAKTKPREEQYNSTYRVVSVLTVLHQDWLNGKEYKCKVSNKAL
PAPIEKTISKAKGQPREPQVYTLPPSRDELTKNQVSLTCLVKGFY
PSDIAVEWESNGQPENNYKTTPPVLDSDGSFFLYSKLTVDKSRWQ
QGNVFSCSVLHEALHSHYTQKSLSLSPGK
SEQ ID NO: 127
KKVVYGKKGDTVELTCTASQKKNIQFHWKNSNQIKILGNQGSFLT
KGPSKLNDRVDSVRSLWDQGNFPLIIKNLKPEDSDTYICEVEDQK
EEVQLVVVGGGGGSQPQLQESGPTLVEASETLSLTCAVSGDSTAA
CNSFWGWVRQPPGKGLEWVGSLSHCASYWNRGWTYHNPSLKSRLT
LALDTPKNLVFLKLNSVTAADTATYYCARFGGEVLRYTDWPKPAW
VDLWGRGTLVTVSSASTKGPSVFPLAPSSKSTSGGTAALGCLVKD
YFPEPVTVSWNSGALTSGVHTFPAVLQSSGLYSLSSVVTVPSSSL
GTQTYICNVNHKPSNTKVDKKVEPKSCDKTHTCPPCPAPELLGGP
SVFLFPPKPKDTLMISRTPEVTCVVVDVSHEDPEVKFNWYVDGVE
VHNAKTKPREEQYNSTYRVVSVLTVLHQDWLNGKEYKCKVSNKAL
PAPIEKTISKAKGQPREPQVYTLPPSRDELTKNQVSLTCLVKGFY
PSDIAVEWESNGQPENNYKTTPPVLDSDGSFFLYSKLTVDKSRWQ
QGNVFSCSVLHEALHSHYTQKSLSLSPGK
SEQ ID NO: 128
KKVVYGKKGDTVELTCTASQKKNIQFHWKNSNQIKILGNQGSFLT
KGPSKLNDRVDSNRSLWDQGNFPLIIKNLKPEDSDTYICEVEDQK
EEVQLVVVGGGGGSQPQLQESGPTLVEASETLSLTCAVSGDSTAA
CNSFWGWVRQPPGKGLEWVGSLSHCASYWNRGWTYHNPSLKSRLT
LALDTPKNLVFLKLNSVTAADTATYYCARFGGEVLRYTDWPKPAW
VDLWGRGTLVTVSSASTKGPSVFPLAPSSKSTSGGTAALGCLVKD
YFPEPVTVSWNSGALTSGVHTFPAVLQSSGLYSLSSVVTVPSSSL
GTQTYICNVNHKPSNTKVDKKVEPKSCDKTHTCPPCPAPELLGGP
SVFLFPPKPKDTLMISRTPEVTCVVVDVSHEDPEVKFNWYVDGVE
VHNAKTKPREEQYNSTYRVVSVLTVLHQDWLNGKEYKCKVSNKAL
PAPIEKTISKAKGQPREPQVYTLPPSRDELTKNQVSLTCLVKGFY
PSDIAVEWESNGQPENNYKTTPPVLDSDGSFFLYSKLTVDKSRWQ
QGNVFSCSVLHEALHSHYTQKSLSLSPGK
SEQ ID NO: 129
KKVVYGKKGDTVELTCTASQKKNIQFHWKNSNQIKILGNQGSFLT
KGPSKLNDRVDSTRSLWDQGNFPLIIKNLKPEDSDTYICEVEDQK
EEVQLVVVGGGGGSQPQLQESGPTLVEASETLSLTCAVSGDSTAA
CNSFWGWVRQPPGKGLEWVGSLSHCASYWNRGWTYHNPSLKSRLT
LALDTPKNLVFLKLNSVTAADTATYYCARFGGEVLRYTDWPKPAW
VDLWGRGTLVTVSSASTKGPSVFPLAPSSKSTSGGTAALGCLVKD
YFPEPVTVSWNSGALTSGVHTFPAVLQSSGLYSLSSVVTVPSSSL
GTQTYICNVNHKPSNTKVDKKVEPKSCDKTHTCPPCPAPELLGGP
SVFLFPPKPKDTLMISRTPEVTCVVVDVSHEDPEVKFNWYVDGVE
VHNAKTKPREEQYNSTYRVVSVLTVLHQDWLNGKEYKCKVSNKAL
PAPIEKTISKAKGQPREPQVYTLPPSRDELTKNQVSLTCLVKGFY
PSDIAVEWESNGQPENNYKTTPPVLDSDGSFFLYSKLTVDKSRWQ
QGNVFSCSVLHEALHSHYTQKSLSLSPGK
SEQ ID NO: 130
KKVVYGKKGDTVELTCTASQKKNIQFHWKNSNQIKILGNQGSFLT
KGPSKLNDRVDSRRSMWDQGNFPLIIKNLKPEDSDTYICEVEDQK
EEVQLVVVGGGGGSQPQLQESGPTLVEASETLSLTCAVSGDSTAA
CNSFWGWVRQPPGKGLEWVGSLSHCASYWNRGWTYHNPSLKSRLT
LALDTPKNLVFLKLNSVTAADTATYYCARFGGEVLRYTDWPKPAW
VDLWGRGTLVTVSSASTKGPSVFPLAPSSKSTSGGTAALGCLVKD
YFPEPVTVSWNSGALTSGVHTFPAVLQSSGLYSLSSVVTVPSSSL
GTQTYICNVNHKPSNTKVDKKVEPKSCDKTHTCPPCPAPELLGGP
SVFLFPPKPKDTLMISRTPEVTCVVVDVSHEDPEVKFNWYVDGVE
VHNAKTKPREEQYNSTYRVVSVLTVLHQDWLNGKEYKCKVSNKAL
PAPIEKTISKAKGQPREPQVYTLPPSRDELTKNQVSLTCLVKGFY
PSDIAVEWESNGQPENNYKTTPPVLDSDGSFFLYSKLTVDKSRWQ
QGNVFSCSVLHEALHSHYTQKSLSLSPGK
SEQ ID NO: 131
KKVVYGKKGDTVCLTCTASQKKNIQFHWKNSNQIKILGNQGSFLT
KGPSKLNDRVDSRRSLWDQGNFPLCIKNLKPEDSDTYICEVEDQK
EEVQLVVVGGGGGSQPQLQESGPTLVEASETLSLTCAVSGDSTAA
CNSFWGWVRQPPGKGLEWVGSLSHCASYWNRGWTYHNPSLKSRLT
LALDTPKNLVFLKLNSVTAADTATYYCARFGGEVLRYTDWPKPAW
VDLWGRGTLVTVSSASTKGPSVFPLAPSSKSTSGGTAALGCLVKD
YFPEPVTVSWNSGALTSGVHTFPAVLQSSGLYSLSSVVTVPSSSL
GTQTYICNVNHKPSNTKVDKKVEPKSCDKTHTCPPCPAPELLGGP
SVFLFPPKPKDTLMISRTPEVTCVVVDVSHEDPEVKFNWYVDGVE
VHNAKTKPREEQYNSTYRVVSVLTVLHQDWLNGKEYKCKVSNKAL
PAPIEKTISKAKGQPREPQVYTLPPSRDELTKNQVSLTCLVKGFY
PSDIAVEWESNGQPENNYKTTPPVLDSDGSFFLYSKLTVDKSRWQ
QGNVFSCSVLHEALHSHYTQKSLSLSPGK
SEQ ID NO: 132
KKVVYGKKGDTVELTCTASQKKNIQFCWKNSNQIKILCNQGSFLT
KGPSKLNDRVDSRRSLWDQGNFPLIIKNLKPEDSDTYICEVEDQK
EEVQLVVVGGGGGSQPQLQESGPTLVEASETLSLTCAVSGDSTAA
CNSFWGWVRQPPGKGLEWVGSLSHCASYWNRGWTYHNPSLKSRLT
LALDTPKNLVFLKLNSVTAADTATYYCARFGGEVLRYTDWPKPAW
VDLWGRGTLVTVSSASTKGPSVFPLAPSSKSTSGGTAALGCLVKD
YFPEPVTVSWNSGALTSGVHTFPAVLQSSGLYSLSSVVTVPSSSL
GTQTYICNVNHKPSNTKVDKKVEPKSCDKTHTCPPCPAPELLGGP
SVFLFPPKPKDTLMISRTPEVTCVVVDVSHEDPEVKFNWYVDGVE
VHNAKTKPREEQYNSTYRVVSVLTVLHQDWLNGKEYKCKVSNKAL
PAPIEKTISKAKGQPREPQVYTLPPSRDELTKNQVSLTCLVKGFY
PSDIAVEWESNGQPENNYKTTPPVLDSDGSFFLYSKLTVDKSRWQ
QGNVFSCSVLHEALHSHYTQKSLSLSPGK
SEQ ID NO: 133
KKVVYGKKGDTVELTCTASQCKNIQFHWKNSNQIKILGNQGSFLT
KGPSKLNDRVDSRRSLWDQCNFPLIIKNLKPEDSDTYICEVEDQK
EEVQLVVVGGGGGSQPQLQESGPTLVEASETLSLTCAVSGDSTAA
CNSFWGWVRQPPGKGLEWVGSLSHCASYWNRGWTYHNPSLKSRLT
LALDTPKNLVFLKLNSVTAADTATYYCARFGGEVLRYTDWPKPAW
VDLWGRGTLVTVSSASTKGPSVFPLAPSSKSTSGGTAALGCLVKD
YFPEPVTVSWNSGALTSGVHTFPAVLQSSGLYSLSSVVTVPSSSL
GTQTYICNVNHKPSNTKVDKKVEPKSCDKTHTCPPCPAPELLGGP
SVFLFPPKPKDTLMISRTPEVTCVVVDVSHEDPEVKFNWYVDGVE
VHNAKTKPREEQYNSTYRVVSVLTVLHQDWLNGKEYKCKVSNKAL
PAPIEKTISKAKGQPREPQVYTLPPSRDELTKNQVSLTCLVKGFY
PSDIAVEWESNGQPENNYKTTPPVLDSDGSFFLYSKLTVDKSRWQ
QGNVFSCSVLHEALHSHYTQKSLSLSPGK
SEQ ID NO: 134
KKVVYGKKGDTVELTCTASQKKNIQFHWKNSNQIKILGNQGSFLT
KGPSKLNDRVDSRRSLWDQGNFPLIIKNLKPEDSDTYICEVGDQK
EEVQLVVVGGGGGSQPQLQESGPTLVEASETLSLTCAVSGDSTAA
CNSFWGWVRQPPGKGLEWVGSLSHCASYWNRGWTYHNPSLKSRLT
LALDTPKNLVFLKLNSVTAADTATYYCARFGGEVLRYTDWPKPAW
VDLWGRGTLVTVSSASTKGPSVFPLAPSSKSTSGGTAALGCLVKD
YFPEPVTVSWNSGALTSGVHTFPAVLQSSGLYSLSSVVTVPSSSL
GTQTYICNVNHKPSNTKVDKKVEPKSCDKTHTCPPCPAPELLGGP
SVFLFPPKPKDTLMISRTPEVTCVVVDVSHEDPEVKFNWYVDGVE
VHNAKTKPREEQYNSTYRVVSVLTVLHQDWLNGKEYKCKVSNKAL
PAPIEKTISKAKGQPREPQVYTLPPSRDELTKNQVSLTCLVKGFY
PSDIAVEWESNGQPENNYKTTPPVLDSDGSFFLYSKLTVDKSRWQ
QGNVFSCSVLHEALHSHYTQKSLSLSPGK
SEQ ID NO: 135
KKVVYGKKGDTVELTCTASQKKNIQFHWKNSNQIKILGNQGSFLT
KGPSKLWDRVDSRRSLWDQGNFPLIIKNLKPEDSDTYICEVEDQK
EEVQLVVVGGGGGSQPQLQESGPTLVEASETLSLTCAVSGDSTAA
CNSFWGWVRQPPGKGLEWVGSLSHCASYWNRGWTYHNPSLKSRLT
LALDTPKNLVFLKLNSVTAADTATYYCARFGGEVLRYTDWPKPAW
VDLWGRGTLVTVSSASTKGPSVFPLAPSSKSTSGGTAALGCLVKD
YFPEPVTVSWNSGALTSGVHTFPAVLQSSGLYSLSSVVTVPSSSL
GTQTYICNVNHKPSNTKVDKKVEPKSCDKTHTCPPCPAPELLGGP
SVFLFPPKPKDTLMISRTPEVTCVVVDVSHEDPEVKFNWYVDGVE
VHNAKTKPREEQYNSTYRVVSVLTVLHQDWLNGKEYKCKVSNKAL
PAPIEKTISKAKGQPREPQVYTLPPSRDELTKNQVSLTCLVKGFY
PSDIAVEWESNGQPENNYKTTPPVLDSDGSFFLYSKLTVDKSRWQ
QGNVFSCSVLHEALHSHYTQKSLSLSPGK
SEQ ID NO: 136
KKVVYGKCGDTVELTCTASQKKNIQFHWKNSNQIKILGNQGSFLT
KGPSKLNDRVDSRRSLWDQGNFPLIIKNLKPEDSDTYICEVEDQK
EEVQLVVVCGGGGSQMQLQESGPGLVKPSETLSLTCSVSGASISD
SYWSWIRRSPGKGLEWIGYVHKSGDTNYSPSLKSRVNLSLDTSKN
QVSLSLVAATAADSGKYYCARTLHGRRIYGIVAFNEWFTYFYMDV
WGNGTQVTVSSASTKGPSVFPLAPSSKSTSGGTAALGCLVKDYFP
EPVTVSWNSGALTSGVHTFPAVLQSSGLYSLSSVVTVPSSSLGTQ
TYICNVNHKPSNTKVDKRVEPKSCDKTHTCPPCPAPELLGGPSVF
LFPPKPKDTLMISRTPEVTCVVVDVSHEDPEVKFNWYVDGVEVHN
AKTKPREEQYNSTYRVVSVLTVLHQDWLNGKEYKCKVSNKALPAP
IEKTISKAKGQPREPQVYTLPPSRDELTKNQVSLTCLVKGFYPSD
IAVEWESNGQPENNYKTTPPVLDSDGSFFLYSKLTVDKSRWQQGN
VFSCSVLHEALHSHYTQKSLSLSPGK
SEQ ID NO: 137
KKVVYGKCGDTVELTCTASQKKNIQFHWKNSNQIKILGNQGSFLT
KGPSKLNDRVDSRRSLWDQGNFPLIIKNLKPEDSDTYICEVEDQK
EEVQLVVVCGGGGSGGGGSQMQLQESGPGLVKPSETLSLTCSVSG
ASISDSYWSWIRRSPGKGLEWIGYVHKSGDTNYSPSLKSRVNLSL
DTSKNQVSLSLVAATAADSGKYYCARTLHGRRIYGIVAFNEWFTY
FYMDVWGNGTQVTVSSASTKGPSVFPLAPSSKSTSGGTAALGCLV
KDYFPEPVTVSWNSGALTSGVHTFPAVLQSSGLYSLSSVVTVPSS
SLGTQTYICNVNHKPSNTKVDKRVEPKSCDKTHTCPPCPAPELLG
GPSVFLFPPKPKDTLMISRTPEVTCVVVDVSHEDPEVKFNWYVDG
VEVHNAKTKPREEQYNSTYRVVSVLTVLHQDWLNGKEYKCKVSNK
ALPAPIEKTISKAKGQPREPQVYTLPPSRDELTKNQVSLTCLVKG
FYPSDIAVEWESNGQPENNYKTTPPVLDSDGSFFLYSKLTVDKSR
WQQGNVFSCSVLHEALHSHYTQKSLSLSPGK
SEQ ID NO: 138
KKVVYGKCGDTVELTCTASQKKNIQFHWKNSNQIKILGNQGSFLT
KGPSKLNDRVDSRRSLWDQGNFPLIIKNLKPEDSDTYICEVEDQK
EEVQLVVVCGGGGSGGGGSGGGGSQMQLQESGPGLVKPSETLSLT
CSVSGASISDSYWSWIRRSPGKGLEWIGYVHKSGDTNYSPSLKSR
VNLSLDTSKNQVSLSLVAATAADSGKYYCARTLHGRRIYGIVAFN
EWFTYFYMDVWGNGTQVTVSSASTKGPSVFPLAPSSKSTSGGTAA
LGCLVKDYFPEPVTVSWNSGALTSGVHTFPAVLQSSGLYSLSSVV
TVPSSSLGTQTYICNVNHKPSNTKVDKRVEPKSCDKTHTCPPCPA
PELLGGPSVFLFPPKPKDTLMISRTPEVTCVVVDVSHEDPEVKFN
WYVDGVEVHNAKTKPREEQYNSTYRVVSVLTVLHQDWLNGKEYKC
KVSNKALPAPIEKTISKAKGQPREPQVYTLPPSRDELTKNQVSLT
CLVKGFYPSDIAVEWESNGQPENNYKTTPPVLDSDGSFFLYSKLT
VDKSRWQQGNVFSCSVLHEALHSHYTQKSLSLSPGK
SEQ ID NO: 139
KKVVYGKCGDTVELTCTASQKKNIQFHWKNSNQIKILGNQGSFLT
KGPSKLNDRVDSRRSLWDQGNFPLIIKNLKPEDSDTYICEVEDQK
EEVQLVVVCGGGGSGGGGSGGGGSGGGGSQMQLQESGPGLVKPSE
TLSLTCSVSGASISDSYWSWIRRSPGKGLEWIGYVHKSGDTNYSP
SLKSRVNLSLDTSKNQVSLSLVAATAADSGKYYCARTLHGRRIYG
IVAFNEWFTYFYMDVWGNGTQVTVSSASTKGPSVFPLAPSSKSTS
GGTAALGCLVKDYFPEPVTVSWNSGALTSGVHTFPAVLQSSGLYS
LSSVVTVPSSSLGTQTYICNVNHKPSNTKVDKRVEPKSCDKTHTC
PPCPAPELLGGPSVFLFPPKPKDTLMISRTPEVTCVVVDVSHEDP
EVKFNWYVDGVEVHNAKTKPREEQYNSTYRVVSVLTVLHQDWLNG
KEYKCKVSNKALPAPIEKTISKAKGQPREPQVYTLPPSRDELTKN
QVSLTCLVKGFYPSDIAVEWESNGQPENNYKTTPPVLDSDGSFFL
YSKLTVDKSRWQQGNVFSCSVLHEALHSHYTQKSLSLSPGK
SEQ ID NO: 140
QMQLQESGPGLVKPSETLSLTCSVSGASISDSYWSWIRRSPGKGL
EWIGYVHKSGDTNYSPSLKSRVNLSLDTSKNQVSLSLVAATAADS
GKYYCARTLHGRRIYGIVAFNEWFTYFYMDVWGNGTQVTVSSAST
KGPSVFPLAPSSKSTSGGTAALGCLVKDYFPEPVTVSWNSGALTS
GVHTFPAVLQSSGLYSLSSVVTVPSSSLGTQTYICNVNHKPSNTK
VDKRVEPKSCGGGGSKKVVYGKCGDTVELTCTASQKKNIQFHWKN
SNQIKILGNQGSFLTKGPSKLNDRVDSRRSLWDQGNFPLIIKNLK
PEDSDTYICEVEDQKEEVQLVVVCGGGGSDKTHTCPPCPAPELLG
GPSVFLFPPKPKDTLMISRTPEVTCVVVDVSHEDPEVKFNWYVDG
VEVHNAKTKPREEQYNSTYRVVSVLTVLHQDWLNGKEYKCKVSNK
ALPAPIEKTISKAKGQPREPQVYTLPPSRDELTKNQVSLTCLVKG
FYPSDIAVEWESNGQPENNYKTTPPVLDSDGSFFLYSKLTVDKSR
WQQGNVFSCSVLHEALHSHYTQKSLSLSPGK
SEQ ID NO: 141
QMQLQESGPGLVKPSETLSLTCSVSGASISDSYWSWIRRSPGKGL
EWIGYVHKSGDTNYSPSLKSRVNLSLDTSKNQVSLSLVAATAADS
GKYYCARTLHGRRIYGIVAFNEWFTYFYMDVWGNGTQVTVSSAST
KGPSVFPLAPSSKSTSGGTAALGCLVKDYFPEPVTVSWNSGALTS
GVHTFPAVLQSSGLYSLSSVVTVPSSSLGTQTYICNVNHKPSNTK
VDKRVEPKSCDKTHTCPPCPAPELLGGPSVFLFPPKPKDTLMISR
TPEVTCVVVDVSHEDPEVKFNWYVDGVEVHNAKTKPREEQYNSTY
RVVSVLTVLHQDWLNGKEYKCKVSNKALPAPIEKTISKAKGQPRE
PQVYTLPPSRDELTKNQVSLTCLVKGFYPSDIAVEWESNGQPENN
YKTTPPVLDSDGSFFLYSKLTVDKSRWQQGNVFSCSVLHEALHSH
YTQKSLSLSPGGGGGSKKVVYGKCGDTVELTCTASQKKNIQFHWK
NSNQIKILGNQGSFLTKGPSKLNDRVDSRRSLWDQGNFPLIIKNL
KPEDSDTYICEVEDQKEEVQLVVVC
SEQ ID NO: 142
KKVVYGKCGDTVELTCTASQKKNIQFHWKNSNQIKILGNQGSFLT
KGPSKLNDRVDSRRSLWDQGNFPLIIKNLKPEDSDTYICEVEDQK
EEVQLVVVCGGGGSSDISVAPGETARISCGEKSLGSRAVQWYQHR
AGQAPSLIIYNNQDRPSGIPERFSGSPDSPFGTTATLTITSVEAG
DEADYYCHIWDSRVPTKWVFGGGTTLTVLGQPKAAPSVTLFPPSS
EELQANKATLVCLISDFYPGAVTVAWKADSSPVKAGVETTTPSKQ
SNNKYAASSYLSLTPEQWKSHKSYSCQVTHEGSTVEKTVAPTECS
SEQ ID NO: 143
SDISVAPGETARISCGEKSLGSRAVQWYQHRAGQAPSLIIYNNQD
RPSGIPERFSGSPDSPFGTTATLTITSVEAGDEADYYCHIWDSRV
PTKWVFGGGTTLTVLGQPKAAPSVTLFPPSSEELQANKATLVCLI
SDFYPGAVTVAWKADSSPVKAGVETTTPSKQSNNKYAASSYLSLT
PEQWKSHKSYSCQVTHEGSTVEKTVAPTECSGGGGSKKVVYGKCG
DTVELTCTASQKKNIQFHWKNSNQIKILGNQGSFLTKGPSKLNDR
VDSRRSLWDQGNFPLIIKNLKPEDSDTYICEVEDQKEEVQLVVVC
SEQ ID NO: 144
KKVVYGKCGDTVELTCTASQKKNIQFHWKNSNQIKILGNQGSFLT
KGPSKLNDRVDSRRSLWDQGNFPLIIKNLKPEDSDTYICEVEDQK
EEVQLVVVCGGGGSQVQLRESGPGLVKPSETLSLSCTVSNDSRPS
DHSWTWVRQSPGKALEWIGDIHYNGATTYNPSLRSRVRIELDQSI
PRFSLKMTSMTAADTGMYYCARNAIRIYGVVALGEWFHYGMDVWG
QGTAVTVSSASTKGPSVFPLAPSSKSTSGGTAALGCLVKDYFPEP
VTVSWNSGALTSGVHTFPAVLQSSGLYSLSSVVTVPSSSLGTQTY
ICNVNHKPSNTKVDKRVEPKSCDKTHTCPPCPAPELLGGPSVFLF
PPKPKDTLMISRTPEVTCVVVDVSHEDPEVKFNWYVDGVEVHNAK
TKPREEQYNSTYRVVSVLTVLHQDWLNGKEYKCKVSNKALPAPIE
KTISKAKGQPREPQVYTLPPSRDELTKNQVSLTCLVKGFYPSDIA
VEWESNGQPENNYKTTPPVLDSDGSFFLYSKLTVDKSRWQQGNVF
SCSVLHEALHSHYTQKSLSLSPGK
SEQ ID NO: 145
KKVVYGKCGDTVELTCTASQKKNIQFHWKNSNQIKILGNQGSFLT
KGPSKLNDRVDSRRSLWDQGNFPLIIKNLKPEDSDTYICEVEDQK
EEVQLVVVCGGGGSGGGGSGGGGSGGGGSQVQLRESGPGLVKPSE
TLSLSCTVSNDSRPSDHSWTWVRQSPGKALEWIGDIHYNGATTYN
PSLRSRVRIELDQSIPRFSLKMTSMTAADTGMYYCARNAIRIYGV
VALGEWFHYGMDVWGQGTAVTVSSASTKGPSVFPLAPSSKSTSGG
TAALGCLVKDYFPEPVTVSWNSGALTSGVHTFPAVLQSSGLYSLS
SWVTVPSSSLGTQTYICNVNHKPSNTKVDKRVEPKSCDKTHTCPP
CPAPELLGGPSVFLFPPKPKDTLMISRTPEVTCVVVDVSHEDPEV
KFNWYVDGVEVHNAKTKPREEQYNSTYRVVSVLTVLHQDWLNGKE
YKCKVSNKALPAPIEKTISKAKGQPREPQVYTLPPSRDELTKNQV
SLTCLVKGFYPSDIAVEWESNGQPENNYKTTPPVLDSDGSFFLYS
KLTVDKSRWQQGNVFSCSVLHEALHSHYTQKSLSLSPGK
SEQ ID NO: 146
KKVVYGKCGDTVELTCTASQKKNIQFHWKNSNQIKILGNQGSFLT
KGPSKLNDRVDSRRSLWDQGNFPLIIKNLKPEDSDTYICEVEDQK
EEVQLVVVCGGGGSQVQLQESGPGLVKPSETLSVTCSVSGDSMNN
YYWTWIRQSPGKGLEWIGYISDRESATYNPSLNSRVVISRDTSKN
QLSLKLNSVTPADTAVYYCATARRGQRIYGVVSFGEFFYYYSMDV
WGKGTTVTVSSASTKGPSVFPLAPSSKSTSGGTAALGCLVKDYFP
EPVTVSWNSGALTSGVHTFPAVLQSSGLYSLSSVVTVPSSSLGTQ
TYICNVNHKPSNTKVDKRVEPKSCDKTHTCPPCPAPELLGGPSVF
LFPPKPKDTLMISRTPEVTCVVVDVSHEDPEVKFNWYVDGVEVHN
AKTKPREEQYNSTYRVVSVLTVLHQDWLNGKEYKCKVSNKALPAP
IEKTISKAKGQPREPQVYTLPPSRDELTKNQVSLTCLVKGFYPSD
IAVEWESNGQPENNYKTTPPVLDSDGSFFLYSKLTVDKSRWQQGN
VFSCSVLHEALHSHYTQKSLSLSPGK
SEQ ID NO: 147
KKVVYGKCGDTVELTCTASQKKNIQFHWKNSNQIKILGNQGSFLT
KGPSKLNDRVDSRRSLWDQGNFPLIIKNLKPEDSDTYICEVEDQK
EEVQLVVVCGGGGGGGGSGGGGSGGGGSQVQLQESGPGLVKPSET
LSVTCSVSGDSMNNYYWTWIRQSPGKGLEWIGYISDRESATYNPS
LNSRVVISRDTSKNQLSLKLNSVTPADTAVYYCATARRGQRIYGV
VSFGEFFYYYSMDVWGKGTTVTVSSASTKGPSVFPLAPSSKSTSG
GTAALGCLVKDYFPEPVTVSWNSGALTSGVHTFPAVLQSSGLYSL
SSVVTVPSSSLGTQTYICNVNHKPSNTKVDKRVEPKSCDKTHTCP
PCPAPELLGGPSVFLFPPKPKDTLMISRTPEVTCVVVDVSHEDPE
VKFNWYVDGVEVHNAKTKPREEQYNSTYRVVSVLTVLHQDWLNGK
EYKCKVSNKALPAPIEKTISKAKGQPREPQVYTLPPSRDELTKNQ
VSLTCLVKGFYPSDIAVEWESNGQPENNYKTTPPVLDSDGSFFLY
SKLTVDKSRWQQGNVFSCSVLHEALHSHYTQKSLSLSPGK
SEQ ID NO: 148
KKVVYGKCGDTVELTCTASQKKNIQFHWKNSNQIKILGNQGSFLT
KGPSKLNDRVDSRRSLWDQGNFPLIIKNLKPEDSDTYICEVEDQK
EEVQLVVVCGGGGSQLQMQESGPGLVKPSETLSLSCTVSGDSIRG
GEWGDKDYHWGWVRHSAGKGLEWIGSIHWRGTTHYKESLRRRVSM
SIDTSRNWFSLRLASVTAADTAVYFCARHRHHDVFMLVPIAGWFD
VWGPGVQVTVSSASTKGPSVFPLAPSSKSTSGGTAALGCLVKDYF
PEPVTVSWNSGALTSGVHTFPAVLQSSGLYSLSSVVTVPSSSLGT
QTYICNVNHKPSNTKVDKRVEPKSCDKTHTCPPCPAPELLGGPSV
FLFPPKPKDTLMISRTPEVTCVVVDVSHEDPEVKFNWYVDGVEVH
NAKTKPREEQYNSTYRVVSVLTVLHQDWLNGKEYKCKVSNKALPA
PIEKTISKAKGQPREPQVYTLPPSRDELTKNQVSLTCLVKGFYPS
DIAVEWESNGQPENNYKTTPPVLDSDGSFFLYSKLTVDKSRWQQG
NVFSCSVLHEALHSHYTQKSLSLSPGK
SEQ ID NO: 149
KKVVYGKCGDTVELTCTASQKKNIQFHWKNSNQIKILGNQGSFLT
KGPSKLNDRVDSRRSLWDQGNFPLIIKNLKPEDSDTYICEVEDQK
EEVQLVVVCGGGGSGGGGSGGGGSGGGGSQLQMQESGPGLVKPSE
TLSLSCTVSGDSIRGGEWGDKDYHWGWVRHSAGKGLEWIGSIHWR
GTTHYKESLRRRVSMSIDTSRNWFSLRLASVTAADTAVYFCARHR
HHDVFMLVPIAGWFDVWGPGVQVTVSSASTKGPSVFPLAPSSKST
SGGTAALGCLVKDYFPEPVTVSWNSGALTSGVHTFPAVLQSSGLY
SLSSWVTVPSSSLGTQTYICNVNHKPSNTKVDKRVEPKSCDKTHT
CPPCPAPELLGGPSVFLFPPKPKDTLMISRTPEVTCVVVDVSHED
PEVKFNWYVDGVEVHNAKTKPREEQYNSTYRVVSVLTVLHQDWLN
GKEYKCKVSNKALPAPIEKTISKAKGQPREPQVYTLPPSRDELTK
NQVSLTCLVKGFYPSDIAVEWESNGQPENNYKTTPPVLDSDGSFF
LYSKLTVDKSRWQQGNVFSCSVLHEALHSHYTQKSLSLSPGK
SEQ ID NO: 150
KKVVLGKKGDTVELTCTASQKKNIQFHWKNSNQIKILGNQGSFLT
KGPSKLNDRVDSRRSLWDQGNFPLIIKNLKIEDSDTYICEVEDQK
EEVQLVVFGLTANSDTHLLQGQSLTLTLESPPGSSPSVQCRSPRG
KNIQGGKTLSVSQLELQDSGTWTCTVLQNQKKVEFKIDIVVLAFG
GGGSGGGGSGGGGSGGGGSQVQLVQSGAQMKNPGASVKVSCAPSG
YTFTDFYIHWLRQAPGQGLQWMGWMNPQTGRTNTARNFQGRVTMT
RDTSIGTAYMELRSLTSDDTAIYYCTTGGWISLYYDSSYYPNFDH
WGQGTLLTVSSASTKGPSVFPLAPSSKSTSGGTAALGCLVKDYFP
EPVTVSWNSGALTSGVHTFPAVLQSSGLYSLSSVVTVPSSSLGTQ
TYICNVNHKPSNTKVDKKVEPKSCDKTHTCPPCPAPELLGGPSVF
LFPPKPKDTLMISRTPEVTCVVVDVSHEDPEVKFNWYVDGVEVHN
AKTKPREEQYNSTYRVVSVLTVLHQDWLNGKEYKCKVSNKALPAP
IEKTISKAKGQPREPQVYTLPPSRDELTKNQVSLTCLVKGFYPSD
IAVEWESNGQPENNYKTTPPVLDSDGSFFLYSKLTVDKSRWQQGN
VFSCSVLHEALHSHYTQKSLSLSPGK
SEQ ID NO: 151
KKVVYGKKGDTVELTCTASQKKNIQFHWKNSNQIKILGNQGSFLT
KGPSKLNDRVDSRRSLWDQGNFPLIIKNLKPEDSDTYICEVEDQK
EEVQLVVVGGGGGSGGGGSGGGGSGGGGSQVQLVQSGAQMKNPGA
SVKVSCAPSGYTFTDFYIHWLRQAPGQGLQWMGWMNPQTGRTNTA
RNFQGRVTMTRDTSIGTAYMELRSLTSDDTAIYYCTTGGWISLYY
DSSYYPNFDHWGQGTLLTVSSASTKGPSVFPLAPSSKSTSGGTAA
LGCLVKDYFPEPVTVSWNSGALTSGVHTFPAVLQSSGLYSLSSWV
TVPSSSLGTQTYICNVNHKPSNTKVDKKVEPKSCDKTHTCPPCPA
PELLGGPSVFLFPPKPKDTLMISRTPEVTCVVVDVSHEDPEVKFN
WYVDGVEVHNAKTKPREEQYNSTYRVVSVLTVLHQDWLNGKEYKC
KVSNKALPAPIEKTISKAKGQPREPQVYTLPPSRDELTKNQVSLT
CLVKGFYPSDIAVEWESNGQPENNYKTTPPVLDSDGSFFLYSKLT
VDKSRWQQGNVFSCSVLHEALHSHYTQKSLSLSPGK
SEQ ID NO: 152
KKVVYGKKGDTVELTCTASQKKNIQFHWKNSNQIKILGNQGSFLT
KGPSKLNDRVDSRRSLWDQGNFPLIIKNLKPEDSDTYICEVEDQK
EEVQLVVVGGGGGSGGGGSGGGGSGGGGSQPQLQESGPTLVEASE
TLSLTCAVSGDSTAACNSFWGWVRQPPGKCLEWVGSLSHCASYWN
RGWTYHNPSLKSRLTLALDTPKNLVFLKLNSVTAADTATYYCARF
GGEVLRYTDWPKPAWVDLWGRGTLVTVSSGGGGSGGGGSGGGGSG
GGGSQSALTQPPSASGSPGQSITISCTGTSNNFVSWYQQHAGKAP
KLVIYDVNKRPSGVPDRFSGSKSGNTASLTVSGLQTDDEAVYYCG
SLVGNWDVIFGCGTKLTVLGGGGSGGGGSDKTHTCPPCPAPELLG
GPSVFLFPPKPKDTLMISRTPEVTCVVVDVSHEDPEVKFNWYVDG
VEVHNAKTKPREEQYNSTYRVVSVLTVLHQDWLNGKEYKCKVSNK
ALPAPIEKTISKAKGQPREPQVYTLPPSRDELTKNQVSLTCLVKG
FYPSDIAVEWESNGQPENNYKTTPPVLDSDGSFFLYSKLTVDKSR
WQQGNVFSCSVLHEALHSHYTQKSLSLSPGK
SEQ ID NO: 153
KKVVYGKKGDTVELTCTASQKKNIQFHWKNSNQIKILGNQGSFLT
KGPSKLNDRVDSRRSLWDQGNFPLIIKNLKPEDSDTYICEVEDQK
EEVQLVVVGGGGGSGGGGSGGGGSQPQLQESGPTLVEASETLSLT
CAVSGDSTAACNSFWGWVRQPPGKCLEWVGSLSHCASYWNRGWTY
HNPSLKSRLTLALDTPKNLVFLKLNSVTAADTATYYCARFGGEVL
RYTDWPKPAWVDLWGRGTLVTVSSGGGGSGGGGSGGGGSGGGGSQ
SALTQPPSASGSPGQSITISCTGTSNNFVSWYQQHAGKAPKLVIY
DVNKRPSGVPDRFSGSKSGNTASLTVSGLQTDDEAVYYCGSLVGN
WDVIFGCGTKLTVLGGGGSGGGGSDKTHTCPPCPAPELLGGPSVF
LFPPKPKDTLMISRTPEVTCVVVDVSHEDPEVKFNWYVDGVEVHN
AKTKPREEQYNSTYRVVSVLTVLHQDWLNGKEYKCKVSNKALPAP
IEKTISKAKGQPREPQVYTLPPSRDELTKNQVSLTCLVKGFYPSD
IAVEWESNGQPENNYKTTPPVLDSDGSFFLYSKLTVDKSRWQQGN
VFSCSVLHEALHSHYTQKSLSLSPGK
SEQ ID NO: 154
KKVVYGKKGDTVELTCTASQKKNIQFHWKNSNQIKILGNQGSFLT
KGPSKLNDRVDSRRSLWDQGNFPLIIKNLKPEDSDTYICEVEDQK
EEVQLVVVGGGGGSGGGGSQPQLQESGPTLVEASETLSLTCAVSG
DSTAACNSFWGWVRQPPGKCLEWVGSLSHCASYWNRGWTYHNPSL
KSRLTLALDTPKNLVFLKLNSVTAADTATYYCARFGGEVLRYTDW
PKPAWVDLWGRGTLVTVSSGGGGSGGGGSGGGGSGGGGSQSALTQ
PPSASGSPGQSITISCTGTSNNFVSWYQQHAGKAPKLVIYDVNKR
PSGVPDRFSGSKSGNTASLTVSGLQTDDEAVYYCGSLVGNWDVIF
GCGTKLTVLGGGGSGGGGSDKTHTCPPCPAPELLGGPSVFLFPPK
PKDTLMISRTPEVTCVVVDVSHEDPEVKFNWYVDGVEVHNAKTKP
REEQYNSTYRVVSVLTVLHQDWLNGKEYKCKVSNKALPAPIEKTI
SKAKGQPREPQVYTLPPSRDELTKNQVSLTCLVKGFYPSDIAVEW
ESNGQPENNYKTTPPVLDSDGSFFLYSKLTVDKSRWQQGNVFSCS
VLHEALHSHYTQKSLSLSPGK
SEQ ID NO: 155
KKVVYGKKGDTVELTCTASQKKNIQFHWKNSNQIKILGNQGSFLT
KGPSKLNDRVDSRRSLWDQGNFPLIIKNLKPEDSDTYICEVEDQK
EEVQLVVVGGGGGSQPQLQESGPTLVEASETLSLTCAVSGDSTAA
CNSFWGWVRQPPGKCLEWVGSLSHCASYWNRGWTYHNPSLKSRLT
LALDTPKNLVFLKLNSVTAADTATYYCARFGGEVLRYTDWPKPAW
VDLWGRGTLVTVSSGGGGSGGGGSGGGGSGGGGSQSALTQPPSAS
GSPGQSITISCTGTSNNFVSWYQQHAGKAPKLVIYDVNKRPSGVP
DRFSGSKSGNTASLTVSGLQTDDEAVYYCGSLVGNWDVIFGCGTK
LTVLGGGGSGGGGSDKTHTCPPCPAPELLGGPSVFLFPPKPKDTL
MISRTPEVTCVVVDVSHEDPEVKFNWYVDGVEVHNAKTKPREEQY
NSTYRVVSVLTVLHQDWLNGKEYKCKVSNKALPAPIEKTISKAKG
QPREPQVYTLPPSRDELTKNQVSLTCLVKGFYPSDIAVEWESNGQ
PENNYKTTPPVLDSDGSFFLYSKLTVDKSRWQQGNVFSCSVLHEA
LHSHYTQKSLSLSPGK
SEQ ID NO: 156
DKTHTCPPCPAPELLGGPSVFLFPPKPKDTLMISRTPEVTCVVVD
VSHEDPEVKFNWYVDGVEVHNAKTKPREEQYNSTYRVVSVLTVLH
QDWLNGKEYKCKVSNKALPAPIEKTISKAKGQPREPQVYTLPPSR
DELTKNQVSLTCLVKGFYPSDIAVEWESNGQPENNYKTTPPVLDS
DGSFFLYSKLTVDKSRWQQGNVFSCSVLHEALHSHYTQKSLSLSP
GKGGGGSGGGGSGGGGSGGGGSKKVVYGKKGDTVELTCTASQKKN
IQFHWKNSNQIKILGNQGSFLTKGPSKLNDRVDSRRSLWDQGNFP
LIIKNLKPEDSDTYICEVEDQKEEVQLVVVGGGGGSGGGGSGGGG
SQPQLQESGPTLVEASETLSLTCAVSGDSTAACNSFWGWVRQPPG
KCLEWVGSLSHCASYWNRGWTYHNPSLKSRLTLALDTPKNLVFLK
LNSVTAADTATYYCARFGGEVLRYTDWPKPAWVDLWGRGTLVTVS
SGGGGSGGGGSGGGGSGGGGSQSALTQPPSASGSPGQSITISCTG
TSNNFVSWYQQHAGKAPKLVIYDVNKRPSGVPDRFSGSKSGNTAS
LTVSGLQTDDEAVYYCGSLVGNWDVIFGCGTKLTVL
SEQ ID NO: 157
QSALTQPPSASGSPGQSITISCTGTSNNFVSWYQQHAGKAPKLVI
YDVNKRPSGVPDRFSGSKSGNTASLTVSGLQTDDEAVYYCGSLVG
NWDVIFGCGTKLTVLGGGGSGGGGSGGGGSGGGGSQPQLQESGPT
LVEASETLSLTCAVSGDSTAACNSFWGWVRQPPGKCLEWVGSLSH
CASYWNRGWTYHNPSLKSRLTLALDTPKNLVFLKLNSVTAADTAT
YYCARFGGEVLRYTDWPKPAWVDLWGRGTLVTVSSGGGGGGGGSG
GGGSGGGGSKKVVYGKKGDTVELTCTASQKKNIQFHWKNSNQIKI
LGNQGSFLTKGPSKLNDRVDSRRSLWDQGNFPLIIKNLKPEDSDT
YICEVEDQKEEVQLVWVGGGGGSGGGGSGGGGSDKTHTCPPCPAP
ELLGGPSVFLFPPKPKDTLMISRTPEVTCVVVDVSHEDPEVKFNW
YVDGVEVHNAKTKPREEQYNSTYRVVSVLTVLHQDWLNGKEYKCK
VSNKALPAPIEKTISKAKGQPREPQVYTLPPSRDELTKNQVSLTC
LVKGFYPSDIAVEWESNGQPENNYKTTPPVLDSDGSFFLYSKLTV
DKSRWQQGNVFSCSVLHEALHSHYTQKSLSLSPGK
SEQ ID NO: 158
KKVVYGKCGDTVELTCTASQKKNIQFHWKNSNQIKILGNQGSFLT
KGPSKLNDRVDSRRSLWDQGNFPLIIKNLKPEDSDTYICEVEDQK
EEVQLVVVCGGGGSGGGGSGGGGSGGGGSDKTHTCPPCPAPELLG
GPSVFLFPPKPKDTLMISRTPEVTCVVVDVSHEDPEVKFNWYVDG
VEVHNAKTKPREEQYNSTYRVVSVLTVLHQDWLNGKEYKCKVSNK
ALPAPIEKTISKAKGQPREPQVYTLPPSRDELTKNQVSLTCLVKG
FYPSDIAVEWESNGQPENNYKTTPPVLDSDGSFFLYSKLTVDKSR
WQQGNVFSCSVLHEALHSHYTQKSLSLSPGK
SEQ ID NO: 159
DNYWS
SEQ ID NO: 160
YVHDSGDTNYNPSLKS
SEQ ID NO: 161
TKHGRRIYGVVAFKEWFTYFYMDV
SEQ ID NO: 162
GEESLGSRSVI
SEQ ID NO: 163
NNNDRPS
SEQ ID NO: 164
HIWDSRRPTNWV
SEQ ID NO: 165
DAYWS
SEQ ID NO: 166
YVHHSGDTNYNPSLKR
SEQ ID NO: 167
ALHGKRIYGIVALGELFTYFYMDV
SEQ ID NO: 168
GKESIGSRAVQ
SEQ ID NO: 169
NNQDRPA
SEQ ID NO: 170
HIYDARGGTNWV
SEQ ID NO: 171
ACTYFWG
SEQ ID NO: 172
SLSHCQSFWGSGWTFHNPSLKS
SEQ ID NO: 173
FDGEVLVYNHWPKPAWVDL
SEQ ID NO: 174
NGTATNFVS
SEQ ID NO: 175
GVDKRPP
SEQ ID NO: 176
GSLVGNWDVI
SEQ ID NO: 177
ACDYFWG
SEQ ID NO: 178
GLSHCAGYYNTGWTYHNPSLKS
SEQ ID NO: 179
FDGEVLVYHDWPKPAWVDL
SEQ ID NO: 180
TGTSNRFVS
SEQ ID NO: 181
GVNKRPS
SEQ ID NO: 182
SSLVGNWDVI
SEQ ID NO: 183
RCNYFWG
SEQ ID NO: 184
SLSHCRSYYNTDWTYHNPSLKS
SEQ ID NO: 185
FGGEVLVYRDWPKPAWVDL
SEQ ID NO: 186
TGTSNNFVS
SEQ ID NO: 187
EVNKRPS
SEQ ID NO: 188
SSLVGNWDVI
SEQ ID NO: 189
TGHYYWG
SEQ ID NO: 190
HIHYTTAVLHNPSLKS
SEQ ID NO: 191
SGGDILYYYEWQKPHWFSP
SEQ ID NO: 192
NGTSSDIGGWNFVS
SEQ ID NO: 193
EVNKRPS
SEQ ID NO: 194
SSLFGRWDVV
SEQ ID NO: 185
TGHHYWG
SEQ ID NO: 196
:HIHYNTAVLHNPALKS
SEQ ID NO: 197
SGGDILYYIEWQKPHWFYP
SEQ ID NO: 198
SGTGSDIGSWNFVS
SEQ ID NO: 199
EVNRRRS
SEQ ID NO: 200
SSLSGRWDIV
SEQ ID NO: 201
GTDWGENDFHYG
SEQ ID NO: 202
SIHWRGRTTHYKTSFRS
SEQ ID NO: 203
HKYHDIFRVVPVAGWFDP
SEQ ID NO: 204
RASQNVKNNLA
SEQ ID NO: 205
DASSRAG
SEQ ID NO: 206
QQYEEWPRT
SEQ ID NO: 207
GGEWGDSDYHWG
SEQ ID NO: 208
SIHWRGTTHYNAPFRG
SEQ ID NO: 209
HKYHDIVMVVPIAGWFDP
SEQ ID NO: 210
RASQSVKNNLA
SEQ ID NO: 211
DTSSRAS
SEQ ID NO: 212
QQYEEWPRT
SEQ ID NO: 213
DVWLN
SEQ ID NO: 214
RIKSRTDGGTTDYAASVKG
SEQ ID NO: 215
DGFIMIRGVSEDYYYYYMDV
SEQ ID NO: 216
SGSSSNIGNNYVL
SEQ ID NO: 217
GNNKRPS
SEQ ID NO: 218
ATWDSGLSADWV
SEQ ID NO: 219
SYVMH
SEQ ID NO: 220
AISSDGETTYHANSVKG
SEQ ID NO: 221
DRYYETSGSNAFDV
SEQ ID NO: 222
QASQDISNYLN
SEQ ID NO: 223
TASNLET
SEQ ID NO: 224
QQYDNLGDLS
SEQ ID NO: 225
NFAIH
SEQ ID NO: 226
GRVPVVGIYKYGKKFHD
SEQ ID NO: 227
WRGCGMCPYDTSSYYNDASDV
SEQ ID NO: 228
RASQNISSSWIA
SEQ ID NO: 229
AASARAA
SEQ ID NO: 230
QYYGGSFFT
SEQ ID NO: 231
AHTMN
SEQ ID NO: 232
SISTSSTYRDYADAVKG
SEQ ID NO: 233
KGSDRLSDNDPFDA
SEQ ID NO: 234
RASQSIETWLA
SEQ ID NO: 235
KASTLKT
SEQ ID NO: 236
QHYAGYSAT
SEQ ID NO: 237
SSYWS
SEQ ID NO: 238
YTHHSGDTNYAPSLKS
SEQ ID NO: 239
TLHGRRIYGVVAFNEFFTYFYWEV
SEQ ID NO: 240
GGESIGSRAVQ
SEQ ID NO: 241
NNQDRPP
SEQ ID NO: 242
HIWDSRRPTNWV
SEQ ID NO: 243
SSYWS
SEQ ID NO: 244
YTHHSGDTNYAPSLKS
SEQ ID NO: 245
TLHGRRIYGVVAFNEYYTYFYWPT
SEQ ID NO: 246
GGESIGSRAVQ
SEQ ID NO: 247
NNQDRPP
SEQ ID NO: 248
HIWDSRRPTNWE
SEQ ID NO: 249
SSYWS
SEQ ID NO: 250
YTHHSGDTNYAPSLKS
SEQ ID NO: 251
TLHGRRIYGVVAFNEYYTYFYWPT
SEQ ID NO: 252
TGTSSDIGASDYVS
SEQ ID NO: 253
DVTKRPS
SEQ ID NO: 254
SSDAGRHTLL
SEQ ID NO: 255
QVHLQESGPGLVKPSETLSLTCNVSGTLVRDNYWSWIRQPLGKQP
EWIGYVHDSGDTNYNPSLKSRVHLSLDKSKNLVSLRLTGVTAADS
AIYYCATTKHGRRIYGVVAFKEWFTYFYMDVWGKGTSVTVSS
SEQ ID NO: 256
TFVSVAPGQTARITCGEESLGSRSVIWYQQRPGQAPSLIIYNNND
RPSGIPDRFSGSPGSTFGTTATLTITSVEAGDEADYYCHIWDSRR
PTNWVFGEGTTLIVL
SEQ ID NO: 257
QVHLQESGPGLVKPSETLSLTCNVSGTLVRDNYWSWIRQPLGKQP
EWIGYVHDSGDTNYNPSLKSRVHLSLDKSKNLVSLRLTGVTAADS
AIYYCATTKHGRRIYGVVAFKEWFTYFYMDVWGKGTSVTVSSAST
KGPSVFPLAPSSKSTSGGTAALGCLVKDYFPEPVTVSWNSGALTS
GVHTFPAVLQSSGLYSLSSVVTVPSSSLGTQTYICNVNHKPSNTK
VDKKVEPKSCDKTHTCPPCPAPELLGGPSVFLFPPKPKDTLMISR
TPEVTCVVVDVSHEDPEVKFNWYVDGVEVHNAKTKPREEQYNSTY
RVVSVLTVLHQDWLNGKEYKCKVSNKALPAPIEKTISKAKGQPRE
PQVYTLPPSRDELTKNQVSLTCLVKGFYPSDIAVEWESNGQPENN
YKTTPPVLDSDGSFFLYSKLTVDKSRWQQGNVFSCSVMHEALHNH
YTQKSLSLSPGK
SEQ ID NO: 258
QVHLQESGPGLVKPSETLSLTCNVSGTLVRDNYWSWIRQPLGKQP
EWIGYVHDSGDTNYNPSLKSRVHLSLDKSKNLVSLRLTGVTAADS
AIYYCATTKHGRRIYGVVAFKEWFTYFYMDVWGKGTSVTVSSAST
KGPSVFPLAPSSKSTSGGTAALGCLVKDYFPEPVTVSWNSGALTS
GVHTFPAVLQSSGLYSLSSVVTVPSSSLGTQTYICNVNHKPSNTK
VDKKVEPKSCDKTHTCPPCPAPELLGGPSVFLFPPKPKDTLMISR
TPEVTCVVVDVSHEDPEVKFNWYVDGVEVHNAKTKPREEQYNSTY
RVVSVLTVLHQDWLNGKEYKCKVSNKALPAPIEKTISKAKGQPRE
PQVYTLPPSRDELTKNQVSLTCLVKGFYPSDIAVEWESNGQPENN
YKTTPPVLDSDGSFFLYSKLTVDKSRWQQGNVFSCSVLHEALHSH
YTQKSLSLSPGK
SEQ ID NO: 259
TFVSVAPGQTARITCGEESLGSRSVIWYQQRPGQAPSLIIYNNND
RPSGIPDRFSGSPGSTFGTTATLTITSVEAGDEADYYCHIWDSRR
PTNWVFGEGTTLIVLGQPKAAPSVTLFPPSSEELQANKATLVCLI
SDFYPGAVTVAWKADSSPVKAGVETTTPSKQSNNKYAASSYLSLT
PEQWKSHRSYSCQVTHEGSTVEKTVAPTECS
SEQ ID NO: 260
QLHLQESGPGLVKPPETLSLTCSVSGASINDAYWSWIRQSPGKRP
EWVGYVHHSGDTNYNPSLKRRVTFSLDTAKNEVSLKLVDLTAADS
ATYFCARALHGKRIYGIVALGELFTYFYMDVWGKGTAVTVSS
SEQ ID NO: 261
SSMSVSPGETAKISCGKESIGSRAVQWYQQKPGQPPSLIIYNNQD
RPAGVPERFSASPDFRPGTTATLTITNVDAEDEADYYCHIYDARG
GTNWVFDRGTTLTVL
SEQ ID NO: 262
QLHLQESGPGLVKPPETLSLTCSVSGASINDAYWSWIRQSPGKRP
EWVGYVHHSGDTNYNPSLKRRVTFSLDTAKNEVSLKLVDLTAADS
ATYFCARALHGKRIYGIVALGELFTYFYMDVWGKGTAVTVSSAST
KGPSVFPLAPSSKSTSGGTAALGCLVKDYFPEPVTVSWNSGALTS
GVHTFPAVLQSSGLYSLSSVVTVPSSSLGTQTYICNVNHKPSNTK
VDKKVEPKSCDKTHTCPPCPAPELLGGPSVFLFPPKPKDTLMISR
TPEVTCVVVDVSHEDPEVKFNWYVDGVEVHNAKTKPREEQYNSTY
RVVSVLTVLHQDWLNGKEYKCKVSNKALPAPIEKTISKAKGQPRE
PQVYTLPPSRDELTKNQVSLTCLVKGFYPSDIAVEWESNGQPENN
YKTTPPVLDSDGSFFLYSKLTVDKSRWQQGNVFSCSVMHEALHNH
YTQKSLSLSPGK
SEQ ID NO: 263
QLHLQESGPGLVKPPETLSLTCSVSGASINDAYWSWIRQSPGKRP
EWVGYVHHSGDTNYNPSLKRRVTFSLDTAKNEVSLKLVDLTAADS
ATYFCARALHGKRIYGIVALGELFTYFYMDVWGKGTAVTVSSAST
KGPSVFPLAPSSKSTSGGTAALGCLVKDYFPEPVTVSWNSGALTS
GVHTFPAVLQSSGLYSLSSVVTVPSSSLGTQTYICNVNHKPSNTK
VDKKVEPKSCDKTHTCPPCPAPELLGGPSVFLFPPKPKDTLMISR
TPEVTCVVVDVSHEDPEVKFNWYVDGVEVHNAKTKPREEQYNSTY
RVVSVLTVLHQDWLNGKEYKCKVSNKALPAPIEKTISKAKGQPRE
PQVYTLPPSRDELTKNQVSLTCLVKGFYPSDIAVEWESNGQPENN
YKTTPPVLDSDGSFFLYSKLTVDKSRWQQGNVFSCSVLHEALHSH
YTQKSLSLSPGK
SEQ ID NO: 264
SSMSVSPGETAKISCGKESIGSRAVQWYQQKPGQPPSLIIYNNQD
RPAGVPERFSASPDFRPGTTATLTITNVDAEDEADYYCHIYDARG
GTNWVFDRGTTLTVLGQPKAAPSVTLFPPSSEELQANKATLVCLI
SDFYPGAVTVAWKADSSPVKAGVETTTPSKQSNNKYAASSYLSLT
PEQWKSHRSYSCQVTHEGSTVEKTVAPTECS
SEQ ID NO: 265
QSQLQESGPRLVEASETLSLTCNVSGESTGACTYFWGWVRQAPGK
GLEWIGSLSHCQSFWGSGWTFHNPSLKSRLTISLDTPKNQVFLKL
TSLTAADTATYYCARFDGEVLVYNHWPKPAWVDLWGRGIPVTVTV
SS
SEQ ID NO: 266
QSALTQPPSASGSPGQSITISCNGTATNFVSWYQQFPDKAPKLII
FGVDKRPPGVPDRFSGSRSGTTASLTVSRLQTDDEAVYYCGSLVG
NWDVIFGGGTTLTVL
SEQ ID NO: 267
QSQLQESGPRLVEASETLSLTCNVSGESTGACTYFWGWVRQAPGK
GLEWIGSLSHCQSFWGSGWTFHNPSLKSRLTISLDTPKNQVFLKL
TSLTAADTATYYCARFDGEVLVYNHWPKPAWVDLWGRGIPVTVTV
SSASTKGPSVFPLAPSSKSTSGGTAALGCLVKDYFPEPVTVSWNS
GALTSGVHTFPAVLQSSGLYSLSSVVTVPSSSLGTQTYICNVNHK
PSNTKVDKKVEPKSCDKTHTCPPCPAPELLGGPSVFLFPPKPKDT
LMISRTPEVTCVVVDVSHEDPEVKFNWYVDGVEVHNAKTKPREEQ
YNSTYRVVSVLTVLHQDWLNGKEYKCKVSNKALPAPIEKTISKAK
GQPREPQVYTLPPSRDELTKNQVSLTCLVKGFYPSDIAVEWESNG
QPENNYKTTPPVLDSDGSFFLYSKLTVDKSRWQQGNVFSCSVMHE
ALHNHYTQKSLSLSPGK
SEQ ID NO: 268
QSQLQESGPRLVEASETLSLTCNVSGESTGACTYFWGWVRQAPGK
GLEWIGSLSHCQSFWGSGWTFHNPSLKSRLTISLDTPKNQVFLKL
TSLTAADTATYYCARFDGEVLVYNHWPKPAWVDLWGRGIPVTVTV
SSASTKGPSVFPLAPSSKSTSGGTAALGCLVKDYFPEPVTVSWNS
GALTSGVHTFPAVLQSSGLYSLSSVVTVPSSSLGTQTYICNVNHK
PSNTKVDKKVEPKSCDKTHTCPPCPAPELLGGPSVFLFPPKPKDT
LMISRTPEVTCVVVDVSHEDPEVKFNWYVDGVEVHNAKTKPREEQ
YNSTYRVVSVLTVLHQDWLNGKEYKCKVSNKALPAPIEKTISKAK
GQPREPQVYTLPPSRDELTKNQVSLTCLVKGFYPSDIAVEWESNG
QPENNYKTTPPVLDSDGSFFLYSKLTVDKSRWQQGNVFSCSVLHE
ALHSHYTQKSLSLSPGK
SEQ ID NO: 269
QSALTQPPSASGSPGQSITISCNGTATNFVSWYQQFPDKAPKLII
FGVDKRPPGVPDRFSGSRSGTTASLTVSRLQTDDEAVYYCGSLVG
NWDVIFGGGTTLTVLGQPKAAPSVTLFPPSSEELQANKATLVCLI
SDFYPGAVTVAWKADSSPVKAGVETTTPSKQSNNKYAASSYLSLT
PEQWKSHRSYSCQVTHEGSTVEKTVAPTECS
SEQ ID NO: 270
QPQLQESGPGLVEASETLSLTCTVSGDSTAACDYFWGWVRQPPGK
GLEWIGGLSHCAGYYNTGWTYHNPSLKSRLTISLDTPKNQVFLKL
NSVTAADTAIYYCARFDGEVLVYHDWPKPAWVDLWGRGTLVTVTV
SS
SEQ ID NO: 271
QSALTQPPSASGSPGQSISISCTGTSNRFVSWYQQHPGKAPKLVI
YGVNKRPSGVPDRFSGSKSGNTASLTVSGLQTDDEAVYYCSSLVG
NWDVIFGGGTKLTVL
SEQ ID NO: 272
QPQLQESGPGLVEASETLSLTCTVSGDSTAACDYFWGWVRQPPGK
GLEWIGGLSHCAGYYNTGWTYHNPSLKSRLTISLDTPKNQVFLKL
NSVTAADTAIYYCARFDGEVLVYHDWPKPAWVDLWGRGTLVTVTV
SSASTKGPSVFPLAPSSKSTSGGTAALGCLVKDYFPEPVTVSWNS
GALTSGVHTFPAVLQSSGLYSLSSVVTVPSSSLGTQTYICNVNHK
PSNTKVDKKVEPKSCDKTHTCPPCPAPELLGGPSVFLFPPKPKDT
LMISRTPEVTCVVVDVSHEDPEVKFNWYVDGVEVHNAKTKPREEQ
YNSTYRVVSVLTVLHQDWLNGKEYKCKVSNKALPAPIEKTISKAK
GQPREPQVYTLPPSRDELTKNQVSLTCLVKGFYPSDIAVEWESNG
QPENNYKTTPPVLDSDGSFFLYSKLTVDKSRWQQGNVFSCSVMHE
ALHNHYTQKSLSLSPGK
SEQ ID NO: 273
QPQLQESGPGLVEASETLSLTCTVSGDSTAACDYFWGWVRQPPGK
GLEWIGGLSHCAGYYNTGWTYHNPSLKSRLTISLDTPKNQVFLKL
NSVTAADTAIYYCARFDGEVLVYHDWPKPAWVDLWGRGTLVTVTV
SSASTKGPSVFPLAPSSKSTSGGTAALGCLVKDYFPEPVTVSWNS
GALTSGVHTFPAVLQSSGLYSLSSVVTVPSSSLGTQTYICNVNHK
PSNTKVDKKVEPKSCDKTHTCPPCPAPELLGGPSVFLFPPKPKDT
LMISRTPEVTCVVVDVSHEDPEVKFNWYVDGVEVHNAKTKPREEQ
YNSTYRVVSVLTVLHQDWLNGKEYKCKVSNKALPAPIEKTISKAK
GQPREPQVYTLPPSRDELTKNQVSLTCLVKGFYPSDIAVEWESNG
QPENNYKTTPPVLDSDGSFFLYSKLTVDKSRWQQGNVFSCSVLHE
ALHSHYTQKSLSLSPGK
SEQ ID NO: 274
QSALTQPPSASGSPGQSISISCTGTSNRFVSWYQQHPGKAPKLVI
YGVNKRPSGVPDRFSGSKSGNTASLTVSGLQTDDEAVYYCSSLVG
NWDVIFGGGTKLTVLGQPKAAPSVTLFPPSSEELQANKATLVCLI
SDFYPGAVTVAWKADSSPVKAGVETTTPSKQSNNKYAASSYLSLT
PEQWKSHRSYSCQVTHEGSTVEKTVAPTECS
SEQ ID NO: 275
QPQLQESGPGLVEASETLSLTCTVSGDSTGRCNYFWGWVRQPPGK
GLEWIGSLSHCRSYYNTDWTYHNPSLKSRLTISLDTPKNQVFLRL
TSVTAADTATYYCARFGGEVLVYRDWPKPAWVDLWGRGTLVTVSS
SEQ ID NO: 276
QSALTQPPSASGSPGQSITISCTGTSNNFVSWYQQYPGKAPKLVI
YEVNKRPSGVPDRFSGSKSGSTASLTVSGLQADDEGVYYCSSLVG
NWDVIFGGGTKLTVL
SEQ ID NO: 277
QPQLQESGPGLVEASETLSLTCTVSGDSTGRCNYFWGWVRQPPGK
GLEWIGSLSHCRSYYNTDWTYHNPSLKSRLTISLDTPKNQVFLRL
TSVTAADTATYYCARFGGEVLVYRDWPKPAWVDLWGRGTLVTVSS
ASTKGPSVFPLAPSSKSTSGGTAALGCLVKDYFPEPVTVSWNSGA
LTSGVHTFPAVLQSSGLYSLSSVVTVPSSSLGTQTYICNVNHKPS
NTKVDKKVEPKSCDKTHTCPPCPAPELLGGPSVFLFPPKPKDTLM
ISRTPEVTCVVVDVSHEDPEVKFNWYVDGVEVHNAKTKPREEQYN
STYRVVSVLTVLHQDWLNGKEYKCKVSNKALPAPIEKTISKAKGQ
PREPQVYTLPPSRDELTKNQVSLTCLVKGFYPSDIAVEWESNGQP
ENNYKTTPPVLDSDGSFFLYSKLTVDKSRWQQGNVFSCSVMHEAL
HNHYTQKSLSLSPGK
SEQ ID NO: 278
QPQLQESGPGLVEASETLSLTCTVSGDSTGRCNYFWGWVRQPPGK
GLEWIGSLSHCRSYYNTDWTYHNPSLKSRLTISLDTPKNQVFLRL
TSVTAADTATYYCARFGGEVLVYRDWPKPAWVDLWGRGTLVTVSS
ASTKGPSVFPLAPSSKSTSGGTAALGCLVKDYFPEPVTVSWNSGA
LTSGVHTFPAVLQSSGLYSLSSVVTVPSSSLGTQTYICNVNHKPS
NTKVDKKVEPKSCDKTHTCPPCPAPELLGGPSVFLFPPKPKDTLM
ISRTPEVTCVVVDVSHEDPEVKFNWYVDGVEVHNAKTKPREEQYN
STYRVVSVLTVLHQDWLNGKEYKCKVSNKALPAPIEKTISKAKGQ
PREPQVYTLPPSRDELTKNQVSLTCLVKGFYPSDIAVEWESNGQP
ENNYKTTPPVLDSDGSFFLYSKLTVDKSRWQQGNVFSCSVLHEAL
HSHYTQKSLSLSPGK
SEQ ID NO: 279
QSALTQPPSASGSPGQSITISCTGTSNNFVSWYQQYPGKAPKLVI
YEVNKRPSGVPDRFSGSKSGSTASLTVSGLQADDEGVYYCSSLVG
NWDVIFGGGTKLTVLGQPKAAPSVTLFPPSSEELQANKATLVCLI
SDFYPGAVTVAWKADSSPVKAGVETTTPSKQSNNKYAASSYLSLT
PEQWKSHRSYSCQVTHEGSTVEKTVAPTECS
SEQ ID NO: 280
QVQLQESGPGLVKPAETLSLTCSVSGESINTGHYYWGWVRQVPGK
GLEWIGHIHYTTAVLHNPSLKSRLTIKIYTLRNQITLRLSNVTAA
DTAVYHCVRSGGDILYYYEWQKPHWFSPWGPGIHVTVSS
SEQ ID NO: 281
QSALTQPPSASGSLGQSVTISCNGTSSDIGGWNFVSWYQQFPGRA
PRLIIFEVNKRPSGVPGRFSGSKSGNSASLTVSGLQSDDEGQYFC
SSLFGRWDVVFGGGTKLTVL
SEQ ID NO: 282
QVQLQESGPGLVKPAETLSLTCSVSGESINTGHYYWGWVRQVPGK
GLEWIGHIHYTTAVLHNPSLKSRLTIKIYTLRNQITLRLSNVTAA
DTAVYHCVRSGGDILYYYEWQKPHWFSPWGPGIHVTVSSASTKGP
SVFPLAPSSKSTSGGTAALGCLVKDYFPEPVTVSWNSGALTSGVH
TFPAVLQSSGLYSLSSVVTVPSSSLGTQTYICNVNHKPSNTKVDK
KVEPKSCDKTHTCPPCPAPELLGGPSVFLFPPKPKDTLMISRTPE
VTCVVVDVSHEDPEVKFNWYVDGVEVHNAKTKPREEQYNSTYRVV
SVLTVLHQDWLNGKEYKCKVSNKALPAPIEKTISKAKGQPREPQV
YTLPPSRDELTKNQVSLTCLVKGFYPSDIAVEWESNGQPENNYKT
TPPVLDSDGSFFLYSKLTVDKSRWQQGNVFSCSVMHEALHNHYTQ
KSLSLSPGK
SEQ ID NO: 283
QVQLQESGPGLVKPAETLSLTCSVSGESINTGHYYWGWVRQVPGK
GLEWIGHIHYTTAVLHNPSLKSRLTIKIYTLRNQITLRLSNVTAA
DTAVYHCVRSGGDILYYYEWQKPHWFSPWGPGIHVTVSSASTKGP
SVFPLAPSSKSTSGGTAALGCLVKDYFPEPVTVSWNSGALTSGVH
TFPAVLQSSGLYSLSSVVTVPSSSLGTQTYICNVNHKPSNTKVDK
KVEPKSCDKTHTCPPCPAPELLGGPSVFLFPPKPKDTLMISRTPE
VTCVVVDVSHEDPEVKFNWYVDGVEVHNAKTKPREEQYNSTYRVV
SVLTVLHQDWLNGKEYKCKVSNKALPAPIEKTISKAKGQPREPQV
YTLPPSRDELTKNQVSLTCLVKGFYPSDIAVEWESNGQPENNYKT
TPPVLDSDGSFFLYSKLTVDKSRWQQGNVFSCSVLHEALHSHYTQ
KSLSLSPGK
SEQ ID NO: 284
QSALTQPPSASGSLGQSVTISCNGTSSDIGGWNFVSWYQQFPGRA
PRLIIFEVNKRPSGVPGRFSGSKSGNSASLTVSGLQSDDEGQYFC
SSLFGRWDVVFGGGTKLTVLGQPKAAPSVTLFPPSSEELQANKAT
LVCLISDFYPGAVTVAWKADSSPVKAGVETTTPSKQSNNKYAASS
YLSLTPEQWKSHRSYSCQVTHEGSTVEKTVAPTECS
SEQ ID NO: 285
QVQLQESGPGLVKPSETLSLTCTVSGDSINTGHHYWGWVRQVPGK
GPEWIAHIHYNTAVLHNPALKSRVTISIFTLKNLITLSLSNVTAA
DTAVYFCVRSGGDILYYIEWQKPHWFYPWGPGILVTVSS
SEQ ID NO: 286
QSALTQPPSASGSLGQSLTISCSGTGSDIGSWNFVSWYQQFPGRA
PNLIIFEVNRRRSGVPDRFSGSKSGNTASLTVSGLRSEDEAEYFC
SSLSGRWDIVFGGGTKVTVL
SEQ ID NO: 287
QVQLQESGPGLVKPSETLSLTCTVSGDSINTGHHYWGWVRQVPGK
GPEWIAHIHYNTAVLHNPALKSRVTISIFTLKNLITLSLSNVTAA
DTAVYFCVRSGGDILYYIEWQKPHWFYPWGPGILVTVSSASTKGP
SVFPLAPSSKSTSGGTAALGCLVKDYFPEPVTVSWNSGALTSGVH
TFPAVLQSSGLYSLSSVVTVPSSSLGTQTYICNVNHKPSNTKVDK
KVEPKSCDKTHTCPPCPAPELLGGPSVFLFPPKPKDTLMISRTPE
VTCVVVDVSHEDPEVKFNWYVDGVEVHNAKTKPREEQYNSTYRVV
SVLTVLHQDWLNGKEYKCKVSNKALPAPIEKTISKAKGQPREPQV
YTLPPSRDELTKNQVSLTCLVKGFYPSDIAVEWESNGQPENNYKT
TPPVLDSDGSFFLYSKLTVDKSRWQQGNVFSCSVMHEALHNHYTQ
KSLSLSPGK
SEQ ID NO: 288
QVQLQESGPGLVKPSETLSLTCTVSGDSINTGHHYWGWVRQVPGK
GPEWIAHIHYNTAVLHNPALKSRVTISIFTLKNLITLSLSNVTAA
DTAVYFCVRSGGDILYYIEWQKPHWFYPWGPGILVTVSSASTKGP
SVFPLAPSSKSTSGGTAALGCLVKDYFPEPVTVSWNSGALTSGVH
TFPAVLQSSGLYSLSSVVTVPSSSLGTQTYICNVNHKPSNTKVDK
KVEPKSCDKTHTCPPCPAPELLGGPSVFLFPPKPKDTLMISRTPE
VTCVVVDVSHEDPEVKFNWYVDGVEVHNAKTKPREEQYNSTYRVV
SVLTVLHQDWLNGKEYKCKVSNKALPAPIEKTISKAKGQPREPQV
YTLPPSRDELTKNQVSLTCLVKGFYPSDIAVEWESNGQPENNYKT
TPPVLDSDGSFFLYSKLTVDKSRWQQGNVFSCSVLHEALHSHYTQ
KSLSLSPGK
SEQ ID NO: 289
QSALTQPPSASGSLGQSLTISCSGTGSDIGSWNFVSWYQQFPGRA
PNLIIFEVNRRRSGVPDRFSGSKSGNTASLTVSGLRSEDEAEYFC
SSLSGRWDIVFGGGTKVTVLGQPKAAPSVTLFPPSSEELQANKAT
LVCLISDFYPGAVTVAWKADSSPVKAGVETTTPSKQSNNKYAASS
YLSLTPEQWKSHRSYSCQVTHEGSTVEKTVAPTECS
SEQ ID NO: 290
QLQLQESGPGLVKPSETLSLTCTVSGGSMRGTDWGENDFHYGWIR
QSSAKGLEWIGSIHWRGRTTHYKTSFRSRATLSIDTSNNRFSLTF
SFVTAADTAVYYCARHKYHDIFRVVPVAGWFDPWGQGLLVTVSS
SEQ ID NO: 291
EIVMTQSPPTLSVSPGETATLSCRASQNVKNNLAWYQLKPGQAPR
LLIFDASSRAGGIPDRFSGSGYGTDFTLTVNSVQSEDFGDYFCQQ
YEEWPRTFGQGTKVDIK
SEQ ID NO: 292
QLQLQESGPGLVKPSETLSLTCTVSGGSMRGTDWGENDFHYGWIR
QSSAKGLEWIGSIHWRGRTTHYKTSFRSRATLSIDTSNNRFSLTF
SFVTAADTAVYYCARHKYHDIFRVVPVAGWFDPWGQGLLVTVSSA
STKGPSVFPLAPSSKSTSGGTAALGCLVKDYFPEPVTVSWNSGAL
TSGVHTFPAVLQSSGLYSLSSVVTVPSSSLGTQTYICNVNHKPSN
TKVDKKVEPKSCDKTHTCPPCPAPELLGGPSVFLFPPKPKDTLMI
SRTPEVTCVVVDVSHEDPEVKFNWYVDGVEVHNAKTKPREEQYNS
TYRVVSVLTVLHQDWLNGKEYKCKVSNKALPAPIEKTISKAKGQP
REPQVYTLPPSRDELTKNQVSLTCLVKGFYPSDIAVEWESNGQPE
NNYKTTPPVLDSDGSFFLYSKLTVDKSRWQQGNVFSCSVMHEALH
NHYTQKSLSLSPGK
SEQ ID NO: 293
QLQLQESGPGLVKPSETLSLTCTVSGGSMRGTDWGENDFHYGWIR
QSSAKGLEWIGSIHWRGRTTHYKTSFRSRATLSIDTSNNRFSLTF
SFVTAADTAVYYCARHKYHDIFRVVPVAGWFDPWGQGLLVTVSSA
STKGPSVFPLAPSSKSTSGGTAALGCLVKDYFPEPVTVSWNSGAL
TSGVHTFPAVLQSSGLYSLSSVVTVPSSSLGTQTYICNVNHKPSN
TKVDKKVEPKSCDKTHTCPPCPAPELLGGPSVFLFPPKPKDTLMI
SRTPEVTCVVVDVSHEDPEVKFNWYVDGVEVHNAKTKPREEQYNS
TYRVVSVLTVLHQDWLNGKEYKCKVSNKALPAPIEKTISKAKGQP
REPQVYTLPPSRDELTKNQVSLTCLVKGFYPSDIAVEWESNGQPE
NNYKTTPPVLDSDGSFFLYSKLTVDKSRWQQGNVFSCSVLHEALH
SHYTQKSLSLSPGK
SEQ ID NO: 294
EIVMTQSPPTLSVSPGETATLSCRASQNVKNNLAWYQLKPGQAPR
LLIFDASSRAGGIPDRFSGSGYGTDFTLTVNSVQSEDFGDYFCQQ
YEEWPRTFGQGTKVDIKRTVAAPSVFIFPPSDEQLKSGTASVVCL
LNNFYPREAKVQWKVDNALQSGNSQESVTEQDSKDSTYSLSSTLT
LSKADYEKHKVYACEVTHQGLSSPVTKSFNRGEC
SEQ ID NO: 295
EVHLEESGPGLVRPSETLSLTCTASGGSIRGGEWGDSDYHWGWVR
HSPEKGLEWIGSIHWRGTTHYNAPFRGRGRLSIDLSRNQFSLRLT
SVTAEDTAVYYCVKHKYHDIVMVVPIAGWFDPWGQGLQVTVSS
SEQ ID NO: 296
EIMMTQSPAILSVSPGDRATLSCRASQSVKNNLAWYQKRPGQAPR
LLIFDTSSRASGIPARFSGGGSGTEFTLTVNSMQSEDFATYYCQQ
YEEWPRTFGQGTKVEIK
SEQ ID NO: 297
EVHLEESGPGLVRPSETLSLTCTASGGSIRGGEWGDSDYHWGWVR
HSPEKGLEWIGSIHWRGTTHYNAPFRGRGRLSIDLSRNQFSLRLT
SVTAEDTAVYYCVKHKYHDIVMVVPIAGWFDPWGQGLQVTVSSAS
TKGPSVFPLAPSSKSTSGGTAALGCLVKDYFPEPVTVSWNSGALT
SGVHTFPAVLQSSGLYSLSSVVTVPSSSLGTQTYICNVNHKPSNT
KVDKKVEPKSCDKTHTCPPCPAPELLGGPSVFLFPPKPKDTLMIS
RTPEVTCVVVDVSHEDPEVKFNWYVDGVEVHNAKTKPREEQYNST
YRVVSVLTVLHQDWLNGKEYKCKVSNKALPAPIEKTISKAKGQPR
EPQVYTLPPSRDELTKNQVSLTCLVKGFYPSDIAVEWESNGQPEN
NYKTTPPVLDSDGSFFLYSKLTVDKSRWQQGNVFSCSVMHEALHN
HYTQKSLSLSPGK
SEQ ID NO: 298
EVHLEESGPGLVRPSETLSLTCTASGGSIRGGEWGDSDYHWGWVR
HSPEKGLEWIGSIHWRGTTHYNAPFRGRGRLSIDLSRNQFSLRLT
SVTAEDTAVYYCVKHKYHDIVMVVPIAGWFDPWGQGLQVTVSSAS
TKGPSVFPLAPSSKSTSGGTAALGCLVKDYFPEPVTVSWNSGALT
SGVHTFPAVLQSSGLYSLSSVVTVPSSSLGTQTYICNVNHKPSNT
KVDKKVEPKSCDKTHTCPPCPAPELLGGPSVFLFPPKPKDTLMIS
RTPEVTCVWDVSHEDPEVKFNWYVDGVEVHNAKTKPREEQYNSTY
RVVSVLTVLHQDWLNGKEYKCKVSNKALPAPIEKTISKAKGQPRE
PQVYTLPPSRDELTKNQVSLTCLVKGFYPSDIAVEWESNGQPENN
YKTTPPVLDSDGSFFLYSKLTVDKSRWQQGNVFSCSVLHEALHSH
YTQKSLSLSPGK
SEQ ID NO: 299
EIMMTQSPAILSVSPGDRATLSCRASQSVKNNLAWYQKRPGQAPR
LLIFDTSSRASGIPARFSGGGSGTEFTLTVNSMQSEDFATYYCQQ
YEEWPRTFGQGTKVEIKRTVAAPSVFIFPPSDEQLKSGTASVVCL
LNNFYPREAKVQWKVDNALQSGNSQESVTEQDSKDSTYSLSSTLT
LSKADYEKHKVYACEVTHQGLSSPVTKSFNRGEC
SEQ ID NO: 300
EVOLVESGGGLVKPGGSLRLTCVASGFTFSDVWLNWVRQAPGKGL
EWVGRIKSRTDGGTTDYAASVKGRFTISRDDSKNTLYLQMNSLKT
EDTAVYSCTTDGFIMIRGVSEDYYYYYMDVWGKGTTVTVSS
SEQ ID NO: 301
QSVLTQPPSVSAAPGQKVTISCSGSSSNIGNNYVLWYQQFPGTAP
KLLIYGNNKRPSGIPDRFSGSKSGTSATLGITGLQTGDEADYFCA
TWDSGLSADWVFGGGTKLTVL
SEQ ID NO: 302
EVOLVESGGGLVKPGGSLRLTCVASGFTFSDVWLNWVRQAPGKGL
EWVGRIKSRTDGGTTDYAASVKGRFTISRDDSKNTLYLQMNSLKT
EDTAVYSCTTDGFIMIRGVSEDYYYYYMDVWGKGTTVTVSSASTK
GPSVFPLAPSSKSTSGGTAALGCLVKDYFPEPVTVSWNSGALTSG
VHTFPAVLQSSGLYSLSSVVTVPSSSLGTQTYICNVNHKPSNTKV
DKKVEPKSCDKTHTCPPCPAPELLGGPSVFLFPPKPKDTLMISRT
PEVTCVVVDVSHEDPEVKFNWYVDGVEVHNAKTKPREEQYNSTYR
VVSVLTVLHQDWLNGKEYKCKVSNKALPAPIEKTISKAKGQPREP
QVYTLPPSRDELTKNQVSLTCLVKGFYPSDIAVEWESNGQPENNY
KTTPPVLDSDGSFFLYSKLTVDKSRWQQGNVFSCSVMHEALHNHY
TQKSLSLSPGK
SEQ ID NO: 303
EVOLVESGGGLVKPGGSLRLTCVASGFTFSDVWLNWVRQAPGKGL
EWVGRIKSRTDGGTTDYAASVKGRFTISRDDSKNTLYLQMNSLKT
EDTAVYSCTTDGFIMIRGVSEDYYYYYMDVWGKGTTVTVSSASTK
GPSVFPLAPSSKSTSGGTAALGCLVKDYFPEPVTVSWNSGALTSG
VHTFPAVLQSSGLYSLSSVVTVPSSSLGTQTYICNVNHKPSNTKV
DKKVEPKSCDKTHTCPPCPAPELLGGPSVFLFPPKPKDTLMISRT
PEVTCVVVDVSHEDPEVKFNWYVDGVEVHNAKTKPREEQYNSTYR
VVSVLTVLHQDWLNGKEYKCKVSNKALPAPIEKTISKAKGQPREP
QVYTLPPSRDELTKNQVSLTCLVKGFYPSDIAVEWESNGQPENNY
KTTPPVLDSDGSFFLYSKLTVDKSRWQQGNVFSCSVLHEALHSHY
TQKSLSLSPGK
SEQ ID NO: 304
QSVLTQPPSVSAAPGQKVTISCSGSSSNIGNNYVLWYQQFPGTAP
KLLIYGNNKRPSGIPDRFSGSKSGTSATLGITGLQTGDEADYFCA
TWDSGLSADWVFGGGTKLTVLGQPKAAPSVTLFPPSSEELQANKA
TLVCLISDFYPGAVTVAWKADSSPVKAGVETTTPSKQSNNKYAAS
SYLSLTPEQWKSHRSYSCQVTHEGSTVEKTVAPTECS
SEQ ID NO: 305
QVQLVQSGGGLVQPGGSLRLSCAAFGFNFSSYVMHWVRQAPGQGL
EYLSAISSDGETTYHANSVKGRFTSSRDNSKNTLFLQMGSLRTED
VAVYYCARDRYYETSGSNAFDVWGQGTMVVVSS
SEQ ID NO: 306
NSVLTQSPSSLSASVGDRVTITCQASQDISNYLNWYQHKPGKAPK
LLIYTASNLETGVPSRFSGGGSGTHFSFTITSLQPEDAATYFCQQ
YDNLGDLSFGGGTKVEIK
SEQ ID NO: 307
QVQLVQSGGGLVQPGGSLRLSCAAFGFNFSSYVMHWVRQAPGQGL
EYLSAISSDGETTYHANSVKGRFTSSRDNSKNTLFLQMGSLRTED
VAVYYCARDRYYETSGSNAFDVWGQGTMVVVSSASTKGPSVFPLA
PSSKSTSGGTAALGCLVKDYFPEPVTVSWNSGALTSGVHTFPAVL
QSSGLYSLSSVVTVPSSSLGTQTYICNVNHKPSNTKVDKKVEPKS
CDKTHTCPPCPAPELLGGPSVFLFPPKPKDTLMISRTPEVTCVVV
DVSHEDPEVKFNWYVDGVEVHNAKTKPREEQYNSTYRVVSVLTVL
HQDWLNGKEYKCKVSNKALPAPIEKTISKAKGQPREPQVYTLPPS
RDELTKNQVSLTCLVKGFYPSDIAVEWESNGQPENNYKTTPPVLD
SDGSFFLYSKLTVDKSRWQQGNVFSCSVMHEALHNHYTQKSLSLS
PGK
SEQ ID NO: 308
QVQLVQSGGGLVQPGGSLRLSCAAFGFNFSSYVMHWVRQAPGQGL
EYLSAISSDGETTYHANSVKGRFTSSRDNSKNTLFLQMGSLRTED
VAVYYCARDRYYETSGSNAFDVWGQGTMVVVSSASTKGPSVFPLA
PSSKSTSGGTAALGCLVKDYFPEPVTVSWNSGALTSGVHTFPAVL
QSSGLYSLSSVVTVPSSSLGTQTYICNVNHKPSNTKVDKKVEPKS
CDKTHTCPPCPAPELLGGPSVFLFPPKPKDTLMISRTPEVTCVVV
DVSHEDPEVKFNWYVDGVEVHNAKTKPREEQYNSTYRVVSVLTVL
HQDWLNGKEYKCKVSNKALPAPIEKTISKAKGQPREPQVYTLPPS
RDELTKNQVSLTCLVKGFYPSDIAVEWESNGQPENNYKTTPPVLD
SDGSFFLYSKLTVDKSRWQQGNVFSCSVLHEALHSHYTQKSLSLS
PGK
SEQ ID NO: 309
NSVLTQSPSSLSASVGDRVTITCQASQDISNYLNWYQHKPGKAPK
LLIYTASNLETGVPSRFSGGGSGTHFSFTITSLQPEDAATYFCQQ
YDNLGDLSFGGGTKVEIKRTVAAPSVFIFPPSDEQLKSGTASVVC
LLNNFYPREAKVQWKVDNALQSGNSQESVTEQDSKDSTYSLSSTL
TLSKADYEKHKVYACEVTHQGLSSPVTKSFNRGEC
SEQ ID NO: 310
QVQLVQSGAEVRKPGSSVTISCKPVGGTFTNFAIHWVRQAPGQGL
EWVGGRVPVVGIYKYGKKFHDRLRLYEDDPMKTVFLELRSLTSDD
TGVYYCTRWRGCGMCPYDTSSYYNDASDVWGPGTKVIVSA
SEQ ID NO: 311
EIVLTQSPVTLSLSSGETGTLSCRASQNISSSWIAWYQQRRGQVP
RLLISAASARAAGIPDRFTGRGSGTDFTLTITRLEPEDFGVYSCQ
YYGGSFFTFGPGTQVDVK
SEQ ID NO: 312
QVQLVQSGAEVRKPGSSVTISCKPVGGTFTNFAIHWVRQAPGQGL
EWVGGRVPVVGIYKYGKKFHDRLRLYEDDPMKTVFLELRSLTSDD
TGVYYCTRWRGCGMCPYDTSSYYNDASDVWGPGTKVIVSAASTKG
PSVFPLAPSSKSTSGGTAALGCLVKDYFPEPVTVSWNSGALTSGV
HTFPAVLQSSGLYSLSSVVTVPSSSLGTQTYICNVNHKPSNTKVD
KKVEPKSCDKTHTCPPCPAPELLGGPSVFLFPPKPKDTLMISRTP
EVTCVVVDVSHEDPEVKFNWYVDGVEVHNAKTKPREEQYNSTYRV
VSVLTVLHQDWLNGKEYKCKVSNKALPAPIEKTISKAKGQPREPQ
VYTLPPSRDELTKNQVSLTCLVKGFYPSDIAVEWESNGQPENNYK
TTPPVLDSDGSFFLYSKLTVDKSRWQQGNVFSCSVMHEALHNHYT
QKSLSLSPGK
SEQ ID NO: 313
QVQLVQSGAEVRKPGSSVTISCKPVGGTFTNFAIHWVRQAPGQGL
EWVGGRVPVVGIYKYGKKFHDRLRLYEDDPMKTVFLELRSLTSDD
TGVYYCTRWRGCGMCPYDTSSYYNDASDVWGPGTKVIVSAASTKG
PSVFPLAPSSKSTSGGTAALGCLVKDYFPEPVTVSWNSGALTSGV
HTFPAVLQSSGLYSLSSVVTVPSSSLGTQTYICNVNHKPSNTKVD
KKVEPKSCDKTHTCPPCPAPELLGGPSVFLFPPKPKDTLMISRTP
EVTCVVVDVSHEDPEVKFNWYVDGVEVHNAKTKPREEQYNSTYRV
VSVLTVLHQDWLNGKEYKCKVSNKALPAPIEKTISKAKGQPREPQ
VYTLPPSRDELTKNQVSLTCLVKGFYPSDIAVEWESNGQPENNYK
TTPPVLDSDGSFFLYSKLTVDKSRWQQGNVFSCSVLHEALHSHYT
QKSLSLSPGK
SEQ ID NO: 314
EIVLTQSPVTLSLSSGETGTLSCRASQNISSSWIAWYQQRRGQVP
RLLISAASARAAGIPDRFTGRGSGTDFTLTITRLEPEDFGVYSCQ
YYGGSFFTFGPGTQVDVKRTVAAPSVFIFPPSDEQLKSGTASVVC
LLNNFYPREAKVQWKVDNALQSGNSQESVTEQDSKDSTYSLSSTL
TLSKADYEKHKVYACEVTHQGLSSPVTKSFNRGEC
SEQ ID NO: 315
EVQLVESGGGLVKAGGSLILSCGVSNFRISAHTMNWVRRVPGGGL
EWVASISTSSTYRDYADAVKGRFTVSRDDLEDFVYLQMHKMRVED
TAIYYCARKGSDRLSDNDPFDAWGPGTVVTVSP
SEQ ID NO: 316
DVVMTQSPSTLSASVGDTITITCRASQSIETWLAWYQQKPGKAPK
LLIYKASTLKTGVPSRFSGSGSGTEFTLTISGLQFDDFATYHCQH
YAGYSATFGQGTRVEIK
SEQ ID NO: 317
EVOLVESGGGLVKAGGSLILSCGVSNFRISAHTMNWVRRVPGGGL
EWVASISTSSTYRDYADAVKGRFTVSRDDLEDFVYLQMHKMRVED
TAIYYCARKGSDRLSDNDPFDAWGPGTVVTVSPASTKGPSVFPLA
PSSKSTSGGTAALGCLVKDYFPEPVTVSWNSGALTSGVHTFPAVL
QSSGLYSLSSWVTVPSSSLGTQTYICNVNHKPSNTKVDKKVEPKS
CDKTHTCPPCPAPELLGGPSVFLFPPKPKDTLMISRTPEVTCVVV
DVSHEDPEVKFNWYVDGVEVHNAKTKPREEQYNSTYRVVSVLTVL
HQDWLNGKEYKCKVSNKALPAPIEKTISKAKGQPREPQVYTLPPS
RDELTKNQVSLTCLVKGFYPSDIAVEWESNGQPENNYKTTPPVLD
SDGSFFLYSKLTVDKSRWQQGNVFSCSVMHEALHNHYTQKSLSLS
PGK
SEQ ID NO: 318
EVOLVESGGGLVKAGGSLILSCGVSNFRISAHTMNWVRRVPGGGL
EWVASISTSSTYRDYADAVKGRFTVSRDDLEDFVYLQMHKMRVED
TAIYYCARKGSDRLSDNDPFDAWGPGTVVTVSPASTKGPSVFPLA
PSSKSTSGGTAALGCLVKDYFPEPVTVSWNSGALTSGVHTFPAVL
QSSGLYSLSSWVTVPSSSLGTQTYICNVNHKPSNTKVDKKVEPKS
CDKTHTCPPCPAPELLGGPSVFLFPPKPKDTLMISRTPEVTCVVV
DVSHEDPEVKFNWYVDGVEVHNAKTKPREEQYNSTYRVVSVLTVL
HQDWLNGKEYKCKVSNKALPAPIEKTISKAKGQPREPQVYTLPPS
RDELTKNQVSLTCLVKGFYPSDIAVEWESNGQPENNYKTTPPVLD
SDGSFFLYSKLTVDKSRWQQGNVFSCSVLHEALHSHYTQKSLSLS
PGK
SEQ ID NO: 319
DVVMTQSPSTLSASVGDTITITCRASQSIETWLAWYQQKPGKAPK
LLIYKASTLKTGVPSRFSGSGSGTEFTLTISGLQFDDFATYHCQH
YAGYSATFGQGTRVEIKRTVAAPSVFIFPPSDEQLKSGTASWVCL
LNNFYPREAKVQWKVDNALQSGNSQESVTEQDSKDSTYSLSSTLT
LSKADYEKHKVYACEVTHQGLSSPVTKSFNRGEC
SEQ ID NO: 320
QMQLQESGPGLVKPGETLSLTCSVSGASISSSYWSWLRETPGKGL
EWIGYTHHSGDTNYAPSLKSRVHLGLHPSKNQVSLSLTSVTAADT
AVYYCARTLHGRRIYGVVAFNEFFTYFYWEVWGKGTQVTVSS
SEQ ID NO: 321
SDISVAPGETVRISCGGESIGSRAVQWYQHRAGQAPKLIIYNNQD
RPPGIPERFSGSPDIDFGTTATLTITNVEAGDEATYYCHIWDSRR
PTNWVFGGGTTLTVL
SEQ ID NO: 322
QMQLQESGPGLVKPGETLSLTCSVSGASISSSYWSWLRETPGKGL
EWIGYTHHSGDTNYAPSLKSRVHLGLHPSKNQVSLSLTSVTAADT
AVYYCARTLHGRRIYGVVAFNEFFTYFYWEVWGKGTQVTVSSAST
KGPSVFPLAPSSKSTSGGTAALGCLVKDYFPEPVTVSWNSGALTS
GVHTFPAVLQSSGLYSLSSVVTVPSSSLGTQTYICNVNHKPSNTK
VDKKVEPKSCDKTHTCPPCPAPELLGGPSVFLFPPKPKDTLMISR
TPEVTCVVVDVSHEDPEVKFNWYVDGVEVHNAKTKPREEQYNSTY
RVVSVLTVLHQDWLNGKEYKCKVSNKALPAPIEKTISKAKGQPRE
PQVYTLPPSRDELTKNQVSLTCLVKGFYPSDIAVEWESNGQPENN
YKTTPPVLDSDGSFFLYSKLTVDKSRWQQGNVFSCSVMHEALHNH
YTQKSLSLSPGK
SEQ ID NO: 323
QMQLQESGPGLVKPGETLSLTCSVSGASISSSYWSWLRETPGKGL
EWIGYTHHSGDTNYAPSLKSRVHLGLHPSKNQVSLSLTSVTAADT
AVYYCARTLHGRRIYGVVAFNEFFTYFYWEVWGKGTQVTVSSAST
KGPSVFPLAPSSKSTSGGTAALGCLVKDYFPEPVTVSWNSGALTS
GVHTFPAVLQSSGLYSLSSVVTVPSSSLGTQTYICNVNHKPSNTK
VDKKVEPKSCDKTHTCPPCPAPELLGGPSVFLFPPKPKDTLMISR
TPEVTCVVVDVSHEDPEVKFNWYVDGVEVHNAKTKPREEQYNSTY
RVVSVLTVLHQDWLNGKEYKCKVSNKALPAPIEKTISKAKGQPRE
PQVYTLPPSRDELTKNQVSLTCLVKGFYPSDIAVEWESNGQPENN
YKTTPPVLDSDGSFFLYSKLTVDKSRWQQGNVFSCSVLHEALHSH
YTQKSLSLSPGK
SEQ ID NO: 324
SDISVAPGETVRISCGGESIGSRAVQWYQHRAGQAPKLIIYNNQD
RPPGIPERFSGSPDIDFGTTATLTITNVEAGDEATYYCHIWDSRR
PTNWVFGGGTTLTVLGQPKAAPSVTLFPPSSEELQANKATLVCLI
SDFYPGAVTVAWKADSSPVKAGVETTTPSKQSNNKYAASSYLSLT
PEQWKSHRSYSCQVTHEGSTVEKTVAPTECS
SEQ ID NO: 325
QMQLQESGPGLVKPGETLSLTCSVSGASISSSYWSWLRETPGKGL
EWIGYTHHSGDTNYAPSLKSRVHLGLHPSKNQVSLSLTSVTAADT
AVYYCARTLHGRRIYGVVAFNEYYTYFYWPTWGKGTQVTVSS
SEQ ID NO: 326
SDISVAPGETVRITCGGESIGSRAVQWYQHRPGQAPRLIIYNNQD
RPPGIPERFSGSPDIDFGTTATLTISNVEAGDEATYYCHIWDSRR
PTNWELGPGTTLTVL
SEQ ID NO: 327
QMQLQESGPGLVKPGETLSLTCSVSGASISSSYWSWLRETPGKGL
EWIGYTHHSGDTNYAPSLKSRVHLGLHPSKNQVSLSLTSVTAADT
AVYYCARTLHGRRIYGVVAFNEYYTYFYWPTWGKGTQVTVSSAST
KGPSVFPLAPSSKSTSGGTAALGCLVKDYFPEPVTVSWNSGALTS
GVHTFPAVLQSSGLYSLSSVVTVPSSSLGTQTYICNVNHKPSNTK
VDKKVEPKSCDKTHTCPPCPAPELLGGPSVFLFPPKPKDTLMISR
TPEVTCVVVDVSHEDPEVKFNWYVDGVEVHNAKTKPREEQYNSTY
RVVSVLTVLHQDWLNGKEYKCKVSNKALPAPIEKTISKAKGQPRE
PQVYTLPPSRDELTKNQVSLTCLVKGFYPSDIAVEWESNGQPENN
YKTTPPVLDSDGSFFLYSKLTVDKSRWQQGNVFSCSVMHEALHNH
YTQKSLSLSPGK
SEQ ID NO: 328
QMQLQESGPGLVKPGETLSLTCSVSGASISSSYWSWLRETPGKGL
EWIGYTHHSGDTNYAPSLKSRVHLGLHPSKNQVSLSLTSVTAADT
AVYYCARTLHGRRIYGVVAFNEYYTYFYWPTWGKGTQVTVSSAST
KGPSVFPLAPSSKSTSGGTAALGCLVKDYFPEPVTVSWNSGALTS
GVHTFPAVLQSSGLYSLSSVVTVPSSSLGTQTYICNVNHKPSNTK
VDKKVEPKSCDKTHTCPPCPAPELLGGPSVFLFPPKPKDTLMISR
TPEVTCVVVDVSHEDPEVKFNWYVDGVEVHNAKTKPREEQYNSTY
RVVSVLTVLHQDWLNGKEYKCKVSNKALPAPIEKTISKAKGQPRE
PQVYTLPPSRDELTKNQVSLTCLVKGFYPSDIAVEWESNGQPENN
YKTTPPVLDSDGSFFLYSKLTVDKSRWQQGNVFSCSVLHEALHSH
YTQKSLSLSPGK
SEQ ID NO: 329
SDISVAPGETVRITCGGESIGSRAVQWYQHRPGQAPRLIIYNNQD
RPPGIPERFSGSPDIDFGTTATLTISNVEAGDEATYYCHIWDSRR
PTNWELGPGTTLTVLGQPKAAPSVTLFPPSSEELQANKATLVCLI
SDFYPGAVTVAWKADSSPVKAGVETTTPSKQSNNKYAASSYLSLT
PEQWKSHRSYSCQVTHEGSTVEKTVAPTECS
SEQ ID NO: 330
QMQLQESGPGLVKPGETLSLTCSVSGASISSSYWSWLRETPGKGL
EWIGYTHHSGDTNYAPSLKSRVTIGLDPSKNQVSLSLTSVTAADT
AVYYCARTLHGRRIYGVVAFNEYYTYFYWPTWGKGTQVTVSS
SEQ ID NO: 331
QMQLQESGPGLVKPGETLSLTCSVSGASISSSYWSWLRETPGKGL
EWIGYTHHSGDTNYAPSLKSRVTIGLDPSKNQVSLSLTSVTAADT
AVYYCARTLHGRRIYGVVAFNEYYTYFYWPTWGKGTQVTVSSAST
KGPSVFPLAPSSKSTSGGTAALGCLVKDYFPEPVTVSWNSGALTS
GVHTFPAVLQSSGLYSLSSVVTVPSSSLGTQTYICNVNHKPSNTK
VDKKVEPKSCDKTHTCPPCPAPELLGGPSVFLFPPKPKDTLMISR
TPEVTCVVVDVSHEDPEVKFNWYVDGVEVHNAKTKPREEQYNSTY
RVVSVLTVLHQDWLNGKEYKCKVSNKALPAPIEKTISKAKGQPRE
PQVYTLPPSRDELTKNQVSLTCLVKGFYPSDIAVEWESNGQPENN
YKTTPPVLDSDGSFFLYSKLTVDKSRWQQGNVFSCSVMHEALHNH
YTQKSLSLSPGK
SEQ ID NO: 332
QMQLQESGPGLVKPGETLSLTCSVSGASISSSYWSWLRETPGKGL
EWIGYTHHSGDTNYAPSLKSRVTIGLDPSKNQVSLSLTSVTAADT
AVYYCARTLHGRRIYGVVAFNEYYTYFYWPTWGKGTQVTVSSAST
KGPSVFPLAPSSKSTSGGTAALGCLVKDYFPEPVTVSWNSGALTS
GVHTFPAVLQSSGLYSLSSVVTVPSSSLGTQTYICNVNHKPSNTK
VDKKVEPKSCDKTHTCPPCPAPELLGGPSVFLFPPKPKDTLMISR
TPEVTCVVVDVSHEDPEVKFNWYVDGVEVHNAKTKPREEQYNSTY
RVVSVLTVLHQDWLNGKEYKCKVSNKALPAPIEKTISKAKGQPRE
PQVYTLPPSRDELTKNQVSLTCLVKGFYPSDIAVEWESNGQPENN
YKTTPPVLDSDGSFFLYSKLTVDKSRWQQGNVFSCSVLHEALHSH
YTQKSLSLSPGK
SEQ ID NO: 333
QMQLQESGPGLVKPGETLSLTCSVSGASISSSYWSWLRETPGKGL
EWIGYTHHSGDTNYAPSLKSRVTIGLDPSKNQVSLSLTSVTAADT
AVYYCARTLHGRRIYGVVAFNEYYTYFYWPTWGKGTQVTVSS
SEQ ID NO: 334
QSVLTQPPSASGSPGQSVTISCTGTSSDIGASDYVSWYQQYPGEA
PKVIIYDVTKRPSGVPDRFSGSKSGTTASLTVSGLQAEDEADYYC
SSDAGRHTLLFGGGTKVTVL
SEQ ID NO: 335
QVQLLESGPGLVRPSETLTLTCSVFNSRVSGYYYSWIRQPPGRGL
EWIASTHFSLRPSRNPSLLSRVTTSIDTERYQVFLNMRSVTAADT
AVYFCARGDASGWRADYFPHWGQGTLVVVSSASTKGPSVFPLAPS
SKSTSGGTAALGCLVKDYFPEPVTVSWNSGALTSGVHTFPAVLQS
SGLYSLSSVVTVPSSSLGTQTYICNVNHKPSNTKVDKKVEPKSCD
KTHTCPPCPAPELLGGPSVFLFPPKPKDTLMISRTPEVTCVVVDV
SHEDPEVKFNWYVDGVEVHNAKTKPREEQYNSTYRVVSVLTVLHQ
DWLNGKEYKCKVSNKALPAPIEKTISKAKGQPREPQVYTLPPSRD
ELTKNQVSLTCLVKGFYPSDIAVEWESNGQPENNYKTTPPVLDSD
GSFFLYSKLTVDKSRWQQGNVFSCSVMHEALHNHYTQKSLSLSPG
K
SEQ ID NO: 336
QVQLLESGPGLVRPSETLTLTCSVFNSRVSGYYYSWIRQPPGRGL
EWIASTHFSLRPSRNPSLLSRVTTSIDTERYQVFLNMRSVTAADT
AVYFCARGDASGWRADYFPHWGQGTLVVVSSASTKGPSVFPLAPS
SKSTSGGTAALGCLVKDYFPEPVTVSWNSGALTSGVHTFPAVLQS
SGLYSLSSVVTVPSSSLGTQTYICNVNHKPSNTKVDKKVEPKSCD
KTHTCPPCPAPELLGGPSVFLFPPKPKDTLMISRTPEVTCVVVDV
SHEDPEVKFNWYVDGVEVHNAKTKPREEQYNSTYRVVSVLTVLHQ
DWLNGKEYKCKVSNKALPAPIEKTISKAKGQPREPQVYTLPPSRD
ELTKNQVSLTCLVKGFYPSDIAVEWESNGQPENNYKTTPPVLDSD
GSFFLYSKLTVDKSRWQQGNVFSCSVLHEALHSHYTQKSLSLSPG
K
SEQ ID NO: 337
QSVLTQPPSASGSPGQSVTISCTGTSSDIGASDYVSWYQQYPGEA
PKVIIYDVTKRPSGVPDRFSGSKSGTTASLTVSGLQAEDEADYYC
SSDAGRHTLLFGGGTKVTVLGQPKAAPSVTLFPPSSEELQANKAT
LVCLISDFYPGAVTVAWKADSSPVKAGVETTTPSKQSNNKYAASS
YLSLTPEQWKSHRSYSCQVTHEGSTVEKTVAPTECS
SEQ ID NO: 338
KKVVYGKCGDTVELTCTASQKKNIQFHWKNSNQIKILGNQGSFLT
KGPSKLNDRVDSRRSLWDQGNFPLIIKNLKPEDSDTYICEVEDQK
EEVQLVVVCGGGGSQVHLQESGPGLVKPSETLSLTCNVSGTLVRD
NYWSWIRQPLGKQPEWIGYVHDSGDTNYNPSLKSRVHLSLDKSKN
LVSLRLTGVTAADSAIYYCATTKHGRRIYGVVAFKEWFTYFYMDV
WGKGTSVTVSSASTKGPSVFPLAPSSKSTSGGTAALGCLVKDYFP
EPVTVSWNSGALTSGVHTFPAVLQSSGLYSLSSVVTVPSSSLGTQ
TYICNVNHKPSNTKVDKKVEPKSCDKTHTCPPCPAPELLGGPSVF
LFPPKPKDTLMISRTPEVTCVVVDVSHEDPEVKFNWYVDGVEVHN
AKTKPREEQYNSTYRVVSVLTVLHQDWLNGKEYKCKVSNKALPAP
IEKTISKAKGQPREPQVYTLPPSRDELTKNQVSLTCLVKGFYPSD
IAVEWESNGQPENNYKTTPPVLDSDGSFFLYSKLTVDKSRWQQGN
VFSCSVLHEALHSHYTQKSLSLSPGK
SEQ ID NO: 339
KKVVYGKCGDTVELTCTASQKKNIQFHWKNSNQIKILGNQGSFLT
KGPSKLNDRVDSRRSLWDQGNFPLIIKNLKPEDSDTYICEVEDQK
EEVQLVVVCGGGGSQLHLQESGPGLVKPPETLSLTCSVSGASIND
AYWSWIRQSPGKRPEWVGYVHHSGDTNYNPSLKRRVTFSLDTAKN
EVSLKLVDLTAADSATYFCARALHGKRIYGIVALGELFTYFYMDV
WGKGTAVTVSSASTKGPSVFPLAPSSKSTSGGTAALGCLVKDYFP
EPVTVSWNSGALTSGVHTFPAVLQSSGLYSLSSVVTVPSSSLGTQ
TYICNVNHKPSNTKVDKKVEPKSCDKTHTCPPCPAPELLGGPSVF
LFPPKPKDTLMISRTPEVTCVVVDVSHEDPEVKFNWYVDGVEVHN
AKTKPREEQYNSTYRVVSVLTVLHQDWLNGKEYKCKVSNKALPAP
IEKTISKAKGQPREPQVYTLPPSRDELTKNQVSLTCLVKGFYPSD
IAVEWESNGQPENNYKTTPPVLDSDGSFFLYSKLTVDKSRWQQGN
VFSCSVLHEALHSHYTQKSLSLSPGK
SEQ ID NO: 340
KKVVYGKCGDTVELTCTASQKKNIQFHWKNSNQIKILGNQGSFLT
KGPSKLNDRVDSRRSLWDQGNFPLIIKNLKPEDSDTYICEVEDQK
EEVQLVVVCGGGGSQSQLQESGPRLVEASETLSLTCNVSGESTGA
CTYFWGWVRQAPGKGLEWIGSLSHCQSFWGSGWTFHNPSLKSRLT
ISLDTPKNQVFLKLTSLTAADTATYYCARFDGEVLVYNHWPKPAW
VDLWGRGIPVTVTVSSASTKGPSVFPLAPSSKSTSGGTAALGCLV
KDYFPEPVTVSWNSGALTSGVHTFPAVLQSSGLYSLSSVVTVPSS
SLGTQTYICNVNHKPSNTKVDKKVEPKSCDKTHTCPPCPAPELLG
GPSVFLFPPKPKDTLMISRTPEVTCVVVDVSHEDPEVKFNWYVDG
VEVHNAKTKPREEQYNSTYRVVSVLTVLHQDWLNGKEYKCKVSNK
ALPAPIEKTISKAKGQPREPQVYTLPPSRDELTKNQVSLTCLVKG
FYPSDIAVEWESNGQPENNYKTTPPVLDSDGSFFLYSKLTVDKSR
WQQGNVFSCSVLHEALHSHYTQKSLSLSPGK
SEQ ID NO: 341
KKVVYGKCGDTVELTCTASQKKNIQFHWKNSNQIKILGNQGSFLT
KGPSKLNDRVDSRRSLWDQGNFPLIIKNLKPEDSDTYICEVEDQK
EEVQLVVVCGGGGSQPQLQESGPGLVEASETLSLTCTVSGDSTAA
CDYFWGWVRQPPGKGLEWIGGLSHCAGYYNTGWTYHNPSLKSRLT
ISLDTPKNQVFLKLNSVTAADTAIYYCARFDGEVLVYHDWPKPAW
VDLWGRGTLVTVTVSSASTKGPSVFPLAPSSKSTSGGTAALGCLV
KDYFPEPVTVSWNSGALTSGVHTFPAVLQSSGLYSLSSVVTVPSS
SLGTQTYICNVNHKPSNTKVDKKVEPKSCDKTHTCPPCPAPELLG
GPSVFLFPPKPKDTLMISRTPEVTCVVVDVSHEDPEVKFNWYVDG
VEVHNAKTKPREEQYNSTYRVVSVLTVLHQDWLNGKEYKCKVSNK
ALPAPIEKTISKAKGQPREPQVYTLPPSRDELTKNQVSLTCLVKG
FYPSDIAVEWESNGQPENNYKTTPPVLDSDGSFFLYSKLTVDKSR
WQQGNVFSCSVLHEALHSHYTQKSLSLSPGK
SEQ ID NO: 342
KKVVYGKCGDTVELTCTASQKKNIQFHWKNSNQIKILGNQGSFLT
KGPSKLNDRVDSRRSLWDQGNFPLIIKNLKPEDSDTYICEVEDQK
EEVQLVVVCGGGGSQPQLQESGPGLVEASETLSLTCTVSGDSTGR
CNYFWGWVRQPPGKGLEWIGSLSHCRSYYNTDWTYHNPSLKSRLT
ISLDTPKNQVFLRLTSVTAADTATYYCARFGGEVLVYRDWPKPAW
VDLWGRGTLVTVSSASTKGPSVFPLAPSSKSTSGGTAALGCLVKD
YFPEPVTVSWNSGALTSGVHTFPAVLQSSGLYSLSSVVTVPSSSL
GTQTYICNVNHKPSNTKVDKKVEPKSCDKTHTCPPCPAPELLGGP
SVFLFPPKPKDTLMISRTPEVTCVVVDVSHEDPEVKFNWYVDGVE
VHNAKTKPREEQYNSTYRVVSVLTVLHQDWLNGKEYKCKVSNKAL
PAPIEKTISKAKGQPREPQVYTLPPSRDELTKNQVSLTCLVKGFY
PSDIAVEWESNGQPENNYKTTPPVLDSDGSFFLYSKLTVDKSRWQ
QGNVFSCSVLHEALHSHYTQKSLSLSPGK
SEQ ID NO: 343
KKVVYGKCGDTVELTCTASQKKNIQFHWKNSNQIKILGNQGSFLT
KGPSKLNDRVDSRRSLWDQGNFPLIIKNLKPEDSDTYICEVEDQK
EEVQLVVVCGGGGSQVQLQESGPGLVKPAETLSLTCSVSGESINT
GHYYWGWVRQVPGKGLEWIGHIHYTTAVLHNPSLKSRLTIKIYTL
RNQITLRLSNVTAADTAVYHCVRSGGDILYYYEWQKPHWFSPWGP
GIHVTVSSASTKGPSVFPLAPSSKSTSGGTAALGCLVKDYFPEPV
TVSWNSGALTSGVHTFPAVLQSSGLYSLSSVVTVPSSSLGTQTYI
CNVNHKPSNTKVDKKVEPKSCDKTHTCPPCPAPELLGGPSVFLFP
PKPKDTLMISRTPEVTCVVVDVSHEDPEVKFNWYVDGVEVHNAKT
KPREEQYNSTYRVVSVLTVLHQDWLNGKEYKCKVSNKALPAPIEK
TISKAKGQPREPQVYTLPPSRDELTKNQVSLTCLVKGFYPSDIAV
EWESNGQPENNYKTTPPVLDSDGSFFLYSKLTVDKSRWQQGNVFS
CSVLHEALHSHYTQKSLSLSPGK
SEQ ID NO: 344
QVQLQESGPGLVKPAETLSLTCSVSGESINTGHYYWGWVRQVPGK
GLEWIGHIHYTTAVLHNPSLKSRLTIKIYTLRNQITLRLSNVTAA
DTAVYHCVRSGGDILYYYEWQKPHWFSPWGPGIHVTVSSASTKGP
SVFPLAPSSKSTSGGTAALGCLVKDYFPEPVTVSWNSGALTSGVH
TFPAVLQSSGLYSLSSVVTVPSSSLGTQTYICNVNHKPSNTKVDK
KVEPKSCDKTHTCPPCPAPELLGGPSVFLFPPKPKDTLMISRTPE
VTCVVVDVSHEDPEVKFNWYVDGVEVHNAKTKPREEQYNSTYRVV
SVLTVLHQDWLNGKEYKCKVSNKALPAPIEKTISKAKGQPREPQV
YTLPPSRDELTKNQVSLTCLVKGFYPSDIAVEWESNGQPENNYKT
TPPVLDSDGSFFLYSKLTVDKSRWQQGNVFSCSVLHEALHSHYTQ
KSLSLSPGKGGGGSKKVVYGKCGDTVELTCTASQKKNIQFHWKNS
NQIKILGNQGSFLTKGPSKLNDRVDSRRSLWDQGNFPLIIKNLKP
EDSDTYICEVEDQKEEVQLVWVC
SEQ ID NO: 345
KKVVYGKCGDTVELTCTASQKKNIQFHWKNSNQIKILGNQGSFLT
KGPSKLNDRVDSRRSLWDQGNFPLIIKNLKPEDSDTYICEVEDQK
EEVQLVVVCGGGGSQSALTQPPSASGSLGQSVTISCNGTSSDIGG
WNFVSWYQQFPGRAPRLIIFEVNKRPSGVPGRFSGSKSGNSASLT
VSGLQSDDEGQYFCSSLFGRWDVVFGGGTKLTVLGQPKAAPSVTL
FPPSSEELQANKATLVCLISDFYPGAVTVAWKADSSPVKAGVETT
TPSKQSNNKYAASSYLSLTPEQWKSHRSYSCQVTHEGSTVEKTVA
PTECS
SEQ ID NO: 346
QSALTQPPSASGSLGQSVTISCNGTSSDIGGWNFVSWYQQFPGRA
PRLIIFEVNKRPSGVPGRFSGSKSGNSASLTVSGLQSDDEGQYFC
SSLFGRWDVVFGGGTKLTVLGQPKAAPSVTLFPPSSEELQANKAT
LVCLISDFYPGAVTVAWKADSSPVKAGVETTTPSKQSNNKYAASS
YLSLTPEQWKSHRSYSCQVTHEGSTVEKTVAPTECSGGGGSKKVV
YGKCGDTVELTCTASQKKNIQFHWKNSNQIKILGNQGSFLTKGPS
KLNDRVDSRRSLWDQGNFPLIIKNLKPEDSDTYICEVEDQKEEVQ
LVVVC
SEQ ID NO: 347
KKVVYGKCGDTVELTCTASQKKNIQFHWKNSNQIKILGNQGSFLT
KGPSKLNDRVDSRRSLWDQGNFPLIIKNLKPEDSDTYICEVEDQK
EEVQLVVVCGGGGSQVQLQESGPGLVKPSETLSLTCTVSGDSINT
GHHYWGWVRQVPGKGPEWIAHIHYNTAVLHNPALKSRVTISIFTL
KNLITLSLSNVTAADTAVYFCVRSGGDILYYIEWQKPHWFYPWGP
GILVTVSSASTKGPSVFPLAPSSKSTSGGTAALGCLVKDYFPEPV
TVSWNSGALTSGVHTFPAVLQSSGLYSLSSVVTVPSSSLGTQTYI
CNVNHKPSNTKVDKKVEPKSCDKTHTCPPCPAPELLGGPSVFLFP
PKPKDTLMISRTPEVTCVVVDVSHEDPEVKFNWYVDGVEVHNAKT
KPREEQYNSTYRVVSVLTVLHQDWLNGKEYKCKVSNKALPAPIEK
TISKAKGQPREPQVYTLPPSRDELTKNQVSLTCLVKGFYPSDIAV
EWESNGQPENNYKTTPPVLDSDGSFFLYSKLTVDKSRWQQGNVFS
CSVLHEALHSHYTQKSLSLSPGK
SEQ ID NO: 348
KKVVYGKCGDTVELTCTASQKKNIQFHWKNSNQIKILGNQGSFLT
KGPSKLNDRVDSRRSLWDQGNFPLIIKNLKPEDSDTYICEVEDQK
EEVQLVVVCGGGGSQLQLQESGPGLVKPSETLSLTCTVSGGSMRG
TDWGENDFHYGWIRQSSAKGLEWIGSIHWRGRTTHYKTSFRSRAT
LSIDTSNNRFSLTFSFVTAADTAVYYCARHKYHDIFRVVPVAGWF
DPWGQGLLVTVSSASTKGPSVFPLAPSSKSTSGGTAALGCLVKDY
FPEPVTVSWNSGALTSGVHTFPAVLQSSGLYSLSSVVTVPSSSLG
TQTYICNVNHKPSNTKVDKKVEPKSCDKTHTCPPCPAPELLGGPS
VFLFPPKPKDTLMISRTPEVTCVVVDVSHEDPEVKFNWYVDGVEV
HNAKTKPREEQYNSTYRVVSVLTVLHQDWLNGKEYKCKVSNKALP
APIEKTISKAKGQPREPQVYTLPPSRDELTKNQVSLTCLVKGFYP
SDIAVEWESNGQPENNYKTTPPVLDSDGSFFLYSKLTVDKSRWQQ
GNVFSCSVLHEALHSHYTQKSLSLSPGK
SEQ ID NO: 349
KKVVYGKCGDTVELTCTASQKKNIQFHWKNSNQIKILGNQGSFLT
KGPSKLNDRVDSRRSLWDQGNFPLIIKNLKPEDSDTYICEVEDQK
EEVQLVVVCGGGGSEVHLEESGPGLVRPSETLSLTCTASGGSIRG
GEWGDSDYHWGWVRHSPEKGLEWIGSIHWRGTTHYNAPFRGRGRL
SIDLSRNQFSLRLTSVTAEDTAVYYCVKHKYHDIVMVVPIAGWFD
PWGQGLQVTVSSASTKGPSVFPLAPSSKSTSGGTAALGCLVKDYF
PEPVTVSWNSGALTSGVHTFPAVLQSSGLYSLSSVVTVPSSSLGT
QTYICNVNHKPSNTKVDKKVEPKSCDKTHTCPPCPAPELLGGPSV
FLFPPKPKDTLMISRTPEVTCVVVDVSHEDPEVKFNWYVDGVEVH
NAKTKPREEQYNSTYRVVSVLTVLHQDWLNGKEYKCKVSNKALPA
PIEKTISKAKGQPREPQVYTLPPSRDELTKNQVSLTCLVKGFYPS
DIAVEWESNGQPENNYKTTPPVLDSDGSFFLYSKLTVDKSRWQQG
NVFSCSVLHEALHSHYTQKSLSLSPGK
SEQ ID NO: 350
KKVVYGKCGDTVELTCTASQKKNIQFHWKNSNQIKILGNQGSFLT
KGPSKLNDRVDSRRSLWDQGNFPLIIKNLKPEDSDTYICEVEDQK
EEVQLVVVCGGGGSEVQLVESGGGLVKPGGSLRLTCVASGFTFSD
VWLNWVRQAPGKGLEWVGRIKSRTDGGTTDYAASVKGRFTISRDD
SKNTLYLQMNSLKTEDTAVYSCTTDGFIMIRGVSEDYYYYYMDVW
GKGTTVTVSSASTKGPSVFPLAPSSKSTSGGTAALGCLVKDYFPE
PVTVSWNSGALTSGVHTFPAVLQSSGLYSLSSVVTVPSSSLGTQT
YICNVNHKPSNTKVDKKVEPKSCDKTHTCPPCPAPELLGGPSVFL
FPPKPKDTLMISRTPEVTCVVVDVSHEDPEVKFNWYVDGVEVHNA
KTKPREEQYNSTYRVVSVLTVLHQDWLNGKEYKCKVSNKALPAPI
EKTISKAKGQPREPQVYTLPPSRDELTKNQVSLTCLVKGFYPSDI
AVEWESNGQPENNYKTTPPVLDSDGSFFLYSKLTVDKSRWQQGNV
FSCSVLHEALHSHYTQKSLSLSPGK
SEQ ID NO: 351
KKVVYGKCGDTVELTCTASQKKNIQFHWKNSNQIKILGNQGSFLT
KGPSKLNDRVDSRRSLWDQGNFPLIIKNLKPEDSDTYICEVEDQK
EEVQLVVVCGGGGSQVQLVQSGGGLVQPGGSLRLSCAAFGFNFSS
YVMHWVRQAPGQGLEYLSAISSDGETTYHANSVKGRFTSSRDNSK
NTLFLQMGSLRTEDVAVYYCARDRYYETSGSNAFDVWGQGTMVVV
SSASTKGPSVFPLAPSSKSTSGGTAALGCLVKDYFPEPVTVSWNS
GALTSGVHTFPAVLQSSGLYSLSSVVTVPSSSLGTQTYICNVNHK
PSNTKVDKKVEPKSCDKTHTCPPCPAPELLGGPSVFLFPPKPKDT
LMISRTPEVTCVVVDVSHEDPEVKFNWYVDGVEVHNAKTKPREEQ
YNSTYRVVSVLTVLHQDWLNGKEYKCKVSNKALPAPIEKTISKAK
GQPREPQVYTLPPSRDELTKNQVSLTCLVKGFYPSDIAVEWESNG
QPENNYKTTPPVLDSDGSFFLYSKLTVDKSRWQQGNVFSCSVLHE
ALHSHYTQKSLSLSPGK
SEQ ID NO: 352
KKVVYGKCGDTVELTCTASQKKNIQFHWKNSNQIKILGNQGSFLT
KGPSKLNDRVDSRRSLWDQGNFPLIIKNLKPEDSDTYICEVEDQK
EEVQLVVVCGGGGSQVQLVQSGAEVRKPGSSVTISCKPVGGTFTN
FAIHWVRQAPGQGLEWVGGRVPVVGIYKYGKKFHDRLRLYEDDPM
KTVFLELRSLTSDDTGVYYCTRWRGCGMCPYDTSSYYNDASDVWG
PGTKVIVSAASTKGPSVFPLAPSSKSTSGGTAALGCLVKDYFPEP
VTVSWNSGALTSGVHTFPAVLQSSGLYSLSSVVTVPSSSLGTQTY
ICNVNHKPSNTKVDKKVEPKSCDKTHTCPPCPAPELLGGPSVFLF
PPKPKDTLMISRTPEVTCVVVDVSHEDPEVKFNWYVDGVEVHNAK
TKPREEQYNSTYRVVSVLTVLHQDWLNGKEYKCKVSNKALPAPIE
KTISKAKGQPREPQVYTLPPSRDELTKNQVSLTCLVKGFYPSDIA
VEWESNGQPENNYKTTPPVLDSDGSFFLYSKLTVDKSRWQQGNVF
SCSVLHEALHSHYTQKSLSLSPGK
SEQ ID NO: 353
KKVVYGKCGDTVELTCTASQKKNIQFHWKNSNQIKILGNQGSFLT
KGPSKLNDRVDSRRSLWDQGNFPLIIKNLKPEDSDTYICEVEDQK
EEVQLVVVCGGGGSEVQLVESGGGLVKAGGSLILSCGVSNFRISA
HTMNWVRRVPGGGLEWVASISTSSTYRDYADAVKGRFTVSRDDLE
DFVYLQMHKMRVEDTAIYYCARKGSDRLSDNDPFDAWGPGTVVTV
SPASTKGPSVFPLAPSSKSTSGGTAALGCLVKDYFPEPVTVSWNS
GALTSGVHTFPAVLQSSGLYSLSSVVTVPSSSLGTQTYICNVNHK
PSNTKVDKKVEPKSCDKTHTCPPCPAPELLGGPSVFLFPPKPKDT
LMISRTPEVTCVVVDVSHEDPEVKFNWYVDGVEVHNAKTKPREEQ
YNSTYRVVSVLTVLHQDWLNGKEYKCKVSNKALPAPIEKTISKAK
GQPREPQVYTLPPSRDELTKNQVSLTCLVKGFYPSDIAVEWESNG
QPENNYKTTPPVLDSDGSFFLYSKLTVDKSRWQQGNVFSCSVLHE
ALHSHYTQKSLSLSPGK
SEQ ID NO: 354
KKVVYGKCGDTVELTCTASQKKNIQFHWKNSNQIKILGNQGSFLT
KGPSKLNDRVDSRRSLWDQGNFPLIIKNLKPEDSDTYICEVEDQK
EEVQLVVVCGGGGSQMQLQESGPGLVKPGETLSLTCSVSGASISS
SYWSWLRETPGKGLEWIGYTHHSGDTNYAPSLKSRVHLGLHPSKN
QVSLSLTSVTAADTAVYYCARTLHGRRIYGVVAFNEFFTYFYWEV
WGKGTQVTVSSASTKGPSVFPLAPSSKSTSGGTAALGCLVKDYFP
EPVTVSWNSGALTSGVHTFPAVLQSSGLYSLSSVVTVPSSSLGTQ
TYICNVNHKPSNTKVDKKVEPKSCDKTHTCPPCPAPELLGGPSVF
LFPPKPKDTLMISRTPEVTCVVVDVSHEDPEVKFNWYVDGVEVHN
AKTKPREEQYNSTYRVVSVLTVLHQDWLNGKEYKCKVSNKALPAP
IEKTISKAKGQPREPQVYTLPPSRDELTKNQVSLTCLVKGFYPSD
IAVEWESNGQPENNYKTTPPVLDSDGSFFLYSKLTVDKSRWQQGN
VFSCSVLHEALHSHYTQKSLSLSPGK
SEQ ID NO: 355
KKVVYGKCGDTVELTCTASQKKNIQFHWKNSNQIKILGNQGSFLT
KGPSKLNDRVDSRRSLWDQGNFPLIIKNLKPEDSDTYICEVEDQK
EEVQLVVVCGGGGSQMQLQESGPGLVKPGETLSLTCSVSGASISS
SYWSWLRETPGKGLEWIGYTHHSGDTNYAPSLKSRVHLGLHPSKN
QVSLSLTSVTAADTAVYYCARTLHGRRIYGVVAFNEYYTYFYWPT
WGKGTQVTVSSASTKGPSVFPLAPSSKSTSGGTAALGCLVKDYFP
EPVTVSWNSGALTSGVHTFPAVLQSSGLYSLSSVVTVPSSSLGTQ
TYICNVNHKPSNTKVDKKVEPKSCDKTHTCPPCPAPELLGGPSVF
LFPPKPKDTLMISRTPEVTCVVVDVSHEDPEVKFNWYVDGVEVHN
AKTKPREEQYNSTYRVVSVLTVLHQDWLNGKEYKCKVSNKALPAP
IEKTISKAKGQPREPQVYTLPPSRDELTKNQVSLTCLVKGFYPSD
IAVEWESNGQPENNYKTTPPVLDSDGSFFLYSKLTVDKSRWQQGN
VFSCSVLHEALHSHYTQKSLSLSPGK
SEQ ID NO: 356
KKVVYGKCGDTVELTCTASQKKNIQFHWKNSNQIKILGNQGSFLT
KGPSKLNDRVDSRRSLWDQGNFPLIIKNLKPEDSDTYICEVEDQK
EEVQLVVVCGGGGSQMQLQESGPGLVKPGETLSLTCSVSGASISS
SYWSWLRETPGKGLEWIGYTHHSGDTNYAPSLKSRVTIGLDPSKN
QVSLSLTSVTAADTAVYYCARTLHGRRIYGVVAFNEYYTYFYWPT
WGKGTQVTVSSASTKGPSVFPLAPSSKSTSGGTAALGCLVKDYFP
EPVTVSWNSGALTSGVHTFPAVLQSSGLYSLSSVVTVPSSSLGTQ
TYICNVNHKPSNTKVDKKVEPKSCDKTHTCPPCPAPELLGGPSVF
LFPPKPKDTLMISRTPEVTCVVVDVSHEDPEVKFNWYVDGVEVHN
AKTKPREEQYNSTYRVVSVLTVLHQDWLNGKEYKCKVSNKALPAP
IEKTISKAKGQPREPQVYTLPPSRDELTKNQVSLTCLVKGFYPSD
IAVEWESNGQPENNYKTTPPVLDSDGSFFLYSKLTVDKSRWQQGN
VFSCSVLHEALHSHYTQKSLSLSPGK
SEQ ID NO: 357
KKVVYGKCGDTVELTCTASQKKNIQFHWKNSNQIKILGNQGSFLT
KGPSKLNDRVDSRRSLWDQGNFPLIIKNLKPEDSDTYICEVEDQK
EEVQLVVVCGGGGSQVQLLESGPGLVRPSETLTLTCSVFNSRVSG
YYYSWIRQPPGRGLEWIASTHFSLRPSRNPSLLSRVTTSIDTERY
QVFLNMRSVTAADTAVYFCARGDASGWRADYFPHWGQGTLVVVSS
ASTKGPSVFPLAPSSKSTSGGTAALGCLVKDYFPEPVTVSWNSGA
LTSGVHTFPAVLQSSGLYSLSSVVTVPSSSLGTQTYICNVNHKPS
NTKVDKKVEPKSCDKTHTCPPCPAPELLGGPSVFLFPPKPKDTLM
ISRTPEVTCVVVDVSHEDPEVKFNWYVDGVEVHNAKTKPREEQYN
STYRVVSVLTVLHQDWLNGKEYKCKVSNKALPAPIEKTISKAKGQ
PREPQVYTLPPSRDELTKNQVSLTCLVKGFYPSDIAVEWESNGQP
ENNYKTTPPVLDSDGSFFLYSKLTVDKSRWQQGNVFSCSVLHEAL
HSHYTQKSLSLSPGK
SEQ ID NO: 358
KKVVYGKCGDTVELTCTASQKKNIQFHWKNSNQIKILGNQGSFLT
KGPSKLNDRVDSRRSLWDQGNFPLIIKNLKPEDSDTYICEVEDQK
EEVQLVVVCGGGGSQSVLTQPPSASGSPGQSVTISCTGTSSDIGA
SDYVSWYQQYPGEAPKVIIYDVTKRPSGVPDRFSGSKSGTTASLT
VSGLQAEDEADYYCSSDAGRHTLLFGGGTKVTVLGQPKAAPSVTL
FPPSSEELQANKATLVCLISDFYPGAVTVAWKADSSPVKAGVETT
TPSKQSNNKYAASSYLSLTPEQWKSHRSYSCQVTHEGSTVEKTVA
PTECS
SEQ ID NO: 359
QVQLLESGPGLVRPSETLTLTCSVFNSRVSGYYYSWIRQPPGRGL
EWIASTHFSLRPSRNPSLLSRVTTSIDTERYQVFLNMRSVTAADT
AVYFCARGDASGWRADYFPHWGQGTLVVVSSASTKGPSVFPLAPS
SKSTSGGTAALGCLVKDYFPEPVTVSWNSGALTSGVHTFPAVLQS
SGLYSLSSWVTVPSSSLGTQTYICNVNHKPSNTKVDKKVEPKSCD
KTHTCPPCPAPELLGGPSVFLFPPKPKDTLMISRTPEVTCVVVDV
SHEDPEVKFNWYVDGVEVHNAKTKPREEQYNSTYRVVSVLTVLHQ
DWLNGKEYKCKVSNKALPAPIEKTISKAKGQPREPQVYTLPPSRD
ELTKNQVSLTCLVKGFYPSDIAVEWESNGQPENNYKTTPPVLDSD
GSFFLYSKLTVDKSRWQQGNVFSCSVLHEALHSHYTQKSLSLSPG
KGGGGSKKVVYGKCGDTVELTCTASQKKNIQFHWKNSNQIKILGN
QGSFLTKGPSKLNDRVDSRRSLWDQGNFPLIIKNLKPEDSDTYIC
EVEDQKEEVQLVVVC
SEQ ID NO: 360
QSVLTQPPSASGSPGQSVTISCTGTSSDIGASDYVSWYQQYPGEA
PKVIIYDVTKRPSGVPDRFSGSKSGTTASLTVSGLQAEDEADYYC
SSDAGRHTLLFGGGTKVTVLGQPKAAPSVTLFPPSSEELQANKAT
LVCLISDFYPGAVTVAWKADSSPVKAGVETTTPSKQSNNKYAASS
YLSLTPEQWKSHRSYSCQVTHEGSTVEKTVAPTECSGGGGSKKVV
YGKCGDTVELTCTASQKKNIQFHWKNSNQIKILGNQGSFLTKGPS
KLNDRVDSRRSLWDQGNFPLIIKNLKPEDSDTYICEVEDQKEEVQ
LVVVC
SEQ ID NO: 361
CTRPNNNTRKSIHIGPGRAFYTTGEIIGDIRQAHC
SEQ ID NO: 362
KKVVYGKCGDTVELTCTASQKKNIQFHWKNSNQIKILGNQGSFLT
KGPSKLNDRVDSRRSLWDQGNFPLIIKNLKPEDSDTYICEVEDQK
EEVQLVVVCGGGGSQVQLVQSGAQMKNPGASVKVSCAPSGYTFTD
FYIHWLRQAPGQGLQWMGWMNPQTGRTNTARNFQGRVTMTRDTSI
GTAYMELRSLTSDDTAIYYCTTGGWISLYYDSSYYPNFDHWGQGT
LLTVSSASTKGPSVFPLAPSSKSTSGGTAALGCLVKDYFPEPVTV
SWNSGALTSGVHTFPAVLQSSGLYSLSSVVTVPSSSLGTQTYICN
VNHKPSNTKVDKKVEPKSCDKTHTCPPCPAPELLGGPSVFLFPPK
PKDTLMISRTPEVTCVVVDVSHEDPEVKFNWYVDGVEVHNAKTKP
REEQYNSTYRVVSVLTVLHQDWLNGKEYKCKVSNKALPAPIEKTI
SKAKGQPREPQVYTLPPSRDELTKNQVSLTCLVKGFYPSDIAVEW
ESNGQPENNYKTTPPVLDSDGSFFLYSKLTVDKSRWQQGNVFSCS
VLHEALHSHYTQKSLSLSPGK
SEQ ID NO: 363
MKVMGTKKNYQHLWRWGIMLLGMLMMSSAAEQLWVTVYYGVPVWR
EANTTLFCASDAKAYDTEVHNVWATHACVPTDPNPQEVVMGNVTE
DFNMWKNNMVEQMHEDIISLWDQSLKPCVKLTPLCVTLHCTNVTI
SSTNGSTANVTMREEMKNCSFNTTTVIRDKIQKEYALFYKLDIVP
IEGKNTNTSYRLINCNTSVITQACPKVSFEPIPIHYCAPAGFAIL
KCNNKTFNGKGPCRNVSTVQCTHGIKPVVSTQLLLNGSLAEEDII
IRSENFTNNGKNIIVQLKEPVKINCTRPGNNTRRSINIGPGRAFY
ATGAIIGDIRKAHCNISTEQWNNTLTQIVDKLREQFGNKTIIFNQ
SSGGDPEVVMHTFNCGGEFFYCNSTQLFNSTWFNNGTSTWNSTAD
NITLPCRIKQVINMWQEVGKAMYAPPIRGQIDCSSNITGLILTRD
GGSNSSQNETFRPGGGNMKDNWRSELYKYKVVKIEPLGIAPTRAK
RRVVQREKRAVTLGAVFLGFLGAAGSTMGAASLTLTVQARLLLSG
IVQQQSNLLRAIEAQQHMLQLTVWGIKQLQARVLAIERYLKDQQL
LGIWGCSGKLICTTTVPWNTSWSNKSYDYIWNNMTWMQWEREIDN
YTGFIYTLIEESQNQQEKNELELLELDKWASLWNWFNITNWLWYI
KLFIMIIGGLVGLRIVCAVLSIVNRVRQGYSPLSFQTRLPNPRGP
DRPEETEGEGGERDRDRSARLVNGFLAIIWDDLRSLCLFSYHRLR
DLLLIVARVVEILGRRGWEILKYWWNLLKYWSQELKNSAVSLLNV
TAIAVAEGTDRVIEIVQRAVRAILHIPTRIRQGFERALL
SEQ ID NO: 364
AEQLWVIVYYGVPVWREANTTLFCASDAKAYDTEVHNVWATHACV
PTDPNPQEVVMGNVTEDFNMWKNNMVEQMHEDIISLWDQSLKPCV
KLTPLCVTLHCTNVTISSTNGSTANVTMREEMKNCSFNTTTVIRD
KIQKEYALFYKLDIVPIEGKNTNTSYRLINCNTSVITQACPKVSF
EPIPIHYCAPAGFAILKCNNKTFNGKGPCRNVSTVQCTHGIKPVV
STQLLLNGSLAEEDIIIRSENFTNNGKNIIVQLKEPVKINCTRPG
NNTRRSINIGPGRAFYATGAIIGDIRKAHCNISTEQWNNTLTQIV
DKLREQFGNKTIIFNQSSGGDPEVVMHTFNCGGEFFYCNSTQLFN
STWENNGTSTWNSTADNITLPCRIKQVINMWQEVGKAMYAPPIRG
QIDCSSNITGLILTRDGGSNSSQNETFRPGGGNMKDNWRSELYKY
KVVKIEPLGIAPTRAKRRVVQREKR
SEQ ID NO: 365
SDISVAPGETVRITCGGESIGSRAVQWYQHRPGQAPRLIIYNNQD
RPPGIPERFSGSPDIDFGTTATLTISNVEAGDEATYYCHIWDSRR
PTNWELGPGTTLTVL
SEQ ID NO: 366
SDISVAPGETVRITCGGESIGSRAVQWYQHRPGQAPRLIIYNNQD
RPPGIPERFSGSPDIDFGTTATLTISNVEAGDEATYYCHIWDSRR
PTNWELGPGTTLTVLGQPKAAPSVTLFPPSSEELQANKATLVCLI
SDFYPGAVTVAWKADSSPVKAGVETTTPSKQSNNKYAASSYLSLT
PEQWKSHRSYSCQVTHEGSTVEKTVAPTECS

1. A bispecific anti-human immunodeficiency virus (HIV) envelope surface glycoprotein 120 (gp120)-binding protein comprising (i) an anti-V3 broadly neutralizing antibody (bNAb) comprising two heavy chains and two light chains; and (ii) at least one cluster of differentiation 4 (CD4) domain, wherein the at least one CD4 domain is attached directly or via a linker to the N-terminus or the C-terminus of one of the anti-V3 bNAb heavy chains or light chains, wherein the two heavy chains comprise a CDRH1 of SEQ ID NO:22, a CDRH2 of SEQ ID NO: 23, a CDRH3 of SEQ ID NO:24, and the two light chains comprise a CDRL1 of SEQ ID NO: 25, a CDRL2 of SEQ ID NO:26 and a CDRL3 of SEQ ID NO:27.

2. The bispecific protein as claimed in claim 1, wherein the two heavy chains comprise a variable heavy (VH) domain of SEQ ID NO:58 and the two light chains comprise a variable light (VL) domain of SEQ ID NO:59 or SEQ ID NO:60.

3. The bispecific protein as claimed in claim 1, wherein one of the at least one CD4 domain is attached via the linker to the N-terminus or the C-terminus of one anti-V3 bNAb heavy chain.

4. The bispecific protein as claimed in claim 1, wherein one of the at least one CD4 domain is attached via the linker to the N-terminus of one anti-V3 bNAb heavy chain.

5. The bispecific protein as claimed in claim 1, wherein the at least one CD4 domain comprises two CD4 domains, wherein a first CD4 domain is attached via a first linker to the N-terminus of one anti-V3 bNAb heavy chain and a second CD4 domain is attached via a second linker to the N-terminus of the other anti-V3 bNAb heavy chain.

6. The bispecific protein as claimed in claim 1, wherein the linker is selected from the group consisting of SEQ ID NOs: 90 to 95.

7. The bispecific protein as claimed in claim 6, wherein the linker is SEQ ID NO:90.

8. The bispecific protein as claimed in claim 1, wherein the at least one CD4 domain is selected from the group consisting of SEQ ID NOs: 1-21.

9. The bispecific protein as claimed in claim 8, wherein the at least one CD4 domain is SEQ ID NO: 11.

10. The bispecific protein as claimed in claim 1, having two identical heavy chains and two identical light chains, comprising or consisting of:
a heavy chain that is at least 95% identical to SEQ ID NO:121 and
a light chain that is at least 95% identical to SEQ ID NO:63.

11. The bispecific protein as claimed in claim 1, wherein the bispecific protein consists of two heavy chains of SEQ ID NO: 121 and two light chains of SEQ ID NO:63.

12. A pharmaceutical composition comprising the anti-HIV gp120-binding protein as defined in claim 1 and a pharmaceutically acceptable excipient.

13. A method of treating or preventing an HIV infection in a human comprising administering to the human the anti-HIV gp120-binding protein according to claim 1, whereby viral load in the human is decreased.

14. A nucleic acid sequence that encodes the anti-HIV gp120-binding protein according to claim 1.

15. An expression vector that comprises the nucleic acid sequence of claim 14.

16. A host cell that comprises the nucleic acid sequence of claim 14.

17. A host cell that comprises two expression vectors:
a first expression vector comprising a first nucleic acid sequence encoding a heavy chain of SEQ ID NO:121; and
a second expression vector comprising a second nucleic acid sequence encoding a light chain of SEQ ID NO:63.

18. A method of producing an anti-HIV gp120-binding protein, comprising culturing the host cell as defined in claim 17 under conditions suitable for expression of said nucleic acid sequence, whereby the anti-HIV gp120-binding protein is produced.

19. An anti-HIV gp120-binding protein produced by the method of claim 18.

20. A soluble cluster of differentiation 4 (CD4) domain comprising one or more stabilizing mutations selected from the group consisting of: K8C, K8I, K8V, T11C, E13C, K21C, Q25E, H27C, H27D, G38C, N52W, R58N, R58T, R58V, L61M, G65C, 170C, K72C, E87G, E91H, E91Q, and G99C.

21. The soluble CD4 domain of claim 20, wherein the soluble CD4 domain has a melting temperature (Tm) of about 90° C.

22. The soluble CD4 domain of claim 20, comprising any one of SEQ ID NOs: 5-21.

23. The soluble CD4 domain of claim 20, comprising SEQ ID NO:11.

24. The soluble CD4 domain of claim 20, wherein the soluble CD4 domain is fused directly or via a linker to a human fragment crystallizable (Fc) domain.

25. An anti-human immunodeficiency virus (HIV) envelope surface glycoprotein 120 (gp120)-binding protein comprising
a) means for binding one or more amino acid residues of the cluster of differentiation 4 (CD4)-binding site of HIV gp120; and
b) means for binding a cluster of mannose glycans centered on N332 of HIV gp120.

26. The binding protein of claim 25, wherein a) comprises at least one CD4 domain selected from the group consisting of SEQ ID NOs: 1-21.

27. The bispecific protein as claimed in claim 25, wherein the at least one CD4 domain is SEQ ID NO: 11.

28. The binding protein of claim 25, wherein b) comprises a broadly neutralizing antibody.

29. The binding protein of claim 25, wherein a) is attached to b) via a linker.

30. The binding protein of claim 29, wherein the linker is selected from the group consisting of SEQ ID NOs: 90 to 95.
